(19) United States
(12) Patent Application Publication
(10) Pub. No.: US 2026/0284049 A1
(43) Pub. Date: Sep. 24, 2026

(54) USE OF MILVEXIAN IN THE TREATMENT AND PREVENTION OF THROMBOTIC CONDITIONS IN PATIENTS WITH CARDIOVASCULAR OR CEREBROVASCULAR DISEASE

(71) Applicants: BRISTOL-MYERS SQUIBB COMPANY, Princeton, NJ (US); JANSSEN PHARMACEUTICA NV, Beerse (BE)

(72) Inventors: Danshi LI, Princeton, NJ (US); Christopher NESSEL, Bridgewater, NJ (US); Alexei PLOTNIKOV, Bridgewater, NJ (US); Elliot BARNATHAN, Spring House, PA (US); John STRONY, Raritan, NJ (US); Gary PETERS, Raritan, NJ (US); Madhu CHINTALA, Spring House, PA (US); Joseph LUETTGEN, Princeton, NJ (US); Puneet MOHAN, Princeton, NJ (US); Jay HORROW, Princeton, NJ (US)

(21) Appl. No.: 19/476,320

(22) PCT Filed: Apr. 19, 2024
(86) PCT No.: PCT/US2024/025410

§ 371 Date: Oct. 17, 2025

Related U.S. Application Data
(60) Provisional application No. 63/497,088, filed on Apr. 19, 2023.
(60) Provisional application No. 63/497,101, filed on Apr. 19, 2023.
(60) Provisional application No. 63/497,111, filed on Apr. 19, 2023.
(60) Provisional application No. 63/578,508, filed on Aug. 24, 2023.
(60) Provisional application No. 63/582,313, filed on Sep. 13, 2023.

(51) Int. Cl. A61K 31/513; A61K 9/20; A61K 9/28; A61K 31/4365; A61K 31/519; A61K 31/616; A61P 7/02

(52) U.S. Cl. CPC A61K 31/513; A61K 9/2013; A61K 9/2018; A61K 9/2054; A61K 9/28; A61K 31/4365; A61K 31/519; A61K 31/616; A61P 7/02

(57) ABSTRACT

A Factor XIa inhibitor having therapeutic properties useful in methods for preventing a thrombotic condition in human patients with a cardiovascular or cerebrovascular disease.

### CROSS-REFERENCE TO RELATED APPLICATIONS

This application claims the benefit of U.S. Provisional Application No. 63/497,088, filed Apr. 19, 2023, U.S. Provisional Application No. 63/497,101, filed Apr. 19, 2023, U.S. Provisional Application No. 63/578,508, filed Aug. 24, 2023, and U.S. Provisional Application No. 63/582,313, filed Sep. 13, 2023, U.S. Provisional Application No. 63/497,111, filed Apr. 19, 2023. The entirety of each aforementioned application is incorporated by reference herein.

### TECHNICAL FIELD

The disclosure pertains to the use of milvexian for treating or preventing thrombotic conditions in human patients with cardiovascular or cerebrovascular disease without significantly impairing normal blood clotting process.

### BACKGROUND

Thromboembolism and its associated complications remain a huge healthcare burden worldwide. Despite the availability of current therapies, stroke continues to be a leading cause of death and disability worldwide. In 2019 alone, it resulted in 143 million disability-adjusted life-years and 6.55 million deaths, contributing to a significant portion of healthcare expenditure in Western countries (Katan et al., Global burden of stroke, Semin Neurol., 2018, vol. 38, pp. 208-211). Thrombi may partially or totally obstruct arteries or veins, leading to local ischemic complications and they can embolize to the cerebral arteries and lungs, where they may cause stroke or other life-threatening conditions. Thrombosis contributes to cardiovascular morbidity and mortality, most notably in coronary artery disease (CAD), atrial fibrillation (AF), stroke, peripheral arterial disease (PAD), deep venous thromboembolism (DVT), pulmonary embolism (PE), acute myocardial infarction (AMI), and venous thromboembolism (VTE). Arterial and venous thrombosis are among the most frequent causes of mortality and morbidity. Arterial thrombosis is the cause of myocardial infarction (MI) and stroke, while venous thrombosis (VT) leads to venous thromboembolism (VTE) and pulmonary embolism (PE). Ischemic heart disease and stroke collectively are responsible for nearly 25% of all deaths worldwide (Lozano et al. Global and regional mortality from 235 causes of death for 20 age groups in 1990 and 2010: A systematic analysis for the Global Burden of Disease Study 2010. Lancet. 2012; 380:2095-2128); whereas estimates for the incidence rate for VTE, comprising deep vein thrombosis (DVT) and pulmonary embolism (PE), range from 115 to 269 per 100,000 people worldwide (Day I.S.C.f.W.T. Thrombosis: A major contributor to the global disease burden. J. Thromb. Haemost. 2014; 12:1580-1590).

The overall composition of venous thrombi differed significantly from arterial thrombi. RBCs and fibrin fibers were the major components of venous thrombi, comprising on average 63% and 35% of the volume, respectively, while arterial thrombi were mostly composed of fibrin and platelets. Platelets play an important role in the development of arterial thrombi formed at relatively high wall shear rates (about 10² to 10⁵ s⁻¹), generating what are often termed “white” thrombi. In patients with coronary artery disease, thrombi commonly arise from the rupture of atherosclerotic plaque and exposure of procoagulant components, such as collagen and lipid-rich activated macrophages bearing tissue factor, leading to myocardial infarction if thrombi become obstructive. Similar events may lead to in situ arterial thrombosis in the cerebral or other circulations. In contrast, venous thrombi formed under low shear rate (10 to 100 s⁻¹) are mainly composed of red blood cells (RBCs) and fibrin, i.e. “red” thrombi. The formation of venous thrombi is generally attributed to a combination of hypercoagulability together with injured or activated endothelium and impaired blood flow (Virchow's triad). Overall composition of pulmonary emboli differed significantly from arterial thrombi, but was not significantly different from venous thrombi (Cheynysh et al., The distinctive structure and composition of arterial and venous thrombi and pulmonary emboli, Scientific Reports, 2020, vol. 10, pp. 5112). Therefore, antiplatelet therapy is considered the favorite choice in preventing arterial thrombosis, whereas anticoagulant therapy (vitamin K anticoagulant, heparin, or direct Factor Xa inhibitors) is the recommended therapy in venous thrombosis. However, there are conditions where similarities between arterial and venous thrombosis exist: (1) in a special situation where fibrin-rich thrombi occur in the left atrial appendage (receiving oxygen-rich venous blood from the post-capillary pulmonary circulation having characteristics of low pressure and low shear) of patients with atrial fibrillation (AF) (Wysokinski et al., Atrial fibrillation and thrombosis: immunohistochemical differences between in situ and embolized thrombi. J Thromb Haemost, 2004, vol. 2, pp. 1637-1644), and in the coronary artery system of patients with myocardial infarction (MI) (Yamashita et al., Detection of von Willebrand factor and tissue factor in platelets-fibrin rich coronary thrombi in acute myocardial infarction. Am J Cardiol 97 (1): 26-28); (2) aspirin may have an effect in the prevention of venous thromboembolism (VTE), which implies that platelets inevitably play a role in the formation of thrombi in the venous system, and (3) subjects with retinal vein occlusion (RVO) commonly have associated major cardiovascular (CV) risk factors (mainly arterial hypertension) (Janssen et al., Retinal vein occlusion: a form of venous thrombosis or a complication of atherosclerosis? A meta-analysis of thrombophilic factors. Thromb Haemost. 2005, vol. 93, pp. 1021-1026).

The two major pathways for triggering blood clotting cascade are well known; (1) the tissue factor pathway and (2) the contact pathway. Both pathways trigger a series of cascading events that generate a blood clot with the purpose to separate and seal the triggering agent from blood, thereby preventing its further contact with plasma components and arresting the thrombotic process (FIG. 1). Hemostasis is the normal, physiological process by which the clotting cascade seals up vascular damage to limit blood loss following injury. Platelets are the primary hemostasis agents and coagulation is secondary hemostasis (strengthens the platelet plug). Thrombosis, on the other hand, encompasses various pathological conditions where the normal physiological clotting processes end up generating blood clot(s) inside the vascular lumen that are disruptive to the normal flow of blood. Thrombin generation and fibrin formation are the culminating steps in both hemostasis and thrombosis, but with important differences in the pathways involved (FIG. 1).

Hemostasis is commonly triggered when tissue factor (TF) within the adventitial layer of blood vessels gets exposed to blood. Injury to vasculature that can lead to bleeding activates a series of soluble plasma proteins that act together in a cascade of enzyme activation events and culminate in the formation of platelet-fibrin clot(s). Because of the relatively high concentration of TF in such scenarios, the generation of thrombin is rapid and intense, quickly forming a hemostatic plug that seals the inciting TF away from blood. This disrupts the amplification of the coagulation processes through feedback mechanisms to the point of becoming pathological.

The concentration of TF in thrombosis is lower relative to hemostasis, but its duration of contact with blood components often lasts longer. Whether triggered by TF from disruption of an atherosclerotic plaque or activated monocytes/macrophages recruited to the site of injury or inflammation, or by implanted medical devices or neutrophil extracellular traps (NETs), these scenarios depend on the feedback mechanisms of the coagulation cascade for the growth and stabilization of the thrombus (FIG. 1). This clot or thrombus can impede the flow of blood to the distal tissues and organs, leading to ischemia and necrosis, manifesting as clinical events including acute coronary syndrome (ACS), stroke, or deep vein thrombosis (DVT) (Badimon et al., Factor XI/XIa Inhibition: The Arsenal in Development for a New Therapeutic Target in Cardio- and Cerebrovascular Disease, J Cardiovasc Dev Dis., 2022, vol. 9, p. 437).

The TF pathway is understood to play a larger role in the ‘initiation’ and ‘propagation’ phases of coagulation, functioning more in normal hemostasis than in thrombosis. FXI has a dual role, one in the contact pathway where it is directly downstream from FXII and another in the amplification pathway, where it is activated by thrombin (and FXIIa, if present. The contact pathway does appear, however, to have an important role in thrombotic disorders. Increased activity of plasma FXII, FXI, or kallikrein has been associated with atherosclerosis (Colhoun et al., Activated factor XII levels and factor XII 46C>T genotype in relation to coronary artery calcification in patients with type 1 diabetes and healthy subjects. Atherosclerosis. 2002; 163:363-369), and myocardial infarction (MI) (Doggen et al., Levels of intrinsic coagulation factors and the risk of myocardial infarction among men: Opposite and synergistic effects of factors XI and XII. Blood. 2006; 108:4045-4051),

An analysis of genetically determined FXI has shown the highest levels of FXI are associated with a heightened risk of ischemic stroke (Gill et al. Genetically determined FXI (Factor XI) levels and risk of stroke. Stroke 2018; 49 (11): 2761-2763); whereas severe FXI deficiency has been associated with reduced risk of stroke and deep vein thrombosis (Salomon et al., Reduced incidence of ischemic stroke in patients with severe factor XI deficiency. Blood. 2008; 111:4113-4117; Salomon et al., Patients with severe factor XI deficiency have a reduced incidence of deep-vein thrombosis. Thromb. Haemost. 2011; 105:269-273). Congenital deficiency of FXI appears to provide protection from both arterial and venous thrombotic events and is rarely associated with unprovoked major bleeding (ie, there is less bleeding than with other clotting factor deficiencies like Factor X). (Gailani et al., Factor XI as a therapeutic target. Arterioscler Thromb Vasc Biol. 2016; 36 (7): 1316-1322; Peyvandi et al. European Network of Rare Bleeding Disorders Group. Coagulation factor activity and clinical bleeding severity in rare bleeding disorders: results from the European Network of Rare Bleeding Disorders. J Thromb Haemost. 2012; (4): 615-621). Significant inhibition of atherosclerosis was observed in an atherosclerosis mouse model with severe FXI deficiency (apoE/FXI double knock out mice) compared with apoE knockout mice, suggesting pharmacologic Factor XI inhibition may have therapeutic potential in humans (Shnerb Ganor 2016).

The high human and financial cost of thromboembolic events underscore the need for newer and better therapeutic options for the management of thrombotic disorders. The challenge is in developing an agent that has potent antithrombotic effects but minimal bleeding risk, as it requires a very fine balancing act in modulating the hemostatic processes. The clinical benefits of current anticoagulant options for clinical treatment of thrombotic disorders are well established. Non-cardioembolic strokes, specifically those caused by large-artery extracranial atherosclerosis or intracranial small vessel disease, are commonly treated with single or dual antiplatelet therapy (SAPT/DAPT) (Greco et al., Antithrombotic Therapy for Primary and Secondary Prevention of Ischemic Stroke, JACC, 2023, vol. 82, pp. 1538-1557). The last few years have provided a much-improved treatment option in direct oral anticoagulants (DOACs) that are convenient in administration while being potent and equally effective to VKA, often with a lower risk of bleeding. Despite major advancements in the development of safer and more effective anticoagulant agents (e.g., direct oral anticoagulant (DOAC)), bleeding complications remain a significant concern in the treatment of thromboembolic diseases, e.g., approximately 5% in elderly patients with atrial fibrillation (AF) (Ruff et al. Comparison of the efficacy and safety of new oral anticoagulants with warfarin in patients with atrial fibrillation: A meta-analysis of randomised trials. Lancet. 2014; 383:955-962). This is partly why an unacceptably high proportion of AF patients (nearly 30%) do not receive the prophylactic anticoagulation they require. Even among those that do receive anticoagulation therapy, nearly half do not receive the proper doses (Alamneh et al., Suboptimal Use of Oral Anticoagulants in Atrial Fibrillation: Has the Introduction of Direct Oral Anticoagulants Improved Prescribing Practices? Am. J. Cardiovasc. Drugs. 2016; 16:183-200).

Improving the benefit-to-risk ratio therefore remains a viable goal for antithrombotic drug discovery. This requires selecting a molecular target with an enhanced difference between hemostasis and thrombosis. Given the larger role FXI is thought to play in thrombosis than in hemostasis, novel approaches to inhibit its generation and activity are being explored as new therapeutic strategies. These include: (a) antisense Oligonucleotides (ASOs) that act on the liver to knockdown hepatic synthesis of FXI (e.g., IONIS-FXI_{RX}, and IONIS-FXI-L_{RX}), (b) small molecules that target the FXI active site or the heparin allosteric site on FXIa (e.g., asundexian, milvexian, ONO-7648, EP-7041, BMS962212, sulfated pentagalloyl glucoside (SPGG)) (for SPGG, see Horani et al., J Thromb Haemost. 2019 vol. 12, pp. 2110-2122), (c) monoclonal antibodies that act by blocking the activation or inhibiting the activity (e.g., Xisomab, abelacimab, osocimab, MK-2060, REGN9933, BAY1831865), and (d) aptamers (Badimon et al., supra). In addition to their varying mechanisms of action, these strategies also differ in their routes of administration (oral vs. parenteral), the onset of action, and the duration of effect. Parenteral administration is a requirement for ASOs, aptamers and monoclonal antibodies, whereas small molecule agents offer the option of either parenteral or oral administration. The varied onset and duration of action may present a broad set of treatment options depending on the pathology at hand; acute thrombotic events requiring quick-acting agents. Similarly, for conditions presenting a high risk of bleeding complications such as trauma or surgery, shorter-acting agents would be preferable.

Despite major advances in understanding the mechanistic pathways of platelet function and the interaction of coagulation and thrombosis, challenges in the treatment of vascular occlusive diseases continue to persist. This is due to the complexity of these diverse diseases and the impact of immunological and inflammatory processes on haemostasis and thrombosis. Despite the successful development of new classes of DOACs, there is a great-unmet clinical need for developing more effective and safe antithrombotic agents. Pharmacological advances have greatly impacted thrombotic outcomes but have led to the unwanted side effect of bleeding. Compounding this issue is the lack of clear biomarkers to balance risk and benefit of treatments, particularly when used in combination.

Milvexian (BMS-986177/JNJ-70033093) is a direct-acting, high-affinity inhibitor of human coagulation FXIa (Dilger et al. Discovery of milvexian, a high-affinity, orally bioavailable inhibitor of factor XIa in clinical studies for antithrombotic therapy. J Med Chem 2022; 65 (3): 1770-85). Milvexian is a macrocyclic compound having the structure of Formula (I):

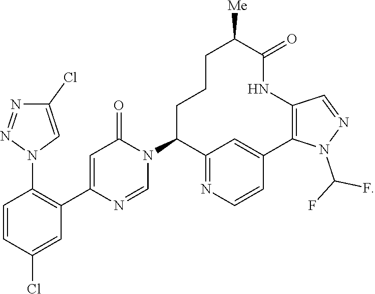 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

Milvexian is also known by its chemical name (5R,9S)-9-(4-(5-chloro-2-(4-chloro-1H-1,2,3-triazol-1-yl)phenyl)-6-oxopyrimidin-1(6H)-yl)-2¹-(difluoromethyl)-5-methyl-2¹H-3-aza-1(4,2)-pyridina-2(5,4)-pyrazolacyclonaphan-4-one.

Milvexian and a method of preparing milvexian are described in U.S. Pat. No. 9,453,018, which is hereby incorporated by reference in its entirety. Solvates, crystalline forms, and amorphous forms of milvexian are also known in the art. See, e.g., WO2021207659 and WO2022081473. An amorphous solid dispersion composition of milvexian in one or more polymers has been described in WO2020210629, which is hereby incorporated by reference in its entirety.

4,114 participants have been included and exposed to the study intervention (milvexian, placebo, or comparator) in the milvexian clinical program. Of the 4,114 participants, 3,229 received milvexian, of which 660 participants were exposed to milvexian in the Phase 1 studies and 2,569 participants were exposed to milvexian in the Phase 2 and 2a studies. Four types of serious bleeding in the milvexian clinical development program were assessed as adverse drug reactions: gastrointestinal bleeding, procedural hemorrhage, nervous system disorder bleeding (hemorrhagic transformation of ischemic stroke and subdural hematoma) and hematuria. The result of Phase II clinical trial of milvexian in patients undergoing TKR (total knee replacement) was published in 2021. Weitz et al.; Milvexian for the Prevention of Venous Thromboembolism. N. Engl. J. Med. 2021, 385, 2161-2172. The results of Phase II clinical trial using milvexian in addition to a single or dual antiplatelet therapy (SAPT/DAPT) for preventing non-cardioembolic stroke specifically those caused by large-artery extracranial atherosclerosis or intracranial small vessel disease, was published in 2023 (Sharma et al., Safety and efficacy of factor XIa inhibition with milvexian for secondary stroke prevention (AXIOMATIC-SSP): a phase 2, international, randomised, double-blind, placebo-controlled, dose-finding trial, The Lancet Neurology, 2023, vol. 23, pp-46-59). Milvexian in two Phase II clinical trials (AXIOMATIC-TKR and AXIOMATIC-SSP) focused on different patient populations.

### SUMMARY

This disclosure provides treatment regimen comprising a FXIa inhibitor milvexian and optionally one or more antiplatelet therapy to treat and prevent thrombus formation and embolism, thus reducing the risk of arterial and/or venous thrombosis in patients with a history of cardiovascular or cerebrovascular disease, without impairing hemostasis by reducing thrombin generation.

The methods of the disclosure fill the need for anticoagulation in patients with cardiovascular or cerebrovascular disease and at increased risk of bleeding.

In some aspects, the disclosure provides methods of treating or preventing a thrombotic condition in a human patient with a cardiovascular or cerebrovascular disease, wherein the method comprises administering to the human patient an immediate release tablet comprising 25 mg, 50 mg, or 100 mg of milvexian (or a pharmaceutically acceptable salt of solvate thereof), and a pharmaceutically acceptable excipient, optionally the immediate release tablet is administered together with an antiplatelet therapy, wherein the immediate release tablet is administered twice daily.

In some aspects of the disclosed methods, the milvexian (or a pharmaceutically acceptable salt or solvate thereof) is orally administered as a solid pharmaceutical composition comprising milvexian (or a pharmaceutically acceptable salt or solvate thereof) and a pharmaceutically acceptable excipient.

In other aspects of the disclosed methods, the administration does not result in a statistically significant increase in major bleeding complications.

### BRIEF DESCRIPTION OF THE DRAWINGS

FIG. 1 shows coagulation pathways. Legend: FXII, factor XII; FXIIa, activated factor XII; FXI, factor XI; FXIa, activated factor XI; FIX, factor IX; FIXa, activated factor IX; FVIIa, activated factor VII; FVII, factor VII; FX, factor X; FXa, activated factor X. See, Kakkar et al., FXI inhibition: The Holy Grail of Haemostasis-Sparing Anticoaulation, E M J, 2021, vol. 6, pp. 12-20; and Fredenburgh et al., FXIa as a Target for New Anticoagulants, Hamostaseologie, 2021, vol. 41, pp. 104-110.

FIG. 2 illustrates the clot formation associated with atrial fibrillation and adverse vascular events, e.g. ischemic stroke.

FIG. 3 activated partial thromboplastin time as set forth in Example 2. The mean (±SD) percent change from baseline aPTT versus time by treatment is presented in FIG. 3. A dose-dependent increase in aPTT percent change from baseline was observed with milvexian over the dose range from 25 mg QD to 200 mg BID. Summary statistics for aPTT measurements and percent change from baseline in the PD population is provided in Table 6. Nominal time points were used for PD biomarker data analysis. The 4-hour time point includes all tests collected from 0.5 to 6 hours, the 12-hour time point includes all tests collected from 6 to 12 hours, and the 24-hour time point includes all tests collected from 12 to 72 hours.

FIG. 4. shows Kaplan-Meier Plot of Time to Ischemic Stroke and Undetermined Stroke-All Randomized Subjects. See Example 2.

FIG. 5 shows Kaplan-Meier Plot of Time to Ischemic Stroke and Undetermined Stroke-All Randomized Subjects. See Example 2

FIG. 6 illustrates median of milvexian plasma concentration (ng/mL) by nominal sampling time Day 1 through Day 14, linear scale, by treatment Groups of 25 mg, 50 mg and 200 mg QD dosing and 25 mg, 50 mg, 100 mg and 200 mg BID dosing. Subjects who received twice daily and once daily doses of milvexian from 25 mg to 200 mg exhibited increased milvexian concentration with increase in dose. For example, Day 4 predose concentration of 1,570.71 ng/mL was achieved at 100 mg twice daily dose vs 3,699.01 ng/mL at 200 mg twice daily dose. A similar, increase in dose dependent concentration was observed with the once daily regimens, eg, 220.23 ng/ml at 50 mg once daily vs 1,228.63 ng/ml at 200 mg once daily, at Day 4 predose Group; PK Analysis Set (AXIOMATIC-TKR) The observed concentration on Day 4 (12 hours after dosing) was higher for 50 mg and 200 mg once daily compared to 25 mg and 100 mg twice daily, respectively; but the values were lower for once daily dosing compared to twice daily dosing at Day 7, 12 hours after dosing. On Day 4, Tmax was approximately 2 hours after dosing for milvexian 25 mg and 100 mg twice daily vs approximately 4 hours after dosing for milvexian 50 mg and 200 mg once daily. These values were consistent with the median Tmax of 2-to-3 hours for once daily, and approximately 3 hours twice daily observed in healthy volunteers. Mean trough plasma concentrations of milvexian achieved steady state condition approximately on Day 4 following 25 mg, 50 mg, or 100 mg doses after twice daily administration to patients as a spray-dried amorphous solid dispersion formulation in capsule (25 mg or 100 mg strength as described in WO2020210629). (AXIOMATIC-TKR Phase II). See Example 3.

FIG. 7 illustrates median of aPTT ratio to baseline, by nominal sampling time Day 1 through Day 14 by treatment Groups of 25 mg, 50 mg and 200 mg QD dosing and 25 mg, 50 mg, 100 mg and 200 mg BID dosing. A dose related increase in aPTT was observed with milvexian. In this study, the VTE reduction observed with milvexian 50 mg twice daily was superior to enoxaparin and was associated with an approximately 2.3-fold increase in aPTT. Higher milvexian doses (eg, greater than 50 mg twice daily) that resulted in even lower VTE rates accompanied by further increases in aPTT, with the maximum 200 mg twice daily dose resulting in an approximately 3.4-fold increase in aPTT ratio. (AXIOMATIC-TKR Phase II). See Example 3.

FIG. 8 illustrates median of FXI clotting activity % change from baseline, by nominal sampling time Day 1 through Day 14 by treatment Groups of 25 mg, 50 mg and 200 mg QD dosing and 25 mg, 50 mg, 100 mg and 200 mg BID dosing. A dose dependent decrease in FXI clotting activity was observed across the milvexian dose range tested. The VTE reduction observed with milvexian 50 mg twice daily was superior to enoxaparin and was associated with an approximately 18% mean reduction in FXI clotting activity. At the highest dose (200 mg twice daily), an approximately 79% mean reduction in FXI clotting activity was observed. (AXIOMATIC-TKR Phase II). See Example 3.

FIG. 9A illustrates the highest concentration of thrombin as measured by mean peak height ratio to baseline, FIG. 9B illustrates median of endogenous thrombin potential (ETP) area under the curve ratio to baseline, and FIG. 9C illustrates median of lag time ratio to baseline in the TGA measurements, by nominal sampling time Day 1 through Day 14 by treatment Groups of 25 mg, 50 mg and 200 mg QD dosing and 25 mg, 50 mg, 100 mg and 200 mg BID dosing. A clear and robust dose dependent reduction in peak thrombin generation was observed upon milvexian treatment, mean peak height ratio to baseline was 0.18 on Day 10-to-14 for 200 mg twice daily as compared to 0.27 for 100 mg twice daily (FIG. 9A). There were dose related reductions in peak thrombin and area under the curve (total thrombin generation) (FIG. 9B), and there was a dose related prolongation in the lag time which indicates a delay in time clot initiation (FIG. 9C). (AXIOMATIC-TKR Phase II). See Example 3.

FIG. 10A shows the Day 1 milvexian plasma concentration as a function of time after BID administration of a film-coated direct compression tablet (2×100 mg) of the disclosure compared to the Day 1 milvexian plasma concentration as a function of time after BID administration of a milvexian-containing capsule (2×100 mg). See Example 4.

FIG. 10B shows the Day 5 milvexian plasma concentration as a function of time after BID administration of a film-coated direct compression tablet (2×100 mg) of the disclosure compared to the Day 5 milvexian plasma concentration as a function of time after BID administration of a milvexian-containing capsule (2×100 mg). See Example 4.

FIG. 10C shows the Day 1 milvexian plasma concentration as a function of time after BID administration of a film-coated direct compression tablet (1×25 mg) of the disclosure compared to the Day 1 milvexian plasma concentration as a function of time after BID administration of a milvexian-containing capsule (1×25 mg). See Example 4.

FIG. 10D shows the Day 5 milvexian plasma concentration as a function of time after BID administration of a film-coated direct compression tablet (1×25 mg) of the disclosure compared to the Day 5 milvexian plasma concentration as a function of time after BID administration of a milvexian-containing capsule (1×25 mg). See Example 4.

FIG. 11 shows a odds ratio plot resulting from the model-based meta-analysis described in the Dose Selection section of Example 1B.

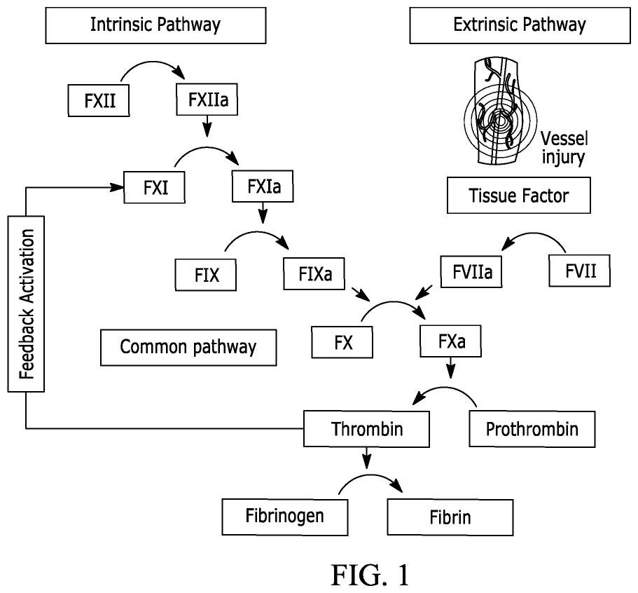

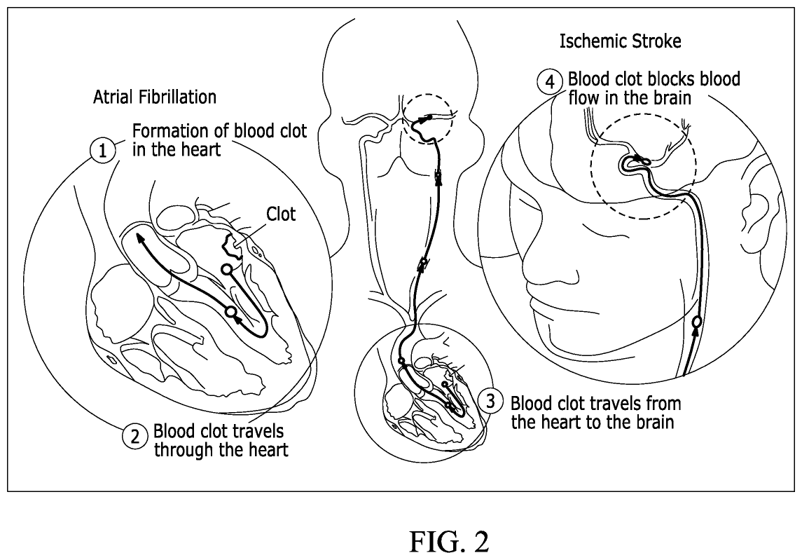

### DETAILED DESCRIPTION OF ILLUSTRATIVE EMBODIMENTS

As used herein, the term “atrial fibrillation” (AF) refers to a supraventricular tachyarrhythmia characterized by uncoordinated atrial activation with consequent deterioration of atrial mechanical function. As used herein, atrial fibrillation also includes atrial flutter. AF features atrial wavelets propagating in different directions, causing disorganized atrial depolarization without effective atrial contraction. On the electrocardiogram, AF is described by the replacement of consistent P waves by rapid oscillations (brillatory (‘f’) waves) that vary in size, shape, and timing, at a rate of 350-600 beats/min, associated with an irregular, frequently rapid ventricular response when atrioventricular conduction is intact. The ventricular response to AF depends on the electrophysiological properties of the atrioventricular node, the level of vagal and sympathetic tone, and the action of drugs. By convention, an episode lasting at least 30 seconds or for an entire 12-lead electrocardiogram is considered diagnostic for clinical AF. The most recent classification proposed by the European Society of Cardiology (ESC) 3 is the following: (1) First diagnosed AF: when a patient presents AF for the first time, irrespective of the duration of the arrhythmia or the presence and severity of AF-related symptoms. (2) Paroxysmal AF: when AF is self-terminating, usually within 48 hours. Although AF paroxysms may continue for up to 7 days, the 48-hour time point is clinically important because after this the likelihood of spontaneous conversion is low and anticoagulation must be considered. (3) Persistent AF: when AF episodes either last longer than 7 days or require termination by either pharmacological or electrical cardioversion. (4) Long-standing persistent AF: when AF has lasted for at least 1 year when adopting a rhythm control strategy is decided. (5) Permanent AF: when the presence of AF is accepted by the patient (and the physician). Atrial flutter occurs when certain electrical signals do not reach the ventricles of the heart. The rapid heartbeat associated with atrial flutter also increases the risk of developing blood clots and stroke. Often, AFib and atrial flutter occur at the same time.

As used herein, the terms “patients with atrial fibrillation”, “patients with atrial fibrillation or flutter”, “patients having a history of atrial fibrillation or atrial flutter”, “patients with a history of or a current atrial fibrillation or atrial flutter” or “patients with a recent history of or a current atrial fibrillation or atrial flutter” or “patients with paroxysmal or persistent atrial fibrillation or atrial flutter” or “patients with a history of, or a current paroxysmal or persistent atrial fibrillation or atrial flutter” or “patients with a recent history of, or a current paroxysmal or persistent atrial fibrillation or atrial flutter” or “patients with paroxysmal or intermittent atrial fibrillation or atrial flutter and a recent episode of atrial fibrillation or atrial flutter, who are in sinus rhythm or who will be cardioverted” or “patients with paroxysmal or persistent atrial fibrillation or atrial flutter and a recent episode of atrial fibrillation or atrial flutter, who are in sinus rhythm or who will be cardioverted” means a patient who, in the past, has presented one or more episodes of atrial fibrillation or atrial flutter and/or who is suffering from atrial fibrillation or atrial flutter at the time the milvexian or a pharmaceutically acceptable salt thereof is used and is eligible for anticoagulation. More particularly, this term means patients with documentation of having been in both atrial fibrillation or flutter and sinus rhythm within the last 6 months preceding the start of treatment. Patients could be either in sinus rhythm, or in atrial fibrillation or atrial flutter at the time the milvexian or a pharmaceutically acceptable salt thereof is initiated. In some embodiments, the patient has a recent history of, or a current, non-permanent atrial fibrillation or atrial flutter. In some embodiments, the patient has paroxysmal atrial fibrillation (i.e., AF that occurs intermittently and stops on its own within seven days). In some embodiments, the patient has persistent atrial fibrillation (i.e., AF that lasts longer than seven days, and may require electric shocks to the heart to restore normal rhythm). In some embodiments, the patient has long-standing persistent atrial fibrillation (i.e., AF that is persistent, but lasts longer than 1 year). In some embodiments, the patient has permanent/chronic atrial fibrillation (i.e., the patient is always in AF, and all attempts for restoring sinus rhythm have failed). In some embodiments, the patients, who have a recent history of atrial fibrillation or atrial flutter, also have concomitant coronary artery disease (CAD) and/or peripheral artery disease (PAD). In some embodiments, the patients, who have a recent history of atrial fibrillation or atrial flutter that is paroxysmal, or sustained, or not reversible, were diagnosed within one year prior to the first dose of milvexian treatment regimen as described herein. Among the patients, notably patients having a history of atrial fibrillation or atrial flutter, the patients further have one or more of Category (A) risk factor selected from (i) age greater than or equal to 75 years, or (ii) history of a clinical symptomatic stroke (e.g., History of symptomatic or silent stroke of any type (ischemic, hemorrhagic, lacunar, or undetermined, or cerebral microbleeds (CMB). If ischemic stroke, it must be >7 days prior to the first dose of milvexian treatment regimen as described herein. Hemorrhagic strokes/transformations must occurred equal to or longer than 3 months); and/or two or more of concomitant or comorbid Category (B) cardiovascular risk factor selected from the group consisting of: (i) age between 65 and 74 years; (ii) hypertension (e.g., use of antihypertensive medications within 6 months before screening, or persistent SBP >140 mmHg or DBP >90 mmHg); (iii) diabetes mellitus (e.g., history of diabetes mellitus and current use of antidiabetic medication(s)); (iv) atherosclerotic vascular diseases (hardening of the arteries, CAD, MI, PAD, PCI, CABG); and (v) congestive heart failure (e.g., symptomatic heart failure including history of hospitalization with heart failure as primary cause regardless of ejection fraction (EF), or EF <40% regardless of history of heart failure hospitalization (most recent and assessed less than 1 year prior to the first dose of milvexian treatment regimen as described herein)). A normal ejection fraction is 50% or higher. An ejection fraction below 40% means the heart isn't pumping enough blood and may be failing. The terms “persistent” and “intermittent” are used interchangeably. More particularly, this term means patients who are medically stable and appropriate for chronic antithrombotic treatment. The term CMBs are defined as rounded foci of <10 mm in size that appear hypointense and distinct from vascular flow voids, leptomeningeal hemosiderosis, or non-hemorrhagic subcortical mineralization on T2*-weighted MRI.

Patients in “permanent atrial fibrillation or flutter” are patients that have all scheduled ECGs in this rhythm throughout the period the dronedarone or a pharmaceutically acceptable salt thereof is administered.

In some embodiments, the term “cerebrovascular disorders” refers to the neurologic problems resulting from the disruption of the blood flow to the brain that leads to damage or death of brain cells from the lack of oxygen. In some embodiments, the cerebrovascular disorders include transient ischemic attack. In some embodiments, the cerebrovascular disorders include stroke. How a stroke or transient ischemic attack affects the body depends on precisely where in the brain the blood supply was cut off.

In some embodiments, the term “stroke” refers to an acute episode of neurologic dysfunction that caused the death of brain tissue (cerebral infarction) resulting from lack of blood flow and insufficient oxygen to the brain. A stroke can either be ischemic or hemorrhagic. In an ischemic stroke, the blood supply to part of the brain is cut off because a blood clot has blocked a blood vessel due to either a thrombus formed locally in an abnormal artery (e.g., atherosclerosis) or an embolus that formed upstream and traveled through the bloodstream. In a hemorrhagic stroke, a blood vessel bursts, preventing normal blood flow and allowing blood to leak into an area of the brain and destroy it. Most strokes begin suddenly, develop rapidly and cause brain damage within minutes (complete stroke). Less commonly, strokes may continue to worsen for several hours to days as a steadily enlarging area of brain tissue dies (stroke in evolution). Ischemic stroke may be lacunar or non-lacunar in nature. Ischemic stroke may be cardioembolic ischemic stroke if the ischemic stroke is attributable to arterial occlusion from embolus that presumably arose from the blood clots formed in the heart or on one of its valves. In some embodiments, the ischemic stroke is non-cardioembolic ischemic stroke. Ischemic stroke was defined based on the 2013 guidelines (Sacco et al., An updated definition of stroke for the 21st century Stroke, 44 (2013), pp. 2064-2089).

In some embodiments, a “stroke” is ischemic, hemorrhagic, or of an unknown cause. The severity of stroke is measured using the National Institutes of Health Stroke Scale (NIHSS) scores in clinical trials (Kamel et al., Validation of the International Classification of Diseases, Tenth Revision Code for the National Institutes of Health Stroke Scale Score. Circ Cardiovasc Qual Outcomes, 2023, vol. 16, e009215). Stroke severity is categorized as follows: 1-4 for minor stroke, 5-15 for moderate stroke, 16-20 for moderate to severe stroke, and 21-42 for severe stroke.

In some embodiments, ischemic stroke refers to a neurological deficit attributable to a non-lacunar, acute brain infarction detected by neuroimaging (CT or MRI) and relevant to the clinical symptoms.

In some embodiments, the ischemic stroke is further characterized by a National Institutes of Health Stroke Score (NIHSS)≤7.

In other embodiments, the ischemic stroke is further characterized by a National Institutes of Health Stroke Score (NIHSS) of 8-15.

In other embodiments, the ischemic stroke is further characterized by a National Institutes of Health Stroke Score (NIHSS)≤15.

In other embodiments, an ischemic stroke is further characterized by evidence of relevant intracranial or cervical arterial atherosclerotic plaque, ulceration or thrombus in a feeding artery documented by imaging (either Doppler ultrasound or CTA or MRA or catheter angiography).

In still other embodiments, an ischemic stroke is further characterized by a Modified Rankin Score. For example, in some embodiments an ischemic stroke is further characterized by a Modified Rankin Score (mRS) of ≤3, of ≤4, of ≤5, or of ≤6.

In some embodiments, MI is defined in accordance with the 4th Universal Definition of MI, excluding type 2 MI (Thygesen et al., Fourth universal definition of myocardial infarction (2018) Eur. Heart J., 40 (2019), pp. 237-269).

In some embodiments, Cardiovascular death is coded when the primary cause of death was MI, stroke, thromboembolism of any other vascular bed, heart failure, primary arrhythmia or a cardiovascular procedure.

As used herein, the term ‘activated partial thromboplastin time (aPTT)” refers to a measure of the intrinsic and final common pathways of the coagulation cascade. It represents the time, in seconds, for plasma to clot after addition of phospholipid, an intrinsic pathway activator, and calcium. The name ‘Activated Partial Thromboplastin Time’ comes from the original form of the test in which only the phospholipid concentration of the test was controlled (as opposed to the phospholipid and the surface activator concentrations) and the name ‘partial thromboplastin’ was applied at the time to phospholipid preparations that accelerated clotting but did not correct the prolonged clotting times of hemophilic plasma. The term ‘partial’ means phospholipid is present but no tissue factor. The normal and reference ranges vary depending on reagent and instrument combinations, particularly with the phospholipid composition.

In some embodiments, aPTT is measured as follows: Plasma samples are incubated with Actin FS aPTT assay reagent containing a standard amount of phospholipid and contact activator (ellagic acid) which activates the intrinsic coagulation pathway. After incubating for 3 minutes, calcium chloride is added to initiate coagulation and formation of a fibrin clot is measured optically. The time to clot formation (measured in seconds) is reported as the Activated Partial Thromboplastin Time (aPTT).

Administration of multiple oral doses of milvexian to healthy human subjects resulted in a dose- and concentration-dependent prolongation of aPTT. The maximal mean change of aPTT from baseline was approximately a 1.1- to 4.1-fold increase after QD doses of 5 to 500 mg for 2 weeks and a 3.4-fold increase after BID dose of 200 mg for 2 weeks. Prothrombin time was not impacted by the administration of a single or multiple doses of milvexian to human subjects, with a maximal mean percent change from baseline of approximately 5%. As used herein, a prothrombin time (PT) test measures how long it takes for a clot to form in a blood sample).

As used herein, “Factor XI Clotting Activity” is determined utilizing an aPTT-based 1-stage clotting time assay. Serial dilutions of normal pooled plasma are mixed with FXI-depleted plasma and the clotting times are measured according to standard aPTT protocol, to establish a reference range. Subject test plasma is treated in the same way and compared with the reference plasma.

In some embodiments, the Factor XI Clotting Activity is measured as follows: Factor XI (FXI) activity is measured using a modification of the activated partial thromboplastin time (aPTT) using Actin FS (Siemens Healthcare) on the Siemens BCS® XP analyzer. A 6-point calibration curve (~5-150%) is prepared using a secondary calibrator (Standard Human Plasma, Siemens Healthcare Diagnostics Inc.) with a known concentration of human FXI assigned by the manufacturer. The reference standard, at approximately 100%, is diluted by the BCS® XP analyzer in saline to generate pre-selected calibration levels of FXI. The calibration curve is plotted with FXI activity in percent (%) on the x-axis and clotting time in seconds on the y-axis. A log/lin regression curve fit is used. The samples to be tested are mixed with FXI deficient plasma (containing less than 1% FXI and at least 75% of all the other factors) to normalize all other factors. APTT reagent (Actin FS) is added and the mixture is incubated. Following incubation, calcium chloride is added to the mixture and the time to clot formation (measured optically) is compared to the time on the calibration curve. Samples are tested at the base dilution (1:10) prepared by the BCS® XP in saline.

As used herein, “Thrombin Generation Assay (TGA)” is a global coagulation assay that evaluates the thrombogenic capacity of a plasma sample and has been proposed that it may better reflect prothrombotic or hemorrhagic states than conventional clotting assays. In the traditional TGA, coagulation of citrated plasma is initiated through the extrinsic pathway by adding tissue factor, phospholipids, and calcium. For this study, in the in-vitro thrombin generation assay (TGA) using human platelet-rich plasma, and a kaolin slurry was used instead to initiate coagulation through the contact activation pathway. Thrombin generation is continuously monitored through the product released by cleavage of a thrombin-specific fluorogenic substrate. The generated thrombogram is used to determine several relevant parameters, including endogenous thrombin potential, defined as area under the thrombin concentration vs. time curve. In some embodiments, administration of a single dose or multiple oral doses of milvexian to a human subject results in inhibition of thrombin generation via the intrinsic pathway, but only minimal inhibition of thrombin generation when initiated by the extrinsic pathway.

As used herein, “preventing” refers to reducing the risk of occurrence. In some embodiments, preventing encompasses eliminating the risk of occurrence (i.e., reducing the risk of occurrence to zero). As such, “prevention” covers the preventive treatment aimed at reducing the probability of the occurrence of a clinical disease-state. In some embodiments, the treatment regimen as described herein is given to patients at risk of developing thromboembolic disease to prevent formation of an occlusive thrombus (primary prevention). In some embodiments, the treatment regimen as described herein is given to patients for secondary prevention, following an initial thrombotic episode, for example, secondary prevention of cardiovascular events in patients with a history of acute myocardial infarction or acute coronary syndrome. In a clinical setting, a combination of aspirin and clopidogrel (or other thienopyridines) may be used to prevent a second thrombotic event.

In some embodiments, “preventing” is synonymous with “reducing the risk” or “reducing the incidence rate” of an adverse atherosclerotic event (e.g., a MACE) occurring. Reducing the risk or reducing the incidence rate means that there is numerical and/or a statistically-significant reduction or lowering in occurrence of the adverse atherosclerotic event by at least 1% or greater. Preferably, this reduction is by 2% or greater, 3% or greater, 4% or greater, 5% or greater, 6% or greater, 7% or greater, 10% or greater, 20% or greater, 26% or greater, 34% or greater, 50% or greater, 64% or greater and 74% or greater. These reductions include confidence intervals equals or greater than 50%, equals or greater than 75%, equals or greater than 80%, equals or greater than 90%, equals or greater than 95%, equals or greater than 98% and equals or greater than 99%. Confidence intervals of equals or greater than 95% are preferred.

Within the scope of this disclosure, “prophylaxis” is the protective treatment of a disease state to reduce and/or minimize the risk and/or reduction in the risk of recurrence of a disease state by administering to a patient a therapeutically effective amount of milvexian or a pharmaceutically acceptable salt, or a solvate thereof. Patients may be selected for prophylaxis therapy based on factors that are known to increase risk of suffering a clinical disease state compared to the general population. For prophylaxis treatment, conditions of the clinical disease state may or may not be presented yet. “Prophylaxis” treatment can be divided into (a) primary prophylaxis and (b) secondary prophylaxis. Primary prophylaxis is defined as treatment to reduce or minimize the risk of a disease state in a patient that has not yet presented with a clinical disease state, whereas secondary prophylaxis is defined as minimizing or reducing the risk of a recurrence or second occurrence of the same or similar clinical disease state.

As used herein, the term “treating” refers to ameliorating the signs or symptoms of a disease or condition in a patient and/or preventing a disease or condition in a patient. As used herein, unless otherwise noted, the terms “treating”, “treatment” and the like, shall include the management and care of a patient for the purpose of combating a disease, condition, or disorder and includes the administration of milvexian to prevent the onset of the symptoms or complications, alleviate the symptoms or complications, or eliminate the disease, condition, or disorder. As such, “treating” or “treatment” cover the treatment of a disease-state in a human, and include: (a) inhibiting the disease-state, i.e., arresting its development; and/or (b) relieving the disease-state, i.e., causing regression of the disease state.

As used herein, a “risk factor” is a demographic factor that influences the underlying risk of an event independently of any drug treatment.

As used herein, the term “atherosclerotic event” refers to a major adverse cardiovascular events (MACE), a major adverse vascular event (MAVE), arrhythmogenic cardiomyopathy, a major adverse limb events (MALE), or a combination thereof.

As used herein, the term “major adverse cardiovascular event” or “MACE” refers to cardiovascular death, nonfatal myocardial infarction, ischemic stroke, or combinations thereof.

As used herein, the term “major adverse vascular event” or “MAVE” refers to cardiovascular death, nonfatal myocardial infarction, ischemic stroke, MALE, symptomatic VTE, or combinations thereof.

As used herein, the term “major adverse limb event” or “MALE” refers to acute limb ischemia (ALI), major nontraumatic vascular amputation, or combinations thereof.

As used herein, the term “symptomatic VTE” refers to pulmonary embolism, deep vein thrombosis, or combinations thereof.

As used herein, “pharmaceutically acceptable salt” refers to derivatives wherein a compound is modified by making an acid or a basic salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic. The pharmaceutically acceptable salts of milvexian can be synthesized using conventional chemical methods. Generally, such salts can be prepared by reacting milvexian with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, Pa. (1990), the disclosure of which is hereby incorporated by reference.

As used herein, the term “standard of care” refers to a treatment process that is generally accepted by medical experts as a proper treatment for a certain type of disease (e.g., acute coronary syndrome) and that is widely used by healthcare professionals.

As used herein “safe” means that the regimen provides a net clinical benefit such that the risk reduction for an atherosclerotic event in a human outweighs the increased risk of an adverse event, for example, serious bleeding, as compared to antiplatelet therapy alone, as determined by a regulatory agency.

As used herein “effective” means that the regimen provides risk reduction and treatment for an atherosclerotic event as compared to antiplatelet therapy alone.

Absolute risk reduction (ARR) is the percentage of patients in the control group (i.e., the group not receiving the regimen) who have a bad outcome minus the percentage of patients in the treatment group (i.e., the group receiving the regimen) who have a bad outcome. For example, if the incidence rate in the control group is 20% (i.e., if 20% of the patients in the control group experience a bad outcome), and the incidence rate in the treatment group is 12% (i.e. 12% of the patients in experience a bad outcome), then the absolute risk reduction (ARR) is 8% (i.e., 20%-12%).

“Relative Risk” (RR) is the proportion of bad outcomes (e.g., suffering an ischemic stroke) in the treatment group (i.e., the group receiving the regimen) divided by the proportion of bad outcomes in the control group (i.e., the group not receiving the regimen). For example, if 20% of the patients in the control group experience a bad outcome, and 12% of the patients in the treatment group experience a bad outcome, then the RR=0.12/0.20=0.6. Thus, an RR of less than 1 indicates a reduction in risk.

As used herein, the term “Relative Risk Reduction” (RRR) refers to how much the regimen reduced the risk of bad outcomes relative to the control group who did not receive the regimen. For example, if 20% of the patients in the control group experience a bad outcome, and 12% of the patients in the treatment group experience a bad outcome, then the RRR=(1−RR)*100%=[1−(0.12/0.20)]*100%=0.4*100%=40%. Thus, an RRR of greater than 0% indicates a reduction in risk.

In some embodiments, the “Bleeding Academic Research Consortium (BARC) criteria are as set forth in Roxana Mehran, et al. Standardized Bleeding Definitions for Cardiovascular Clinical Trials, Circulation. 2011; 123:2736-2747, which is incorporated by reference herein.

In other embodiments, the “Bleeding Academic Research Consortium (BARC) criteria are as set forth in Pascal Vranckx, et al. Validation of BARC Bleeding Criteria in Patients With Acute Coronary Syndromes J Am Coll Cardiol 2016; 67:2135-44, which is incorporated by reference herein.

In some embodiments, the GUSTO criteria are as set forth in “An international randomized trial comparing four thrombolytic strategies for acute myocardial infarction.” The GUSTO investigators. N Engl J Med 1993; 329:673-82, which is incorporated by reference herein.

In some embodiments, the GUSTO criteria are as set forth in Pascal Vranckx, et al. Validation of BARC Bleeding Criteria in Patients With Acute Coronary Syndromes J Am Coll Cardiol 2016; 67:2135-44, which is incorporated by reference herein.

In some embodiments, the TIMI criteria are as set forth in Rao AK, Pratt C, Berke A, et al. Thrombolysis in Myocardial Infarction (TIMI) Trial—phase I: hemorrhagic manifestations and changes in plasma fibrinogen and the fibrinolytic system in patients treated with recombinant tissue plasminogen activator and streptokinase. J Am Coll Cardiol 1988; 11:1-11. which is incorporated by reference herein.

In some embodiments, the TIMI criteria are as set forth in Pascal Vranckx, et al. Validation of BARC Bleeding Criteria in Patients With Acute Coronary Syndromes J Am Coll Cardiol 2016; 67:2135-44, which is incorporated by reference herein.

In some embodiments, the “Bleeding Academic Research Consortium (BARC) Type 3 criteria” are: a. Overt bleeding plus hemoglobin drop of 3 to <5 g/dL (provided hemoglobin drop is related to bleed); transfusion with overt bleeding; b. Overt bleeding plus hemoglobin drop ≥5 g/dL (provided hemoglobin drop is related to bleed); cardiac tamponade; bleeding requiring surgical intervention for control; bleeding requiring IV vasoactive agents; or c. Intracranial hemorrhage confirmed by autopsy, imaging, or lumbar puncture; intraocular bleed compromising vision. See, e.g., Roxana Mehran, et al. Standardized Bleeding Definitions for Cardiovascular Clinical Trials, Circulation. 2011; 123:2736-2747.

In some embodiments, the “Bleeding Academic Research Consortium (BARC) Type 5 criteria” are: a. Probable fatal bleeding; or b. Definite fatal bleeding (overt or autopsy or imaging confirmation). See, e.g., Roxana Mehran, et al. Standardized Bleeding Definitions for Cardiovascular Clinical Trials, Circulation. 2011; 123:2736-2747.

In some embodiments, the “Bleeding Academic Research Consortium (BARC) Type 2 criteria” are: any clinically overt sign of hemorrhage that “is actionable” and requires diagnostic studies, hospitalization, or treatment by a health care professional. See, e.g., Roxana Mehran, et al. Standardized Bleeding Definitions for Cardiovascular Clinical Trials, Circulation. 2011; 123:2736-2747.

In some embodiments, the “ISTH criteria (major bleeding or clinically-relevant non-major bleeding (CRNM)) criteria” are as follows:

ISTH major bleeding in non-surgical patients is defined as having a symptomatic presentation and:
i. Fatal bleeding, and/or
ii. Bleeding in a critical area or organ, such as intracranial, intraspinal, intraocular, retroperitoneal, intra-articular or pericardial, or intramuscular with compartment syndrome, and/or
iii. Bleeding causing a fall in hemoglobin level of 20 g L⁻¹ (1.24 mmol L⁻¹) or more, or leading to transfusion of two or more units of whole blood or red cell

ISTH CRNM bleeding is any sign or symptom of hemorrhage (e.g., more bleeding than would be expected for a clinical circumstance, including bleeding found by imaging alone) that does not fit the criteria for the ISTH definition of major bleeding but does meet at least one of the following criteria:
i. requiring medical intervention by a healthcare professional
ii. leading to hospitalization or increased level of care
iii. prompting a face to face (i.e., not just a telephone or electronic communication) evaluation

It will be understood that the use of terms of approximation, such as “approximately” or “about,” when referring to, for example, amounts, durations of time, extents of effects, and the like, may be a number that is within 5% (greater or less than), or within 7.5% (greater or less than), or within 10% (greater or less than), or within 12.5% (greater or less than), or within 15% (greater or less than), or within 17.5% (greater or less than), or within 20% (greater or less than) of the specified number.

In some aspects, the disclosure provides methods of treating or preventing a thrombotic condition in a human patient with a cardiovascular or cerebrovascular disease, wherein the method comprises administering to the human patient an immediate release tablet comprising 25 mg, 50 mg, or 100 mg of milvexian (or a pharmaceutically acceptable salt of solvate thereof), and a pharmaceutically acceptable excipient, optionally the immediate release tablet is administered together with an antiplatelet therapy, wherein the immediate release tablet is administered twice daily.

In some embodiments, the thrombotic condition is a thromboembolic disorder selected from an arterial thromboembolic disorder; a venous thromboembolic disorder; or a thromboembolic disorder in the chambers of the heart or in the peripheral circulation.

In some embodiments, the thrombotic condition is a thromboembolic disorder.

In some embodiments, the thrombotic condition is an arterial thromboembolic disorder.

In some embodiments, the arterial thromboembolic disorder is coronary arterial thrombosis, cerebral arterial thrombosis, arterial embolism, cerebral embolism, acute ischemic stroke, transient ischemic attack (TIA), myocardial infarction, stroke, acute coronary syndrome, atherosclerosis, peripheral occlusive arterial disease, cardiovascular death, or combinations thereof.

In some embodiments, the thrombotic condition is a venous thromboembolic disorder.

In some embodiments, the venous thromboembolic disorder is deep vein thromboembolism, venous thromboembolism, pulmonary embolism, death, or combinations thereof.

In some embodiments, the thrombotic condition is a thromboembolic disorder in the chambers of the heart or in the peripheral circulation.

In some embodiments, the thrombotic condition is unstable angina, an acute coronary syndrome, atrial fibrillation, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, or thrombosis resulting from medical implants, devices, or procedures in which blood is exposed to an artificial surface that promotes thrombosis.

In some embodiments, the thrombotic condition is stroke and/or non-central nervous system (CNS) systemic embolism.

In some embodiments, the thrombotic condition is unstable angina.

In some embodiments, the thrombotic condition is an acute coronary syndrome.

In some embodiments, the thrombotic condition is a chronic coronary disease.

In some embodiments, the thrombotic condition is a chronic coronary syndrome.

In some embodiments, the thrombotic condition is atrial fibrillation.

In some embodiments, the thrombotic condition is myocardial infarction.

In some embodiments, the thrombotic condition is transient ischemic attack.

In some embodiments, the thrombotic condition is stroke.

In some embodiments, the thrombotic condition is atherosclerosis.

In some embodiments, the thrombotic condition is peripheral occlusive arterial disease.

In some embodiments, the thrombotic condition is venous thrombosis.

In some embodiments, the thrombotic condition is deep vein thrombosis.

In some embodiments, the thrombotic condition is thrombophlebitis.

In some embodiments, the thrombotic condition is arterial embolism.

In some embodiments, the thrombotic condition is coronary arterial thrombosis.

In some embodiments, the thrombotic condition is cerebral arterial thrombosis.

In some embodiments, the thrombotic condition is cerebral embolism.

In some embodiments, the thrombotic condition is kidney embolism.

In some embodiments, the thrombotic condition is pulmonary embolism.

In some embodiments, the thrombotic condition is thrombosis resulting from medical implants, devices, or procedures in which blood is exposed to an artificial surface that promotes thrombosis.

In some embodiments, the thrombotic condition comprises arterial thromboembolic disorder associated with an acute coronary syndrome. In some embodiments, the thrombotic condition comprises arterial thromboembolic disorder associated with a chronic coronary disease. In some embodiments, the thrombotic condition comprises arterial thromboembolic disorder associated with chronic coronary syndrome.

In some aspects, the methods of the disclosure are performed on a human patient with a cardiovascular or cerebrovascular disease.

In some embodiments, the cardiovascular or cerebrovascular disease is a cerebrovascular disease. In some embodiments, the cerebrovascular disease is selected from non-cardioembolic ischemic stroke, or transient ischemic attack (TIA).

In other embodiments, the cardiovascular or cerebrovascular disease is a cardiovascular disease.

In some embodiments, the cardiovascular disease is atrial fibrillation or flutter.

In other embodiments, the cardiovascular disease is acute coronary syndrome. In other embodiments, the cardiovascular disease is chronic coronary disease. In other embodiments, the cardiovascular disease is chronic coronary syndrome.

In some aspects, the disclosure is directed to methods for preventing adverse cerebrovascular events or adverse cardiovascular events in a human patient with acute coronary syndrome (ACS), wherein the method comprises measuring the patient's baseline FXI clotting activity, and then administering to the human patient a regimen comprising: (i) a pharmaceutical composition comprising 25 mg of milvexian (or pharmaceutically acceptable salt or solvate thereof); and (ii) an antiplatelet therapy selected from the group consisting of aspirin, a P2Y12 inhibitor, and combination thereof; wherein the pharmaceutical composition is administered twice daily.

In some embodiments, the antiplatelet therapy is a dual antiplatelet therapy (DAPT) comprising aspirin 75-100 mg QD and a P2Y12 inhibitor. In some embodiments, the antiplatelet therapy is a single antiplatelet therapy (SAPT). In some embodiments, the method comprises administering an antiplatelet therapy consists of a dual antiplatelet therapy and a single antiplatelet therapy. In some embodiments, the antiplatelet therapy comprises DAPT.

In some embodiments, the DAPT comprises aspirin and prasugrel. In some embodiments, the DAPT comprises aspirin and prasugrel administered for 12 months.

In some embodiments, the DAPT comprises aspirin and clopidogrel. In some embodiments, the aspirin and clopidogrel is administered for 21 days to 12 months. In some embodiments, the DAPT comprises aspirin and clopidogrel. In some embodiments, the aspirin and clopidogrel is administered for 21 days. In some embodiments, the aspirin and clopidogrel is administered for 6 months. In some embodiments, the aspirin and clopidogrel is administered for 12 months.

In some embodiments, the DAPT comprises aspirin and ticagrelor. In some embodiments, the DAPT comprises aspirin and ticagrelor administered for 12 months. In some embodiments, the DAPT comprises aspirin 75-100 mg QD and a P2Y12 inhibitor administered first for 12 months followed by aspirin monotherapy for 6 months to 12 months.

In some embodiments, the DAPT comprises aspirin 75-100 mg QD and a P2Y12 inhibitor administered first for 21 days followed by aspirin monotherapy for 6 months.

In some embodiments, the DAPT comprises aspirin 75-100 mg QD and a P2Y12 inhibitor administered first for 21 days followed by aspirin monotherapy for 12 months.

In some embodiments, the DAPT comprises aspirin 75-100 mg QD and clopidogrel 75 mg QD administered first for 21 days followed by aspirin monotherapy for 6 months.

In some embodiments, the DAPT comprises aspirin 75-100 mg QD and ticagrelor 90 mg BID administered first for 12 months followed by aspirin 75-100 mg QD for 12 months.

In some embodiments, the DAPT comprises aspirin 75-100 mg QD and ticagrelor 90 mg BID administered first for 12 months followed by ticagrelor 90 mg BID for 23 months.

In some embodiments, the DAPT comprise aspirin 75-100 mg QD and 75 mg ticagrelor BID. In some embodiments, the aspirin 75-100 mg QD and 75 mg ticagrelor BID is administered for 12 months.

In some embodiments, the DAPT comprises aspirin 81-100 mg QD and 90 mg ticagrelor BID. In some embodiments, the aspirin 81-100 mg QD and 90 mg ticagrelor BID is administered for 3 years.

In some embodiments, the antiplatelet therapy comprises aspirin administered for 30 days to 90 days followed by clopidogrel monotherapy. In some embodiments, the antiplatelet therapy comprises aspirin administered for 60 days followed by clopidogrel monotherapy. In some embodiments, the antiplatelet therapy comprises aspirin administered for 90 days followed by clopidogrel monotherapy.

In some embodiments, the antiplatelet therapy comprises SAPT.

In some embodiments, the SAPT comprises aspirin. In some embodiments, aspirin monotherapy is administered once daily. In some embodiments, aspirin monotherapy is administered once daily for 3 months to 3 years. In some embodiments, aspirin monotherapy is administered once daily for 3 months. In some embodiments, aspirin monotherapy is administered once daily for 6 months. In some embodiments, aspirin monotherapy is administered once daily for 12 months. In some embodiments, aspirin monotherapy is administered once daily for 24 months. In some embodiments, aspirin monotherapy is administered once daily for 36 months. In some embodiments, aspirin monotherapy is administered twice daily.

In some embodiments, the SAPT comprises a P2Y12 inhibitor. In some embodiments, the P2Y12 inhibitor is selected from prasugrel, clopidogrel, selatogrel, or ticagrelor. In some embodiments, the P2Y12 inhibitor monotherapy is administered for 3 months to 6 months.

In some embodiments, the SAPT comprises clopidogrel. In some embodiments, the SAPT comprises ticagrelor. In some embodiments, the SAPT comprises ticagrelor monotherapy followed by aspirin 20 mg twice daily.

In some embodiments, the SAPT comprises aspirin 75-100 mg QD. In some embodiments, the aspirin 75-100 mg QD is administered for 12 months.

In some embodiments, the SAPT comprises ticagrelor 90 mg BID. In some embodiments, the ticagrelor 90 mg BID is administered for 3 years.

In some embodiments, the SAPT comprises clopidogrel. In some embodiments, aspirin monotherapy is administered first followed by clopidogrel monotherapy for at least additional 2 months.

In some embodiments, the methods of the disclosure are directed to primary prevention of adverse cerebrovascular events or adverse cardiovascular events.

In some embodiments, the methods of the disclosure are directed to secondary prevention of adverse cerebrovascular events or adverse cardiovascular events.

In some embodiments of the disclosed methods, the adverse cerebrovascular event or adverse cardiovascular events comprise one or more of stroke, heart attack, or death.

In some embodiments of the disclosed methods, the adverse cerebrovascular event or adverse cardiovascular event occurs in an organ selected from heart, brain, limb, blood supply system, or vessel.

In some embodiments of the disclosed methods, the adverse cerebrovascular event or adverse cardiovascular event comprises one or more major adverse cardiovascular event (MACE) selected from the group consisting of cardiovascular death, nonfatal myocardial infarction, ischemic stroke, and combinations thereof.

In other embodiments of the disclosed methods, the adverse cerebrovascular event or adverse cardiovascular event comprises one or more major adverse vascular events (MAVE) selected from the group consisting of MACE, major adverse limb events (MALE) including one or more selected from acute limb ischemia, chronic limb ischemia, or major amputation; symptomatic venous thromboembolic events, and combinations thereof.

In some embodiments of the disclosed methods, the adverse cerebrovascular event or adverse cardiovascular event is selected from the group consisting of arrhythmogenic cardiomyopathy, nonfatal myocardial infarction, ischemic stroke, and combinations thereof.

In some embodiments of the disclosed methods, the adverse cerebrovascular event or adverse cardiovascular evens comprises cardiovascular death.

In some embodiments of the disclosed methods, the adverse cerebrovascular event or adverse cardiovascular event comprises arrhythmogenic cardiomyopathy.

In some aspects, the disclosure is directed to methods for preventing adverse cerebrovascular events or adverse cardiovascular events in a human patient with chronic coronary syndrome (CCS) or chronic coronary diseases (CCD), wherein the method comprises measuring the patient's baseline FXI clotting activity, and then administering to the human patient a regimen comprising: (i) milvexian at about 25 mg to about 100 mg twice daily (BID); and (ii) an antiplatelet therapy. A used herein, “chronic coronary disease” (CCD) is heterogeneous group of conditions that includes obstructive and nonobstructive CAD with or without previous myocardial infarction (MI) or revascularization, ischemic heart disease diagnosed only by noninvasive testing, and chronic angina syndromes with varying underlying causes. The care of patients with CCD is a continuum from post-acute care in patients presenting with chest pain, acute coronary syndromes (ACS), or both to outpatient CCD-related management (Virani et al., 2023 AHA/ACC/ACCP/ASPC/NLA/PCNA Guideline for the Management of Patients With Chronic Coronary Disease, Circulation, 2023, Vol. 148, pages e9-e119). As used herein, “chronic coronary syndromes” (CCS) refers to clinical scenarios in patients with suspected or established CCS including: (i) patients with suspected CAD and ‘stable’ anginal symptoms, and/or dyspnoea; (ii) patients with new onset of heart failure (HF) or left ventricular (LV) dysfunction and suspected CAD; (iii) asymptomatic and symptomatic patients with stabilized symptoms <1 year after an ACS, or patients with recent revascularization; (iv) asymptomatic and symptomatic patients >1 year after initial diagnosis or revascularization; (v) patients with angina and suspected vasospastic or microvascular disease (see section 6); and (vi) asymptomatic subjects in whom CAD is detected at screening (Wijns et al., 2019 Guidelines on Chronic Coronary Syndromes ESC Clinical Practice Guidelines, European Heart Journal, 2019, 00, pp. 1-71).

In some embodiments, the human patient with chronic coronary syndrome (CCS) or chronic coronary diseases (CCD) also has atrial fibrillation.

In some embodiments, administering of milvexian is at 25.0 mg BID. In some embodiments, administering of milvexian is at 50.0 mg BID. In some embodiments, administering of milvexian is at 75.0 mg BID. In some embodiments, administering of milvexian is at 100.0 mg BID.

In some embodiments, the antiplatelet therapy for CCS or CCD comprises SAPT for secondary prevention in a patient with CCS or CCD. In some embodiments, the SAPT for secondary prevention in patient with CCS or CCD comprises aspirin 75-100 mg QD for 6 months. In some embodiments, the SAPT for secondary prevention in patient with CCS or CCD comprises clopidogrel 75 mg QD for 6 months. In some embodiments, the SAPT for CCS or CCD comprises clopidogrel 75 mg QD for 24 months. In some embodiments, the SAPT for CCS or CCD comprises aspirin 325 mg QD for 24 months. In some embodiments, the SAPT for CCS or CCD comprises ticagrelor 90 mg BID and aspirin administered for 3 years. In some embodiments, the SAPT for CCS or CCD comprises ticagrelor 60 mg BID and aspirin administered for 3 years. In some embodiments, the antiplatelet therapy for CCS or CCD comprises 60 mg prasugrel followed by DAPT comprising aspirin and clopidogrel. In some embodiments, the antiplatelet therapy for CCS or CCD comprises 600 mg clopidogrel followed by DAPT comprising aspirin and clopidogrel.

In some embodiments, the antiplatelet therapy for CCS comprises ticagrelor 180 mg followed by DAPT comprising aspirin and ticagrelor 90 mg BID for 30 days. In some embodiments, the antiplatelet therapy for CCS or CCD comprises clopidogrel 300 mg to 600 mg followed by DAPT comprising aspirin and clopidogrel 75 mg QD for 30 days.

In some embodiments, the antiplatelet therapy for CCS or CCD comprises DAPT. In some embodiments, the DAPT for CCS or CCD comprises aspirin and a second antithrombotic drug. In some embodiments, aspirin and a P2Y12 inhibitor for CCS or CCD is administered for 6 months.

In some embodiments, the antiplatelet therapy for CCS or CCD comprises DAPT. In some embodiments, the antiplatelet therapy for CCS or CCD comprises DAPT administered for 3 months to 12 months. In some embodiments, the antiplatelet therapy for CCS or CCD comprises DAPT administered for 3 months. In some embodiments, the antiplatelet therapy for CCS or CCD comprises DAPT administered for 12 months. In some embodiments, the antiplatelet therapy for CCS or CCD comprises DAPT administered for 60 days followed by P2Y12 monotherapy. In some embodiments, the antiplatelet therapy for CCS or CCD comprises DAPT administered for 60 days followed by ticagrelor monotherapy for 23 months. In some embodiments, the antiplatelet therapy for CCS or CCD comprises DAPT administered for 60 days followed by aspirin monotherapy for 12 months. In some embodiments, the antiplatelet therapy for CCS or CCD comprises aspirin and clopidogrel administered for 60 days followed by clopidogrel monotherapy for 12 months. In some embodiments, the antiplatelet therapy for CCS or CCD comprises aspirin and prasugrel administered for 60 days followed by clopidogrel monotherapy for 12 months.

In some embodiments, the antiplatelet therapy for CCS or CCD comprises aspirin and clopidogrel administered for 12 months followed by aspirin monotherapy for 18 months. In some embodiments, the antiplatelet therapy for CCS or CCD comprises aspirin and prasugrel administered for 12 months followed by aspirin monotherapy for 18 months. In some embodiments, the antiplatelet therapy for CCS or CCD comprises aspirin and clopidogrel administered for 30 months. In some embodiments, the antiplatelet therapy for CCS or CCD comprises aspirin and prasugrel administered for 30 months.

In some embodiments, the DAPT for CCS or CCD comprises aspirin and ticagrelor administered for 3 months followed by ticagrelor monotherapy. In some embodiments, the DAPT for CCS or CCD comprises aspirin and ticagrelor administered for 12 months. In some embodiments, the DAPT for CCS or CCD comprises aspirin 75-100 mg QD and clopidogrel 75 mg BID administered for 6 months.

In some embodiments, the antiplatelet therapy for CCS or CCD comprises DAPT followed by SAPT. In some embodiments, the antiplatelet therapy for CCS or CCD comprises DAPT administered first for 1 month to 12 months followed by SAPT. In some embodiments, the antiplatelet therapy for CCS or CCD comprises DAPT administered first for 3 months to 6 months followed by SAPT. In some embodiments, the antiplatelet therapy for CCS or CCD comprises DAPT administered first for 3 months followed by SAPT. In some embodiments, the antiplatelet therapy for CCS or CCD comprises DAPT administered first for 6 months followed by SAPT. In some embodiments, the antiplatelet therapy for CCS or CCD comprises DAPT administered first for 1 month followed by aspirin monotherapy. In some embodiments, the antiplatelet therapy for CCS or CCD comprises DAPT administered first for 3 months followed by P2Y12 inhibitor monotherapy.

In some embodiments, the antiplatelet therapy for CCS or CCD comprises aspirin 75-100 mg QD and clopidogrel 75 mg BID administered for 12 months followed by aspirin 75-100 mg QD for 12 months.

In some embodiments, the antiplatelet therapy for CCS or CCD comprises a triple therapy administered for 1 week followed by a dual therapy comprising clopidogrel and dabigatran for up to 6 months. In some embodiments, the antiplatelet therapy for CCS or CCD comprises a triple therapy administered for 1 week followed by a dual therapy comprising clopidogrel and dabigatran for up to 6 months. In some embodiments, the antiplatelet therapy for CCS or CCD comprises a triple therapy administered for 1 week followed by a dual therapy comprising clopidogrel and a FXa inhibitor for up to 6 months. In some embodiments, the antiplatelet therapy for CCS or CCD comprises a triple therapy administered for 1 week followed by a dual therapy comprising clopidogrel and apixaban for up to 6 months. In some embodiments, the antiplatelet therapy for CCS or CCD comprises a triple therapy administered for 1 week followed by a dual therapy comprising clopidogrel and rivaroxaban for up to 6 months. In some embodiments, the antiplatelet therapy for CCS or CCD comprises a triple therapy administered for 1 week followed by a dual therapy comprising clopidogrel and edoxaban for up to 6 months.

In some embodiments, the methods of the disclosure are directed to primary prevention of adverse cerebrovascular events or adverse cardiovascular events.

In some embodiments, the methods of the disclosure are directed to secondary prevention of adverse cerebrovascular events or adverse cardiovascular events.

In some embodiments of the disclosed methods, the adverse cerebrovascular event or adverse cardiovascular events comprise one or more of stroke, heart attack, or death.

In some embodiments of the disclosed methods, the adverse cerebrovascular event or adverse cardiovascular event occurs in an organ selected from heart, brain, limb, blood supply system, or vessel.

In some embodiments of the disclosed methods, the adverse cerebrovascular event or adverse cardiovascular event comprises one or more major adverse cardiovascular event (MACE) selected from the group consisting of cardiovascular death, nonfatal myocardial infarction, ischemic stroke, and combinations thereof.

In other embodiments of the disclosed methods, the adverse cerebrovascular event or adverse cardiovascular event comprises one or more major adverse vascular events (MAVE) selected from the group consisting of MACE, major adverse limb events (MALE) including one or more selected from acute limb ischemia, chronic limb ischemia, or major amputation; symptomatic venous thromboembolic events, and combinations thereof.

In some embodiments of the disclosed methods, the adverse cerebrovascular event or adverse cardiovascular event is selected from the group consisting of arrhythmogenic cardiomyopathy, nonfatal myocardial infarction, ischemic stroke, and combinations thereof.

In some embodiments of the disclosed methods, the adverse cerebrovascular event or adverse cardiovascular evens comprises cardiovascular death.

In some embodiments of the disclosed methods, the adverse cerebrovascular event or adverse cardiovascular event comprises arrhythmogenic cardiomyopathy.

In some aspects, the disclosure is directed to methods for treating or preventing adverse cerebrovascular event or adverse cardiovascular event in a human patient with acute coronary syndrome.

In some embodiments, the human patient is a female. In other embodiments, the human patient is a male.

In some embodiments, the human patient is at least 40 years old.

In other embodiments, the human patient is at least 50 years old.

In other embodiments, the human patient is at least 60 years old.

In other embodiments, the human patient is at least 70 years old.

In some aspects, the methods of the disclosure comprise administering to the human patient a regimen comprising: (i) a pharmaceutical composition comprising 25 mg of milvexian (or pharmaceutically acceptable salt or solvate thereof), or comprising 12.5 mg to 200 mg of milvexian (or pharmaceutically acceptable salt or solvate thereof); and (ii) an antiplatelet therapy selected from the group consisting of aspirin, a P2Y12 inhibitor, and combination thereof.

In some aspects, the methods of the disclosure comprise administering to the human patient a regimen comprising: (i) a pharmaceutical composition comprising 25 mg of milvexian (or pharmaceutically acceptable salt or solvate thereof) twice daily; and (ii) an antiplatelet therapy selected from the group consisting of aspirin, a P2Y12 inhibitor, and combination thereof.

In some embodiments, the pharmaceutical composition comprises milvexian.

In some embodiments, the pharmaceutical composition comprises a solvate of milvexian.

In other embodiments, the pharmaceutical composition a pharmaceutically acceptable salt of milvexian.

In embodiments of the disclosed methods in which the pharmaceutical composition comprises a solvate or a pharmaceutically acceptable salt of milvexian, then the amount specified is on a milvexian basis. That is, an amount of the solvate or the pharmaceutically acceptable salt in the pharmaceutical composition contains the specified amount of milvexian. For example, a pharmaceutical composition comprising 25 mg of milvexian (or pharmaceutically acceptable salt or solvate thereof) refers to a pharmaceutical composition comprising either 25 mg of milvexian, or an amount of a pharmaceutically acceptable salt or solvate of milvexian equivalent to 25 mg of milvexian.

In some embodiments of the disclosed methods, the regimen comprises a pharmaceutical composition comprising 12.5 mg of milvexian (or pharmaceutically acceptable salt or solvate thereof) administered twice daily.

In some embodiments of the disclosed methods, the regimen comprises a pharmaceutical composition comprising 25 mg of milvexian (or pharmaceutically acceptable salt or solvate thereof) administered twice daily.

In some embodiments of the disclosed methods, the regimen comprises a pharmaceutical composition comprising 50 mg of milvexian (or pharmaceutically acceptable salt or solvate thereof) administered twice daily.

In some embodiments of the disclosed methods, the regimen comprises a pharmaceutical composition comprising 100 mg of milvexian (or pharmaceutically acceptable salt or solvate thereof) administered twice daily.

In some embodiments of the disclosed methods, the regimen comprises a pharmaceutical composition comprising 12.5 mg to 200 mg of milvexian (or pharmaceutically acceptable salt or solvate thereof) administered twice daily.

In some embodiments of the methods of the disclosure in which the human patient is administered the composition comprising milvexian (or a pharmaceutically acceptable salt or solvate thereof, on a milvexian basis) twice daily, one of the two administrations is made in the morning, and the other of the two administrations is made in the evening, and the administrations are made at approximately (i.e., within ±1 hour) the same time each day.

In some aspects of the disclosed methods, optionally the immediate release milvexian tablet is administered together with an antiplatelet therapy.

In some aspects, the regimen used in the disclosed methods comprises administering an antiplatelet therapy selected from the group consisting of aspirin, a P2Y12 inhibitor, and combination thereof.

In some embodiments of the disclosed methods, the antiplatelet therapy comprises single antiplatelet therapy (SAPT).

In some embodiments of the disclosed methods, the antiplatelet therapy comprises dual antiplatelet therapy (DAPT).

In some embodiments, the antiplatelet therapy comprises dual antiplatelet therapy (DAPT) for 21 days, followed by single antiplatelet therapy (SAPT) thereafter.

In some embodiments of the disclosed methods, the regimen comprises aspirin and clopidogrel combination therapy from day 1 to day 21, followed by aspirin monotherapy from day 22 to day 90 day.

In some embodiments of the disclosed methods, the regimen comprises DAPT for >90 days (with or without de-escalation to SAPT).

In some embodiments of the disclosed methods, the regimen comprises DAPT for >90 days (with de-escalation to SAPT).

In other embodiments of the disclosed methods, the regimen comprises DAPT for >90 days (without de-escalation to SAPT).

In some embodiments of the disclosed methods, the regimen comprises DAPT for ≤90 days with de-escalation to SAPT.

In some embodiments of the disclosed methods, the regimen comprises SAPT.

In some embodiments of the disclosed methods, the regimen comprises administering aspirin and/or clopidogrel without milvexian dose adjustment.

In some embodiments, the single antiplatelet therapy is aspirin.

In some embodiments of the single antiplatelet therapy, aspirin is administered in an amount of 50-150 mg daily, such as, for example, one of: 50 mg, 55 mg, 60 mg, 65 mg, 70 mg, 75 mg, 80 mg, 81 mg, 85 mg, 90 mg, 95 mg, 100 mg, 105 mg, 110 mg, 115 mg, 120 mg, 125 mg, 130 mg, 135 mg, 140 mg, 145 mg, or 150 mg, daily.

In some embodiments of the single antiplatelet therapy, the aspirin is administered in an amount of 75-100 mg daily, such as, for example, one of: 75 mg, 80 mg, 81 mg, 85 mg, 90 mg, 95 mg, or 100 mg, daily.

In some embodiments of the single antiplatelet therapy, the aspirin is administered in an amount of 75 mg daily.

In some embodiments of the single antiplatelet therapy, the aspirin is administered in an amount of 81 mg daily.

In some embodiments of the single antiplatelet therapy, the aspirin is administered in an amount of 100 mg daily.

In other embodiments, the single antiplatelet therapy is a P2Y12 inhibitor.

In some embodiments of the single antiplatelet therapy, the P2Y12 inhibitor is clopidogrel, ticagrelor, or prasugrel.

In some embodiments of the single antiplatelet therapy, the P2Y12 inhibitor is clopidogrel.

In some embodiments of the single antiplatelet therapy, the clopidogrel is administered in an amount of 75 mg to 600 mg daily, such as, for example, one of: 75 mg, 150 mg, 225 mg, 300 mg, 375 mg, 450 mg, 525 mg, or 600 mg daily.

In some embodiments of the single antiplatelet therapy, the P2Y12 inhibitor is ticagrelor.

In some embodiments of the single antiplatelet therapy, the P2Y12 inhibitor is prasugrel.

In some embodiments, the single antiplatelet therapy is ticlopidine.

In some embodiments, the ticlopidine is administered in amounts an amount of 250-500 mg daily, such as, for example, 150 mg, or 500 mg daily.

In other embodiments of the disclosed methods, the antiplatelet therapy comprises dual antiplatelet therapy.

In some embodiments, the dual antiplatelet therapy is aspirin and ticlopidine.

In some embodiments, the dual antiplatelet therapy is aspirin and a P2Y12 inhibitor.

In some embodiments of the dual antiplatelet therapy, the aspirin is administered in an amount of 50-150 mg daily, such as, for example, one of: 50 mg, 55 mg, 60 mg, 65 mg, 70 mg, 75 mg, 80 mg, 81 mg, 85 mg, 90 mg, 95 mg, 100 mg, 105 mg, 110 mg, 115 mg, 120 mg, 125 mg, 130 mg, 135 mg, 140 mg, 145 mg, or 150 mg, daily.

In some embodiments of the dual antiplatelet therapy, the aspirin is administered in an amount of 75-100 mg daily, such as, for example, one of 75 mg, 80 mg, 81 mg, 85 mg, 90 mg, 95 mg, or 100 mg, daily.

In some embodiments of the dual antiplatelet therapy, the aspirin is administered in an amount of 75 mg daily.

In some embodiments of the dual antiplatelet therapy, the aspirin is administered in an amount of 81 mg daily.

In some embodiments of the dual antiplatelet therapy, the aspirin is administered in an amount of 100 mg daily.

In some embodiments of the dual antiplatelet therapy, the P2Y12 inhibitor is clopidogrel, ticagrelor, or prasugrel.

In some embodiments of the dual antiplatelet therapy, the P2Y12 inhibitor is clopidogrel.

In some embodiments of the dual antiplatelet therapy, the clopidogrel is administered in an amount of 75 mg to 600 mg daily, such as, for example, one of: 75 mg, 150 mg, 225 mg, 300 mg, 375 mg, 450 mg, 525 mg, or 600 mg daily.

In some embodiments of the dual antiplatelet therapy, the clopidogrel is administered as a loading dose.

In some embodiments, the loading dose is between 300-600 mg daily.

In some embodiments of the dual antiplatelet therapy, the clopidogrel is administered in an amount of 75 mg daily.

In some embodiments of the dual antiplatelet therapy, the clopidogrel is administered in an amount of 150 mg daily.

In some embodiments of the dual antiplatelet therapy, the clopidogrel is administered in an amount of 225 mg daily.

In some embodiments of the dual antiplatelet therapy, the clopidogrel is administered in an amount of 300 mg daily.

In some embodiments of the dual antiplatelet therapy, the clopidogrel is administered in an amount of 375 mg daily.

In some embodiments of the dual antiplatelet therapy, the clopidogrel is administered in an amount of 450 mg daily.

In some embodiments of the dual antiplatelet therapy, the clopidogrel is administered in an amount of 525 mg daily.

In some embodiments of the dual antiplatelet therapy, the clopidogrel is administered in an amount of 600 mg daily.

In some aspects, the disclosure is directed to methods for preventing adverse cardiovascular events in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof;
wherein the pharmaceutical composition is administered twice daily; and
wherein the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; and transient ischemic attack (TIA).

In some embodiments of such methods, the administration of the regimen results in a relative risk reduction (RRR) in the occurrence of symptomatic venous thromboembolism (VTE: (pulmonary embolism (PE), deep vein thrombosis (DVT) in the patient of at least 25%, such as, for example, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%, without increased bleeding, versus a placebo group.

In other embodiments of such methods, the adverse cardiovascular event is one or more of CV death, MI, or ischemic stroke.

In some aspects, the disclosure is directed to methods for reducing incidence rate of the one or more of adverse thrombotic event selected from new ischemic stroke, MI, or all-cause death in a human patient diagnosed with acute coronary syndrome, wherein the method comprises administering to the human patient a regimen comprising: (i) a pharmaceutical composition comprising about 25 mg to about 100 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the regimen is administered twice daily.

In some embodiments of such methods, administration of the regimen results in a relative risk is 0.85 or less relative to the placebo, such as, for example, 0.85, 0.84, 0.83, 0.82, 0.81, 0.8, 0.79, 0.78, 0.77, 0.76, 0.75, 0.74, 0.73, 0.72, 0.71, or 0.7, relative to the placebo.

In other aspects, the disclosure is directed to methods for preventing ischemic stroke in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

In some embodiments of such methods, administration of the regimen results in a relative risk reduction (RRR) in the occurrence of clinical ischemic stroke events in the patient of at least 25%, without increased bleeding, versus a placebo group, such as, for example, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%, without increased bleeding, versus a placebo group.

In some embodiments of such methods, the clinical benefit of reducing the incidence rates of the clinical ischemic stroke by the regimen is maintained throughout a treatment period of 90 days.

In some aspects, the disclosure provides methods for preventing one or more of stroke and non-central nervous system (CNS) systemic embolism events in a human patient with a history of atrial fibrillation or flutter, wherein the method comprises administering to the human patient a composition comprising 50 mg, or 100 mg, of milvexian (or a pharmaceutically acceptable salt or solvate thereof, on a milvexian basis); wherein the composition is administered twice daily.

In other aspects, the disclosure provides methods for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a composition comprising 50 mg, or 100 mg, of milvexian (or a pharmaceutically acceptable salt or solvate thereof, on a milvexian basis); wherein the composition is administered twice daily.

In some aspects, the methods of the disclosure are performed on a human patient.

In some embodiments, the human patient is at least 18 years old.

In other embodiments, the human patient is at least 75 years old.

In other embodiments, the human patient is between 64-74 years old, such as, for example, 64 years old, 65 years old, 66 years old, 67 years old, 68 years old, 69 years old, 70 years old, 71 years old, 72 years old, 73 years old, or 74 years old.

In some aspects, the patient has a history of atrial fibrillation or flutter.

In some embodiments, the patient has a history of paroxysmal atrial fibrillation.

In other embodiments, the patient has a history of sustained atrial fibrillation not due to a reversible cause.

In some embodiments, the patient has a history of persistent atrial fibrillation (i.e., AF that lasts longer than seven days, and may require electric shocks to the heart to restore normal rhythm).

In some embodiments, the patient has a history of long-standing persistent atrial fibrillation (i.e., AF that is persistent, but lasts longer than 1 year).

In some embodiments, the patient has a history of permanent/chronic atrial fibrillation (i.e., the patient is always in AF, and all attempts for restoring sinus rhythm have failed).

In some embodiments, the patient has a history of paroxysmal, persistent, or long-standing persistent atrial fibrillation.

In some embodiments, the patient's atrial fibrillation is shown by ECG evidence (eg, 12-lead ECG, rhythm strip, Holter, pacemaker interrogation). In some embodiments, the patient has medical evidence of atrial fibrillation within 1 year before and at least 1 day before the ECG evidence.

In some embodiments, the methods of the disclosure are performed on a patient following electrical cardioversion or ablation.

In some embodiments, the patient has a history of atrial fibrillation.

In other embodiments, the patient has a history of atrial flutter.

In other embodiments, the patient has a history of both atrial fibrillation and atrial flutter.

In some aspects, the methods of the disclosure are directed to preventing one or more of stroke and non-central nervous system (CNS) systemic embolism events.

In some embodiments, the methods of the disclosure prevent stroke.

In some embodiments, the methods of the disclosure prevent non-central nervous system (CNS) systemic embolism events.

In some embodiments, the methods of the disclosure prevent stroke and non-central nervous system (CNS) systemic embolism events.

In some aspects, the methods of the disclosure are directed to preventing major adverse cardiovascular events. In some embodiments, each event is selected from the group consisting of cardiovascular death, myocardial infarction, stroke, and non-central nervous system (CNS) systemic embolism.

In some embodiments, the methods of the disclosure are directed to preventing non-central nervous system (CNS) systemic embolism.

In some embodiments, the methods of the disclosure are directed to preventing non-fatal myocardial infarction.

In other embodiments, the methods of the disclosure are directed to preventing non-fatal stroke.

In other embodiments, the methods of the disclosure are directed to preventing cardiovascular death.

In other embodiments, the methods of the disclosure are directed to preventing non-fatal myocardial infarction, non-fatal stroke, and cardiovascular mortality.

In some aspects, the methods of the disclosure are directed to preventing one or more of all-cause death, myocardial infarction, stroke, and non-CNS systemic embolism.

In some embodiments, the methods of the disclosure are directed to preventing all-cause death.

In some embodiments, the methods of the disclosure are directed to preventing myocardial infarction.

In some embodiments, the methods of the disclosure are directed to preventing stroke.

In some embodiments, the methods of the disclosure are directed to preventing non-CNS systemic embolism.

In some aspects, the methods of the disclosure are directed to preventing cardiovascular death, myocardial infarction, stroke, unanticipated revascularization (including amputation for ischemic limb), or urgent hospitalization for vascular cause of ischemic nature (including DVT and PE) in a human patient with a history of atrial fibrillation or flutter.

In some embodiments, the methods of the disclosure are directed to preventing cardiovascular death in a human patient with atrial fibrillation or flutter.

In some embodiments, the methods of the disclosure are directed to preventing myocardial infarction in a human patient with atrial fibrillation or flutter.

In some embodiments, the methods of the disclosure are directed to preventing stroke in a human patient with atrial fibrillation or flutter.

In some embodiments, the methods of the disclosure are directed to preventing a non-CNS embolism in a human patient with atrial fibrillation or flutter.

In some embodiments, the methods of the disclosure are directed to preventing unanticipated revascularization (including amputation for ischemic limb) in a human patient with atrial fibrillation or flutter.

In some embodiments, the methods of the disclosure are directed to preventing urgent hospitalization for vascular cause of ischemic nature (including DVT and PE) in a human patient with atrial fibrillation or flutter.

In some embodiments, the methods of the disclosure are directed to preventing a condition that is stroke or non-CNS systemic embolism in a human patient with atrial fibrillation or flutter.

In some embodiments, the methods of the disclosure are directed to preventing cardiovascular death, myocardial infarction, stroke, or non-CNS embolism in a human patient with atrial fibrillation or flutter.

In some embodiments, the methods of the disclosure are directed to preventing a condition that is all-cause death, myocardial infarction, stroke, or non-CNS embolism in a human patient with atrial fibrillation or flutter.

In some embodiments, the methods of the disclosure are directed to preventing a condition that is cardiovascular death, myocardial infarction, stroke, any unanticipated revascularization (including amputation for ischemic limb), or urgent hospitalization for vascular cause of ischemic nature (including thrombotic events: deep vein thrombosis (DVT) and pulmonary embolism [PE]) in a human patient with atrial fibrillation or flutter.

In some aspects of the methods of the disclosure, the patient is administered a pharmaceutical composition comprising 50 mg, or 100 mg, of milvexian (or a pharmaceutically acceptable salt or solvate thereof, on a milvexian basis), wherein the composition is administered twice daily.

In embodiments of the disclosed methods in which the pharmaceutical composition comprises a solvate or a pharmaceutically acceptable salt of milvexian, then the amount specified is on a milvexian basis. That is, an amount of the solvate or the pharmaceutically acceptable salt in the pharmaceutical composition contains the specified amount of milvexian. For example, a pharmaceutical composition comprising 100 mg of milvexian (or pharmaceutically acceptable salt or solvate thereof) refers to a pharmaceutical composition comprising either 100 mg of milvexian, or an amount of a pharmaceutically acceptable salt or solvate of milvexian equivalent to 100 mg of milvexian.

In some embodiments the patient is administered a pharmaceutical composition comprising 50 mg of milvexian, wherein the composition is administered twice daily.

In some embodiments the patient is administered a pharmaceutical composition comprising 50 mg (on a milvexian basis) of a pharmaceutically acceptable salt or solvate of milvexian, wherein the composition is administered twice daily.

In some embodiments the patient is administered a pharmaceutical composition comprising 50 mg (on a milvexian basis) of a pharmaceutically acceptable salt of milvexian, wherein the composition is administered twice daily.

In some embodiments, the patient is administered a pharmaceutical composition comprising 50 mg (on a milvexian basis) of a pharmaceutically acceptable solvate of milvexian, wherein the composition is administered twice daily.

In some embodiments the patient is administered a pharmaceutical composition comprising 100 mg of milvexian, wherein the composition is administered twice daily.

In some embodiments the patient is administered a pharmaceutical composition comprising 100 mg (on a milvexian basis) of a pharmaceutically acceptable salt or solvate of milvexian, wherein the composition is administered twice daily.

In some embodiments the patient is administered a pharmaceutical composition comprising 100 mg (on a milvexian basis) of a pharmaceutically acceptable salt of milvexian, wherein the composition is administered twice daily.

In some embodiments the patient is administered a pharmaceutical composition comprising 100 mg (on a milvexian basis) of a pharmaceutically acceptable solvate of milvexian, wherein the composition is administered twice daily.

In some embodiments of the methods of the disclosure in which the human patient is administered the pharmaceutical composition comprising milvexian (or a pharmaceutically acceptable salt or solvate thereof, on a milvexian basis) the first administration is made within 7 days of an acute coronary syndrome.

In some aspects of the methods of the disclosure in which the human patient is administered the pharmaceutical composition comprising milvexian (or a pharmaceutically acceptable salt or solvate thereof, on a milvexian basis) twice daily, one of the two administrations is made in the morning, and the other of the two administrations is made in the evening.

In some embodiments, each of the morning administration and the evening administration is made at the same time each day.

In some aspects of the methods of the disclosure, the pharmaceutical composition comprising milvexian (or a pharmaceutically acceptable salt or solvate thereof, on a milvexian basis) is administered twice daily for at least 13 weeks.

In some embodiments, the pharmaceutical composition comprising milvexian (or a pharmaceutically acceptable salt or solvate thereof, on a milvexian basis) is administered twice daily for 13 weeks.

In other embodiments, the pharmaceutical composition comprising milvexian (or a pharmaceutically acceptable salt or solvate thereof, on a milvexian basis) is administered twice daily for more than 13 weeks.

In other embodiments, the pharmaceutical composition comprising milvexian (or a pharmaceutically acceptable salt or solvate thereof, on a milvexian basis) is administered twice daily for at least 26 weeks, at least 52 weeks, at least 78 weeks, at least 104 weeks, at least 130 weeks, at least 156 weeks, at least 182 weeks, or at least 208 weeks.

In some embodiments, the pharmaceutical composition comprising milvexian (or a pharmaceutically acceptable salt or solvate thereof, on a milvexian basis) is administered twice daily chronically, i.e., indefinitely.

In some aspects of the methods of the disclosure, the human patient is administered a pharmaceutical composition comprising 12.5 mg to 200 mg of milvexian (or a pharmaceutically acceptable salt or solvate thereof, on a milvexian basis) twice daily.

In some embodiments the patient is administered a pharmaceutical composition comprising 12.5 mg to 200 mg of milvexian, wherein the composition is administered twice daily.

In some embodiments the patient is administered a pharmaceutical composition comprising 12.5 mg to 200 mg (on a milvexian basis) of a pharmaceutically acceptable salt or solvate of milvexian, wherein the composition is administered twice daily.

In some embodiments the patient is administered a pharmaceutical composition comprising 12.5 mg to 200 mg (on a milvexian basis) of a pharmaceutically acceptable salt of milvexian, wherein the composition is administered twice daily.

In some embodiments the patient is administered a pharmaceutical composition comprising 12.5 mg to 200 mg (on a milvexian basis) of a pharmaceutically acceptable solvate of milvexian, wherein the composition is administered twice daily.

In some embodiments the patient is administered a pharmaceutical composition comprising 25 mg of milvexian, wherein the composition is administered twice daily.

In some embodiments the patient is administered a pharmaceutical composition comprising 25 mg (on a milvexian basis) of a pharmaceutically acceptable salt or solvate of milvexian, wherein the composition is administered twice daily.

In some embodiments the patient is administered a pharmaceutical composition comprising 25 mg (on a milvexian basis) of a pharmaceutically acceptable salt of milvexian, wherein the composition is administered twice daily.

In some embodiments the patient is administered a pharmaceutical composition comprising 25 mg (on a milvexian basis) of a pharmaceutically acceptable solvate of milvexian, wherein the composition is administered twice daily.

In some aspects, the disclosure provides methods of preventing one or more of stroke and non-central nervous system (CNS) systemic embolism events in a patient with a history of atrial fibrillation or flutter, wherein the method comprises administering to the patient an immediate release film coated tablet comprising 50 mg or 100 mg of milvexian (or a pharmaceutically acceptable salt thereof, on a milvexian basis) and one or more pharmaceutically acceptable excipients, wherein the immediate release film-coated tablet is administered twice a day with or without a meal, wherein the patient is at least 18 years old; wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of: (i) age between 65 and 74 years; (ii) hypertension; (iii) diabetes mellitus; (iv) history of coronary artery disease (CAD) (including post myocardial infarction or acute coronary syndrome event with or without percutaneous coronary intervention (PCI)) or peripheral artery disease (PAD); and (v) history of heart failure.

In other aspects, the disclosure provides methods of preventing one or more major adverse cardiovascular events in a patient with a history of atrial fibrillation or flutter, wherein each event is selected from the group consisting of cardiovascular death, myocardial infarction, stroke, and non-central nervous system (CNS) systemic embolism, said method comprising administering to the patient an immediate release film-coated tablet comprising 50 mg or 100 mg of milvexian, or a pharmaceutically acceptable salt thereof, and one or more pharmaceutically acceptable excipient, wherein the immediate release film-coated tablet is administered twice a day with or without a meal, wherein the patient is at least 18 years old; wherein the patient further has one or more of Category (A) risk factor selected from (i) age greater than or equal to 75 years or (ii) history of ischemic stroke; or two or more of concomitant or comorbid Category (B) cardiovascular risk factor selected from the group consisting of: (i) age between 65 and 74 years; (ii) hypertension; (iii) diabetes mellitus; (iv) history of coronary artery disease (CAD) (including post myocardial infarction or acute coronary syndrome event with or without percutaneous coronary intervention (PCI)) or peripheral artery disease (PAD); and (v) history of heart failure.

In some embodiments of such methods, the major adverse cardiovascular event is cardiovascular death.

In some embodiments of the disclosed methods, the patient's baseline FXI clotting activity is measured. As used herein, the term “baseline FXI clotting activity” refers to the patient's FXI clotting activity before being administered the regimen comprising a pharmaceutical composition comprising milvexian (or pharmaceutically acceptable salt or solvate thereof).

In some embodiments, Factor XI (FXI) activity is measured using a modification of the activated partial thromboplastin time (aPTT) using Actin FS (Siemens Healthcare) on the Siemens BCS®XP analyzer. A 6-point calibration curve (~5-150%) is prepared using a secondary calibrator (Standard Human Plasma, Siemens Healthcare Diagnostics Inc.) with a known concentration of human FXI assigned by the manufacturer. The reference standard, at approximately 100%, is diluted by the BCS®XP analyzer in saline to generate pre-selected calibration levels of FXI. The calibration curve is plotted with FXI activity in percent (%) on the x-axis and clotting time in seconds on the y-axis. A log/lin regression curve fit is used. The samples to be tested are mixed with FXI deficient plasma (containing less than 1% FXI and at least 75% of all the other factors) to normalize all other factors. APTT reagent (Actin FS) is added and the mixture is incubated. Following incubation, calcium chloride is added to the mixture and the time to clot formation (measured optically) is compared to the time on the calibration curve. Samples are tested at the base dilution (1:10) prepared by the BCS®XP in saline.

In some embodiments of the methods of the disclosure, administration of the immediate release tablet comprising milvexian (or a pharmaceutically acceptable salt of solvate thereof), or a regimen comprising the immediate release tablet, reduces the patient's FXI clotting activity by about 7% to about 70% relative to baseline.

In other aspects, in the methods of the disclosure, the administration reduces the patient's FXI clotting activity by about 7% to about 20% relative to baseline.

In some embodiments, in the methods of the disclosure, the administration reduces the patient's FXI clotting activity by about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, or about 68%, about 69%, or about 70%, relative to baseline.

In other embodiments, in the methods of the disclosure, the administration reduces the patient's FXI clotting activity by about 7%, about 8%, about 9%, about 10%, about 11%, about 12%, about 13%, about 14%, about 15%, about 16%, about 17%, about 18%, about 19%, or about 20%, relative to baseline.

In other embodiments, in the methods of the disclosure, the administration reduces the patient's FXI clotting activity by an amount shown in the Examples herein.

In some aspects of the disclosed methods, administration of the immediate release tablet comprising milvexian (or a pharmaceutically acceptable salt of solvate thereof), or a regimen comprising the immediate release tablet, results is a prolongation of activated partial thromboplastin time (aPTT) ranging from about 27% to about 64% relative to baseline, or results in a prolongation of activated partial thromboplastin time (aPTT) in the patient with a ratio to baseline of 2.1 to 2.6.

In some aspects of the disclosed methods, the administration results in a prolongation of activated partial thromboplastin time (aPTT) ranging from about 27% to about 64% relative to baseline.

In some embodiments of the disclosed methods, the administration results in a prolongation of activated partial thromboplastin time (aPTT) ranging from about 27% to about 64%, such as, for example, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, or about 64%, relative to baseline. As used here “baseline” refers to the patient's aPTT prior to any administration of the regimen.

In other embodiments, the administration results in a prolongation of activated partial thromboplastin time (aPTT) in the patient with a ratio to baseline of 2.1 to 2.6., such as, for example, 2.1, 2.2, 2.3, 2.4, 2.5, or 2.6.

In some embodiments of the disclosed methods, the administration results in a prolongation of activated partial thromboplastin time (aPTT) in an amount shown in the Examples herein.

In some embodiments, aPTT is determined as follows: Plasma samples are incubated with Actin FS aPTT assay reagent containing a standard amount of phospholipid and contact activator (ellagic acid) which activates the intrinsic coagulation pathway. After incubating for 3 minutes, calcium chloride is added to initiate coagulation and formation of a fibrin clot is measured optically. The time to clot formation (measured in seconds) is reported as the Activated Partial Thromboplastin Time (aPTT).

In some aspects of the methods of the disclosure, the pharmaceutical composition comprising milvexian (or pharmaceutically acceptable salt or solvate thereof) is a solid oral pharmaceutical composition.

In some embodiments of the methods of the disclosure, the solid oral pharmaceutical composition comprises a spray-dried amorphous solid dispersion (SDP) consisting essentially of milvexian free form and a pH-dependent enterosoluble polymer.

In some embodiments of the methods of the disclosure, the SDP comprises the milvexian free form and the pH-dependent enterosoluble polymer in a weight ratio of 3:1 (milvexian: polymer).

In some embodiments of the methods of the disclosure, the pH-dependent enterosoluble polymer is cellulose acetate trimellitate (CAT), cellulose acetate phthalate (CAP), Hydroxypropyl methylcellulose phthalate (HPMCP), hydroxypropyl methylcellulose ES grade (HPMC ES), hydroxypropyl methyl cellulose acetate succinate (HPMC-AS) LF, LG, MF, MG or HF Grades such as Aqoat®, Polyvinyl acetate phthalate (PVAP) such as Sureteric® and Opadry® and Shellac resins such as SSB® Aquagold, or polyvinylpyrrolidone (PVP).

In some embodiments of the methods of the disclosure, the pH-dependent enterosoluble polymer is hydroxypropyl methyl cellulose-AS MG.

In some embodiments of the methods of the disclosure, the solid oral pharmaceutical composition further comprises a binder, such as, for example, microcrystalline cellulose (MCC), silicified microcrystalline cellulose (SMCC), or a combination thereof.

In some embodiments of the methods of the disclosure, the solid oral pharmaceutical composition further comprises a filler, such as, for example, lactose monohydrate.

In some embodiments of the methods of the disclosure, the solid oral pharmaceutical composition further comprises a disintegrant.

In some embodiments of the methods of the disclosure, the solid oral pharmaceutical composition further comprises a lubricant.

In some embodiments of the methods of the disclosure, the solid oral pharmaceutical composition is a tablet.

In some embodiments of the methods of the disclosure, the solid oral pharmaceutical composition is an immediate release tablet.

In some embodiments of the methods of the disclosure, the solid oral pharmaceutical composition is a direct compression tablet.

In some embodiments of the methods of the disclosure, the solid oral pharmaceutical composition is a roller compaction tablet.

In some embodiments of the methods of the disclosure, the solid oral pharmaceutical composition is a film-coated tablet.

In some embodiments of the methods of the disclosure, the film coating comprises polyvinyl alcohol, titanium dioxide, polyethylene glycol-polyvinyl alcohol graft copolymer, and talc.

In some embodiments of the methods of the disclosure, the film coating comprises polyethylene glycol-polyvinyl alcohol graft copolymer.

In some embodiments, the film coating comprises polyvinyl alcohol, iron oxide, macrogol (PEG) polyvinyl alcohol grafted copolymer, and talc.

In some embodiments, the film coating comprises OpadryQX 321A220063 Yellow, a film coating material that comprises polyvinyl alcohol, iron oxide, macrogol (PEG) polyvinyl alcohol grafted copolymer, and talc.

In some embodiments, the solid oral pharmaceutical composition is a tablet having the composition and/or properties shown in Table A:

TABLE A
Film-coated Tablet of Milvexian at 100 mg or 25 mg Strength

| | Amount | | |
|---|---|---|---|
| | | Amount | Amount |
| | | per dose | per dose |
| Component | wt. % | (25 mg) | (100 mg) |
| SDP^{b} | 22.22 | 33.33 | 133.3 |
| Silicified Microcrystalline cellulose | 43.07 | 64.6 | 258.4 |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Core Tablet Weight | 100 | 150 | 600 |
| OpadryQX 321A220063 Yellow^{c} | 3.0 | 4.5 | 18.0 |
| | | 154.5 | 618 |
| Tablet hardness | | Average 60N to | Average 140N to |
| | | 120N (Ph. | 220N (Ph. |
| | | Eur. 2.9.8), | Eur. 2.9.8), |
| | | preferably 90N | preferably 180N |
| Tablet friability | | <0.5% | <0.5% |
| Disintegration time in water | | 13 | 18 |
| (seconds) | | | |

^{b}SDP: spray-dried amorphous solid dispersion prepared according to the composition and method described in WO 2020210629. SDP consists essentially of milvexian free form and hypromellose acetate succinate (HPMCAS-MG) in a weight ratio of 3:1 (milvexian:HPMCAS-MG).
^{c}OpadryQX 321A220063 Yellow: a film coating material comprises polyvinyl alcohol, iron oxide, macrogol (PEG) polyvinyl alcohol grafted copolymer, and talc.

As used herein, the term “disintegration time” refers to the time required for the tablet to break into particles under a given set of conditions. In some embodiments, the disintegration time is determined using the apparatus described in Eur. Ph. (PTZ-E Pharma Test, Hainburg, Germany), in distilled water at 37° C. using disks.

In some embodiments in which the solid pharmaceutical composition is a tablet, the tablet has a disintegration time in water of less than 60 seconds at 37° C.

In some embodiments in which the solid pharmaceutical composition is a tablet, the tablet has a disintegration time in water of less than 20 seconds at 37° C.

In other embodiments in which the solid pharmaceutical composition is a tablet, the tablet has a disintegration time in water of less than 15 seconds at 37° C.

In other embodiments in which the solid pharmaceutical composition is a tablet, the tablet has a disintegration time in water of less than 10 seconds at 37° C.

In some embodiments of the methods of the disclosure, the solid pharmaceutical composition is a capsule.

In some aspects of the methods of the disclosure, the human patient to whom the pharmaceutical composition comprising milvexian (or a pharmaceutically acceptable salt or solvate thereof) is administered, is unable to swallow a tablet dosage form.

In some embodiments of the disclosed methods, the solid oral pharmaceutical composition is dispersed in an aqueous medium to form an aqueous dispersion for administration.

In some embodiments of the disclosed methods, the solid oral pharmaceutical composition is a tablet which is dispersed in an aqueous medium to form an aqueous dispersion for administration.

In some embodiments of the disclosed methods, the solid oral pharmaceutical composition is a tablet which is administered orally as an aqueous dispersion by dispersing the tablet in an aqueous medium in less than 1 minute at 37° C.

In those embodiments in which the solid oral pharmaceutical composition is dispersed in an aqueous medium to form an aqueous dispersion, the aqueous medium is water, saline, phosphate buffer, vegetable juice, or a fruit juice, including, for example, apple sauce.

In those embodiments in which the solid oral pharmaceutical composition is a tablet which is dispersed in an aqueous medium to form an aqueous dispersion, the aqueous medium is water, saline, phosphate buffer, vegetable juice, or a fruit juice, including, for example, apple sauce.

In some embodiments of the disclosed methods in which the solid oral pharmaceutical composition is dispersed in an aqueous medium to form an aqueous dispersion for administration, the aqueous dispersion is administered to the human patient via a NG tube or spoon.

In some embodiments of the disclosed methods in which the solid oral pharmaceutical composition is a tablet which is dispersed in an aqueous medium to form an aqueous dispersion for administration, the aqueous dispersion is administered to the human patient via a NG tube or spoon.

In some embodiments of the disclosed methods, oral administration of the pharmaceutical composition comprising milvexian (or a pharmaceutically acceptable salt or solvate thereof) results in a milvexian plasma half-life ranging from about 13 hours to about 16 hours. Methods of determining plasma half-life in human patients are known to those of skill in the art.

In some embodiments of the disclosed methods, administration of the pharmaceutical composition comprising milvexian (or a pharmaceutically acceptable salt or solvate thereof) to the human patient results in milvexian plasma concentrations reaching steady-state at about 3 days to 6 days. As used herein, the term “steady-state” refers to steady-state plasma concentrations as defined by regulatory agencies such as the U.S. Food & Drug Administration (FDA) and the European Medicines Agency (EMA).

In some embodiments, milvexian has a terminal plasma half-life (T_{1/2}) ranging from about 13 hours to about 16 hours.

In some embodiments of the disclosed methods, administration of milvexian 25 mg BID to the human patient results in milvexian plasma concentrations reaching steady-state in about 3 days.

In some embodiments of the disclosed methods, administration of milvexian 100 mg BID to the human patient results in milvexian plasma concentrations reaching steady-state in about 4 days.

In some embodiments of the disclosed methods, administration of milvexian 100 mg BID to the human patient results in milvexian plasma concentrations reaching steady-state in about 5 days.

In some embodiments of the disclosed methods, administration of milvexian 100 mg BID to the human patient results in milvexian plasma concentrations reaching steady-state in about 6 days.

In some embodiments, milvexian has a terminal plasma half-life (t_{1/2}) ranging from about 13 hours to about 16 hours.

In some embodiments, administration of milvexian 100 mg BID as monotherapy to the human patient results in milvexian plasma concentrations reaching steady-state characterized by one or more of the following: (i) a steady state Cₘₐₓ ranging from about 1194 ng/mL to about 2326 ng/mL, (ii) a steady state mean (std) Cₘₐₓ of 1760 (566) ng/ml, (iii) a steady state AUC₀₋₂₄ ranging from about 23300 ng*h/mL to about 49100 ng*h/mL, or (iv) a steady state mean (std) AUC₀₋₂₄ of 36200 (12900) ng*h/mL.

In other embodiments, the steady state after the administration of milvexian 100 mg BID as monotherapy to the human patient is characterized by Cₘₐₓ ranging from about 1194 ng/mL to about 2326 ng/mL.

In other embodiments, the steady state after the administration of milvexian 100 mg BID as monotherapy to the human patient is characterized by mean (std) Cₘₐₓ of 1760 (566) ng/mL.

In other embodiments, the steady state after the administration of milvexian 100 mg BID as monotherapy to the human patient is characterized by AUC₀₋₂₄ ranging from about 23300 ng*h/mL to about 49100 ng*h/mL.

In other embodiments, the steady state after the administration of milvexian 100 mg BID as monotherapy to the human patient is characterized by mean (std) AUC of 36200 (12900) ng*h/mL.

In some embodiments, administration of milvexian 100 mg BID on top of standard care antiplatelet therapy to the human patient results in milvexian plasma concentrations reaching steady-state characterized by one or more of the following: (i) a steady state Cₘₐₓ ranging from about 1134 ng/mL to about 2206 ng/mL, (ii) a steady state Cₘₐₓ at mean (std) of 1670 (536) ng/ml, (iii) a steady state AUC₀₋₂₄ ranging from about 22900 ng*h/mL to about 47100 ng*h/mL, or (iv) a steady state AUC₀₋₂₄ at mean (std) of 35000 (12100) ng*h/mL.

In other embodiments, the steady state after the administration of milvexian 100 mg BID on top of standard care antiplatelet therapy to the human patient is characterized by Cₘₐₓ ranging from about 1134 ng/ml to about 2206 ng/mL.

In other embodiments, the steady state after the administration of milvexian 100 mg BID on top of standard care antiplatelet therapy to the human patient is characterized by mean (std) Cₘₐₓ of 1670 (536) ng/mL.

In other embodiments, the steady state after the administration of milvexian 100 mg BID on top of standard care antiplatelet therapy to the human patient is characterized by AUC₀₋₂₄ ranging from about 22900 ng*h/mL to about 47100 ng*h/mL

In other embodiments, the steady state after the administration of milvexian 100 mg BID on top of standard care antiplatelet therapy to the human patient is characterized by mean (std) AUC of 35000 (12100) ng*h/mL.

In some embodiments, administration of milvexian 25 mg BID as monotherapy to the human patient results in milvexian plasma concentrations reaching steady-state characterized by one or more of the following: (i) a steady state Cₘₐₓ ranging from about 283 ng/mL to about 525 ng/ml, (ii) a steady state mean (std) Cₘₐₓ of 394 (131) ng/mL, (iii) a steady state AUC₀₋₂₄ ranging from about 5230 ng*h/mL to about 10650 ng*h/mL, or (iv) a steady state mean (std) AUC₀₋₂₄ of 7940 (2710) ng*h/mL.

In other embodiments, the steady state after the administration of milvexian 25 mg BID as monotherapy to the human patient is characterized by Cₘₐₓ ranging from about 283 ng/ml to about 525 ng/ml.

In other embodiments, the steady state after the administration of milvexian 25 mg BID as monotherapy to the human patient is characterized by mean (std) Cₘₐₓ of 394 (131) ng/mL.

In other embodiments, the steady state after the administration of milvexian 25 mg BID as monotherapy to the human patient is characterized by AUC₀₋₂₄ ranging from about 5230 ng*h/mL to about 10650 ng*h/mL.

In other embodiments, the steady state after the administration of milvexian 25 mg BID as monotherapy to the human patient is characterized by mean (std) AUC of 7940 (2710) ng*h/mL.

In some embodiments, administration of milvexian 25 mg BID on top of standard care antiplatelet therapy to the human patient results in milvexian plasma concentrations reaching steady-state characterized by one or more of the following: (i) a steady state Cₘₐₓ ranging from about 217 ng/mL to about 475 ng/ml, (ii) a steady state mean (std) Cₘₐₓ of 346 (129) ng/ml, (iii) a steady state AUC₀₋₂₄ ranging from about 4350 ng*h/mL to about 10230 ng*h/mL, or (iv) a steady state mean (std) AUC₀₋₂₄ of 7290 (2940) ng*h/mL.

In other embodiments, the steady state after the administration of milvexian 25 mg BID on top of standard care antiplatelet therapy to the human patient is characterized by Cₘₐₓ ranging from about 217 ng/ml to about 475 ng/ml.

In other embodiments, the steady state after the administration of milvexian 25 mg BID on top of standard care antiplatelet therapy to the human patient is characterized by mean (std) Cₘₐₓ of 346 (129) ng/mL.

In other embodiments, the steady state after the administration of milvexian 25 mg BID on top of standard care antiplatelet therapy to the human patient is characterized by AUC₀₋₂₄ ranging from about 4350 ng*h/mL to about 10230 ng*h/mL.

In other embodiments, the steady state after the administration of milvexian 100 mg BID to the human patient is characterized by mean (std) AUC of 7290 (2940) ng*h/mL.

In some embodiments of the disclosed methods, administration of the pharmaceutical composition comprising milvexian (or a pharmaceutically acceptable salt or solvate thereof) to the human patient results in milvexian plasma concentrations reaching steady-state at about 4 days to 6 days.

In some embodiments of the disclosed methods, administration of the pharmaceutical composition comprising milvexian (or a pharmaceutically acceptable salt or solvate thereof) to the human patient results in milvexian plasma concentrations reaching steady-state at about 4 days to 6 days, and the administration is twice daily.

In some embodiments of the disclosed methods, administration of the pharmaceutical composition comprising milvexian (or a pharmaceutically acceptable salt or solvate thereof) to the human patient results in milvexian plasma concentrations reaching steady-state in about 3 days.

In some embodiments of the disclosed methods, administration of the pharmaceutical composition comprising milvexian (or a pharmaceutically acceptable salt or solvate thereof) to the human patient results in milvexian plasma concentrations reaching steady-state in about 4 days.

In some embodiments of the disclosed methods, administration of the pharmaceutical composition comprising milvexian (or a pharmaceutically acceptable salt or solvate thereof) to the human patient results in milvexian plasma concentrations reaching steady-state in about 5 days.

In some embodiments of the disclosed methods, administration of the pharmaceutical composition comprising milvexian (or a pharmaceutically acceptable salt or solvate thereof) to the human patient results in milvexian plasma concentrations reaching steady-state in about 6 days.

In some embodiments of the disclosed methods, administration of the pharmaceutical composition comprising milvexian (or a pharmaceutically acceptable salt or solvate thereof) to the human patient results in milvexian plasma concentrations reaching steady-state in about 4 days, and the administration is twice daily.

In some embodiments of the disclosed methods, administration of the pharmaceutical composition comprising milvexian (or a pharmaceutically acceptable salt or solvate thereof) to the human patient results in milvexian plasma concentrations reaching steady-state in about 5 days, and the administration is twice daily.

In some embodiments of the disclosed methods, administration of the pharmaceutical composition comprising milvexian (or a pharmaceutically acceptable salt or solvate thereof) to the human patient results in milvexian plasma concentrations reaching steady-state in about 6 days, and the administration is twice daily.

In some embodiments of the disclosed methods, the pharmaceutical composition comprising milvexian (or a pharmaceutically acceptable salt or solvate thereof) is administered without regard to the timing of food intake.

In some aspects of the disclosed methods, the administration does not result in a statistically significant increase in major bleeding complications. In these aspects, “statistically significant increase” refers to an increase from the patient's baseline major bleeding, i.e., the patient's major bleeding before being administered the pharmaceutical composition comprising milvexian (or a pharmaceutically acceptable salt or solvate thereof).

In some embodiments of the methods of the disclosure, the administration does not result in a statistically significant increase in major bleeding complications as assessed under ISTH criteria.

In some embodiments of the methods of the disclosure, the administration does not result in a statistically significant increase in major bleeding complications as assessed by CRNM bleeding criteria.

In some embodiments of the methods of the disclosure, the administration does not result in a statistically significant increase in major bleeding complications as assessed by ISTH CRNM bleeding criteria.

In some embodiments of the methods of the disclosure, the administration does not result in a statistically significant increase in major bleeding complications as assessed by ISTH major or CRNM bleeding criteria.

In some embodiments of the methods of the disclosure, the administration does not result in a statistically significant increase in major bleeding complications as assessed by GUSTO, BARC, or TIMI criteria.

In some embodiments of the methods of the disclosure, the administration does not result in a statistically significant increase in major bleeding complications as assessed by BARC 3c and 5 categories.

In some embodiments of the methods of the disclosure, the administration does not result in a statistically significant increase in major bleeding complications as assessed by BARC 3b, 3c, and 5 categories.

In some embodiments of the methods of the disclosure, the administration does not result in a statistically significant increase in major bleeding complications as assessed by BARC 3b category.

In some embodiments of the methods of the disclosure, the administration does not result in a statistically significant increase in major bleeding complications as assessed by BARC 3c category.

In some embodiments of the methods of the disclosure, the administration does not result in a statistically significant increase in major bleeding complications as assessed by BARC 5 category.

In some embodiments of the methods of the disclosure, the administration does not result in a statistically significant increase in GUSTO severe or life-threatening bleeding.

In some embodiments of the methods of the disclosure, the administration does not result in a statistically significant increase in non-CABG TIMI major bleeding.

In some embodiments of the methods of the disclosure, the administration does not result in a statistically significant increase in clinically relevant nonmajor bleeding.

In some embodiments of the methods of the disclosure, the administration does not result in a statistically significant increase in clinically relevant nonmajor bleeding as assessed by BARC 2, BARC 3a, or BARC 4.

In some embodiments of the methods of the disclosure, the administration does not result in a statistically significant increase in clinically relevant nonmajor bleeding as assessed by ISTH nonmajor clinically relevant bleeding.

In some embodiments of the methods of the disclosure, the administration does not result in a statistically significant increase in clinically relevant nonmajor bleeding as assessed by GUSTO moderate bleeding.

In some embodiments of the methods of the disclosure, the administration does not result in a statistically significant increase in clinically relevant nonmajor bleeding as assessed by TIMI CABG-related major bleeding.

In some embodiments of the methods of the disclosure, the administration does not result in a statistically significant increase in clinically relevant nonmajor bleeding as assessed by TIMI minor bleeding.

In some embodiments of the methods of the disclosure, the administration does not result in a statistically significant increase in clinically relevant nonmajor bleeding as assessed by TIMI bleeding requiring medical attention.

In some embodiments of the methods of the disclosure, the administration does not result in a statistically significant increase in clinically relevant nonmajor bleeding as assessed by TIMI major bleeding.

In some embodiments of the methods of the disclosure, the administration does not result in a statistically significant increase in clinically relevant nonmajor bleeding as assessed by TIMI major bleeding or TIMI minor bleeding.

In some embodiments of the methods of the disclosure, the Relative Risk of bleeding according to the Bleeding Academic Research Consortium (BARC) Type 3 or 5 criteria by the regimen is no greater than 3, such as, for example, no greater than one of: 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with antiplatelet therapy.

In some embodiments of the methods of the disclosure, the Relative Risk of bleeding according to the Bleeding Academic Research Consortium (BARC) Type 3 or 5 criteria by the regimen is no greater than 3, such as, for example, no greater than one of: 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with antiplatelet therapy.

In some embodiments of the disclosed methods, the Relative Risk of serious bleeding according to the Bleeding Academic Research Consortium (BARC) Type 3 or 5 criteria is independent of the amount of milvexian administered.

In other embodiments of the disclosed methods, the human patient does not suffer serious bleeding according to the Bleeding Academic Research Consortium (BARC) Type 3 and 5 criteria.

In some embodiments of the methods of the disclosure, the Relative Risk of bleeding according to the Bleeding Academic Research Consortium (BARC) Type 2 criteria by the regimen is no greater than 2.6, such as, for example, no greater than one of: 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with standard of care.

In some embodiments of the methods of the disclosure, the Relative Risk of bleeding according to the Bleeding Academic Research Consortium (BARC) Type 2 criteria by the regimen is no greater than 2.6, such as, for example, no greater than one of: 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with antiplatelet therapy.

In some embodiments of the disclosed methods, the Relative Risk of bleeding according to the Bleeding Academic Research Consortium (BARC) Type 2 criteria is independent of the amount of milvexian administered.

In other embodiments of the disclosed methods, the human patient does not suffer bleeding according to the Bleeding Academic Research Consortium (BARC) Type 2.

In some embodiments of the methods of the disclosure, the Relative Risk of bleeding according to the ISTH criteria (major bleeding or clinically-relevant non-major bleeding (CRNM)) by the regimen is no greater than 3, such as, for example, no greater than one of 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with standard of care.

In some embodiments of the methods of the disclosure, the Relative Risk of bleeding according to the ISTH criteria (major bleeding or clinically-relevant non-major bleeding (CRNM)) by the regimen is no greater than 3, such as, for example, no greater than one of 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with antiplatelet therapy.

Administration of milvexian according to the disclosure is generally safe and well tolerated. Administration of milvexian, at a dose of 25 mg, 50 mg, or 100 mg results in no clinically significant QTc interval prolongation. For example, administration of milvexian, at a dose of 25 mg, 50 mg, or 100 mg does not result in a ΔΔQTc of 10 msec or longer. In some aspects, administration of milvexian, at a dose of 25 mg, 50 mg, or 100 mg results in a ΔΔQTc of less than 10 msec. In some aspects, administration of milvexian, at a dose of 25 mg, 50 mg, or 100 mg results in ΔΔQTc of less than 9 msec. In some aspects, administration of milvexian, at a dose of 25 mg, 50 mg, or 100 mg results in a ΔΔQTc of less than 8 msec. In some aspects, administration of milvexian, at a dose of 25 mg, 50 mg, or 100 mg results in a ΔΔQTc in QTc of less than 7 msec. In some aspects, administration of milvexian, at a dose of 25 mg, 50 mg, or 100 mg results in a ΔΔQTc of less than 6 msec. In some aspects, administration of milvexian, at a dose of 25 mg, 50 mg, or 100 mg results in a ΔΔQTc of less than 5 msec. In some aspects, administration of milvexian, at a dose of 25 mg, 50 mg, or 100 mg results in a ΔΔQTc of less than 4 msec.

Administration of milvexian, at a dose of 25 mg results in no clinically significant QTc interval prolongation. In some aspects, administration of milvexian, at a dose of 25 mg, results in a ΔΔQTc of less than 10 msec. In some aspects, administration of milvexian, at a dose of 25 mg, results in a ΔΔQTc of less than 9 msec. In some aspects, administration of milvexian, at a dose of 25 mg, results in a ΔΔQTc of less than 8 msec. In some aspects, administration of milvexian, at a dose of 25 mg, results in a ΔΔQTc of less than 7 msec. In some aspects, administration of milvexian, at a dose of 25 mg, results in a ΔΔQTc of less than 6 msec. In some aspects, administration of milvexian, at a dose of 25 mg, results in a ΔΔQTc of less than 5 msec. In some aspects, administration of milvexian, at a dose of 25 mg, results in a ΔΔQTc of less than 4 msec.

Administration of milvexian, at a dose of 50 mg, results in no clinically significant QTc interval prolongation. In some aspects, administration of milvexian, at a dose of 50 mg, results in a ΔΔQTc of less than 10 msec. In some aspects, administration of milvexian, at a dose of 50 mg, results in a ΔΔQTc of less than 9 msec. In some aspects, administration of milvexian, at a dose of 50 mg, results in a ΔΔQTc of less than 8 msec. In some aspects, administration of milvexian, at a dose of 50 mg, results in a ΔΔQTc of less than 7 msec. In some aspects, administration of milvexian, at a dose of 50 mg, results in a ΔΔQTc of less than 6 msec. In some aspects, administration of milvexian, at a dose of 50 mg, results in a ΔΔQTc of less than 5 msec. In some aspects, administration of milvexian, at a dose of 50 mg, results in a ΔΔQTc of less than 4 msec.

Administration of milvexian, at a dose of 100 mg, results in no clinically significant QTc interval prolongation. In some aspects, administration of milvexian, at a dose of 100 mg, results in a ΔΔQTc of less than 10 msec. In some aspects, administration of milvexian, at a dose of 100 mg, results in a ΔΔQTc of less than 9 msec. In some aspects, administration of milvexian, at a dose of 100 mg, results in a ΔΔQTc of less than 8 msec. In some aspects, administration of milvexian, at a dose of 100 mg, results in a ΔΔQTc of less than 7 msec. In some aspects, administration of milvexian, at a dose of 100 mg, results in a ΔΔQTc of less than 6 msec. In some aspects, administration of milvexian, at a dose of 100 mg, results in a ΔΔQTc of less than 5 msec. In some aspects, administration of milvexian, at a dose of 100 mg, results in a ΔΔQTc of less than 4 msec.

The disclosure is directed to a method of preventing stroke and non-CNS systemic embolism in an adult patient with atrial fibrillation (AF), comprising administering to the patient 100 mg BID milvexian. The disclosure is directed to methods of preventing stroke and non-CNS systemic embolism in adult patients with atrial fibrillation (AF), comprising administering 100 mg BID milvexian.

The disclosure is directed to a method of preventing a thrombotic event in an adult patient, after an acute coronary syndrome (ACS), comprising administering 25 mg BID milvexian in combination with antiplatelet therapy. The disclosure is directed to methods of preventing thrombotic events in adult patients, after an acute coronary syndrome (ACS), comprising administering 25 mg BID milvexian in combination with antiplatelet therapy.

Those skilled in the art will recognize, or be able to ascertain using no more than routine experimentation, many equivalents to the embodiments described herein.

All publications, patents and patent applications mentioned in this specification are herein incorporated by reference into the specification to the same extent as if each individual publication, patent or patent application was specifically and individually indicated to be incorporated herein by reference.

### Aspects

It will be understood that references herein to methods of using milvexian or compositions comprising milvexian for treating or preventing the conditions of the disclosure should also be interpreted as references to: (i) the milvexian or compositions comprising milvexian for use in methods of treating or preventing the conditions of the disclosure; and/or (ii) the use of milvexian or compositions comprising milvexian in the manufacture of a medicament for treating or preventing the conditions of the disclosure.

### Aspect Set #1

Aspect 1. Milvexian for use in treating or preventing a thrombotic condition in a human patient with a cardiovascular or cerebrovascular disease, wherein the use comprises administering to the human patient an immediate release tablet comprising 25 mg, 50 mg, or 100 mg of milvexian (or a pharmaceutically acceptable salt of solvate thereof), and a pharmaceutically acceptable excipient, optionally wherein the immediate release tablet is administered together with an antiplatelet therapy, wherein the immediate release tablet is administered twice daily.

Aspect 2. The milvexian for use of aspect 1, wherein administration of the immediate release tablet comprising milvexian (or a pharmaceutically acceptable salt of solvate thereof), or a regimen comprising the immediate release tablet, reduces the patient's FXI clotting activity by about 7% to about 20% relative to baseline, or by about 27% to 64% relative to baseline.

Aspect 3. The milvexian for use of Aspect 1 or aspect 2, wherein administration of the immediate release tablet comprising milvexian (or a pharmaceutically acceptable salt of solvate thereof), or a regimen comprising the immediate release tablet, results is a prolongation of activated partial thromboplastin time (aPTT) ranging from about 27% to about 64% relative to baseline, or results in a prolongation of activated partial thromboplastin time (aPTT) in the patient with a ratio to baseline of 2.1 to 2.6.

Aspect 4. The milvexian for use of any one of the preceding aspects, wherein the administration does not result in a statistically significant increase in major bleeding complications.

Aspect 5. The milvexian for use of any one of the preceding aspects, wherein the immediate release tablet has a disintegration time in water of less than 20 seconds at 37° C.

Aspect 6. The milvexian for use of any one of the preceding aspects, wherein the administration results in a milvexian plasma half-life ranging from about 13 hours to about 16 hours.

Aspect 7. The milvexian for use of any one of the preceding aspects, wherein the administration results in milvexian plasma concentration reaching steady-state at about 3 days to 6 days.

Aspect 8. The milvexian for use of any one of the preceding aspects, wherein the administration results in milvexian plasma concentration reaching steady-state at about 4 days to 6 days.

Aspect 9. The milvexian for use of any one of the preceding aspects, wherein the immediate release tablet is administered without regard to the timing of food intake.

Aspect 10. The milvexian for use of any one of the preceding aspects, wherein the thrombotic condition is a thromboembolic disorder selected from an arterial thromboembolic disorder; a venous thromboembolic disorder; or a thromboembolic disorder in the chambers of the heart or in the peripheral circulation.

Aspect 11. The milvexian for use of any one of the preceding aspects, wherein the thromboembolic disorder is unstable angina, an acute coronary syndrome, atrial fibrillation, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, or thrombosis resulting from medical implants, devices, or procedures in which blood is exposed to an artificial surface that promotes thrombosis.

Aspect 12. The milvexian for use of aspect 10, wherein the thrombotic condition is an arterial thromboembolic disorder.

Aspect 13. The milvexian for use of aspect 12, wherein the arterial thromboembolic disorder is selected from coronary arterial thrombosis, cerebral arterial thrombosis, arterial embolism, cerebral embolism, acute ischemic stroke, transient ischemic attack (TIA), myocardial infarction, stroke, acute coronary syndrome, atherosclerosis, peripheral occlusive arterial disease, cardiovascular death, or combinations thereof.

Aspect 14. The milvexian for use of aspect 10, wherein the thrombotic condition is a venous thromboembolic disorder.

Aspect 15. The milvexian for use of aspect 14, wherein the venous thromboembolic disorder is selected from deep vein thromboembolism, venous thromboembolism, pulmonary embolism, death, or combinations thereof.

Aspect 16. The milvexian for use of any one of aspects 1-9, wherein the thrombotic condition is stroke and/or non-central nervous system (CNS) systemic embolism.

Aspect 17. The milvexian for use of any one of the preceding aspects, wherein the cardiovascular human patient has a cerebrovascular disease.

Aspect 18. The milvexian for use of aspect 17, wherein the cerebrovascular disease is selected from non-cardioembolic ischemic stroke, or transient ischemic attack (TIA).

Aspect 19. The milvexian for use of any one of the preceding aspects, wherein the human patient has a cardiovascular disease.

Aspect 20. The milvexian for use of aspect 19, wherein the cardiovascular disease is atrial fibrillation or flutter.

Aspect 21. The milvexian for use of aspect 19, wherein the cardiovascular disease is acute coronary syndrome.

Aspect 22. The milvexian for use of aspect 11, wherein the thrombotic condition is thrombosis resulting from medical implants, devices, or procedures in which blood is exposed to an artificial surface that promotes thrombosis.

Aspect 23. The milvexian for use of any one of the preceding claims, wherein the immediate release tablet comprises 50 mg, or 100 mg, of milvexian (or a pharmaceutically acceptable salt or solvate thereof).

Aspect 24. The milvexian for use of aspect 10, wherein the thrombotic condition comprises arterial thromboembolic disorder associated with an acute coronary syndrome.

Aspect 25. The milvexian for use of any one of aspects 1-9, wherein the method comprises administering to the human patient a regimen comprising: (i) an immediate release tablet comprising 25 mg of milvexian (or pharmaceutically acceptable salt or solvate thereof); and (ii) an antiplatelet therapy selected from the group consisting of aspirin, a P2Y12 inhibitor, and combination thereof; wherein the immediate release tablet is administered twice daily.

Aspect 26. The milvexian for use of aspect 25, wherein the antiplatelet therapy is a P2Y12 inhibitor.

Aspect 27. The milvexian for use of aspect 26, wherein the P2Y12 inhibitor is clopidogrel, ticagrelor, or prasugrel.

Aspect 28. The nilvexian for use of any one the preceding aspects, wherein the antiplatelet therapy is aspirin.

Aspect 29. The milvexian for use of any one of the preceding aspects, wherein the human patient is treated with aspirin and clopidogrel combination therapy for at least 90 days.

Aspect 30. The milvexian for use of aspect 29, wherein the patient is treated with aspirin and/or clopidogrel without milvexian dose adjustment.

Aspect 31. The milvexian for use of any one of the preceding aspects, wherein the human patient is unable to swallow a tablet dosage form.

Aspect 32. The milvexian for use of any one of the preceding aspects, wherein the immediate release tablet is dispersed in an aqueous medium to form an aqueous dispersion.

Aspect 33. The milvexian for use of aspect 32, wherein the aqueous medium is water, saline, phosphate buffer, vegetable juice, or fruit juice, including, for example, apple sauce.

Aspect 34. The milvexian for use of Aspect 32 or aspect 33, wherein the aqueous dispersion is administered to the human patient via a nasogastric (NG) tube or spoon.

Aspect 35. The milvexian for use of any one of aspects 32-34, wherein the immediate release tablet is administered orally as an aqueous dispersion by dispersing the immediate release tablet in an aqueous medium in less than 1 minute at 37° C.

Aspect 36. The milvexian for use of any one of the preceding aspects, wherein the milvexian administration results in no significant QTc interval prolongation.

### Aspect Set #2

Aspect 1. A method for preventing one or more of stroke and non-central nervous system (CNS) systemic embolism events in a human patient with atrial fibrillation, wherein the method comprises administering to the human patient a pharmaceutical composition comprising about 100 mg of milvexian (or a pharmaceutically acceptable salt or solvate thereof, on a milvexian basis); wherein the pharmaceutical composition is administered twice daily.

Aspect 2. A method for preventing one or more of major adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising about about 100 mg of milvexian (or a pharmaceutically acceptable salt or solvate thereof, on a milvexian basis); wherein the composition is administered twice daily.

Aspect 3. The method of aspect 2, wherein the major adverse cardiovascular event is cardiovascular death.

Aspect 4. The method of any one of aspects 1-3, wherein the patient has atrial fibrillation, and also has concomitant coronary artery disease (CAD) and/or peripheral artery disease (PAD).

Aspect 5. The method of any one of aspects 1-4, wherein the pharmaceutical composition is a solid oral pharmaceutical composition comprising milvexian (or a pharmaceutically acceptable salt or solvate thereof) and one or more pharmaceutically acceptable excipients.

Aspect 6. The method of aspect 5, wherein the solid oral pharmaceutical composition is an immediate release tablet.

Aspect 7. The method of aspect 6, wherein the immediate release tablet has a disintegration time in water of less than 20 seconds at 37° C.

Aspect 8. The method of any one of aspects 5-7, wherein the administration results in a milvexian plasma half-life ranging from about 13 hours to about 16 hours.

Aspect 9. The method of any one of aspects 5-7, wherein the administration results in a milvexian plasma half-life ranging from about 13 hours to about 16 hours during repeat dosing.

Aspect 10. The method of any one of the preceding aspects, wherein the administration results in milvexian plasma concentrations reaching steady-state at about 4 days to 6 days.

Aspect 11. The method of any one of the preceding aspects, wherein the pharmaceutical composition comprising milvexian (or a pharmaceutically acceptable salt or solvate thereof) is administered without regard to the timing of food intake.

Aspect 12. The method of any one of the preceding aspects, wherein the composition comprising milvexian (or a pharmaceutically acceptable salt or solvate thereof) is administered twice daily for at least 13 weeks.

Aspect 13. The method of any one of the preceding aspects, wherein the composition comprising milvexian (or a pharmaceutically acceptable salt or solvate thereof) is administered once daily in the morning and once daily in the evening.

Aspect 14. The method of any one of the preceding aspects, wherein the morning administration is made at the same time each day and the evening administration is made at the same time each day.

Aspect 15. A method of preventing one or more of stroke and non-central nervous system (CNS) systemic embolism events in a patient with atrial fibrillation, wherein the method comprising administering to the patient an immediate release film coated tablet comprising about 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 16. A method of preventing one or more major adverse cardiovascular events in a patient with atrial fibrillation, wherein each event is selected from the group consisting of cardiovascular death, myocardial infarction, stroke, and non-central nervous system (CNS) systemic embolism said method comprising administering to the patient an immediate release film-coated tablet comprising 100 mg of milvexian, or a pharmaceutically acceptable salt thereof, and one or more pharmaceutically acceptable excipient,
wherein the immediate release film-coated tablet is administered twice a day with or without a meal,
wherein the patient is at least 18 years old;
wherein the patient further has one or more of Category (A) risk factor selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 17. The method of aspect 16, wherein the major adverse cardiovascular event is stroke and non-central nervous system (CNS) systemic embolism.

Aspect 18. The method of any one of aspects 15-17, wherein the immediate release film-coated tablet has a disintegration time in water of less than 20 seconds at 37° C.

Aspect 19. The method of any one of aspects 15-18, wherein the administered milvexian has a plasma half-life of 13-16 hours.

Aspect 20. The method of any one of aspects 15-18, wherein the administered milvexian has a plasma half-life of 13-16 hours during repeat dosing.

Aspect 21. The method of any one of aspects 15-20, wherein the composition comprising milvexian (or a pharmaceutically acceptable salt or solvate thereof) is administered twice daily for at least 13 weeks.

Aspect 22. The method of any one of aspects 15-21, wherein the composition comprising milvexian (or a pharmaceutically acceptable salt or solvate thereof) is administered once daily in the morning and once daily in the evening.

Aspect 23. The method of aspect 22, wherein the morning administration is made at the same time each day and the evening administration is made at the same time each day.

Aspect 24. The method of any one of the preceding aspects, wherein the administration reduces FXI clotting activity in the patient by about 27% to about 64% relative to the baseline FXI clotting activity.

Aspect 25. The method of any one of the preceding aspects, wherein the administration results in a prolongation of activated partial thromboplastin time (aPTT) in the patient with a ratio to baseline of 2.1 to 2.6.

Aspect 26. A method for preventing stroke in a human patient with atrial fibrillation, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.

Aspect 27. A method for preventing one or more non-central nervous system (CNS) systemic embolism in a human patient with atrial fibrillation, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.

Aspect 28. A method for preventing one or more of adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian (or a pharmaceutically acceptable salt or solvate thereof, on a milvexian basis); wherein the composition is administered twice daily.

Aspect 29. The method of aspect 28, wherein the major adverse cardiovascular event is cardiovascular death.

Aspect 30. The method of aspect 28, wherein the major adverse cardiovascular event is myocardial infarction.

Aspect 31. The method of aspect 28, wherein the major adverse cardiovascular event is stroke.

Aspect 32. The method of aspect 28, wherein the major adverse cardiovascular event is non-central nervous system (CNS) systemic embolism.

Aspect 33. The method of any one of aspects 26-32, wherein the patient has atrial fibrillation, and also has concomitant coronary artery disease (CAD) and/or peripheral artery disease (PAD).

Aspect 34. The method of aspect 33, wherein the patient has atrial fibrillation, and also has concomitant coronary artery disease (CAD).

Aspect 35. The method of aspect 33, wherein the patient has atrial fibrillation, and also has concomitant peripheral artery disease (PAD).

Aspect 36. The method of any one of aspects 26-35, wherein the pharmaceutical composition is a solid oral pharmaceutical composition comprising milvexian (or a pharmaceutically acceptable salt or solvate thereof) and one or more pharmaceutically acceptable excipients.

Aspect 37. The method of aspect 36, wherein the solid oral pharmaceutical composition is an immediate release tablet.

Aspect 38. The method of aspect 37, wherein the immediate release tablet has a disintegration time in water of less than 20 seconds at 37° C.

Aspect 39. The method of any one of aspects 26-38, wherein the administration results in a milvexian plasma half-life ranging from about 13 hours to about 16 hours.

Aspect 40. The method of any one of aspects 26-39, wherein the administration results in a milvexian plasma half-life ranging from about 13 hours to about 16 hours during repeat dosing.

Aspect 41. The method of any one of aspects 26-40, wherein the administration results in milvexian plasma concentrations reaching steady-state at about 4 days to 6 days.

Aspect 42. The method of any one of aspects 26-41, wherein the pharmaceutical composition comprising milvexian (or a pharmaceutically acceptable salt or solvate thereof) is administered without regard to the timing of food intake.

Aspect 43. The method of any one aspects 26-42, wherein the patient is unable to swallow a tablet dosage form.

Aspect 44. The method of any one aspects 26-43, wherein the pharmaceutical composition is dispersed in an aqueous medium to form an aqueous dispersion.

Aspect 45. The method of aspect 44, wherein the aqueous medium is water, saline, phosphate buffer, vegetable juice, or fruit juice, including, for example, apple sauce.

Aspect 46. The method of aspect 44 or aspect 45, wherein the aqueous dispersion is administered to the human patient via a nasogastric tube or spoon.

Aspect 47. The method of any one of aspects 44-46, wherein the milvexian is administered orally as an aqueous dispersion by dispersing the pharmaceutical composition in an aqueous medium in less than 60 seconds at 37° C.

Aspect 48. The method of any one of any one of aspects 26-47, wherein the composition comprising milvexian (or a pharmaceutically acceptable salt or solvate thereof) is administered twice daily for at least 13 weeks.

Aspect 49. The method of any one of any one of aspects 26-48, wherein the composition comprising milvexian (or a pharmaceutically acceptable salt or solvate thereof) is administered once in the morning and once in the evening.

Aspect 50. The method of aspect 49, wherein the morning administration is made at the same time each day and the evening administration is made at the same time each day.

Aspect 51. The method of any one of aspects 26-50, wherein the administration reduces FXI clotting activity in the patient by about 27% to about 64% relative to the baseline FXI clotting activity.

Aspect 52. The method of any one of any one of aspects 26-51, wherein the administration results in a prolongation of activated partial thromboplastin time (aPTT) in the patient with a ratio to baseline of 2.1 to 2.6.

Aspect 53. A method of preventing stroke in a patient with atrial fibrillation, wherein the method comprising administering to the patient an immediate release film coated tablet comprising about 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 54. A method of preventing non-central nervous system (CNS) systemic embolism events in a patient with atrial fibrillation, wherein the method comprising administering to the patient an immediate release film coated tablet comprising about 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 55. The method of any one of aspects 53-54, wherein the patient has atrial fibrillation, and also has concomitant coronary artery disease (CAD) and/or peripheral artery disease (PAD).

Aspect 56. The method of aspect 55, wherein the patient has atrial fibrillation, and also has concomitant coronary artery disease (CAD).

Aspect 57. The method of aspect 55, wherein the patient has atrial fibrillation, and also has concomitant peripheral artery disease (PAD).

Aspect 58. The method of any one of aspects 53-57, wherein the immediate-release film-coated tablet has a disintegration time in water of less than 20 seconds at 37° C.

Aspect 59. The method of any one of aspects 53-58, wherein the administration results in a milvexian plasma half-life ranging from about 13 hours to about 16 hours.

Aspect 60. The method of any one of aspects 53-59, wherein the administration results in a milvexian plasma half-life ranging from about 13 hours to about 16 hours during repeat dosing.

Aspect 61. The method of any one of aspects 53-60, wherein the administration results in milvexian plasma concentrations reaching steady-state at about 4 days to 6 days.

Aspect 62. The method of any one of aspects 53-61, wherein the immediate-release film-coated tablet is administered without regard to the timing of food intake.

Aspect 63. The method of any one aspects 53-62, wherein the patient is unable to swallow a tablet dosage form.

Aspect 64. The method of any one aspects 53-63, wherein the immediate-release film-coated tablet is dispersed in an aqueous medium to form an aqueous dispersion.

Aspect 65. The method of aspect 64, wherein the aqueous medium is water, saline, phosphate buffer, vegetable juice, or fruit juice, including, for example, apple sauce.

Aspect 66. The method of aspect 65 or aspect 65, wherein the aqueous dispersion is administered to the human patient via a nasogastric tube or spoon.

Aspect 67. The method of any one of aspects 53-66, wherein the milvexian is administered orally as an aqueous dispersion by dispersing the immediate-release film-coated tablet in an aqueous medium in less than 60 seconds at 37° C.

Aspect 68. The method of any one of any one of aspects 53-67, wherein the immediate-release film-coated tablet is administered twice daily for at least 13 weeks.

Aspect 69. The method of any one of any one of aspects 53-68, wherein the immediate-release film-coated tablet is administered once in the morning and once in the evening.

Aspect 70. The method of aspect 69, wherein the morning administration is made at the same time each day and the evening administration is made at the same time each day.

Aspect 71. The method of any one of aspects 53-70, wherein the administration reduces FXI clotting activity in the patient by about 27% to about 64% relative to the baseline FXI clotting activity.

Aspect 72. The method of any one of any one of aspects 53-71, wherein the administration results in a prolongation of activated partial thromboplastin time (aPTT) in the patient with a ratio to baseline of 2.1 to 2.6.

Aspect 73. The method of any one of the preceding aspects, wherein the method further comprises administering a standard-of-care antiplatelet therapy to the patient.

Aspect 74. The method of aspect 73, wherein, the standard-of-care antiplatelet therapy is selected from aspirin, adenosine diphosphate (ADP) receptor inhibitors; adenosine reuptake inhibitors; glycoprotein platelet inhibitors; phosphodiesterase inhibitors; or protease-activated receptor (PAR-1) antagonist.

Aspect 75. The method of any one of aspects 73 and 74, wherein the standard-of-care antiplatelet therapy is selected from aspirin, clopidogrel, ticagrelor, ticlopidine, prasugrel, dipyridamole, abciximab, eptifibatide, tirofiban, cilostazol, or vorapaxar.

Aspect 76. The method of any one of aspects 73-75, wherein the standard-of-care antiplatelet therapy is aspirin, clopidogrel, ticagrelor, or prasugrel.

Aspect 77. The method of any one of aspects 73-76, wherein the standard-of-care antiplatelet therapy is clopidogrel, ticagrelor, or prasugrel.

Aspect 78. The method of any one of aspects 73-77, wherein the standard-of-care antiplatelet therapy comprises a single antiplatelet therapy (SAPT).

Aspect 79. The method of aspect 78, wherein the single antiplatelet therapy comprises aspirin, or a P2Y12 inhibitor selected from clopidogrel, ticagrelor, and prasugrel.

Aspect 80. The method of any one of aspects 73-77, wherein the standard-of-care antiplatelet therapy comprises a dual antiplatelet therapy (DAPT).

Aspect 81. The method of aspect 80, wherein the dual antiplatelet therapy comprises aspirin and a P2Y12 inhibitor selected from clopidogrel, ticagrelor, or prasugrel.

Aspect 82. The method of any one of aspects 80 and 81, wherein the dual antiplatelet therapy comprises a aspirin and a P2Y12 inhibitor is clopidogrel, ticagrelor, or prasugrel.

Aspect 83. The method of any one of aspects 80-82, wherein the dual antiplatelet therapy comprises aspirin and clopidogrel.

Aspect 84. The method of any one of aspects 80-83, wherein the dual antiplatelet therapy (DAPT) is administered for 21 days, followed by single antiplatelet therapy (SAPT) thereafter.

Aspect 85. The method of any one of aspects 80-83, wherein the dual antiplatelet therapy (i.e., aspirin and P2Y 12 inhibitor) is administered for more than 90 days (with or without de-escalation to SAPT).

Aspect 86. Milvexian for use in preventing one or more of stroke and non-central nervous system (CNS) systemic embolism events in a human patient with atrial fibrillation, wherein the method comprises administering to the human patient a pharmaceutical composition comprising about 100 mg of milvexian (or a pharmaceutically acceptable salt or solvate thereof, on a milvexian basis); wherein the pharmaceutical composition is administered twice daily.

Aspect 87. Milvexian for use in preventing one or more of major adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising 100 mg of milvexian (or a pharmaceutically acceptable salt or solvate thereof, on a milvexian basis); wherein the composition is administered twice daily.

Aspect 88. Milvexian for use of aspect 87, wherein the major adverse cardiovascular event is cardiovascular death.

Aspect 89. Milvexian for use of any one of aspects 86-88, wherein the patient has atrial fibrillation, and also has concomitant coronary artery disease (CAD) and/or peripheral artery disease (PAD).

Aspect 90. Milvexian for use of any one of aspects 86-89, wherein the pharmaceutical composition is a solid oral pharmaceutical composition comprising milvexian (or a pharmaceutically acceptable salt or solvate thereof) and one or more pharmaceutically acceptable excipients.

Aspect 91. Milvexian for use of aspect 90, wherein the solid oral pharmaceutical composition is an immediate release tablet.

Aspect 92. Milvexian for use of aspect 91, wherein the immediate release tablet has a disintegration time in water of less than 20 seconds at 37° C.

Aspect 93. Milvexian for use of any one of aspects 86-92, wherein the administration results in a milvexian plasma half-life ranging from about 13 hours to about 16 hours.

Aspect 94. Milvexian for use of any one of aspects 86-93, wherein the administration results in a milvexian plasma half-life ranging from about 13 hours to about 16 hours during repeat dosing.

Aspect 95. Milvexian for use of any one of aspects 86-94, wherein the administration results in milvexian plasma concentrations reaching steady-state at about 4 days to 6 days.

Aspect 96. Milvexian for use of any one of aspects 86-95, wherein the pharmaceutical composition comprising milvexian (or a pharmaceutically acceptable salt or solvate thereof) is administered without regard to the timing of food intake.

Aspect 97. Milvexian for use of any one of aspects 86-96, wherein the composition comprising milvexian (or a pharmaceutically acceptable salt or solvate thereof) is administered twice daily for at least 13 weeks.

Aspect 98. Milvexian for use of any one of aspects 86-97, wherein the composition comprising milvexian (or a pharmaceutically acceptable salt or solvate thereof) is administered once in the morning and once in the evening.

Aspect 99. Milvexian for use of any one of aspects 86-98, wherein the morning administration is made at the same time each day and the evening administration is made at the same time each day.

Aspect 100. Milvexian for use in preventing one or more of stroke and non-central nervous system (CNS) systemic embolism in a patient with atrial fibrillation, wherein the method comprising administering to the patient an immediate release film coated tablet comprising 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (vi) age between 65 and 74 years;
   (vii) hypertension;
   (viii) diabetes mellitus;
   (ix) history of coronary artery disease (CAD) (including post myocardial infarction or acute coronary syndrome event with or without percutaneous coronary intervention (PCI)) or peripheral artery disease (PAD); and
   (x) history of heart failure.

Aspect 101. Milvexian for use in preventing one or more major adverse cardiovascular events in a patient with atrial fibrillation, wherein each event is selected from the group consisting of cardiovascular death, myocardial infarction, stroke, and non-central nervous system (CNS) systemic embolism said method comprising administering to the patient an immediate release film-coated tablet comprising 100 mg of milvexian, or a pharmaceutically acceptable salt thereof, and one or more pharmaceutically acceptable excipient,
wherein the immediate release film-coated tablet is administered twice a day with or without a meal,
wherein the patient is at least 18 years old;
wherein the patient further has one or more of Category (A) risk factor selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 102. Milvexian for use of aspect 101, wherein the major adverse cardiovascular event is cardiovascular death.

Aspect 103. Milvexian for use of any one of aspects 100-102, wherein immediate release film-coated tablet has a disintegration time in water of less than 20 seconds at 37° C.

Aspect 104. Milvexian for use of any one of aspects 100-103, wherein the administered milvexian has a plasma half-life of 13-16 hours.

Aspect 105. Milvexian for use of any one of aspects 100-104, wherein the administered milvexian has a plasma half-life of 13-16 hours during repeat dosing.

Aspect 106. Milvexian for use of any one of aspects 100-105, wherein the composition comprising milvexian (or a pharmaceutically acceptable salt or solvate thereof) is administered twice daily for at least 13 weeks.

Aspect 107. Milvexian for use of any one of aspects 86-106, wherein the composition comprising milvexian (or a pharmaceutically acceptable salt or solvate thereof) is administered once daily in the morning and once daily in the evening.

Aspect 108. Milvexian for use of aspect 107, wherein the morning administration is made at the same time each day and the evening administration is made at the same time each day.

Aspect 109. Milvexian for use of any one of aspects 86-108, wherein the administration reduces FXI clotting activity in the patient by about 27% to about 64% relative to the baseline FXI clotting activity.

Aspect 110. Milvexian for use of any one of aspects 86-109, wherein the administration results in a prolongation of activated partial thromboplastin time (aPTT) in the patient with a ratio to baseline of 2.1 to 2.6.

Aspect 111. Milvexian for use in preventing stroke in a human patient with atrial fibrillation, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.

Aspect 112. Milvexian for use in preventing one or more non-central nervous system (CNS) systemic embolism in a human patient with atrial fibrillation, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.

Aspect 113. Milvexian for use in preventing one or more of major adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian (or a pharmaceutically acceptable salt or solvate thereof, on a milvexian basis); wherein the composition is administered twice daily.

Aspect 114. Milvexian for use of any one of aspects 111-113, wherein the major adverse cardiovascular event is cardiovascular death.

Aspect 115. Milvexian for use of any one of aspects 111-113, wherein the major adverse cardiovascular event is myocardial infarction.

Aspect 116. Milvexian for use of any one of aspects 111-113, wherein the major adverse cardiovascular event is stroke.

Aspect 117. Milvexian for use of any one of aspects 111-113, wherein the major adverse cardiovascular event is non-central nervous system (CNS) systemic embolism.

Aspect 118. Milvexian for use of any one of aspects 111-117, wherein the patient has atrial fibrillation, and also has concomitant coronary artery disease (CAD) and/or peripheral artery disease (PAD).

Aspect 119. Milvexian for use of aspect 118, wherein the patient has atrial fibrillation, and also has concomitant coronary artery disease (CAD).

Aspect 120. Milvexian for use of aspect 118, wherein the patient has atrial fibrillation, and also has concomitant peripheral artery disease (PAD).

Aspect 121. Milvexian for use of any one of aspects 111-120, wherein the pharmaceutical composition is a solid oral pharmaceutical composition comprising milvexian (or a pharmaceutically acceptable salt or solvate thereof) and one or more pharmaceutically acceptable excipients.

Aspect 122. Milvexian for use of aspect 121, wherein the solid oral pharmaceutical composition is an immediate release tablet.

Aspect 123. Milvexian for use of aspect 122, wherein the immediate release tablet has a disintegration time in water of less than 20 seconds at 37° C.

Aspect 124. Milvexian for use of any one of aspects 111-123, wherein the administration results in a milvexian plasma half-life ranging from about 13 hours to about 16 hours.

Aspect 125. Milvexian for use of any one of aspects 111-124, wherein the administration results in a milvexian plasma half-life ranging from about 13 hours to about 16 hours during repeat dosing.

Aspect 126. Milvexian for use of any one of aspects 111-125, wherein the administration results in milvexian plasma concentrations reaching steady-state at about 4 days to 6 days.

Aspect 127. Milvexian for use of any one of aspects 111-126, wherein the pharmaceutical composition comprising milvexian (or a pharmaceutically acceptable salt or solvate thereof) is administered without regard to the timing of food intake.

Aspect 128. Milvexian for use of any one aspects 111-127, wherein the patient is unable to swallow a tablet dosage form.

Aspect 129. Milvexian for use of any one aspects 111-128, wherein the pharmaceutical composition is dispersed in an aqueous medium to form an aqueous dispersion.

Aspect 130. Milvexian for use of aspect 129, wherein the aqueous medium is water, saline, phosphate buffer, vegetable juice, or fruit juice, including, for example, apple sauce.

Aspect 131. Milvexian for use of aspect 129 or aspect 130, wherein the aqueous dispersion is administered to the human patient via a nasogastric tube or spoon.

Aspect 132. Milvexian for use of any one of aspects 129-131, wherein Milvexian is administered orally as an aqueous dispersion by dispersing the pharmaceutical composition in an aqueous medium in less than 60 seconds at 37° C.

Aspect 133. Milvexian for use of any one of any one of aspects 111-132, wherein the composition comprising milvexian (or a pharmaceutically acceptable salt or solvate thereof) is administered twice daily for at least 13 weeks.

Aspect 134. Milvexian for use of any one of any one of aspects 111-133, wherein the composition comprising milvexian (or a pharmaceutically acceptable salt or solvate thereof) is administered once daily in the morning and once daily in the evening.

Aspect 135. Milvexian for use of aspect 134, wherein the morning administration is made at the same time each day and the evening administration is made at the same time each day.

Aspect 136. Milvexian for use of any one of aspects 111-135, wherein the administration reduces FXI clotting activity in the patient by about 27% to about 64% relative to the baseline FXI clotting activity.

Aspect 137. Milvexian for use of any one of any one of aspects 111-136, wherein the administration results in a prolongation of activated partial thromboplastin time (aPTT) in the patient with a ratio to baseline of 2.1 to 2.6.

Aspect 138. Milvexian for use in preventing stroke in a patient with atrial fibrillation, wherein the method comprising administering to the patient an immediate release film coated tablet comprising about 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 139. Milvexian for use in preventing non-central nervous system (CNS) systemic embolism events in a patient with atrial fibrillation, wherein the method comprising administering to the patient an immediate release film coated tablet comprising about 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 140. Milvexian for use of any one of aspects 138-139, wherein the patient has atrial fibrillation, and also has concomitant coronary artery disease (CAD) and/or peripheral artery disease (PAD).

Aspect 141. Milvexian for use of aspect 140, wherein the patient has atrial fibrillation, and also has concomitant coronary artery disease (CAD).

Aspect 142. Milvexian for use of aspect 140, wherein the patient has atrial fibrillation, and also has concomitant peripheral artery disease (PAD).

Aspect 143. Milvexian for use of any one of aspects 138-142, wherein the immediate-release film-coated tablet has a disintegration time in water of less than 20 seconds.

Aspect 144. Milvexian for use of any one of aspects 138-143, wherein the administration results in a milvexian plasma half-life ranging from about 13 hours to about 16 hours.

Aspect 145. Milvexian for use of any one of aspects 138-144, wherein the administration results in a milvexian plasma half-life ranging from about 13 hours to about 16 hours during repeat dosing.

Aspect 146. Milvexian for use of any one of aspects 138-145, wherein the administration results in milvexian plasma concentrations reaching steady-state at about 4 days to 6 days.

Aspect 147. Milvexian for use of any one of aspects 138-146, wherein the immediate-release film-coated tablet is administered without regard to the timing of food intake.

Aspect 148. Milvexian for use of any one aspects 138-147, wherein the patient is unable to swallow a tablet dosage form.

Aspect 149. Milvexian for use of any one aspects 138-148, wherein the immediate-release film-coated tablet is dispersed in an aqueous medium to form an aqueous dispersion.

Aspect 150. Milvexian for use of aspect 149, wherein the aqueous medium is water, saline, phosphate buffer, vegetable juice, or fruit juice, including, for example, apple sauce.

Aspect 151. Milvexian for use of aspect 149 or aspect 150, wherein the aqueous dispersion is administered to the human patient via a nasogastric tube or spoon.

Aspect 152. Milvexian for use of any one of aspects 138-151, wherein the milvexian is administered orally as an aqueous dispersion by dispersing the immediate-release film-coated tablet in an aqueous medium in less than 60 seconds at 37° C.

Aspect 153. Milvexian for use of any one of any one of aspects 138-152, wherein the immediate-release film-coated tablet is administered twice daily for at least 13 weeks.

Aspect 154. Milvexian for use of any one of any one of aspects 138-153, wherein the immediate-release film-coated tablet is administered once daily in the morning and once daily in the evening.

Aspect 155. Milvexian for use of aspect 154, wherein the morning administration is made at the same time each day and the evening administration is made at the same time each day.

Aspect 156. Milvexian for use of any one of aspects 138-155, wherein the administration reduces FXI clotting activity in the patient by about 27% to about 64% relative to the baseline FXI clotting activity.

Aspect 157. Milvexian for use of any one of any one of aspects 138-156, wherein the administration results in a prolongation of activated partial thromboplastin time (aPTT) in the patient with a ratio to baseline of 2.1 to 2.6.

Aspect 158. Milvexian for use of any one of aspects 86-157, wherein the use further comprises administering a standard-of-care antiplatelet therapy to the patient.

Aspect 159. Milvexian for use of aspect 158, wherein, the standard-of-care antiplatelet therapy is selected from aspirin, adenosine diphosphate (ADP) receptor inhibitors; adenosine reuptake inhibitors; glycoprotein platelet inhibitors; phosphodiesterase inhibitors; or protease-activated receptor (PAR-1) antagonist.

Aspect 160. Milvexian for use of aspects 158 and 159, wherein the standard-of-care antiplatelet therapy is selected from aspirin, clopidogrel, ticagrelor, ticlopidine, prasugrel, dipyridamole, abciximab, eptifibatide, tirofiban, cilostazol, or vorapaxar.

Aspect 161. Milvexian for use of any one of aspects 158-160, wherein the standard-of-care antiplatelet therapy is selected from aspirin, clopidogrel, ticagrelor, or prasugrel.

Aspect 162. Milvexian for use of any one of aspects 158-161, wherein the standard-of-care antiplatelet therapy is selected from clopidogrel, ticagrelor, or prasugrel.

Aspect 163. Milvexian for use of any one of aspects 158-162, wherein the standard-of-care antiplatelet therapy comprises a single antiplatelet therapy (SAPT).

Aspect 164. Milvexian for use of aspect 163, wherein the single antiplatelet therapy comprises aspirin, or a P2Y12 inhibitor selected from clopidogrel, ticagrelor, or prasugrel.

Aspect 165. Milvexian for use of any one of aspects 158-164, wherein the standard-of-care antiplatelet therapy comprises a dual antiplatelet therapy (DAPT).

Aspect 166. Milvexian for use of aspect 165, wherein the dual antiplatelet therapy comprises aspirin and a P2Y12 inhibitor selected from clopidogrel, ticagrelor, or prasugrel.

Aspect 167. Milvexian for use of any one of aspects 165 and 166, wherein the dual antiplatelet therapy comprises aspirin and a P2Y12 inhibitor selected from clopidogrel, ticagrelor, or prasugrel.

Aspect 168. Milvexian for use of any one of aspects 165-167, wherein the dual antiplatelet therapy comprises a aspirin and clopidogrel.

Aspect 169. Milvexian for use of any one of aspects 165-168, wherein the dual antiplatelet therapy (DAPT) is administered for 21 days, followed by single antiplatelet therapy (SAPT) thereafter.

Aspect 170. Milvexian for use of any one of aspects 165-168, wherein the dual antiplatelet therapy (i.e., aspirin and P2Y12 inhibitor) is administered for more than 90 days (with or without de-escalation to SAPT).

Aspect 171. A method for preventing one or more of stroke and non-central nervous system (CNS) systemic embolism in a human patient with atrial fibrillation, wherein the method comprises administering to the human patient a pharmaceutical composition comprising about 100 mg of milvexian, or a pharmaceutically acceptable salt thereof; wherein the pharmaceutical composition is administered twice daily.

Aspect 172. A method for preventing one or more of adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian, or a pharmaceutically acceptable salt thereof; wherein the composition is administered twice daily.

Aspect 173. A method for reducing the risk of one or more of stroke and non-central nervous system (CNS) systemic embolism in a human patient with atrial fibrillation, wherein the method comprises administering to the human patient a pharmaceutical composition comprising about 100 mg of milvexian, or a pharmaceutically acceptable salt thereof; wherein the pharmaceutical composition is administered twice daily.

Aspect 174. A method for reducing the risk of one or more of adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian, or a pharmaceutically acceptable salt thereof; wherein the composition is administered twice daily.

Aspect 175. A method for preventing one or more of stroke and non-central nervous system (CNS) systemic embolism events in a human patient with a history of atrial fibrillation, wherein the method comprises administering to the human patient a pharmaceutical composition comprising 100 mg of milvexian (or a pharmaceutically acceptable salt or solvate thereof, on a milvexian basis); wherein the pharmaceutical composition is administered twice daily.

Aspect 176. A method for preventing one or more of major adverse cardiovascular events in a patient with a history of atrial fibrillation, wherein each event is cardiovascular death, myocardial infarction, stroke, or non-central nervous system (CNS) systemic embolism, wherein the method comprises administering to the human patient a pharmaceutical composition comprising 100 mg of milvexian (or a pharmaceutically acceptable salt or solvate thereof, on a milvexian basis); wherein the composition is administered twice daily.

Aspect 177. The method of aspect 176, wherein the major adverse cardiovascular event is cardiovascular death.

Aspect 178. The method of any one of aspects 171-177, wherein the patient has a history of atrial fibrillation, and also has concomitant coronary artery disease (CAD) and/or peripheral artery disease (PAD).

Aspect 179. The method of any one of aspects 171-178, wherein the pharmaceutical composition comprises 100 mg of milvexian (or a pharmaceutically acceptable salt or solvate thereof, on a milvexian basis).

Aspect 180. The method of any one of aspects 171-179, wherein the pharmaceutical composition is a solid oral pharmaceutical composition comprising milvexian (or a pharmaceutically acceptable salt or solvate thereof) and one or more pharmaceutically acceptable excipients.

Aspect 181. The method of aspect 180, wherein the solid oral pharmaceutical composition is an immediate release tablet.

Aspect 182. The method of aspect 181, wherein the immediate release tablet has a disintegration time in water of less than 20 seconds at 37° C.

Aspect 183. The method of any one of aspects 180-182, wherein the administration results in a milvexian plasma half-life ranging from about 13 hours to about 16 hours.

Aspect 184. The method of any one of aspects 171-183, wherein the administration results in milvexian plasma concentrations reaching steady-state at about 4 days to 6 days.

Aspect 185. The method of any one of aspects 171-184, wherein the pharmaceutical composition comprising milvexian (or a pharmaceutically acceptable salt or solvate thereof) is administered without regard to the timing of food intake.

Aspect 186. A method of preventing one or more of stroke and non-central nervous system (CNS) systemic embolism events in a patient with a history of atrial fibrillation, wherein the method comprises administering to the patient an immediate release film coated tablet comprising 100 mg of milvexian or a pharmaceutically acceptable salt thereof and one or more pharmaceutically acceptable excipients, wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke or silent brain infarct;
or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 187. A method of preventing one or more adverse cardiovascular events in a patient with a history of atrial fibrillation, wherein each event is selected from the group consisting of cardiovascular death, myocardial infarction, stroke, and non-central nervous system (CNS) systemic embolism said method comprising administering to the patient an immediate release film-coated tablet comprising 100 mg of milvexian, or a pharmaceutically acceptable salt thereof, and one or more pharmaceutically acceptable excipient,
wherein the immediate release film-coated tablet is administered twice a day with or without a meal,
wherein the patient is at least 18 years old;
wherein the patient further has one or more of Category (A) risk factor selected from (i) age greater than or equal to 75 years or (ii) history of ischemic stroke or silent brain infarct; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 188. The method of aspect 187, wherein the adverse cardiovascular event is cardiovascular death.

Aspect 189. The method of any one of aspects 186-188, wherein immediate release film-coated tablet has a disintegration time in water of less than 20 seconds.

Aspect 190. The method of any one of aspects 186-189, wherein the administered milvexian has a plasma half-life of 13-16 hours.

Aspect 191. The method of any one of aspects 171-190, wherein the administration reduces FXI clotting activity in the patient by about 27% to about 64% relative to the baseline FXI clotting activity.

Aspect 192. The method of any one of aspects 171-191, wherein the administration results in a prolongation of activated partial thromboplastin time (aPTT) in the patient with a ratio to baseline of 2.1 to 2.6.

Aspect 193. The method of any one of aspects 171-192, wherein the composition comprising milvexian (or a pharmaceutically acceptable salt or solvate thereof) is administered twice daily for at least 13 weeks.

Aspect 194. The method of any one of aspects 171-193, wherein the composition comprising milvexian (or a pharmaceutically acceptable salt or solvate thereof) is administered once daily in the morning and once daily in the evening.

Aspect 195. The method of aspect 194, wherein the morning administration is made at the same time each day and the evening administration is made at the same time each day.

Aspect 196. Milvexian for use in a method of preventing one or more of stroke and non-central nervous system (CNS) systemic embolism events in a human patient with a history of atrial fibrillation, wherein the method comprises administering to the human patient a pharmaceutical composition comprising 100 mg, of milvexian (or a pharmaceutically acceptable salt or solvate thereof, on a milvexian basis); wherein the pharmaceutical composition is administered twice daily.

Aspect 197. Milvexian for use in a method for preventing one or more of adverse cardiovascular events in a patient with a history of atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising 100 mg, of milvexian (or a pharmaceutically acceptable salt or solvate thereof, on a milvexian basis); wherein the composition is administered twice daily.

Aspect 198. Milvexian for use of aspect 197, wherein the adverse cardiovascular event is cardiovascular death.

Aspect 199. Milvexian for use of any one of aspects 196-198, wherein the patient has a history of atrial fibrillation, and also has concomitant coronary artery disease (CAD) and/or peripheral artery disease (PAD).

Aspect 200. Milvexian for use of any one of aspects 196-199, wherein the pharmaceutical composition comprises 100 mg of milvexian (or a pharmaceutically acceptable salt or solvate thereof, on a milvexian basis).

Aspect 201. Milvexian for use of any one of aspects 196-200, wherein the pharmaceutical composition is a solid oral pharmaceutical composition comprising milvexian (or a pharmaceutically acceptable salt or solvate thereof) and one or more pharmaceutically acceptable excipients.

Aspect 202. Milvexian for use of aspect 201, wherein the solid oral pharmaceutical composition is an immediate release tablet.

Aspect 203. Milvexian for use of aspect 202, wherein the immediate release tablet has a disintegration time in water of less than 20 seconds at 37° C.

Aspect 204. Milvexian for use of any one of aspects 201-203, wherein the administration results in a milvexian plasma half-life ranging from about 13 hours to about 16 hours.

Aspect 205. Milvexian for use of any one of aspects 196-204, wherein the administration results in milvexian plasma concentrations reaching steady-state at about 6 days.

Aspect 206. Milvexian for use of any one of aspects 196-205, wherein the pharmaceutical composition comprising milvexian (or a pharmaceutically acceptable salt or solvate thereof) is administered without regard to the timing of food intake.

Aspect 207. Mivexian for use in a method of preventing one or more of stroke and non-central nervous system (CNS) systemic embolism events in a patient with a history of atrial fibrillation, wherein the method comprising administering to the patient an immediate release film coated tablet comprising 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke or silent brain infarct; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 208. Milvexian for use in a method of preventing one or more adverse cardiovascular events in a patient with a history of atrial fibrillation, wherein each event is cardiovascular death, myocardial infarction, stroke, or non-central nervous system (CNS) systemic embolism, said method comprising administering to the patient an immediate release film-coated tablet comprising 100 mg of milvexian, or a pharmaceutically acceptable salt thereof, and one or more pharmaceutically acceptable excipient,
wherein the immediate release film-coated tablet is administered twice a day with or without a meal,
wherein the patient is at least 18 years old;
wherein the patient further has one or more of Category (A) risk factor selected from (i) age greater than or equal to 75 years or (ii) history of ischemic stroke or silent brain infarct; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 209. Milvexian for use of aspect 208, wherein the adverse cardiovascular event is cardiovascular death.

Aspect 210. Milvexian for use of any one of aspects 207-209, wherein immediate release film-coated tablet has a disintegration time in water of less than 20 seconds.

Aspect 211. Milvexian for use of any one of aspects 207-210, wherein the administered milvexian has a plasma half-life of 13-16 hours.

Aspect 212. Milvexian for use of any one of aspects 196-211, wherein the administration reduces FXI clotting activity in the patient by about 27% to about 64% relative to the baseline FXI clotting activity.

Aspect 213. Milvexian for use of any one of aspects 196-212, wherein the administration results in a prolongation of activated partial thromboplastin time (aPTT) in the patient with a ratio to baseline of 2.1 to 2.6.

Aspect 214. Milvexian for use of any one of aspects 196-213, wherein the composition comprising milvexian (or a pharmaceutically acceptable salt or solvate thereof) is administered twice daily for at least 13 weeks.

Aspect 215. Milvexian for use of any one of aspects 196-214, wherein the composition comprising milvexian (or a pharmaceutically acceptable salt or solvate thereof) is administered once in the morning and once in the evening.

Aspect 216. Milvexian for use of aspect 215, wherein the morning administration is made at the same time each day and the evening administration is made at the same time each day.

Aspect 217. The method of any one of the preceding aspects, wherein the milvexian administration results in no clinically significant QTc interval prolongation.

Aspect 218. Milvexian for use of any one of the preceding aspects, wherein the milvexian administration results in no clinically significant QTc interval prolongation.

Aspect 219. Milvexian for use of any one of the preceding aspects, wherein the milvexian administration results in no clinically meaningful change to prothrombin time, with a maximal mean percent change from baseline of approximately 5%.

Aspect 220. Milvexian for use of any one of the preceding aspects, wherein the administration results in no clinically relevant effects on any bleeding caused by milvexian exposure as measured by steady state AUC or Cₘₐₓ.

Aspect 221. Milvexian for use of any one of the preceding aspects, wherein the administration results in minimal impairment of hemostasis in the human patient.

Aspect 222. The method of any one of the preceding aspects, wherein the milvexian administration results in no clinically meaningful change to prothrombin time, with a maximal mean percent change from baseline of approximately 5%.

Aspect 223. The method of any one of the preceding aspects, wherein the administration results in no clinically relevant effects on any bleeding caused by milvexian exposure as measured by steady state AUC or Cₘₐₓ.

Aspect 224. The method of any one of the preceding aspects, wherein the administration results in minimal impairment of hemostasis in the human patient.

Aspect 225. The method of any one of aspects 171-195, wherein the regimen comprises an immediate release tablet.

Aspect 226. The method of aspect 225, wherein the immediate release tablet has a disintegration time in water of less than 20 seconds at 37° C.

Aspect 227. The method of any one of aspects 171-195, wherein the administration results in a milvexian plasma terminal half-life ranging from about 13 hours to about 16 hours.

Aspect 228. The method of any one of aspects 171-195, wherein the regimen is administered without regard to the timing of food intake.

Aspect 229. The method of any one of aspects 171-195, wherein the immediate release tablet is dispersed in an aqueous medium to form an aqueous dispersion.

Aspect 230. The method of aspect 229, wherein the aqueous medium comprises water or apple sauce.

Aspect 231. A method of preventing stroke or non-central nervous system (CNS) systemic embolism in a human patient with atrial fibrillation, wherein the method comprises orally administering to the human patient a regimen comprising about 100 mg of milvexian twice daily; wherein the method achieves at least one of the following clinical outcomes: (1) reaches a steady state plasma concentration profile of milvexian in about 6 days; (2) reaches a steady state plasma concentration profile of milvexian characterized by: (i) a steady state Cₘₐₓ ranging from about 1194 ng/ml to about 2326 ng/ml, (ii) a steady state Cₘₐₓ mean (std) of 1760 (566) ng/ml, (iii) a steady state AUC₀₋₂₄ ranging from about 23300 ng*h/mL to about 49100 ng*h/mL, or (iv) a steady state AUC₀₋₂₄ mean (std) of 36200 (12900) ng*h/mL; (3) no clinically significant QTc interval prolongation, (4) no clinically relevant effects on any bleeding caused by milvexian exposure as measured by steady state AUC or Cₘₐₓ; (5) minimal impairment of hemostasis in the human patient, (6) no clinically meaningful change to prothrombin time, with a maximal mean percent change from baseline of approximately 5%.; or (7) any combination of the aforementioned clinical outcomes.

Aspect 232. A method for preventing one or more of stroke, or non-central nervous system (CNS) systemic embolism in a patient with atrial fibrillation, wherein the method comprises orally administering to the human patient a regimen comprising about 100 mg of milvexian twice daily.

Aspect 233. A method for preventing one or more of cardiovascular death, myocardial infarction, stroke, or non-central nervous system (CNS) systemic embolism in a patient with atrial fibrillation, wherein the method comprises orally administering to the human patient a regimen comprising about 100 mg of milvexian twice daily.

Aspect 234. A method for preventing stroke in a human patient with atrial fibrillation, wherein the method comprises orally administering to the human patient a regimen comprising about 100 mg of milvexian twice daily.

Aspect 235. A method for preventing ischemic stroke in a human patient with atrial fibrillation, wherein the method comprises orally administering to the human patient a regimen comprising about 100 mg of milvexian twice daily.

Aspect 236. A method for preventing non-central nervous system (CNS) systemic embolism in a human patient with atrial fibrillation, wherein the method comprises orally administering to the human patient a regimen comprising 100 mg of milvexian twice daily.

Aspect 237. A method for preventing one or more of all cause death, myocardial infarction, stroke, and non-central nervous system (CNS) systemic embolism in a patient with atrial fibrillation, wherein the method comprises orally administering to the human patient a regimen comprising about 100 mg of milvexian twice daily.

Aspect 238. A method for preventing cardiovascular death events in a human patient with atrial fibrillation, wherein the method comprises orally administering to the human patient a regimen comprising about 100 mg of milvexian twice daily.

Aspect 239. A method for preventing all cause death in a human patient with atrial fibrillation, wherein the method comprises orally administering to the human patient a regimen comprising about 100 mg of milvexian twice daily.

Aspect 240. A method for preventing cardiovascular death, myocardial infarction, stroke, acute limb ischemia, deep vein thrombosis, or pulmonary embolism events in a human patient with atrial fibrillation, wherein the method comprises orally administering to the human patient a regimen comprising about 100 mg of milvexian twice daily.

Aspect 241. A method for preventing myocardial infarction in a human patient with atrial fibrillation, wherein the method comprises orally administering to the human patient a regimen comprising about 100 mg of milvexian twice daily.

Aspect 242. A method for preventing acute limb ischemia in a human patient with atrial fibrillation, wherein the method comprises orally administering to the human patient a regimen comprising about 100 mg of milvexian twice daily.

Aspect 243. A method for preventing deep vein thrombosis in a human patient with atrial fibrillation, wherein the method comprises orally administering to the human patient a regimen comprising about 100 mg of milvexian twice daily.

Aspect 244. A method for preventing pulmonary embolism in a human patient with atrial fibrillation, wherein the method comprises orally administering to the human patient a regimen comprising 100 mg milvexian twice daily.

Aspect 245. The method of any one of aspects 231-244, wherein the administration results in a steady state plasma concentration profile of milvexian in about 3 to 6 days.

Aspect 246. The method of any one of aspects 231-245, wherein the administration results in a steady state plasma concentration profile of milvexian in about 6 days.

Aspect 247. The method of any one of aspects 231-246, wherein the administration results in a steady state plasma concentration profile of milvexian characterized by:
(i) a Cₘₐₓ at steady state ranging from about 1194 ng/ml to about 2326 ng/ml,
(ii) a steady state Cₘₐₓ mean (std) of 1760 (566) ng/mL;
(iii) a steady state AUC₀₋₂₄ ranging from about 23300 ng*h/mL to about 49100 ng*h/mL; or
(iv) a steady state AUC₀₋₂₄ mean (std) of 36200 (12900) ng*h/mL.

Aspect 248. The method of any one of aspects 231-247, wherein the administration results in no clinically significant QTc interval prolongation.

Aspect 249. The method of any one of aspects 231-248, wherein the administration results in no clinically relevant effects on any bleeding caused by milvexian exposure as measured by steady state AUC or Cₘₐₓ.

Aspect 250. The method of any one of aspects 231-249, wherein the administration results in minimal impairment of hemostasis in the human patient.

Aspect 251. The method of any one of aspects 231-249, wherein the administration results in no clinically meaningful change to prothrombin time, with a maximal mean percent change from baseline of approximately 5%.

Aspect 252. The method of any one of aspects 231-251, wherein the regimen comprises an immediate release tablet.

Aspect 253. The method of aspect 252, wherein the immediate release tablet has a disintegration time in water of less than 20 seconds at 37° C.

Aspect 254. The method of any one of aspects 231-253, wherein the administration results in a milvexian plasma terminal half-life ranging from about 13 hours to about 16 hours.

Aspect 255. The method of any one of aspects 231-254, wherein the regimen is administered without regard to the timing of food intake.

Aspect 256. The method of any one of aspects 252-255, wherein the immediate release tablet is dispersed in an aqueous medium to form an aqueous dispersion.

Aspect 257. The method of aspect 256, wherein the aqueous medium comprises water or apple sauce.

Aspect 258. Milvexian for use in a method of preventing stroke or non-central nervous system (CNS) systemic embolism in a human patient with atrial fibrillation, wherein the method comprises orally administering to the human patient a regimen comprising about 100 mg of milvexian twice daily; wherein the method achieves at least one of the following clinical outcomes: (1) reaches a steady state plasma concentration profile of milvexian in about 6 days; (2) reaches a steady state plasma concentration profile of milvexian characterized by: (i) a steady state Cₘₐₓ ranging from about 1194 ng/ml to about 2326 ng/mL, (ii) a steady state Cₘₐₓ mean (std) of 1760 (566) ng/ml, (iii) a steady state AUC₀₋₂₄ ranging from about 23300 ng*h/mL to about 49100 ng*h/mL, or (iv) a steady state AUC₀₋₂₄ mean (std) of 36200 (12900) ng*h/mL; (3) a change from baseline in QTc in the patient of less than 10 millisecond, (4) no clinically relevant effects on any bleeding caused by milvexian exposure as measured by steady state AUC or Cₘₐₓ; (5) minimal impairment of hemostasis in the human patient, (6) no clinically meaningful change to prothrombin time, with a maximal mean percent change from baseline of approximately 5%.; or (7) any combination of the aforementioned outcomes.

Aspect 259. Milvexian for use in a method for preventing one or more of stroke, or non-central nervous system (CNS) systemic embolism in a patient with a history of atrial fibrillation, wherein the method comprises orally administering to the human patient a regimen comprising about 100 mg of milvexian twice daily.

Aspect 260. Milvexian for use in a method for preventing one or more of cardiovascular death, myocardial infarction, stroke, or non-central nervous system (CNS) systemic embolism in a patient with atrial fibrillation, wherein the method comprises orally administering to the human patient a regimen comprising about 100 mg of milvexian twice daily.

Aspect 261. Milvexian for use in a method for preventing stroke in a human patient with a history of atrial fibrillation, wherein the method comprises orally administering to the human patient a regimen comprising about 100 mg of milvexian twice daily.

Aspect 262. Milvexian for use in a method for preventing ischemic stroke in a human patient with a history of atrial fibrillation, wherein the method comprises orally administering to the human patient a regimen comprising about 100 mg of milvexian twice daily.

Aspect 263. Milvexian for use in a method for preventing non-central nervous system (CNS) systemic embolism in a human patient with a history of atrial fibrillation, wherein the method comprises orally administering to the human patient a regimen comprising about 100 mg of milvexian twice daily.

Aspect 264. Milvexian for use in a method for preventing one or more of all cause death, myocardial infarction, stroke, or non-central nervous system (CNS) systemic embolism in a patient with atrial fibrillation, wherein the method comprises orally administering to the human patient a regimen comprising about 100 mg of milvexian twice daily.

Aspect 265. Milvexian for use in a method for preventing cardiovascular death in a human patient with atrial fibrillation, wherein the method comprises orally administering to the human patient a regimen comprising about 100 mg of milvexian twice daily.

Aspect 266. Milvexian for use in a method for preventing all cause death in a human patient with atrial fibrillation, wherein the method comprises orally administering to the human patient a regimen comprising about 100 mg of milvexian twice daily.

Aspect 267. Milvexian for use in a method for preventing cardiovascular death, myocardial infarction, stroke, acute limb ischemia, deep vein thrombosis, or pulmonary embolism in a human patient with a history of atrial fibrillation, wherein the method comprises orally administering to the human patient a regimen comprising about 100 mg of milvexian twice daily.

Aspect 268. Milvexian for use in a method for preventing myocardial infarction in a human patient with a history of atrial fibrillation, wherein the method comprises orally administering to the human patient a regimen comprising about 100 mg of milvexian twice daily.

Aspect 269. Milvexian for use in a method for preventing acute limb ischemia in a human patient with a history of atrial fibrillation, wherein the method comprises orally administering to the human patient a regimen comprising about 100 mg of milvexian twice daily.

Aspect 270. Milvexian for use in a method for preventing deep vein thrombosis in a human patient with a history of atrial fibrillation, wherein the method comprises orally administering to the human patient a regimen comprising about 100 mg of milvexian twice daily.

Aspect 271. Milvexian for use in a method for preventing pulmonary embolism in a human patient with a history of atrial fibrillation, wherein the method comprises orally administering to the human patient a regimen comprising about 100 mg of milvexian twice daily.

Aspect 272. Milvexian for use of any one of aspects 258-271, wherein the administration results in a steady state plasma concentration profile of milvexian in about 4 to 6 days.

Aspect 273. Milvexian for use of any one of aspects 258-272, wherein the administration results in a steady state plasma concentration profile of milvexian in about 6 days.

Aspect 274. Mmilvexian for use of any one of aspects 258-273, wherein the administration results in a steady state plasma concentration profile of milvexian characterized by:
(i) a Cₘₐₓ at steady state ranging from about 1194 ng/ml to about 2326 ng/ml,
(ii) a steady state Cₘₐₓ mean (std) of 1760 (566) ng/ml;
(iii) a steady state AUC₀₋₂₄ ranging from about 23300 ng*h/mL to about 49100 ng*h/mL; or
(iv) a steady state AUC₀₋₂₄ mean (std) of 36200 (12900) ng*h/mL.

Aspect 275. Milvexian for use of any one of aspects 258-274, wherein the administration results in no clinically significant QTc interval prolongation.

Aspect 276. Milvexian for use of any one of aspects 258-275, wherein the administration results in no clinically relevant effects on any bleeding caused by milvexian exposure as measured by steady state AUC or Cₘₐₓ.

Aspect 277. Milvexian for use of any one of aspects 258-275, wherein the administration results in minimal impairment of hemostasis in the human patient.

Aspect 278. Milvexian for use of any one of aspects 258-275, wherein the administration results in no clinically meaningful change to prothrombin time, with a maximal mean percent change from baseline of approximately 5%.

Aspect 279. Milvexian for use of any one of aspects 258-278, wherein the regimen comprises an immediate release tablet.

Aspect 280. Milvexian for use of aspect 279, wherein the immediate release tablet has a disintegration time in water of less than 20 seconds at 37° C.

Aspect 281. Milvexian for use of any one of aspects 258-275, wherein the administration results in a milvexian plasma terminal half-life ranging from about 13 hours to about 16 hours.

Aspect 282. Milvexian for use of any one of aspects 258-275, wherein the regimen is administered without regard to the timing of food intake.

Aspect 283. Milvexian for use of any one of aspects 279-282, wherein the immediate release tablet is dispersed in an aqueous medium to form an aqueous dispersion.

Aspect 284. Milvexian for use of aspect 283, wherein the aqueous medium comprises water or apple sauce.

Aspect 285. Any one of aspects 1-284, wherein “100 mg of milvexian” is replaced by “50 mg of milvexian.”

Aspect 286. Any one of aspects 1-284, wherein “100 mg of milvexian” is replaced by “75 mg of milvexian.”

Aspect 287. Any one of aspects 1-284, wherein “100 mg of milvexian” is replaced by “87.5 mg of milvexian.”

### Aspect Set #3

Aspect 1. A method for preventing one or more of stroke and non-central nervous system (CNS) systemic embolism events in a human patient with a history of atrial fibrillation or flutter, wherein the method comprises administering to the human patient a pharmaceutical composition comprising about 50 mg, about 75.0 mg, about 87.5 mg, or about 100 mg of milvexian (or a pharmaceutically acceptable salt or solvate thereof, on a milvexian basis); wherein the pharmaceutical composition is administered twice daily.

Aspect 2. A method for preventing one or more of major adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 25 mg, about 50 mg, about 75.0 mg, about 87.5 mg, or about 100 mg of milvexian (or a pharmaceutically acceptable salt or solvate thereof, on a milvexian basis); wherein the composition is administered twice daily.

Aspect 3. The method of aspect 2, wherein the major adverse cardiovascular event is cardiovascular death.

Aspect 4. The method of any one of aspects 1-3, wherein the patient has a history of atrial fibrillation or flutter, and also has concomitant coronary artery disease (CAD) and/or peripheral artery disease (PAD).

Aspect 5. The method of any one of aspects 1-4, wherein the pharmaceutical composition comprises about 100 mg of milvexian (or a pharmaceutically acceptable salt or solvate thereof, on a milvexian basis).

Aspect 6. The method of any one of aspects 1-4, wherein the pharmaceutical composition comprises about 87.5 mg of milvexian (or a pharmaceutically acceptable salt or solvate thereof, on a milvexian basis).

Aspect 7. The method of any one of aspects 1-4, wherein the pharmaceutical composition comprises about 75 mg of milvexian (or a pharmaceutically acceptable salt or solvate thereof, on a milvexian basis).

Aspect 8. The method of any one of aspects 1-4, wherein the pharmaceutical composition comprises about 50 mg of milvexian (or a pharmaceutically acceptable salt or solvate thereof, on a milvexian basis).

Aspect 9. The method of any one of aspects 1-8, wherein the pharmaceutical composition is a solid oral pharmaceutical composition comprising milvexian (or a pharmaceutically acceptable salt or solvate thereof) and one or more pharmaceutically acceptable excipients.

Aspect 10. The method of aspect 9, wherein the solid oral pharmaceutical composition is an immediate release tablet.

Aspect 11. The method of aspect 10, wherein the immediate release tablet has a disintegration time in water of less than 20 seconds at 37° C.

Aspect 12. The method of any one of aspects 5-11, wherein the administration results in a milvexian plasma half-life ranging from about 13 hours to about 16 hours.

Aspect 13. The method of any one of aspects 5-11, wherein the administration results in a milvexian plasma half-life ranging from about 13 hours to about 16 hours during repeat dosing.

Aspect 14. The method of any one of the preceding aspects, wherein the administration results in milvexian plasma concentrations reaching steady-state at about 3 days to 6 days.

Aspect 15. The method of any one of the preceding aspects, wherein the pharmaceutical composition comprising milvexian (or a pharmaceutically acceptable salt or solvate thereof) is administered without regard to the timing of food intake.

Aspect 16. The method of any one of the preceding aspects, wherein the composition comprising milvexian (or a pharmaceutically acceptable salt or solvate thereof) is administered twice daily for at least 13 weeks.

Aspect 17. The method of any one of the preceding aspects, wherein the composition comprising milvexian (or a pharmaceutically acceptable salt or solvate thereof) is administered once daily in the morning and once daily in the evening.

Aspect 18. The method of any one of the preceding aspects, wherein the morning administration is made at the same time each day and the evening administration is made at the same time each day.

Aspect 19. A method of preventing one or more of stroke and non-central nervous system (CNS) systemic embolism events in a patient with a history of atrial fibrillation or flutter, wherein the method comprising administering to the patient an immediate release film coated tablet comprising about 50 mg, about 75.0 mg, about 87.5 mg, or about 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (xi) age between 65 and 74 years;
   (xii) hypertension;
   (xiii) diabetes mellitus;
   (xiv) history of coronary artery disease (CAD) (including post myocardial infarction or acute coronary syndrome event with or without percutaneous coronary intervention (PCI)) or peripheral artery disease (PAD); and
   (xv) history of heart failure.

Aspect 20. A method of preventing one or more major adverse cardiovascular events in a patient with a history of atrial fibrillation or flutter, wherein each event is selected from the group consisting of cardiovascular death, myocardial infarction, stroke, and non-central nervous system (CNS) systemic embolism said method comprising administering to the patient an immediate release film-coated tablet comprising about 50 mg, about 75.0 mg, about 87.5 mg, or 100 mg of milvexian, or a pharmaceutically acceptable salt thereof, and one or more pharmaceutically acceptable excipient,
wherein the immediate release film-coated tablet is administered twice a day with or without a meal,
wherein the patient is at least 18 years old;
wherein the patient further has one or more of Category (A) risk factor selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 21. The method of aspect 20, wherein the major adverse cardiovascular event is stroke and non-central nervous system (CNS) systemic embolism.

Aspect 22. The method of any one of aspects 19-21, wherein immediate release film-coated tablet has a disintegration time in water of less than 20 seconds.

Aspect 23. The method of any one of aspects 19-22, wherein the administered milvexian has a plasma half-life of 13-16 hours.

Aspect 24. The method of any one of aspects 19-23, wherein the administered milvexian has a plasma half-life of 13-16 hours during repeat dosing.

Aspect 25. The method of any one of aspects 19-24, wherein the composition comprising milvexian (or a pharmaceutically acceptable salt or solvate thereof) is administered twice daily for at least 13 weeks.

Aspect 26. The method of any one of aspects 19-25, wherein the composition comprising milvexian (or a pharmaceutically acceptable salt or solvate thereof) is administered once daily in the morning and once daily in the evening.

Aspect 27. The method of aspect 26, wherein the morning administration is made at the same time each day and the evening administration is made at the same time each day.

Aspect 28. The method of any one of the preceding aspects, wherein the administration reduces FXI clotting activity in the patient by about 27% to about 64% relative to the baseline FXI clotting activity.

Aspect 29. The method of any one of the preceding aspects, wherein the administration results in a prolongation of activated partial thromboplastin time (aPTT) in the patient with a ratio to baseline of 2.1 to 2.6.

Aspect 30. A method for preventing stroke in a human patient with a history of atrial fibrillation or flutter, wherein the method comprises administering to the human patient a pharmaceutical composition comprising about 50 mg of milvexian (or a pharmaceutically acceptable salt or solvate thereof, on a milvexian basis), wherein the pharmaceutical composition is administered twice daily.

Aspect 31. A method for preventing stroke in a human patient with a history of atrial fibrillation or flutter, wherein the method comprises administering to the human patient a pharmaceutical composition comprising about 75 mg of milvexian (or a pharmaceutically acceptable salt or solvate thereof, on a milvexian basis), wherein the pharmaceutical composition is administered twice daily.

Aspect 32. A method for preventing stroke in a human patient with a history of atrial fibrillation or flutter, wherein the method comprises administering to the human patient a pharmaceutical composition comprising about 87.5 mg of milvexian (or a pharmaceutically acceptable salt or solvate thereof, on a milvexian basis), wherein the pharmaceutical composition is administered twice daily.

Aspect 33. A method for preventing stroke in a human patient with a history of atrial fibrillation or flutter, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.

Aspect 34. A method for preventing one or more non-central nervous system (CNS) systemic embolism events in a human patient with a history of atrial fibrillation or flutter, wherein the method comprises administering to the human patient a pharmaceutical composition comprising about 50 mg of milvexian (or a pharmaceutically acceptable salt or solvate thereof, on a milvexian basis), wherein the pharmaceutical composition is administered twice daily.

Aspect 35. A method for preventing one or more non-central nervous system (CNS) systemic embolism events in a human patient with a history of atrial fibrillation or flutter, wherein the method comprises administering to the human patient a pharmaceutical composition comprising about 75 mg of milvexian (or a pharmaceutically acceptable salt or solvate thereof, on a milvexian basis), wherein the pharmaceutical composition is administered twice daily.

Aspect 36. A method for preventing one or more non-central nervous system (CNS) systemic embolism events in a human patient with a history of atrial fibrillation or flutter, wherein the method comprises administering to the human patient a pharmaceutical composition comprising about 87.5 mg of milvexian (or a pharmaceutically acceptable salt or solvate thereof, on a milvexian basis), wherein the pharmaceutical composition is administered twice daily.

Aspect 37. A method for preventing one or more non-central nervous system (CNS) systemic embolism events in a human patient with a history of atrial fibrillation or flutter, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.

Aspect 38. A method for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising 50 mg of milvexian (or a pharmaceutically acceptable salt or solvate thereof, on a milvexian basis), wherein the composition is administered twice daily.

Aspect 39. The method of aspect 38, wherein the adverse cardiovascular event is cardiovascular death.

Aspect 40. The method of aspect 38, wherein the major adverse cardiovascular event is myocardial infarction.

Aspect 41. The method of aspect 38, wherein the major adverse cardiovascular event is stroke.

Aspect 42. The method of aspect 38, wherein the major adverse cardiovascular event is non-central nervous system (CNS) systemic embolism.

Aspect 43. A method for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian (or a pharmaceutically acceptable salt or solvate thereof, on a milvexian basis); wherein the composition is administered twice daily.

Aspect 44. The method of aspect 43, wherein the adverse cardiovascular event is cardiovascular death.

Aspect 45. The method of aspect 43, wherein the adverse cardiovascular event is myocardial infarction.

Aspect 46. The method of aspect 43, wherein the adverse cardiovascular event is stroke.

Aspect 47. The method of aspect 43, wherein the adverse cardiovascular event is non-central nervous system (CNS) systemic embolism.

Aspect 48. The method of any one of aspects 30-47, wherein the patient has a history of atrial fibrillation or flutter, and also has concomitant coronary artery disease (CAD) and/or peripheral artery disease (PAD).

Aspect 49. The method of aspect 48, wherein the patient has a history of atrial fibrillation or flutter, and also has concomitant coronary artery disease (CAD).

Aspect 50. The method of aspect 48, wherein the patient has a history of atrial fibrillation or flutter, and also has concomitant peripheral artery disease (PAD).

Aspect 51. The method of any one of aspects 30-50, wherein the pharmaceutical composition is a solid oral pharmaceutical composition comprising milvexian (or a pharmaceutically acceptable salt or solvate thereof) and one or more pharmaceutically acceptable excipients.

Aspect 52. The method of aspect 51, wherein the solid oral pharmaceutical composition is an immediate release tablet.

Aspect 53. The method of aspect 52, wherein the immediate release tablet has a disintegration time in water of less than 20 seconds.

Aspect 54. The method of any one of aspects 30-53, wherein the administration results in a milvexian plasma half-life ranging from about 13 hours to about 16 hours.

Aspect 55. The method of any one of aspects 30-54, wherein the administration results in a milvexian plasma half-life ranging from about 13 hours to about 16 hours during repeat dosing.

Aspect 56. The method of any one of aspects 30-55, wherein the administration results in milvexian plasma concentrations reaching steady-state at about 3 days to 6 days.

Aspect 57. The method of any one of aspects 30-56, wherein the pharmaceutical composition comprising milvexian (or a pharmaceutically acceptable salt or solvate thereof) is administered without regard to the timing of food intake.

Aspect 58. The method of any one aspects 30-57, wherein the patient is unable to swallow a tablet dosage form.

Aspect 59. The method of any one aspects 30-58, wherein the pharmaceutical composition is dispersed in an aqueous medium to form an aqueous dispersion.

Aspect 60. The method of aspect 59, wherein the aqueous medium is water, saline, phosphate buffer, vegetable juice, or fruit juice, including, for example, apple sauce.

Aspect 61. The method of aspect 59 or aspect 60, wherein the aqueous dispersion is administered to the human patient via a nasogastric tube or spoon.

Aspect 62. The method of any one of aspects 59-61, wherein the milvexian is administered orally as an aqueous dispersion by dispersing the pharmaceutical composition in an aqueous medium in less than 60 seconds.

Aspect 63. The method of any one of any one of aspects 30-62, wherein the composition comprising milvexian (or a pharmaceutically acceptable salt or solvate thereof) is administered twice daily for at least 13 weeks.

Aspect 64. The method of any one of any one of aspects 30-63, wherein the composition comprising milvexian (or a pharmaceutically acceptable salt or solvate thereof) is administered once daily in the morning and once daily in the evening.

Aspect 65. The method of aspect 64, wherein the morning administration is made at the same time each day and the evening administration is made at the same time each day.

Aspect 66. The method of any one of aspects 30-65, wherein the administration reduces FXI clotting activity in the patient by about 27% to about 64% relative to the baseline FXI clotting activity.

Aspect 67. The method of any one of any one of aspects 30-66, wherein the administration results in a prolongation of activated partial thromboplastin time (aPTT) in the patient with a ratio to baseline of 2.1 to 2.6.

Aspect 68. A method of preventing stroke in a patient with a history of atrial fibrillation or flutter, wherein the method comprising administering to the patient an immediate release film coated tablet comprising about 50 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 69. A method of preventing non-central nervous system (CNS) systemic embolism events in a patient with a history of atrial fibrillation or flutter, wherein the method comprising administering to the patient an immediate release film coated tablet comprising about 50 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 70. A method of preventing stroke in a patient with a history of atrial fibrillation or flutter, wherein the method comprising administering to the patient an immediate release film coated tablet comprising about 75 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 71. A method of preventing non-central nervous system (CNS) systemic embolism events in a patient with a history of atrial fibrillation or flutter, wherein the method comprising administering to the patient an immediate release film coated tablet comprising about 75 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 72. A method of preventing stroke in a patient with a history of atrial fibrillation or flutter, wherein the method comprising administering to the patient an immediate release film coated tablet comprising about 87.5 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 73. A method of preventing non-central nervous system (CNS) systemic embolism events in a patient with a history of atrial fibrillation or flutter, wherein the method comprising administering to the patient an immediate release film coated tablet comprising about 87.5 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 74. A method of preventing stroke in a patient with a history of atrial fibrillation or flutter, wherein the method comprising administering to the patient an immediate release film coated tablet comprising about 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 75. A method of preventing non-central nervous system (CNS) systemic embolism events in a patient with a history of atrial fibrillation or flutter, wherein the method comprising administering to the patient an immediate release film coated tablet comprising about 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 76. The method of any one of aspects 68-75, wherein the patient has a history of atrial fibrillation or flutter, and also has concomitant coronary artery disease (CAD) and/or peripheral artery disease (PAD).

Aspect 77. The method of aspect 76, wherein the patient has a history of atrial fibrillation or flutter, and also has concomitant coronary artery disease (CAD).

Aspect 78. The method of aspect 76, wherein the patient has a history of atrial fibrillation or flutter, and also has concomitant peripheral artery disease (PAD).

Aspect 79. The method of any one of aspects 68-78, wherein the immediate-release film-coated tablet has a disintegration time in water of less than 20 seconds at 37° C.

Aspect 80. The method of any one of aspects 68-79, wherein the administration results in a milvexian plasma half-life ranging from about 13 hours to about 16 hours.

Aspect 81. The method of any one of aspects 68-80, wherein the administration results in a milvexian plasma half-life ranging from about 13 hours to about 16 hours during repeat dosing.

Aspect 82. The method of any one of aspects 68-81, wherein the administration results in milvexian plasma concentrations reaching steady-state at about 3 days to 6 days.

Aspect 83. The method of any one of aspects 68-82, wherein the immediate-release film-coated tablet is administered without regard to the timing of food intake.

Aspect 84. The method of any one aspects 68-83, wherein the patient is unable to swallow a tablet dosage form.

Aspect 85. The method of any one aspects 68-84, wherein the immediate-release film-coated tablet is dispersed in an aqueous medium to form an aqueous dispersion.

Aspect 86. The method of aspect 85, wherein the aqueous medium is water, saline, phosphate buffer, vegetable juice, or fruit juice, including, for example, apple sauce.

Aspect 87. The method of aspect 85 or aspect 86, wherein the aqueous dispersion is administered to the human patient via a nasogastric tube or spoon.

Aspect 88. The method of any one of aspects 68-87, wherein the milvexian is administered orally as an aqueous dispersion by dispersing the immediate-release film-coated tablet in an aqueous medium in less than 60 seconds.

Aspect 89. The method of any one of any one of aspects 68-88, wherein the immediate-release film-coated tablet is administered twice daily for at least 13 weeks.

Aspect 90. The method of any one of any one of aspects 68-89, wherein the immediate-release film-coated tablet is administered once daily in the morning and once daily in the evening.

Aspect 91. The method of aspect 90, wherein the morning administration is made at the same time each day and the evening administration is made at the same time each day.

Aspect 92. The method of any one of aspects 68-91, wherein the administration reduces FXI clotting activity in the patient by about 27% to about 64% relative to the baseline FXI clotting activity.

Aspect 93. The method of any one of any one of aspects 68-91, wherein the administration results in a prolongation of activated partial thromboplastin time (aPTT) in the patient with a ratio to baseline of 2.1 to 2.6.

Aspect 94. The method of any one of the preceding aspects, wherein the method further comprises administering a standard-of-care antiplatelet therapy to the patient.

Aspect 95. The method of aspect 94, wherein, the standard-of-care antiplatelet therapy is selected from aspirin, adenosine diphosphate (ADP) receptor inhibitors; adenosine reuptake inhibitors; glycoprotein platelet inhibitors; phosphodiesterase inhibitors; or protease-activated receptor (PAR-1) antagonist.

Aspect 96. The method of any one of aspects 94 and 95, wherein the standard-of-care antiplatelet therapy is selected from aspirin, clopidogrel, ticagrelor, ticlopidine, prasugrel, dipyridamole, abciximab, eptifibatide, tirofiban, cilostazol, or vorapaxar.

Aspect 97. The method of any one of aspects 94-97, wherein the standard-of-care antiplatelet therapy is selected from aspirin, clopidogrel, ticagrelor, or prasugrel.

Aspect 98. The method of any one of aspects 94-97, wherein the standard-of-care antiplatelet therapy is selected from clopidogrel, ticagrelor, or prasugrel.

Aspect 99. The method of any one of aspects 94-98, wherein the standard-of-care antiplatelet therapy comprises a single antiplatelet therapy (SAPT).

Aspect 100. The method of aspect 99, wherein the single antiplatelet therapy comprises a aspirin, or a P2Y12 inhibitor selected from clopidogrel, ticagrelor, or prasugrel.

Aspect 101. The method of any one of aspects 94-98, wherein the standard-of-care antiplatelet therapy comprises a dual antiplatelet therapy (DAPT).

Aspect 102. The method of aspect 101, wherein the dual antiplatelet therapy comprises aspirin and a P2Y12 inhibitor selected from clopidogrel, ticagrelor, or prasugrel.

Aspect 103. The method of any one of aspects 101 and 102, wherein the dual antiplatelet therapy comprises a aspirin and a P2Y12 inhibitor selected from clopidogrel, ticagrelor, or prasugrel.

Aspect 104. The method of any one of aspects 101-103, wherein the dual antiplatelet therapy comprises a aspirin and clopidogrel.

Aspect 105. The method of any one of aspects 101-104, wherein the dual antiplatelet therapy (DAPT) is administered for 21 days, followed by single antiplatelet therapy (SAPT) thereafter.

Aspect 106. The method of any one of aspects 101-104, wherein the dual antiplatelet therapy (ie., aspirin and P2Y12 inhibitor) is administered for more than 90 days (with or without de-escalation to SAPT).

Aspect 107. Milvexian for use in preventing one or more of stroke and non-central nervous system (CNS) systemic embolism events in a human patient with a history of atrial fibrillation or flutter, wherein the method comprises administering to the human patient a pharmaceutical composition comprising about 50 mg, about 75.0 mg, about 87.5 mg, or about 100 mg of milvexian (or a pharmaceutically acceptable salt or solvate thereof, on a milvexian basis); wherein the pharmaceutical composition is administered twice daily.

Aspect 108. Milvexian for use in preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 50 mg, about 75.0 mg, about 87.5 mg, or 100 mg of milvexian (or a pharmaceutically acceptable salt or solvate thereof, on a milvexian basis); wherein the composition is administered twice daily.

Aspect 109. The milvexian for use of aspect 108, wherein the adverse cardiovascular event is cardiovascular death.

Aspect 110. The milvexian for use of any one of aspects 107-109, wherein the patient has a history of atrial fibrillation or flutter, and also has concomitant coronary artery disease (CAD) and/or peripheral artery disease (PAD).

Aspect 111. The milvexian for use of any one of aspects 107-110, wherein the pharmaceutical composition comprises 100 mg of milvexian (or a pharmaceutically acceptable salt or solvate thereof, on a milvexian basis).

Aspect 112. The milvexian for use of any one of aspects 107-110, wherein the pharmaceutical composition comprises 87.5 mg of milvexian (or a pharmaceutically acceptable salt or solvate thereof, on a milvexian basis).

Aspect 113. The milvexian for use of any one of aspects 107-110, wherein the pharmaceutical composition comprises 75 mg of milvexian (or a pharmaceutically acceptable salt or solvate thereof, on a milvexian basis).

Aspect 114. The milvexian for use of any one of aspects 107-110, wherein the pharmaceutical composition comprises 50 mg of milvexian (or a pharmaceutically acceptable salt or solvate thereof, on a milvexian basis).

Aspect 115. The milvexian for use of any one of aspects 107-114, wherein the pharmaceutical composition is a solid oral pharmaceutical composition comprising milvexian (or a pharmaceutically acceptable salt or solvate thereof) and one or more pharmaceutically acceptable excipients.

Aspect 116. The milvexian for use of aspect 115, wherein the solid oral pharmaceutical composition is an immediate release tablet.

Aspect 117. The milvexian for use of aspect 116, wherein the immediate release tablet has a disintegration time in water of less than 20 seconds.

Aspect 118. The milvexian for use of any one of aspects 107-117, wherein the administration results in a milvexian plasma half-life ranging from about 13 hours to about 16 hours.

Aspect 119. The milvexian for use of any one of aspects 107-118, wherein the administration results in a milvexian plasma half-life ranging from about 13 hours to about 16 hours during repeat dosing.

Aspect 120. The milvexian for use of any one of aspects 107-119, wherein the administration results in milvexian plasma concentrations reaching steady-state at about 3 days to 6 days.

Aspect 121. The milvexian for use of any one of aspects 107-120, wherein the pharmaceutical composition comprising milvexian (or a pharmaceutically acceptable salt or solvate thereof) is administered without regard to the timing of food intake.

Aspect 122. The milvexian for use of any one of aspects 107-121, wherein the composition comprising milvexian (or a pharmaceutically acceptable salt or solvate thereof) is administered twice daily for at least 13 weeks.

Aspect 123. The milvexian for use of any one of aspects 107-122, wherein the composition comprising milvexian (or a pharmaceutically acceptable salt or solvate thereof) is administered once daily in the morning and once daily in the evening.

Aspect 124. The milvexian for use of any one of aspects 107-123, wherein the morning administration is made at the same time each day and the evening administration is made at the same time each day.

Aspect 125. Milvexian for use in preventing one or more of stroke and non-central nervous system (CNS) systemic embolism events in a patient with a history of atrial fibrillation or flutter, wherein the method comprising administering to the patient an immediate release film coated tablet comprising about 50 mg, about 75.0 mg, about 87.5 mg, or 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (xvi) age between 65 and 74 years;
   (xvii) hypertension;
   (xviii) diabetes mellitus;
   (xix) history of coronary artery disease (CAD) (including post myocardial infarction or acute coronary syndrome event with or without percutaneous coronary intervention (PCI)) or peripheral artery disease (PAD); and
   (xx) history of heart failure.

Aspect 126. Milvexian for use in preventing one or more major adverse cardiovascular events in a patient with a history of atrial fibrillation or flutter, wherein each event is selected from the group consisting of cardiovascular death, myocardial infarction, stroke, and non-central nervous system (CNS) systemic embolism said method comprising administering to the patient an immediate release film-coated tablet comprising about 50 mg, about 75.0 mg, about 87.5 mg, or 100 mg of milvexian, or a pharmaceutically acceptable salt thereof, and one or more pharmaceutically acceptable excipient,
wherein the immediate release film-coated tablet is administered twice a day with or without a meal,
wherein the patient is at least 18 years old;
wherein the patient further has one or more of Category (A) risk factor selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 127. The milvexian for use of aspect 126, wherein the major adverse cardiovascular event is cardiovascular death.

Aspect 128. The milvexian for use of any one of aspects 125-127, wherein immediate release film-coated tablet has a disintegration time in water of less than 20 seconds.

Aspect 129. The milvexian for use of any one of aspects 125-128, wherein the administered milvexian has a plasma half-life of 13-16 hours.

Aspect 130. The milvexian for use of any one of aspects 125-129, wherein the administered milvexian has a plasma half-life of 13-16 hours during repeat dosing.

Aspect 131. The milvexian for use of any one of aspects 125-130, wherein the composition comprising milvexian (or a pharmaceutically acceptable salt or solvate thereof) is administered twice daily for at least 13 weeks.

Aspect 132. The milvexian for use of any one of aspects 105-131, wherein the composition comprising milvexian (or a pharmaceutically acceptable salt or solvate thereof) is administered once daily in the morning and once daily in the evening.

Aspect 133. The milvexian for use of aspect 132, wherein the morning administration is made at the same time each day and the evening administration is made at the same time each day.

Aspect 134. The milvexian for use of any one of aspects 107-133, wherein the administration reduces FXI clotting activity in the patient by about 27% to about 64% relative to the baseline FXI clotting activity.

Aspect 135. The milvexian for use of any one of aspects 107-134, wherein the administration results in a prolongation of activated partial thromboplastin time (aPTT) in the patient with a ratio to baseline of 2.1 to 2.6.

Aspect 136. Milvexian for use in preventing stroke in a human patient with a history of atrial fibrillation or flutter, wherein the method comprises administering to the human patient a pharmaceutical composition comprising about 50 mg of milvexian (or a pharmaceutically acceptable salt or solvate thereof, on a milvexian basis), wherein the pharmaceutical composition is administered twice daily.

Aspect 137. Milvexian for use in preventing stroke in a human patient with a history of atrial fibrillation or flutter, wherein the method comprises administering to the human patient a pharmaceutical composition comprising about 75 mg of milvexian (or a pharmaceutically acceptable salt or solvate thereof, on a milvexian basis), wherein the pharmaceutical composition is administered twice daily.

Aspect 138. Milvexian for use in preventing stroke in a human patient with a history of atrial fibrillation or flutter, wherein the method comprises administering to the human patient a pharmaceutical composition comprising about 87.5 mg of milvexian (or a pharmaceutically acceptable salt or solvate thereof, on a milvexian basis), wherein the pharmaceutical composition is administered twice daily.

Aspect 139. Milvexian for use in preventing stroke in a human patient with a history of atrial fibrillation or flutter, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.

Aspect 140. Milvexian for use in preventing one or more non-central nervous system (CNS) systemic embolism events in a human patient with a history of atrial fibrillation or flutter, wherein the method comprises administering to the human patient a pharmaceutical composition comprising about 50 mg of milvexian (or a pharmaceutically acceptable salt or solvate thereof, on a milvexian basis), wherein the pharmaceutical composition is administered twice daily.

Aspect 141. Milvexian for use in preventing one or more non-central nervous system (CNS) systemic embolism events in a human patient with a history of atrial fibrillation or flutter, wherein the method comprises administering to the human patient a pharmaceutical composition comprising about 75 mg of milvexian (or a pharmaceutically acceptable salt or solvate thereof, on a milvexian basis), wherein the pharmaceutical composition is administered twice daily.

Aspect 142. Milvexian for use in preventing one or more non-central nervous system (CNS) systemic embolism events in a human patient with a history of atrial fibrillation or flutter, wherein the method comprises administering to the human patient a pharmaceutical composition comprising about 87.5 mg of milvexian (or a pharmaceutically acceptable salt or solvate thereof, on a milvexian basis), wherein the pharmaceutical composition is administered twice daily.

Aspect 143. Milvexian for use in preventing one or more non-central nervous system (CNS) systemic embolism events in a human patient with a history of atrial fibrillation or flutter, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.

Aspect 144. Milvexian for use in preventing one or more of major adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 50 mg of milvexian (or a pharmaceutically acceptable salt or solvate thereof, on a milvexian basis), wherein the composition is administered twice daily.

Aspect 145. Milvexian for use in preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 75 mg of milvexian (or a pharmaceutically acceptable salt or solvate thereof, on a milvexian basis), wherein the composition is administered twice daily.

Aspect 146. Milvexian for use in preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 87.5 mg of milvexian (or a pharmaceutically acceptable salt or solvate thereof, on a milvexian basis), wherein the composition is administered twice daily.

Aspect 147. Milvexian for use in preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian (or a pharmaceutically acceptable salt or solvate thereof, on a milvexian basis); wherein the composition is administered twice daily.

Aspect 148. The milvexian for use of any one of aspects 144-147, wherein the adverse cardiovascular event is cardiovascular death.

Aspect 149. The milvexian for use of any one of aspects 144-147, wherein the adverse cardiovascular event is myocardial infarction.

Aspect 150. The milvexian for use of any one of aspects 144-147, wherein the adverse cardiovascular event is stroke.

Aspect 151. The milvexian for use of any one of aspects 144-147, wherein the adverse cardiovascular event is non-central nervous system (CNS) systemic embolism.

Aspect 152. The milvexian for use of any one of aspects 136-151, wherein the patient has a history of atrial fibrillation or flutter, and also has concomitant coronary artery disease (CAD) and/or peripheral artery disease (PAD).

Aspect 153. The milvexian for use of aspect 152, wherein the patient has a history of atrial fibrillation or flutter, and also has concomitant coronary artery disease (CAD).

Aspect 154. The milvexian for use of aspect 152, wherein the patient has a history of atrial fibrillation or flutter, and also has concomitant peripheral artery disease (PAD).

Aspect 155. The milvexian for use of any one of aspects 136-154, wherein the pharmaceutical composition is a solid oral pharmaceutical composition comprising milvexian (or a pharmaceutically acceptable salt or solvate thereof) and one or more pharmaceutically acceptable excipients.

Aspect 156. The milvexian for use of aspect 155, wherein the solid oral pharmaceutical composition is an immediate release tablet.

Aspect 157. The milvexian for use of aspect 156, wherein the immediate release tablet has a disintegration time in water of less than 20 seconds.

Aspect 158. The milvexian for use of any one of aspects 136-157, wherein the administration results in a milvexian plasma half-life ranging from about 13 hours to about 16 hours.

Aspect 159. The milvexian for use of any one of aspects 136-158, wherein the administration results in a milvexian plasma half-life ranging from about 13 hours to about 16 hours during repeat dosing.

Aspect 160. The milvexian for use of any one of aspects 136-159, wherein the administration results in milvexian plasma concentrations reaching steady-state at about 3 days to 6 days.

Aspect 161. The milvexian for use of any one of aspects 136-160, wherein the pharmaceutical composition comprising milvexian (or a pharmaceutically acceptable salt or solvate thereof) is administered without regard to the timing of food intake.

Aspect 162. The milvexian for use of any one aspects 136-161, wherein the patient is unable to swallow a tablet dosage form.

Aspect 163. The milvexian for use of any one aspects 136-162, wherein the pharmaceutical composition is dispersed in an aqueous medium to form an aqueous dispersion.

Aspect 164. The milvexian for use of aspect 163, wherein the aqueous medium is water, saline, phosphate buffer, vegetable juice, or fruit juice, including, for example, apple sauce.

Aspect 165. The milvexian for use of aspect 163 or aspect 164, wherein the aqueous dispersion is administered to the human patient via a nasogastric tube or spoon.

Aspect 166. The milvexian for use of any one of aspects 163-165, wherein the milvexian is administered orally as an aqueous dispersion by dispersing the pharmaceutical composition in an aqueous medium in less than 60 seconds at 37° C.

Aspect 167. The milvexian for use of any one of any one of aspects 136-166, wherein the composition comprising milvexian (or a pharmaceutically acceptable salt or solvate thereof) is administered twice daily for at least 13 weeks.

Aspect 168. The milvexian for use of any one of any one of aspects 136-167, wherein the composition comprising milvexian (or a pharmaceutically acceptable salt or solvate thereof) is administered once daily in the morning and once daily in the evening.

Aspect 169. The milvexian for use of aspect 168, wherein the morning administration is made at the same time each day and the evening administration is made at the same time each day.

Aspect 170. The milvexian for use of any one of aspects 136-169, wherein the administration reduces FXI clotting activity in the patient by about 27% to about 64% relative to the baseline FXI clotting activity.

Aspect 171. The milvexian for use of any one of any one of aspects 136-170, wherein the administration results in a prolongation of activated partial thromboplastin time (aPTT) in the patient with a ratio to baseline of 2.1 to 2.6.

Aspect 172. Milvexian for use in preventing stroke in a patient with a history of atrial fibrillation or flutter, wherein the method comprising administering to the patient an immediate release film coated tablet comprising about 50 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 173. Milvexian for use in preventing non-central nervous system (CNS) systemic embolism events in a patient with a history of atrial fibrillation or flutter, wherein the method comprising administering to the patient an immediate release film coated tablet comprising about 50 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 174. Milvexian for use in preventing stroke in a patient with a history of atrial fibrillation or flutter, wherein the method comprising administering to the patient an immediate release film coated tablet comprising about 75 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 175. Milvexian for use in preventing non-central nervous system (CNS) systemic embolism events in a patient with a history of atrial fibrillation or flutter, wherein the method comprising administering to the patient an immediate release film coated tablet comprising about 75 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 176. Milvexian for use in preventing stroke in a patient with a history of atrial fibrillation or flutter, wherein the method comprising administering to the patient an immediate release film coated tablet comprising about 87.5 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 177. Milvexian for use in preventing non-central nervous system (CNS) systemic embolism events in a patient with a history of atrial fibrillation or flutter, wherein the method comprising administering to the patient an immediate release film coated tablet comprising about 87.5 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 178. Milvexian for use in preventing stroke in a patient with a history of atrial fibrillation or flutter, wherein the method comprising administering to the patient an immediate release film coated tablet comprising about 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 179. Milvexian for use in preventing non-central nervous system (CNS) systemic embolism events in a patient with a history of atrial fibrillation or flutter, wherein the method comprising administering to the patient an immediate release film coated tablet comprising about 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 180. The milvexian for use of any one of aspects 172-179, wherein the patient has a history of atrial fibrillation or flutter, and also has concomitant coronary artery disease (CAD) and/or peripheral artery disease (PAD).

Aspect 181. The milvexian for use of aspect 180, wherein the patient has a history of atrial fibrillation or flutter, and also has concomitant coronary artery disease (CAD).

Aspect 182. The milvexian for use of aspect 180, wherein the patient has a history of atrial fibrillation or flutter, and also has concomitant peripheral artery disease (PAD).

Aspect 183. The milvexian for use of any one of aspects 172-182, wherein the immediate-release film-coated tablet has a disintegration time in water of less than 20 seconds.

Aspect 184. The milvexian for use of any one of aspects 172-183, wherein the administration results in a milvexian plasma half-life ranging from about 13 hours to about 16 hours.

Aspect 185. The milvexian for use of any one of aspects 172-184, wherein the administration results in a milvexian plasma half-life ranging from about 13 hours to about 16 hours during repeat dosing.

Aspect 186. The milvexian for use of any one of aspects 172-185, wherein the administration results in milvexian plasma concentrations reaching steady-state at about 3 days to 6 days.

Aspect 187. The milvexian for use of any one of aspects 172-186, wherein the immediate-release film-coated tablet is administered without regard to the timing of food intake.

Aspect 188. The milvexian for use of any one aspects 172-187, wherein the patient is unable to swallow a tablet dosage form.

Aspect 189. The milvexian for use of any one aspects 172-188, wherein the immediate-release film-coated tablet is dispersed in an aqueous medium to form an aqueous dispersion.

Aspect 190. The milvexian for use of aspect 189, wherein the aqueous medium is water, saline, phosphate buffer, vegetable juice, or fruit juice, including, for example, apple sauce.

Aspect 191. The milvexian for use of aspect 189 or aspect 190, wherein the aqueous dispersion is administered to the human patient via a nasogastric tube or spoon.

Aspect 192. The milvexian for use of any one of aspects 172-191, wherein the milvexian is administered orally as an aqueous dispersion by dispersing the immediate-release film-coated tablet in an aqueous medium in less than 60 seconds at 37° C.

Aspect 193. The milvexian for use of any one of any one of aspects 172-192, wherein the immediate-release film-coated tablet is administered twice daily for at least 13 weeks.

Aspect 194. The milvexian for use of any one of any one of aspects 172-193, wherein the immediate-release film-coated tablet is administered once daily in the morning and once daily in the evening.

Aspect 195. The milvexian for use of aspect 194, wherein the morning administration is made at the same time each day and the evening administration is made at the same time each day.

Aspect 196. The milvexian for use of any one of aspects 172-195, wherein the administration reduces FXI clotting activity in the patient by about 27% to about 64% relative to the baseline FXI clotting activity.

Aspect 197. The milvexian for use of any one of any one of aspects 172-196, wherein the administration results in a prolongation of activated partial thromboplastin time (aPTT) in the patient with a ratio to baseline of 2.1 to 2.6.

Aspect 198. The milvexian for use of any one of aspects 107-197, wherein the use further comprises administering a standard-of-care antiplatelet therapy to the patient.

Aspect 199. The milvexian for use of aspect 198, wherein, the standard-of-care antiplatelet therapy is selected from aspirin, adenosine diphosphate (ADP) receptor inhibitors; adenosine reuptake inhibitors; glycoprotein platelet inhibitors; phosphodiesterase inhibitors; or protease-activated receptor (PAR-1) antagonist.

Aspect 200. The milvexian for use of aspects 198 and 199, wherein the standard-of-care antiplatelet therapy is selected from aspirin, clopidogrel, ticagrelor, ticlopidine, prasugrel, dipyridamole, abciximab, eptifibatide, tirofiban, cilostazol, or vorapaxar.

Aspect 201. The milvexian for use of any one of aspects 198-200, wherein the standard-of-care antiplatelet therapy is selected from aspirin, clopidogrel, ticagrelor, or prasugrel.

Aspect 202. The milvexian for use of any one of aspects 198-201, wherein the standard-of-care antiplatelet therapy is selected from clopidogrel, ticagrelor, or prasugrel.

Aspect 203. The milvexian for use of any one of aspects 198-202, wherein the standard-of-care antiplatelet therapy comprises a single antiplatelet therapy (SAPT).

Aspect 204. The milvexian for use of aspect 203, wherein the single antiplatelet therapy comprises a aspirin, or a P2Y12 inhibitor selected from clopidogrel, ticagrelor, or prasugrel.

Aspect 205. The milvexian for use of any one of aspects 198-204, wherein the standard-of-care antiplatelet therapy comprises a dual antiplatelet therapy (DAPT).

Aspect 206. The milvexian for use of aspect 205, wherein the dual antiplatelet therapy comprises aspirin and a P2Y12 inhibitor selected from clopidogrel, ticagrelor, or prasugrel.

Aspect 207. The milvexian for use of any one of aspects 205 and 206, wherein the dual antiplatelet therapy comprises a aspirin and a P2Y12 inhibitor selected from clopidogrel, ticagrelor, or prasugrel.

Aspect 208. The milvexian for use of any one of aspects 205-207, wherein the dual antiplatelet therapy comprises a aspirin and clopidogrel.

Aspect 209. The milvexian for use of any one of aspects 205-208, wherein the dual antiplatelet therapy (DAPT) is administered for 21 days, followed by single antiplatelet therapy (SAPT) thereafter.

Aspect 210. The milvexian for use of any one of aspects 205-208, wherein the dual antiplatelet therapy (ie., aspirin and P2Y12 inhibitor) is administered for more than 90 days (with or without de-escalation to SAPT).

Aspect 211. A method for preventing one or more of stroke and non-central nervous system (CNS) systemic embolism events in a human patient with a history of atrial fibrillation or flutter, wherein the method comprises administering to the human patient a pharmaceutical composition comprising about 50 mg of milvexian, or a pharmaceutically acceptable salt thereof; wherein the pharmaceutical composition is administered twice daily.

Aspect 212. A method for preventing one or more of stroke and non-central nervous system (CNS) systemic embolism events in a human patient with a history of atrial fibrillation or flutter, wherein the method comprises administering to the human patient a pharmaceutical composition comprising about 100 mg of milvexian, or a pharmaceutically acceptable salt thereof; wherein the pharmaceutical composition is administered twice daily.

Aspect 213. A method for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 50 mg of milvexian, or a pharmaceutically acceptable salt thereof; wherein the composition is administered twice daily.

Aspect 214. A method for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian, or a pharmaceutically acceptable salt thereof; wherein the composition is administered twice daily.

Aspect 215. A method for reducing the risk of one or more of stroke and non-central nervous system (CNS) systemic embolism events in a human patient with a history of atrial fibrillation or flutter, wherein the method comprises administering to the human patient a pharmaceutical composition comprising about 50 mg of milvexian, or a pharmaceutically acceptable salt thereof; wherein the pharmaceutical composition is administered twice daily.

Aspect 216. A method for reducing the risk of one or more of stroke and non-central nervous system (CNS) systemic embolism events in a human patient with a history of atrial fibrillation or flutter, wherein the method comprises administering to the human patient a pharmaceutical composition comprising about 100 mg of milvexian, or a pharmaceutically acceptable salt thereof; wherein the pharmaceutical composition is administered twice daily.

Aspect 217. A method for reducing the risk of one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 50 mg of milvexian, or a pharmaceutically acceptable salt thereof; wherein the composition is administered twice daily.

Aspect 218. A method for reducing the risk of one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian, or a pharmaceutically acceptable salt thereof; wherein the composition is administered twice daily.

Aspect 219. The method of any one of aspects 211-218, wherein the reduction of risk is measured by hazard ratio (HR) at a value ranging from about 0.5 to about 1.1.

Aspect 220. The method of aspect 219, wherein HR has a value ranging from about 0.65 to about 0.86.

Aspect 221. The method of aspect 220, wherein HR has a value of about 0.84.

### Aspect Set #4

Aspect 1. A method for preventing one or more of stroke and non-central nervous system (CNS) systemic embolism events in a human patient with a history of atrial fibrillation or flutter, wherein the method comprises administering to the human patient a pharmaceutical composition comprising 50 mg, or 100 mg, of milvexian (or a pharmaceutically acceptable salt or solvate thereof, on a milvexian basis); wherein the pharmaceutical composition is administered twice daily.

Aspect 2. A method for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising 50 mg, or 100 mg, of milvexian (or a pharmaceutically acceptable salt or solvate thereof, on a milvexian basis); wherein the composition is administered twice daily.

Aspect 3. The method of aspect 2, wherein the major adverse cardiovascular event is cardiovascular death.

Aspect 4. The method of any one of aspects 1-3, wherein the patient has a history of atrial fibrillation or flutter, and also has concomitant coronary artery disease (CAD) and/or peripheral artery disease (PAD).

Aspect 5. The method of any one of aspects 1-4, wherein the pharmaceutical composition comprises 100 mg of milvexian (or a pharmaceutically acceptable salt or solvate thereof, on a milvexian basis).

Aspect 6. The method of any one of aspects 1-4, wherein the pharmaceutical composition comprises 50 mg of milvexian (or a pharmaceutically acceptable salt or solvate thereof, on a milvexian basis).

Aspect 7. The method of any one of aspects 1-6, wherein the pharmaceutical composition is a solid oral pharmaceutical composition comprising milvexian (or a pharmaceutically acceptable salt or solvate thereof) and one or more pharmaceutically acceptable excipients.

Aspect 8. The method of aspect 7, wherein the solid oral pharmaceutical composition is an immediate release tablet.

Aspect 9. The method of aspect 8, wherein the immediate release tablet has a disintegration time in water of less than 20 seconds.

Aspect 10. The method of any one of aspects 5-9, wherein the administration results in a milvexian plasma half-life ranging from about 13 hours to about 16 hours.

Aspect 11. The method of any one of the preceding aspects, wherein the administration results in milvexian plasma concentrations reaching steady-state at about 3 days to 6 days.

Aspect 12. The method of any one of the preceding aspects, wherein the pharmaceutical composition comprising milvexian (or a pharmaceutically acceptable salt or solvate thereof) is administered without regard to the timing of food intake.

Aspect 13. A method of preventing one or more of stroke and non-central nervous system (CNS) systemic embolism events in a patient with a history of atrial fibrillation or flutter, wherein the method comprising administering to the patient an immediate release film coated tablet comprising 50 mg or 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke or silent brain infarct; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 14. A method of preventing one or more adverse cardiovascular events in a patient with a history of atrial fibrillation or flutter, wherein each event is selected from the group consisting of cardiovascular death, myocardial infarction, stroke, and non-central nervous system (CNS) systemic embolism said method comprising administering to the patient an immediate release film-coated tablet comprising 50 mg or 100 mg of milvexian, or a pharmaceutically acceptable salt thereof, and one or more pharmaceutically acceptable excipient,
wherein the immediate release film-coated tablet is administered twice a day with or without a meal,
wherein the patient is at least 18 years old;
wherein the patient further has one or more of Category (A) risk factor selected from (i) age greater than or equal to 75 years or (ii) history of ischemic stroke or silent brain infarct; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.

Aspect 15. The method of aspect 14, wherein the adverse cardiovascular event is cardiovascular death.

Aspect 16. The method of any one of aspects 13-15, wherein immediate release film-coated tablet has a disintegration time in water of less than 20 seconds at 37° C.

Aspect 17. The method of any one of aspects 13-16, wherein the administered milvexian has a plasma half-life of 13-16 hours.

Aspect 18. The method of any one of the preceding aspects, wherein the administration reduces FXI clotting activity in the patient by about 27% to about 64% relative to the baseline FXI clotting activity.

Aspect 19. The method of any one of the preceding aspects, wherein the administration results in a prolongation of activated partial thromboplastin time (aPTT) in the patient with a ratio to baseline of 2.1 to 2.6.

Aspect 20. The method of any one of the preceding aspects, wherein the composition comprising milvexian (or a pharmaceutically acceptable salt or solvate thereof) is administered twice daily for at least 13 weeks.

Aspect 21. The method of any one of the preceding aspects, wherein the composition comprising milvexian (or a pharmaceutically acceptable salt or solvate thereof) is administered once daily in the morning and once daily in the evening.

Aspect 22. The method of aspect 21, wherein the morning administration is made at the same time each day and the evening administration is made at the same time each day.

### Aspect Set #5

Aspect 1. A method of preventing adverse cerebrovascular events or adverse cardiovascular events in a human patient with acute coronary syndrome, wherein the method comprises administering to the human patient a regimen comprising: (i) a pharmaceutical composition comprising 25 mg of milvexian (or pharmaceutically acceptable salt or solvate thereof); and (ii) an antiplatelet therapy selected from the group consisting of aspirin, a P2Y12 inhibitor, and combinations thereof; wherein the pharmaceutical composition is administered twice daily.

Aspect 2. The method of aspect 1, wherein the antiplatelet therapy is a P2Y12 inhibitor.

Aspect 3. The method of aspect 2, wherein the P2Y12 inhibitor is clopidogrel, ticagrelor, or prasugrel.

Aspect 4. The method of any one of aspects 1-3, wherein the antiplatelet therapy is aspirin.

Aspect 5. The method of any one of aspects 1-4, wherein the human patient is treated with aspirin and clopidogrel combination therapy from day 1 to day 21, followed by aspirin monotherapy for at least 90 days.

Aspect 6. The method of aspect 5, wherein the patient is treated with aspirin and/or clopidogrel without milvexian dose adjustment.

Aspect 7. The method of any one of the preceding aspects, wherein the method is for primary prevention of adverse cerebrovascular events or adverse cardiovascular events.

Aspect 8. The method of any one of the preceding aspects, wherein the method is for secondary prevention of adverse cerebrovascular events or adverse cardiovascular events.

Aspect 9. The method of any one of aspects 1-8, wherein the adverse cerebrovascular events or adverse cardiovascular events comprises one or more of stroke, heart attack, or death.

Aspect 10. The method of any one of aspects 1-9, wherein the adverse cerebrovascular events or adverse cardiovascular events occur in an organ selected from heart, brain, limb, blood supply system, or vessel.

Aspect 11. The method of any one of aspects 1-10, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.

Aspect 12. The method of any one of aspects 1-11, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.

Aspect 13. The method of any one of aspects 1-12, wherein the adverse cerebrovascular events or adverse cardiovascular events are selected from the group consisting of arrhythmogenic cardiomyopathy (ACM), nonfatal myocardial infarction, ischemic stroke, and combinations thereof.

Aspect 14. The method of any one of aspects 1-11, wherein the adverse cerebrovascular events or adverse cardiovascular events comprises cardiovascular death.

Aspect 15. The method of any one of aspects 1-10, wherein the adverse cerebrovascular events or adverse cardiovascular events comprises arrhythmogenic cardiomyopathy (ACM).

Aspect 16. A method for preventing adverse cardiovascular events in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof;
i. wherein the pharmaceutical composition is administered twice daily; and
ii. wherein the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; and transient ischemic attack (TIA).

Aspect 17. The method of aspect 16, wherein the adverse cardiovascular event is one or more of CV death, MI, or ischemic stroke.

Aspect 18. A method for reducing incidence rate of the one or more of adverse thrombotic event selected from new ischemic stroke, MI, or all-cause death in a human patient diagnosed with acute coronary syndrome, wherein the method comprises administering to the human patient a regimen comprising: (i) a pharmaceutical composition comprising about 25 mg to about 100 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

Aspect 19. A method for preventing ischemic stroke in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

Aspect 20. The method of aspect 19, wherein the clinical benefit of reducing the incidence rates of the ischemic stroke by the regimen is maintained throughout a treatment period of 90 days.

Aspect 21. The method of any one of the preceding aspects, wherein administration of the regimen reduces the patient's FXI clotting activity by about 7% to about 20% relative to baseline.

Aspect 22. The method of any one of the preceding aspects, wherein administration of the regimen results is a prolongation of activated partial thromboplastin time (aPTT) ranging from about 27% to about 64% relative to baseline.

Aspect 23. The method of any one of the preceding aspects, where the administration does not result in a statistically significant increase in major bleeding complications.

Aspect 24. The method of any one of the preceding aspects, wherein the pharmaceutical composition comprising milvexian (or pharmaceutically acceptable salt or solvate thereof) is a solid oral pharmaceutical composition.

Aspect 25. The method of aspect 24, wherein the solid oral pharmaceutical composition is a tablet.

Aspect 26. The method of aspect 25, wherein the tablet is an immediate release tablet.

Aspect 27. The method of aspect 25 or aspect 26, wherein the tablet has a disintegration time in water of less than 20 seconds at 37° C.

Aspect 28. The method of any one of the preceding aspects, wherein the administration results in a milvexian plasma half-life ranging from about 13 hours to about 16 hours.

Aspect 29. The method of any one of the preceding aspects, wherein the administration results in milvexian plasma concentration reaching steady-state at about 3 days to 6 days.

Aspect 30. The method of any one of the preceding aspects, wherein the pharmaceutical composition comprising milvexian (or pharmaceutically acceptable salt or solvate thereof) is administered without regard to the timing of food intake.

Aspect 31. The method of any one of the preceding aspects, wherein the human patient is unable to swallow a tablet dosage form.

Aspect 32. The method of any one of aspects 25-31, wherein the tablet is dispersed in an aqueous medium to form an aqueous dispersion.

Aspect 33. The method of aspect 32, wherein the aqueous medium is water, saline, phosphate buffer, vegetable juice, or fruit juice, including, for example, apple sauce.

Aspect 34. The method of aspect 32 or aspect 33, wherein the aqueous dispersion is administered to the human patient via a nasogastric tube or spoon.

Aspect 35. The method of aspect any one of aspects 25-34, wherein the pharmaceutical composition is administered orally as an aqueous dispersion by dispersing the oral tablet in an aqueous medium in less than 1 minute at 37° C.

Aspect 36. A method of preventing adverse cerebrovascular events or adverse cardiovascular events in a human patient with acute coronary syndrome, wherein the method comprises orally administering to the human patient a regimen comprising: (i) a pharmaceutical composition comprising 25 mg of milvexian (or pharmaceutically acceptable salt or solvate thereof); and (ii) an antiplatelet therapy; wherein the pharmaceutical composition is administered twice daily.

Aspect 37. The method of aspect 36, wherein the antiplatelet therapy is a P2Y12 inhibitor.

Aspect 38. The method of aspect 37, wherein the P2Y12 inhibitor is clopidogrel, ticagrelor, or prasugrel.

Aspect 39. The method of any one of aspects 36-38, wherein the antiplatelet therapy is aspirin.

Aspect 40. The method of any one of aspects 36-39, wherein the human patient is treated with aspirin and clopidogrel combination therapy from day 1 to day 21, followed by aspirin monotherapy for at least 90 days.

Aspect 41. The method of aspect 40, wherein the patient is treated with aspirin and/or clopidogrel without milvexian dose adjustment.

Aspect 42. The method of any one of aspects 36-41, wherein the method is for primary prevention of adverse cerebrovascular events or adverse cardiovascular events.

Aspect 43. The method of any one of aspects 36-42, wherein the method is for secondary prevention of adverse cerebrovascular events or adverse cardiovascular events.

Aspect 44. The method of any one of aspects 36-43, wherein the adverse cerebrovascular events or adverse cardiovascular events comprises one or more of stroke, heart attack, or death.

Aspect 45. The method of any one of aspects 36-44, wherein the adverse cerebrovascular events or adverse cardiovascular events occur in an organ selected from heart, brain, limb, blood supply system, or vessel.

Aspect 46. The method of any one of aspects 36-45, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.

Aspect 47. The method of any one of aspects 36-46, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.

Aspect 48. The method of any one of aspects 36-47, wherein the adverse cerebrovascular events or adverse cardiovascular events are selected from the group consisting of arrhythmogenic cardiomyopathy (ACM), nonfatal myocardial infarction, ischemic stroke, and combinations thereof.

Aspect 49. The method of any one of aspects 36-47, wherein the adverse cerebrovascular events or adverse cardiovascular events comprises cardiovascular death.

Aspect 50. The method of any one of aspects 36-49, wherein the adverse cerebrovascular events or adverse cardiovascular events comprises arrhythmogenic cardiomyopathy (ACM).

Aspect 51. A method for preventing adverse cardiovascular events in a human patient diagnosed with acute coronary syndrome, wherein the method comprises administering to the human patient a regimen comprising: (i) 25.0 mg of milvexian twice daily, and (ii) an antiplatelet therapy;
wherein the adverse cardiovascular event is one or more of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; or transient ischemic attack (TIA); and
wherein the method achieves at least one of the following clinical outcomes: (1) reaches a steady state plasma concentration profile of milvexian in about 6 days; (2) reaches a steady state plasma concentration profile of milvexian characterized by: (i) a steady state Cₘₐₓ ranging from about 217 ng/ml to about 475 ng/ml, (ii) a steady state mean (std) Cₘₐₓ of 346 (129) ng/mL, (iii) a steady state AUC₀₋₂₄ ranging from about about 4350 ng*h/mL to about 10230 ng*h/mL, or (iv) a steady state mean (std) AUC₀₋₂₄ of 7290 (2940) ng*h/mL; (3) a change from baseline in QTc in the patient of less than 10 millisecond, (4) no clinically relevant effects on any bleeding caused by milvexian exposure as measured by steady state AUC or Cₘₐₓ; (5) minimal impairment of hemostasis in the human patient, (6) no clinically meaningful change to prothrombin time, with a maximal mean percent change from baseline of approximately 5%.; or (7) any combination of the aforementioned clinical outcomes.

Aspect 52. A method for preventing adverse cardiovascular events in a human patient diagnosed with acute coronary syndrome, wherein the method comprises administering to the human patient a regimen comprising: (i) 25.0 mg of milvexian twice daily, and (ii) an antiplatelet therapy;
wherein the adverse cardiovascular event is one or more of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; or transient ischemic attack (TIA).

Aspect 53. A method for preventing all-cause mortality in a human patient diagnosed with acute coronary syndrome, wherein the method comprises administering to the human patient a regimen comprising: (i) 25.0 mg of milvexian twice daily, and (ii) an antiplatelet therapy.

Aspect 54. A method for preventing cardiovascular death in a human patient diagnosed with acute coronary syndrome, wherein the method comprises administering to the human patient a regimen comprising: (i) 25.0 mg of milvexian twice daily, and (ii) an antiplatelet therapy.

Aspect 55. A method for preventing myocardial infarction in a human patient diagnosed with acute coronary syndrome, wherein the method comprises administering to the human patient a regimen comprising: (i) 25.0 mg of milvexian twice daily, and (ii) an antiplatelet therapy.

Aspect 56. A method for preventing stroke in a human patient diagnosed with acute coronary syndrome, wherein the method comprises administering to the human patient a regimen comprising: (i) 25.0 mg of milvexian twice daily, and (ii) an antiplatelet therapy.

Aspect 57. A method for preventing ischemic stroke in a human patient diagnosed with acute coronary syndrome, wherein the method comprises administering to the human patient a regimen comprising: (i) 25.0 mg of milvexian twice daily, and (ii) an antiplatelet therapy.

Aspect 58. A method for preventing ischemic stroke in a human patient diagnosed with acute coronary syndrome, wherein the method comprises administering to the human patient a regimen comprising: (i) 25.0 mg of milvexian twice daily, and (ii) an antiplatelet therapy.

Aspect 59. A method for preventing unstable angina in a human patient diagnosed with acute coronary syndrome, wherein the method comprises administering to the human patient a regimen comprising: (i) 25.0 mg of milvexian twice daily, and (ii) an antiplatelet therapy.

Aspect 60. A method for preventing acute limb inschemia in a human patient diagnosed with acute coronary syndrome, wherein the method comprises administering to the human patient a regimen comprising: (i) 25.0 mg of milvexian twice daily, and (ii) an antiplatelet therapy.

Aspect 61. A method for preventing major vascular (non-traumatic) limb amputation in a human patient diagnosed with acute coronary syndrome, wherein the method comprises administering to the human patient a regimen comprising: (i) 25.0 mg of milvexian twice daily, and (ii) an antiplatelet therapy.

Aspect 62. A method for preventing symptomatic venous thromboembolism in a human patient diagnosed with acute coronary syndrome, wherein the method comprises administering to the human patient a regimen comprising: (i) 25.0 mg of milvexian twice daily, and (ii) an antiplatelet therapy.

Aspect 63. A method for preventing pulmonary embolism in a human patient diagnosed with acute coronary syndrome, wherein the method comprises administering to the human patient a regimen comprising: (i) 25.0 mg of milvexian twice daily, and (ii) an antiplatelet therapy.

Aspect 64. A method for preventing deep vein thrombosis in a human patient diagnosed with acute coronary syndrome, wherein the method comprises administering to the human patient a regimen comprising: (i) 25.0 mg of milvexian twice daily, and (ii) an antiplatelet therapy.

Aspect 65. A method for preventing deep vein thrombosis in a human patient diagnosed with acute coronary syndrome, wherein the method comprises administering to the human patient a regimen comprising: (i) 25.0 mg of milvexian twice daily, and (ii) an antiplatelet therapy.

Aspect 66. A method for preventing ischemia-driven coronary revascularization in a human patient diagnosed with acute coronary syndrome, wherein the method comprises administering to the human patient a regimen comprising: (i) 25.0 mg of milvexian twice daily, and (ii) an antiplatelet therapy.

Aspect 67. A method for preventing stent thrombosis in a human patient diagnosed with acute coronary syndrome, wherein the method comprises administering to the human patient a regimen comprising: (i) 25.0 mg of milvexian twice daily, and (ii) an antiplatelet therapy.

Aspect 68. A method for preventing hospitalization for any reason, categorized as (a) planned or unplanned, (b) for arterial or venous thrombotic event or neither in a human patient diagnosed with acute coronary syndrome, wherein the method comprises administering to the human patient a regimen comprising: (i) 25.0 mg of milvexian twice daily, and (ii) an antiplatelet therapy.

Aspect 69. A method for preventing transient ischemic attack in a human patient diagnosed with acute coronary syndrome, wherein the method comprises administering to the human patient a regimen comprising: (i) 25.0 mg of milvexian twice daily, and (ii) an antiplatelet therapy.

Aspect 70. A method of preventing adverse cerebrovascular events in a human patient with acute coronary syndrome, wherein the method comprises orally administering twice daily to the human patient a regimen comprising: (i) 25 mg of milvexian; and (ii) an antiplatelet therapy.

Aspect 71. A method of preventing adverse cardiovascular events in a human patient with acute coronary syndrome, wherein the method comprises orally administering twice daily to the human patient a regimen comprising: (i) 25 mg of milvexian; and (ii) an antiplatelet therapy.

Aspect 72. A method of preventing adverse cerebrovascular events in a human patient with acute coronary syndrome, wherein the method comprises orally administering to the human patient a regimen comprising: (i) a pharmaceutical composition comprising 25 mg of milvexian and a pharmaceutically acceptable excipient; and (ii) an antiplatelet therapy; wherein the pharmaceutical composition is administered twice daily.

Aspect 73. A method of preventing adverse cardiovascular events in a human patient with acute coronary syndrome, wherein the method comprises administering to the human patient a regimen comprising: (i) a pharmaceutical composition comprising 25 mg of milvexian and a pharmaceutically acceptable excipient; and (ii) an antiplatelet therapy; wherein the pharmaceutical composition is administered twice daily.

Aspect 74. The method of any one of aspects of 70-73, wherein the adverse cerebrovascular events or cardiovascular events comprises ischemic stroke.

Aspect 75. The method any one of any one of aspects of 70-74, wherein the adverse cerebrovascular events or cardiovascular events comprises one or more of stroke, heart attack, or death.

Aspect 76. The method of any one of any one of aspects 70-75, wherein the adverse cerebrovascular events or adverse cardiovascular events occur in an organ selected from heart, brain, limb, blood supply system, or vessel.

Aspect 77. The method of any one of any one of aspects 70-76, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.

Aspect 78. The method of any one of any one of aspects 70-77, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.

Aspect 79. The method of any one of aspects 70-78, wherein the adverse cerebrovascular events or adverse cardiovascular events are selected from the group consisting of stroke, and combinations thereof.

Aspect 80. The method of any one of aspects 70-79, wherein the adverse cerebrovascular events or adverse cardiovascular events comprises cardiovascular death.

Aspect 81. The method of any one of aspects 70-80, wherein the adverse cerebrovascular events or adverse cardiovascular events comprises arrhythmogenic cardiomyopathy (ACM).

Aspect 82. A method of preventing ischemic stroke in a human patient with acute coronary syndrome, wherein the method comprises orally administering twice daily to the human patient a regimen comprising: (i) 25 mg of milvexian; and (ii) an antiplatelet therapy.

Aspect 83. The method of any one of the preceding aspects, wherein the administration results in a steady state plasma concentration profile of milvexian in about 3 days.

Aspect 84. The method of any one of the preceding aspects, wherein the administration results in a steady state plasma concentration profile of milvexian characterized by:
(i) a steady state Cₘₐₓ ranging from about 217 ng/mL to about 475 ng/ml,
(ii) a steady state mean (std) Cₘₐₓ of 346 (129) ng/ml,
(iii) a steady state AUC₀₋₂₄ ranging from about 4350 ng*h/mL to about 10230 ng*h/mL, and
(iv) a steady state mean (std) AUC₀₋₂₄ of 7290 (2940) ng*h/mL.

Aspect 85. The method of any one of the preceding aspects, wherein the administration results in no clinically significant QTc interval prolongation.

Aspect 86. The method of any one of the preceding aspects, wherein the administration results in no clinically relevant effects on any bleeding caused by milvexian exposure as measured by steady state AUC or Cₘₐₓ.

Aspect 87. The method of any one of the preceding aspects, wherein the administration results in minimal impairment of hemostasis in the human patient.

Aspect 88. The method of any one of the preceding aspects, wherein the regimen comprises an immediate release tablet.

Aspect 89. The method of aspect 88, wherein the immediate release tablet has a disintegration time in water of less than 20 seconds at 37° C.

Aspect 90. The method of any one of the preceding aspects, wherein the administration results in a milvexian plasma terminal half-life ranging from about 13 hours to about 16 hours.

Aspect 91. The method of any one of the preceding aspects, wherein the regimen is administered without regard to the timing of food intake.

Aspect 92. The method of any one of aspects 88-91, wherein the immediate release tablet is dispersed in an aqueous medium to form an aqueous dispersion.

Aspect 93. The method of aspect 92, wherein the aqueous medium comprises water or apple sauce.

Aspect 94. The method of any one of the preceding aspects, wherein the antiplatelet therapy comprises administering a single antiplatelet therapy (SAPT) for a period ranging from about 1 month to about 36 months.

Aspect 95. The method of aspect 94, wherein the antiplatelet therapy comprises administering a SAPT for a period ranging from about 3 months to about 12 months.

Aspect 96. The method of aspect 94, wherein the antiplatelet therapy comprises administering a SAPT for a period ranging from about 6 months to about 12 months.

Aspect 97. The method of aspect 94, wherein the antiplatelet therapy comprises administering a SAPT for 3 months.

Aspect 98. The method of aspect 94, wherein the antiplatelet therapy comprises administering a SAPT for 6 months.

Aspect 99. The method of aspect 94, wherein the antiplatelet therapy comprises administering a SAPT for 12 months.

Aspect 100. The method of aspect 94, wherein the antiplatelet therapy comprises administering a SAPT for 24 months.

Aspect 101. The method of any one of aspects 94-100, wherein the SAPT is selected from aspirin, and a P2Y12 inhibitor.

Aspect 102. The method of any one of aspects 94-101, wherein the SAPT comprises aspirin.

Aspect 103. The method of aspect 102, wherein the aspirin is administered once daily.

Aspect 104. The method of aspect 102 wherein the aspirin is administered once daily for a period of ranging from about 3 months to about 3 years.

Aspect 105. The method of aspect 102, wherein the aspirin is administered once daily for 3 months.

Aspect 106. The method of aspect 102, wherein the aspirin is administered once daily for 6 months.

Aspect 107. The method of aspect 102, wherein the aspirin is administered once daily for 12 months.

Aspect 108. The method of aspect 102, wherein the aspirin is administered once daily for 24 months.

Aspect 109. The method of aspect 102, wherein the aspirin is administered once daily for 36 months.

Aspect 110. The method of aspect 102, wherein the aspirin is administered twice daily.

Aspect 111. The method of aspect 102, wherein the aspirin is administered once daily at an amount of 75 mg to 100 mg.

Aspect 112. The method of aspect 102, wherein the aspirin is administered once daily at an amount of 75 mg to 100 mg for a period of 12 months.

Aspect 113. The method of any one of aspects 94-101, wherein the SAPT comprises a P2Y12 inhibitor.

Aspect 114. The method of any one of aspects 94-101, wherein the SAPT comprises a P2Y12 inhibitor selected from prasugrel, clopidogrel, selatogrel, or ticagrelor.

Aspect 115. The method of aspect 113 or 114, wherein the P2Y12 inhibitor is administered for a period ranging from about 3 months to about 6 months.

Aspect 116. The method of aspect 113 or 114, wherein the P2Y12 inhibitor comprises clopidogrel.

Aspect 117. The method of aspect 113 or 114, wherein the P2Y12 inhibitor comprises ticagrelor.

Aspect 118. The method of aspect 113 or 114, wherein the P2Y12 inhibitor comprises ticagrelor administered at 90 mg twice daily.

Aspect 119. The method of aspect 113 or 114, wherein the P2Y12 inhibitor comprises ticagrelor administered at 90 mg twice daily for a period of 3 years.

Aspect 120. The method of aspect 113 or 114, wherein the P2Y12 inhibitor comprises ticagrelor administered first followed by aspirin monotherapy administered at 20 mg twice daily.

Aspect 121. The method of any one of aspects 113 or 114, wherein the SAPT comprises aspirin monotherapy administered first for a period of ranging from 30 days to 90 days followed by clopidogrel monotherapy.

Aspect 122. The method of any one of aspects 113 or 114, wherein the SAPT comprises aspirin monotherapy administered first for a period of 60 days followed by clopidogrel monotherapy.

Aspect 123. The method of any one of aspects 94-122, wherein the SAPT comprises aspirin monotherapy administered first for a period of 90 days followed by clopidogrel monotherapy.

Aspect 124. The method of any one of aspects 94-123, wherein the SAPT comprises aspirin monotherapy administered first followed by clopidogrel monotherapy for a period of at least 2 months.

Aspect 125. The method of any one of aspects 94-123, wherein the SAPT comprises aspirin monotherapy administered first for a period ranging from 30 days to 90 days followed by clopidogrel monotherapy for a period of at least 2 months.

Aspect 126. The method of any one of aspects 1-93, wherein the antiplatelet therapy comprises a dual antiplatelet therapy (DAPT).

Aspect 127. The method of aspect of 126, wherein the DAPT comprises aspirin and prasugrel.

Aspect 128. The method of aspect of 126, wherein the DAPT comprises aspirin and prasugrel administered for 12 months.

Aspect 129. The method of aspect of 126, wherein the DAPT comprises aspirin and clopidogrel.

Aspect 130. The method of aspect of 126, wherein the DAPT comprises aspirin and clopidogrel administered for a period of 21 days to 12 months.

Aspect 131. The method of aspect of 126, wherein the DAPT comprises aspirin and clopidogrel administered for a period of 21 days.

Aspect 132. The method of aspect of 126, wherein the DAPT comprises aspirin and clopidogrel administered for a period of 6 months.

Aspect 133. The method of aspect of 126, wherein the DAPT comprises aspirin and clopidogrel administered for a period of 12 months.

Aspect 134. The method of aspect of 126, wherein the DAPT comprises aspirin and ticagrelor.

Aspect 135. The method of aspect of 126, wherein the DAPT comprises aspirin and ticagrelor administered for 12 months.

Aspect 136. The method of aspect 126, wherein the antiplatelet therapy comprises a DAPT consisting of aspirin administered once daily at 75 mg to 100 mg and a P2Y12 inhibitor.

Aspect 137. The method of aspect 126, wherein the antiplatelet therapy comprises a DAPT administered first followed by administering a SAPT.

Aspect 138. The method of aspect 126, wherein the antiplatelet therapy comprises a DAPT administered first followed by administering aspirin monotherapy.

Aspect 139. The method of aspect 126, wherein the antiplatelet therapy comprises a DAPT consisting of aspirin 75-100 mg once daily and a P2Y12 inhibitor administered for a first period of 12 months followed by administering aspirin monotherapy for a second period of 6 months.

Aspect 140. The method of aspect 126, wherein the antiplatelet therapy comprises a DAPT consisting of aspirin 75-100 mg once daily and a P2Y12 inhibitor administered for a first period of 21 days followed by administering aspirin monotherapy for a second period of 6 months.

Aspect 141. The method of aspect 126, wherein the antiplatelet therapy comprises a DAPT consisting of aspirin 75-100 mg once daily and clopidogrel 75 mg once daily administered for a first period of 21 days followed by administering aspirin monotherapy for a second period of 6 months.

Aspect 142. The method of aspect 126, wherein the antiplatelet therapy comprises a DAPT consisting of aspirin 75-100 mg once daily and a P2Y12 inhibitor administered for a first period of 21 days followed by administering aspirin monotherapy for a second period of 12 months.

Aspect 143. The method of aspect 126, wherein the antiplatelet therapy comprises a DAPT consisting of aspirin 75-100 mg once daily and ticagrelor 90 mg twice daily.

Aspect 144. The method of aspect 126, wherein the antiplatelet therapy comprises a DAPT consisting of aspirin 75-100 mg once daily and ticagrelor 90 mg twice daily administered for a period of 12 months.

Aspect 145. The method of aspect 126, wherein the antiplatelet therapy comprises a DAPT consisting of aspirin 81-100 mg once daily and ticagrelor 90 mg twice daily.

Aspect 146. The method of aspect 126, wherein the antiplatelet therapy comprises a DAPT consisting of aspirin 81-100 mg once daily and ticagrelor 90 mg twice daily administered for 3 years.

Aspect 147. The method of aspect 126, wherein the antiplatelet therapy comprises a DAPT consisting of aspirin 75-100 mg once daily and ticagrelor 90 mg twice daily administered for a first period of 12 months followed by administering aspirin monotherapy 75-100 mg once daily for a second period of 12 months.

Aspect 148. The method of aspect 126, wherein the antiplatelet therapy comprises a DAPT consisting of aspirin 75-100 mg once daily and ticagrelor 90 mg twice daily administered for a first period of 12 months followed by administering ticagrelor 90 mg twice daily for a second period of 23 months.

Aspect 149. The method of any one of the previous aspects, wherein the clinical benefit of reducing the incidence rates of the ischemic stroke by the regimen is maintained throughout a treatment period of 90 days.

Aspect 150. The method of any one of the previous aspects, wherein the clinical benefit of reducing the incidence rates of the ischemic stroke is maintained throughout a treatment period of 180 days.

Aspect 151. The method of any one of the previous aspects, wherein the clinical benefit of reducing the incidence rates of the ischemic stroke is maintained throughout a treatment period of 12 months.

Aspect 152. The method of any one of the preceding aspects, wherein administration of the regimen reduces the patient's FXI clotting activity by about 7% to about 20% relative to baseline.

Aspect 153. The method of any one of the preceding aspects, wherein administration of the regimen results is a prolongation of activated partial thromboplastin time (aPTT) ranging from about 27% to about 64% relative to baseline.

Aspect 154. The method of any one of the preceding aspects, where the administration does not result in a statistically significant increase in major bleeding complications.

Aspect 155. The method of any one of the preceding aspects, wherein the pharmaceutical composition comprising milvexian (or pharmaceutically acceptable salt or solvate thereof) is a solid oral pharmaceutical composition.

Aspect 156. The method of aspect 155, wherein the solid oral pharmaceutical composition is a tablet.

Aspect 157. The method of aspect 156, wherein the tablet is an immediate release tablet.

Aspect 158. The method of any one of the preceding aspects, wherein the human patient is unable to swallow a tablet dosage form.

Aspect 159. The method of any one of aspects 156-158, wherein the tablet is dispersed in an aqueous medium to form an aqueous dispersion.

Aspect 160. The method of aspect 159, wherein the aqueous medium is water, saline, phosphate buffer, vegetable juice, or fruit juice, including, for example, apple sauce.

Aspect 161. The method of aspect 159 or aspect 160, wherein the aqueous dispersion is administered to the human patient via a nasogastric tube or spoon.

Aspect 162. The method of any one of the preceding aspects, wherein the pharmaceutical composition is administered orally as an aqueous dispersion by dispersing the oral tablet in an aqueous medium in less than 1 minute.

Aspect 163. The method of any one of the preceding aspects, wherein the pharmaceutical composition comprises an amorphous form of milvexian admixed with a polymer.

Aspect 164. The method of any one of the preceding aspects, wherein the pharmaceutical composition comprises a spray dried amorphous solid dispersion consisting of 75% w/w milvexian and 25% w/w of HPMC-AS.

Aspect 165. The method of any one of the preceding aspects, wherein the method results in no clinically significant QTc interval prolongation.

### Aspect Set #6

Aspect 1. Milvexian for use in a method of preventing adverse cerebrovascular events or adverse cardiovascular events in a human patient with acute coronary syndrome, wherein the method comprises administering to the human patient a regimen comprising: (i) a pharmaceutical composition comprising 25 mg of milvexian (or pharmaceutically acceptable salt or solvate thereof); and (ii) an antiplatelet therapy selected from the group consisting of aspirin, a P2Y12 inhibitor, and combinations thereof; wherein the pharmaceutical composition is administered twice daily.

Aspect 2. The milvexian for use of aspect 1, wherein the antiplatelet therapy is a P2Y12 inhibitor.

Aspect 3. The milvexian for use of aspect 2, wherein the P2Y12 inhibitor is clopidogrel, ticagrelor, or prasugrel.

Aspect 4. The milvexian for use of any one of aspects 1-3, wherein the antiplatelet therapy is aspirin.

Aspect 5. The milvexian for use of any one of aspects 1-4, wherein the human patient is treated with aspirin and clopidogrel combination therapy from day 1 to day 21, followed by aspirin monotherapy for at least 90 days.

Aspect 6. The milvexian for use of aspect 5, wherein the patient is treated with aspirin and/or clopidogrel without milvexian dose adjustment.

Aspect 7. The milvexian for use of any one of the preceding aspects, wherein the milvexian for use is for primary prevention of adverse cerebrovascular events or adverse cardiovascular events.

Aspect 8. The milvexian for use of any one of the preceding aspects, wherein the milvexian for use is for secondary prevention of adverse cerebrovascular events or adverse cardiovascular events.

Aspect 9. The milvexian for use of any one of aspects 1-8, wherein the adverse cerebrovascular events or adverse cardiovascular events comprises one or more of stroke, heart attack, or death.

Aspect 10. The milvexian for use of any one of aspects 1-9, wherein the adverse cerebrovascular events or adverse cardiovascular events occur in an organ selected from heart, brain, limb, blood supply system, or vessel.

Aspect 11. The milvexian for use of any one of aspects 1-10, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.

Aspect 12. The milvexian for use of any one of aspects 1-11, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.

Aspect 13. The milvexian for use of any one of aspects 1-12, wherein the adverse cerebrovascular events or adverse cardiovascular events are selected from the group consisting of arrhythmogenic cardiomyopathy (ACM), nonfatal myocardial infarction, ischemic stroke, and combinations thereof.

Aspect 14. The milvexian for use of any one of aspects 1-11, wherein the adverse cerebrovascular events or adverse cardiovascular events comprises cardiovascular death.

Aspect 15. The milvexian for use of any one of aspects 1-10, wherein the adverse cerebrovascular events or adverse cardiovascular events comprises arrhythmogenic cardiomyopathy (ACM).

Aspect 16. Milvexian for use in a method for preventing adverse cardiovascular events in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof,
i. wherein the pharmaceutical composition is administered twice daily; and
ii. wherein the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; and transient ischemic attack (TIA).

Aspect 17. The milvexian for use of aspect 16, wherein the adverse cardiovascular event is one or more of CV death, MI, or ischemic stroke.

Aspect 18. Milvexian for use in a method for reducing incidence rate of the one or more of adverse thrombotic event selected from new ischemic stroke, MI, or all-cause death in a human patient diagnosed with acute coronary syndrome, wherein the method comprises administering to the human patient a regimen comprising: (i) a pharmaceutical composition comprising about 25 mg to about 100 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

Aspect 19. Milvexian for use in a method for preventing ischemic stroke in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

Aspect 20. The milvexian for use of aspect 18 or aspect 19, wherein the clinical benefit of reducing the incidence rates of the ischemic stroke by the regimen is maintained throughout a treatment period of 90 days.

Aspect 21. The milvexian for use of any one of the preceding aspects, wherein administration of the regimen reduces the patient's FXI clotting activity by about 7% to about 20% relative to baseline.

Aspect 22. The milvexian for use of any one of the preceding aspects, wherein administration of the regimen results is a prolongation of activated partial thromboplastin time (aPTT) ranging from about 27% to about 64% relative to baseline.

Aspect 23. The milvexian for use of any one of the preceding aspects, where the administration does not result in a statistically significant increase in major bleeding complications.

Aspect 24. The milvexian for use of any one of the preceding aspects, wherein the pharmaceutical composition comprising milvexian (or pharmaceutically acceptable salt or solvate thereof) is a solid oral pharmaceutical composition.

Aspect 25. The milvexian for use of aspect 24, wherein the solid oral pharmaceutical composition is a tablet.

Aspect 26. The milvexian for use of aspect 25, wherein the tablet is an immediate release tablet.

Aspect 27. The milvexian for use of aspect 25 or aspect 26, wherein the tablet has a disintegration time in water of less than 20 seconds.

Aspect 28. The milvexian for use of any one of the preceding aspects, wherein the administration results in a milvexian plasma half-life ranging from about 13 hours to about 16 hours.

Aspect 29. The milvexian for use of any one of the preceding aspects, wherein the administration results in milvexian plasma concentration reaching steady-state at about 3 days to 6 days.

Aspect 30. The milvexian for use of any one of the preceding aspects, wherein the pharmaceutical composition comprising milvexian (or pharmaceutically acceptable salt or solvate thereof) is administered without regard to the timing of food intake.

Aspect 31. The milvexian for use of any one of the preceding aspects, wherein the human patient is unable to swallow a tablet dosage form.

Aspect 32. The milvexian for use of any one of aspects 25-31, wherein the tablet is dispersed in an aqueous medium to form an aqueous dispersion.

Aspect 33. The milvexian for use of aspect 32, wherein the aqueous medium is water, saline, phosphate buffer, vegetable juice, or fruit juice, including, for example, apple sauce.

Aspect 34. The milvexian for use of aspect 32 or aspect 33, wherein the aqueous dispersion is administered to the human patient via a nasogastric tube or spoon.

Aspect 35. The milvexian for use of aspect any one of aspects 25-34, wherein the pharmaceutical composition is administered orally as an aqueous dispersion by dispersing the oral tablet in an aqueous medium in less than 1 minute.

Aspect 36. Milvexian for use in a method of preventing adverse cerebrovascular events or adverse cardiovascular events in a human patient with acute coronary syndrome, wherein the method comprises orally administering to the human patient a regimen comprising: (i) a pharmaceutical composition comprising 25 mg of milvexian (or pharmaceutically acceptable salt or solvate thereof); and (ii) an antiplatelet therapy; wherein the pharmaceutical composition is administered twice daily.

Aspect 37. The milvexian for use of aspect 36, wherein the antiplatelet therapy is a P2Y12 inhibitor.

Aspect 38. The milvexian for use of aspect 37, wherein the P2Y12 inhibitor is clopidogrel, ticagrelor, or prasugrel.

Aspect 39. The milvexian for use of any one of aspects 36-38, wherein the antiplatelet therapy is aspirin.

Aspect 40. The milvexian for use of any one of aspects 36-39, wherein the human patient is treated with aspirin and clopidogrel combination therapy from day 1 to day 21, followed by aspirin monotherapy for at least 90 days.

Aspect 41. The milvexian for use of aspect 40, wherein the patient is treated with aspirin and/or clopidogrel without milvexian dose adjustment.

Aspect 42. The milvexian for use of any one of aspects 36-41, wherein the milvexian for use is for primary prevention of adverse cerebrovascular events or adverse cardiovascular events.

Aspect 43. The milvexian for use of any one of aspects 36-42, wherein the milvexian for use is for secondary prevention of adverse cerebrovascular events or adverse cardiovascular events.

Aspect 44. The milvexian for use of any one of aspects 36-43, wherein the adverse cerebrovascular events or adverse cardiovascular events comprises one or more of stroke, heart attack, or death.

Aspect 45. The milvexian for use of any one of aspects 36-44, wherein the adverse cerebrovascular events or adverse cardiovascular events occur in an organ selected from heart, brain, limb, blood supply system, or vessel.

Aspect 46. The milvexian for use of any one of aspects 36-45, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.

Aspect 47. The milvexian for use of any one of aspects 36-46, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.

Aspect 48. The milvexian for use of any one of aspects 36-47, wherein the adverse cerebrovascular events or adverse cardiovascular events are selected from the group consisting of arrhythmogenic cardiomyopathy (ACM), nonfatal myocardial infarction, ischemic stroke, and combinations thereof.

Aspect 49. The milvexian for use of any one of aspects 36-47, wherein the adverse cerebrovascular events or adverse cardiovascular events comprises cardiovascular death.

Aspect 50. The milvexian for use of any one of aspects 36-48, wherein the adverse cerebrovascular events or adverse cardiovascular events comprises arrhythmogenic cardiomyopathy (ACM).

Aspect 51. Milvexian for use in a method for preventing adverse cardiovascular events in a human patient diagnosed with acute coronary syndrome, wherein the method comprises administering to the human patient a regimen comprising: (i) 25.0 mg of milvexian twice daily, and (ii) an antiplatelet therapy;
wherein the adverse cardiovascular event is one or more of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; or transient ischemic attack (TIA); and
wherein the method achieves at least one of the following clinical outcomes: (1) reaches a steady state plasma concentration profile of milvexian in about 6 days; (2) reaches a steady state plasma concentration profile of milvexian characterized by: (i) a steady state Cₘₐₓ ranging from about 217 ng/mL to about 475 ng/mL, (ii) a steady state mean (std) Cₘₐₓ of 346 (129) ng/ml, (iii) a steady state AUC₀₋₂₄ ranging from about about 4350 ng*h/mL to about 10230 ng*h/mL, or (iv) a steady state mean (std) AUC₀₋₂₄ of 7290 (2940) ng*h/mL; (3) a change from baseline in QTc in the patient of less than 10 millisecond, (4) no clinically relevant effects on any bleeding caused by milvexian exposure as measured by steady state AUC or Cₘₐₓ; (5) minimal impairment of hemostasis in the human patient, (6) no clinically meaningful change to prothrombin time, with a maximal mean percent change from baseline of approximately 5%.; or (7) any combination of the aforementioned clinical outcomes.

Aspect 52. Milvexian for use in a method for preventing adverse cardiovascular events in a human patient diagnosed with acute coronary syndrome, wherein the method comprises administering to the human patient a regimen comprising: (i) 25.0 mg of milvexian twice daily, and (ii) an antiplatelet therapy;
wherein the adverse cardiovascular event is one or more of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; or transient ischemic attack (TIA).

Aspect 53. Milvexian for use in a method for preventing all-cause mortality in a human patient diagnosed with acute coronary syndrome, wherein the method comprises administering to the human patient a regimen comprising: (i) 25.0 mg of milvexian twice daily, and (ii) an antiplatelet therapy.

Aspect 54. Milvexian for use in a method for preventing cardiovascular death in a human patient diagnosed with acute coronary syndrome, wherein the method comprises administering to the human patient a regimen comprising: (i) 25.0 mg of milvexian twice daily, and (ii) an antiplatelet therapy.

Aspect 55. Milvexian for use in a method for preventing myocardial infarction in a human patient diagnosed with acute coronary syndrome, wherein the method comprises administering to the human patient a regimen comprising: (i) 25.0 mg of milvexian twice daily, and (ii) an antiplatelet therapy.

Aspect 56. Milvexian for use in a method for preventing stroke in a human patient diagnosed with acute coronary syndrome, wherein the method comprises administering to the human patient a regimen comprising: (i) 25.0 mg of milvexian twice daily, and (ii) an antiplatelet therapy.

Aspect 57. Milvexian for use in a method for preventing ischemic stroke in a human patient diagnosed with acute coronary syndrome, wherein the method comprises administering to the human patient a regimen comprising: (i) 25.0 mg of milvexian twice daily, and (ii) an antiplatelet therapy.

Aspect 58. Milvexian for use in a method for preventing ischemic stroke in a human patient diagnosed with acute coronary syndrome, wherein the method comprises administering to the human patient a regimen comprising: (i) 25.0 mg of milvexian twice daily, and (ii) an antiplatelet therapy.

Aspect 59. Milvexian for use in a method for preventing unstable angina in a human patient diagnosed with acute coronary syndrome, wherein the method comprises administering to the human patient a regimen comprising: (i) 25.0 mg of milvexian twice daily, and (ii) an antiplatelet therapy.

Aspect 60. Milvexian for use in a method for preventing acute limb ischemia in a human patient diagnosed with acute coronary syndrome, wherein the method comprises administering to the human patient a regimen comprising: (i) 25.0 mg of milvexian twice daily, and (ii) an antiplatelet therapy.

Aspect 61. Milvexian for use in a method for preventing major vascular (non-traumatic) limb amputation in a human patient diagnosed with acute coronary syndrome, wherein the method comprises administering to the human patient a regimen comprising: (i) 25.0 mg of milvexian twice daily, and (ii) an antiplatelet therapy.

Aspect 62. Milvexian for use in a method for preventing symptomatic venous thromboembolism in a human patient diagnosed with acute coronary syndrome, wherein the method comprises administering to the human patient a regimen comprising: (i) 25.0 mg of milvexian twice daily, and (ii) an antiplatelet therapy.

Aspect 63. Milvexian for use in a method for preventing pulmonary embolism in a human patient diagnosed with acute coronary syndrome, wherein the method comprises administering to the human patient a regimen comprising: (i) 25.0 mg of milvexian twice daily, and (ii) an antiplatelet therapy.

Aspect 64. Milvexian for use in a method for preventing deep vein thrombosis in a human patient diagnosed with acute coronary syndrome, wherein the method comprises administering to the human patient a regimen comprising: (i) 25.0 mg of milvexian twice daily, and (ii) an antiplatelet therapy.

Aspect 65. Milvexian for use in a method for preventing deep vein thrombosis in a human patient diagnosed with acute coronary syndrome, wherein the method comprises administering to the human patient a regimen comprising: (i) 25.0 mg of milvexian twice daily, and (ii) an antiplatelet therapy.

Aspect 66. Milvexian for use in a method for preventing ischemia-driven coronary revascularization in a human patient diagnosed with acute coronary syndrome, wherein the method comprises administering to the human patient a regimen comprising: (i) 25.0 mg of milvexian twice daily, and (ii) an antiplatelet therapy.

Aspect 67. Milvexian for use in a method for preventing stent thrombosis in a human patient diagnosed with acute coronary syndrome, wherein the method comprises administering to the human patient a regimen comprising: (i) 25.0 mg of milvexian twice daily, and (ii) an antiplatelet therapy.

Aspect 68. Milvexian for use in a method for preventing hospitalization for any reason, categorized as (a) planned or unplanned, (b) for arterial or venous thrombotic event or neither in a human patient diagnosed with acute coronary syndrome, wherein the method comprises administering to the human patient a regimen comprising: (i) 25.0 mg of milvexian twice daily, and (ii) an antiplatelet therapy.

Aspect 69. Milvexian for use in a method for preventing transient ischemic attack in a human patient diagnosed with acute coronary syndrome, wherein the method comprises administering to the human patient a regimen comprising: (i) 25.0 mg of milvexian twice daily, and (ii) an antiplatelet therapy.

Aspect 70. Milvexian for use in a method of preventing adverse cerebrovascular events in a human patient with acute coronary syndrome, wherein the method comprises orally administering twice daily to the human patient a regimen comprising: (i) 25 mg of milvexian; and (ii) an antiplatelet therapy.

Aspect 71. Milvexian for use in a method of preventing adverse cardiovascular events in a human patient with acute coronary syndrome, wherein the method comprises orally administering twice daily to the human patient a regimen comprising: (i) 25 mg of milvexian; and (ii) an antiplatelet therapy.

Aspect 72. Milvexian for use in a method of preventing adverse cerebrovascular events in a human patient with acute coronary syndrome, wherein the method comprises orally administering to the human patient a regimen comprising: (i) a pharmaceutical composition comprising 25 mg of milvexian and a pharmaceutically acceptable excipient; and (ii) an antiplatelet therapy; wherein the pharmaceutical composition is administered twice daily.

Aspect 73. Milvexian for use in a method of preventing adverse cardiovascular events in a human patient with acute coronary syndrome, wherein the method comprises administering to the human patient a regimen comprising: (i) a pharmaceutical composition comprising 25 mg of milvexian and a pharmaceutically acceptable excipient; and (ii) an antiplatelet therapy; wherein the pharmaceutical composition is administered twice daily.

Aspect 74. The milvexian for use of any one of aspects of 70-73, wherein the adverse cerebrovascular events or cardiovascular events comprises ischemic stroke.

Aspect 75. The milvexian for use any one of any one of aspects of 70-74, wherein the adverse cerebrovascular events or cardiovascular events comprises one or more of stroke, heart attack, or death.

Aspect 76. The milvexian for use of any one of any one of aspects 70-75, wherein the adverse cerebrovascular events or adverse cardiovascular events occur in an organ selected from heart, brain, limb, blood supply system, or vessel.

Aspect 77. The milvexian for use of any one of any one of aspects 70-76, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.

Aspect 78. The milvexian for use of any one of any one of aspects 70-77, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.

Aspect 79. The milvexian for use of any one of aspects 70-78, wherein the adverse cerebrovascular events or adverse cardiovascular events are selected from the group consisting of arrhythmogenic cardiomyopathy (ACM), nonfatal myocardial infarction, ischemic stroke, and combinations thereof.

Aspect 80. The milvexian for use of any one of aspects 70-79, wherein the adverse cerebrovascular events or adverse cardiovascular events comprises cardiovascular death.

Aspect 81. The milvexian for use of any one of aspects 70-80, wherein the adverse cerebrovascular events or adverse cardiovascular events comprises arrhythmogenic cardiomyopathy (ACM).

Aspect 82. Milvexian for use in a method of preventing ischemic stroke in a human patient with acute coronary syndrome, wherein the method comprises orally administering twice daily to the human patient a regimen comprising: (i) 25 mg of milvexian; and (ii) an antiplatelet therapy.

Aspect 83. The milvexian for use of any one of the preceding aspects, wherein the administration results in a steady state plasma concentration profile of milvexian in about 3 days.

Aspect 84. The milvexian for use of any one of the preceding aspects, wherein the administration results in a steady state plasma concentration profile of milvexian characterized by:
(i) a steady state Cₘₐₓ ranging from about 217 ng/mL to about 475 ng/ml,
(ii) a steady state mean (std) Cₘₐₓ of 346 (129) ng/ml,
(iii) a steady state AUC₀₋₂₄ ranging from about 4350 ng*h/mL to about 10230 ng*h/mL, and
(iv) a steady state mean (std) AUC₀₋₂₄ of 7290 (2940) ng*h/mL.

Aspect 85. The milvexian for use of any one of the preceding aspects, wherein the administration results in no clinically significant QTc interval prolongation.

Aspect 86. The milvexian for use of any one of the preceding aspects, wherein the administration results in no clinically relevant effects on any bleeding caused by milvexian exposure as measured by steady state AUC or Cₘₐₓ.

Aspect 87. The milvexian for use of any one of the preceding aspects, wherein the administration results in minimal impairment of hemostasis in the human patient.

Aspect 88. The milvexian for use of any one of the preceding aspects, wherein the regimen comprises an immediate release tablet.

Aspect 89. The milvexian for use of aspect 88, wherein the immediate release tablet has a disintegration time in water of less than 20 seconds at 37° C.

Aspect 90. The milvexian for use of any one of the preceding aspects, wherein the administration results in a milvexian plasma terminal half-life ranging from about 13 hours to about 16 hours.

Aspect 91. The milvexian for use of any one of the preceding aspects, wherein the regimen is administered without regard to the timing of food intake.

Aspect 92. The milvexian for use of any one of aspects 88-91, wherein the immediate release tablet is dispersed in an aqueous medium to form an aqueous dispersion.

Aspect 93. The milvexian for use of aspect 92, wherein the aqueous medium comprises water or apple sauce.

Aspect 94. The milvexian for use of any one of the preceding aspects, wherein the antiplatelet therapy comprises administering a single antiplatelet therapy (SAPT) for a period ranging from about 1 month to about 36 months.

Aspect 95. The milvexian for use of aspect 94, wherein the antiplatelet therapy comprises administering a SAPT for a period ranging from about 3 months to about 12 months.

Aspect 96. The milvexian for use of aspect 94, wherein the antiplatelet therapy comprises administering a SAPT for a period ranging from about 6 months to about 12 months.

Aspect 97. The milvexian for use of aspect 94, wherein the antiplatelet therapy comprises administering a SAPT for 3 months.

Aspect 98. The milvexian for use of aspect 94, wherein the antiplatelet therapy comprises administering a SAPT for 6 months.

Aspect 99. The milvexian for use of aspect 94, wherein the antiplatelet therapy comprises administering a SAPT for 12 months.

Aspect 100. The milvexian for use of aspect 94, wherein the antiplatelet therapy comprises administering a SAPT for 24 months.

Aspect 101. The milvexian for use of any one of aspects 94-100, wherein the SAPT is selected from aspirin, and a P2Y12 inhibitor.

Aspect 102. The milvexian for use of any one of aspects 94-101, wherein the SAPT comprises aspirin.

Aspect 103. The milvexian for use of aspect 102, wherein the aspirin is administered once daily.

Aspect 104. The milvexian for use of aspect 102 wherein the aspirin is administered once daily for a period of ranging from about 3 months to about 3 years.

Aspect 105. The milvexian for use of aspect 102, wherein the aspirin is administered once daily for 3 months.

Aspect 106. The milvexian for use of aspect 102, wherein the aspirin is administered once daily for 6 months.

Aspect 107. The milvexian for use of aspect 102, wherein the aspirin is administered once daily for 12 months.

Aspect 108. The milvexian for use of aspect 102, wherein the aspirin is administered once daily for 24 months.

Aspect 109. The milvexian for use of aspect 102, wherein the aspirin is administered once daily for 36 months.

Aspect 110. The milvexian for use of aspect 102, wherein the aspirin is administered twice daily.

Aspect 111. The milvexian for use of aspect 102, wherein the aspirin is administered once daily at an amount of 75 mg to 100 mg.

Aspect 112. The milvexian for use of aspect 102, wherein the aspirin is administered once daily at an amount of 75 mg to 100 mg for a period of 12 months.

Aspect 113. The milvexian for use of any one of aspects 94-101, wherein the SAPT comprises a P2Y12 inhibitor.

Aspect 114. The milvexian for use of any one of aspects 94-101, wherein the SAPT comprises a P2Y12 inhibitor selected from prasugrel, clopidogrel, selatogrel, or ticagrelor.

Aspect 115. The milvexian for use of aspect 113 or 114, wherein the P2Y12 inhibitor is administered for a period ranging from about 3 months to about 6 months.

Aspect 116. The milvexian for use of aspect 113 or 114, wherein the P2Y12 inhibitor comprises clopidogrel.

Aspect 117. The milvexian for use of aspect 113 or 114, wherein the P2Y12 inhibitor comprises ticagrelor.

Aspect 118. The milvexian for use of aspect 113 or 114, wherein the P2Y12 inhibitor comprises ticagrelor administered at 90 mg twice daily.

Aspect 119. The milvexian for use of aspect 113 or 114, wherein the P2Y12 inhibitor comprises ticagrelor administered at 90 mg twice daily for a period of 3 years.

Aspect 120. The milvexian for use of aspect 113 or 114, wherein the P2Y12 inhibitor comprises ticagrelor administered first followed by aspirin monotherapy administered at 20 mg twice daily.

Aspect 121. The milvexian for use of any one of aspects 113 or 114, wherein the SAPT comprises aspirin monotherapy administered first for a period of ranging from 30 days to 90 days followed by clopidogrel monotherapy.

Aspect 122. The milvexian for use of any one of aspects 113 or 114, wherein the SAPT comprises aspirin monotherapy administered first for a period of 60 days followed by clopidogrel monotherapy.

Aspect 123. The milvexian for use of any one of aspects 94-122, wherein the SAPT comprises aspirin monotherapy administered first for a period of 90 days followed by clopidogrel monotherapy.

Aspect 124. The milvexian for use of any one of aspects 94-122, wherein the SAPT comprises aspirin monotherapy administered first followed by clopidogrel monotherapy for a period of at least 2 months.

Aspect 125. The milvexian for use of any one of aspects 94-122, wherein the SAPT comprises aspirin monotherapy administered first for a period ranging from 30 days to 90 days followed by clopidogrel monotherapy for a period of at least 2 months.

Aspect 126. The milvexian for use of any one of aspects 1-93, wherein the antiplatelet therapy comprises a dual antiplatelet therapy (DAPT).

Aspect 127. The milvexian for use of aspect of 126, wherein the DAPT comprises aspirin and prasugrel.

Aspect 128. The milvexian for use of aspect of 126, wherein the DAPT comprises aspirin and prasugrel administered for 12 months.

Aspect 129. The milvexian for use of aspect of 126, wherein the DAPT comprises aspirin and clopidogrel.

Aspect 130. The milvexian for use of aspect of 126, wherein the DAPT comprises aspirin and clopidogrel administered for a period of 21 days to 12 months.

Aspect 131. The milvexian for use of aspect of 126, wherein the DAPT comprises aspirin and clopidogrel administered for a period of 21 days.

Aspect 132. The milvexian for use of aspect of 126, wherein the DAPT comprises aspirin and clopidogrel administered for a period of 6 months.

Aspect 133. The milvexian for use of aspect of 126, wherein the DAPT comprises aspirin and clopidogrel administered for a period of 12 months.

Aspect 134. The milvexian for use of aspect of 126, wherein the DAPT comprises aspirin and ticagrelor.

Aspect 135. The milvexian for use of aspect of 126, wherein the DAPT comprises aspirin and ticagrelor administered for 12 months.

Aspect 136. The milvexian for use of aspect 126, wherein the antiplatelet therapy comprises a DAPT consisting of aspirin administered once daily at 75 mg to 100 mg and a P2Y12 inhibitor.

Aspect 137. The milvexian for use of aspect 126, wherein the antiplatelet therapy comprises a DAPT administered first followed by administering a SAPT.

Aspect 138. The milvexian for use of aspect 126, wherein the antiplatelet therapy comprises a DAPT administered first followed by administering aspirin monotherapy.

Aspect 139. The milvexian for use of aspect 126, wherein the antiplatelet therapy comprises a DAPT consisting of aspirin 75-100 mg once daily and a P2Y12 inhibitor administered for a first period of 12 months followed by administering aspirin monotherapy for a second period of 6 months.

Aspect 140. The milvexian for use of aspect 126, wherein the antiplatelet therapy comprises a DAPT consisting of aspirin 75-100 mg once daily and a P2Y12 inhibitor administered for a first period of 21 days followed by administering aspirin monotherapy for a second period of 6 months.

Aspect 141. The milvexian for use of aspect 126, wherein the antiplatelet therapy comprises a DAPT consisting of aspirin 75-100 mg once daily and clopidogrel 75 mg once daily administered for a first period of 21 days followed by administering aspirin monotherapy for a second period of 6 months.

Aspect 142. The milvexian for use of aspect 126, wherein the antiplatelet therapy comprises a DAPT consisting of aspirin 75-100 mg once daily and a P2Y12 inhibitor administered for a first period of 21 days followed by administering aspirin monotherapy for a second period of 12 months.

Aspect 143. The milvexian for use of aspect 126, wherein the antiplatelet therapy comprises a DAPT consisting of aspirin 75-100 mg once daily and ticagrelor 90 mg twice daily.

Aspect 144. The milvexian for use of aspect 126, wherein the antiplatelet therapy comprises a DAPT consisting of aspirin 75-100 mg once daily and ticagrelor 90 mg twice daily administered for a period of 12 months.

Aspect 145. The milvexian for use of aspect 126, wherein the antiplatelet therapy comprises a DAPT consisting of aspirin 81-100 mg once daily and ticagrelor 90 mg twice daily.

Aspect 146. The milvexian for use of aspect 126, wherein the antiplatelet therapy comprises a DAPT consisting of aspirin 81-100 mg once daily and ticagrelor 90 mg twice daily administered for 3 years.

Aspect 147. The milvexian for use of aspect 126, wherein the antiplatelet therapy comprises a DAPT consisting of aspirin 75-100 mg once daily and ticagrelor 90 mg twice daily administered for a first period of 12 months followed by administering aspirin monotherapy 75-100 mg once daily for a second period of 12 months.

Aspect 148. The milvexian for use of aspect 126, wherein the antiplatelet therapy comprises a DAPT consisting of aspirin 75-100 mg once daily and ticagrelor 90 mg twice daily administered for a first period of 12 months followed by administering ticagrelor 90 mg twice daily for a second period of 23 months.

Aspect 149. The milvexian for use of any one of the previous aspects, wherein the clinical benefit of reducing the incidence rates of the ischemic stroke by the regimen is maintained throughout a treatment period of 90 days.

Aspect 150. The milvexian for use of any one of the previous aspects, wherein the clinical benefit of reducing the incidence rates of the ischemic stroke is maintained throughout a treatment period of 180 days.

Aspect 151. The milvexian for use of any one of the previous aspects, wherein the clinical benefit of reducing the incidence rates of the ischemic stroke is maintained throughout a treatment period of 12 months.

Aspect 152. The milvexian for use of any one of the preceding aspects, wherein administration of the regimen reduces the patient's FXI clotting activity by about 7% to about 20% relative to baseline.

Aspect 153. The milvexian for use of any one of the preceding aspects, wherein administration of the regimen results is a prolongation of activated partial thromboplastin time (aPTT) ranging from about 27% to about 64% relative to baseline.

Aspect 154. The milvexian for use of any one of the preceding aspects, where the administration does not result in a statistically significant increase in major bleeding complications.

Aspect 155. The milvexian for use of any one of the preceding aspects, wherein the pharmaceutical composition comprising milvexian (or pharmaceutically acceptable salt or solvate thereof) is a solid oral pharmaceutical composition.

Aspect 156. The milvexian for use of aspect 155, wherein the solid oral pharmaceutical composition is a tablet.

Aspect 157. The milvexian for use of aspect 156, wherein the tablet is an immediate release tablet.

Aspect 158. The milvexian for use of any one of the preceding aspects, wherein the human patient is unable to swallow a tablet dosage form.

Aspect 159. The milvexian for use of any one of aspects 156-158, wherein the tablet is dispersed in an aqueous medium to form an aqueous dispersion.

Aspect 160. The milvexian for use of aspect 159, wherein the aqueous medium is water, saline, phosphate buffer, vegetable juice, or fruit juice, including, for example, apple sauce.

Aspect 161. The milvexian for use of aspect 159 or aspect 160, wherein the aqueous dispersion is administered to the human patient via a nasogastric tube or spoon.

Aspect 162. The milvexian for use of any one of the preceding aspects, wherein the pharmaceutical composition is administered orally as an aqueous dispersion by dispersing the oral tablet in an aqueous medium in less than 1 minute.

Aspect 163. The milvexian for use of any one of the preceding aspects, wherein the pharmaceutical composition comprises an amorphous form of milvexian admixed with a polymer.

Aspect 164. The milvexian for use of any one of the preceding aspects, wherein the pharmaceutical composition comprises a spray dried amorphous solid dispersion consisting of 75% w/w milvexian and 25% w/w of HPMC-AS.

Aspect 165. The milvexian for use of any one of the preceding aspects, wherein the milvexian for use results in no clinically significant QTc interval prolongation.

### Aspect Set #7

Aspect 1. A method of preventing adverse cerebrovascular events or adverse cardiovascular events in a human patient with acute coronary syndrome, wherein the method comprises administering to the human patient a regimen comprising: (i) a pharmaceutical composition comprising 25 mg of milvexian (or pharmaceutically acceptable salt or solvate thereof); and (ii) an antiplatelet therapy selected from the group consisting of aspirin, a P2Y12 inhibitor, and combination thereof; wherein the pharmaceutical composition is administered twice daily.

Aspect 2. The method of aspect 1, wherein the antiplatelet therapy is a P2Y12 inhibitor.

Aspect 3. The method of aspect 2, wherein the P2Y12 inhibitor is clopidogrel, ticagrelor, or prasugrel.

Aspect 4. The method of any one of aspects 1-3, wherein the antiplatelet therapy is aspirin.

Aspect 5. The method of any one of aspects 1-4, wherein the human patient is treated with aspirin and clopidogrel combination therapy from day 1 to day 21, followed by aspirin monotherapy for at least 90 days.

Aspect 6. The method of aspect 5, wherein the patient is treated with aspirin and/or clopidogrel without milvexian dose adjustment.

Aspect 7. The method of any one of the preceding aspects, wherein the method is for primary prevention of adverse cerebrovascular events or adverse cardiovascular events.

Aspect 8. The method of any one of the preceding aspects, wherein the method is for secondary prevention of adverse cerebrovascular events or adverse cardiovascular events.

Aspect 9. The method of any one of aspects 1-8, wherein the adverse cerebrovascular events or adverse cardiovascular events comprises one or more of stroke, heart attack, or death.

Aspect 10. The method of any one of aspects 1-9, wherein the adverse cerebrovascular events or adverse cardiovascular events occur in an organ selected from heart, brain, limb, blood supply system, or vessel.

Aspect 11. The method of any one of aspects 1-10, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.

Aspect 12. The method of any one of aspects 1-11, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.

Aspect 13. The method of any one of aspects 1-12, wherein the adverse cerebrovascular events or adverse cardiovascular events are selected from the group consisting of stroke, and combinations thereof.

Aspect 14. The method of any one of aspects 1-11, wherein the adverse cerebrovascular events or adverse cardiovascular events comprises cardiovascular death.

Aspect 15. The method of any one of aspects 1-10, wherein the adverse cerebrovascular events or adverse cardiovascular events comprises arrhythmogenic cardiomyopathy (ACM).

Aspect 16. A method for preventing adverse cardiovascular events in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof;
wherein the pharmaceutical composition is administered twice daily; and
wherein the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; and transient ischemic attack (TIA).

Aspect 17. The method of aspect 16, wherein administration of the regimen results in a relative risk reduction (RRR) in the occurrence of symptomatic venous thromboembolism (VTE: (pulmonary embolism (PE), deep vein thrombosis (DVT) in the patient of at least 25%, without increased bleeding, versus a placebo group.

Aspect 18. The method of aspect 16, wherein the adverse cardiovascular event is one or more of CV death, MI, or ischemic stroke.

Aspect 19. A method for reducing incidence rate of the one or more of adverse thrombotic event selected from new ischemic stroke, MI, or all-cause death in a human patient diagnosed with acute coronary syndrome, wherein the method comprises administering to the human patient a regimen comprising: (i) a pharmaceutical composition comprising about 25 mg to about 100 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

Aspect 20. The method of aspect 19, wherein administration of the regimen results in a relative risk is 0.85 or less relative to the placebo.

Aspect 21. A method for preventing ischemic stroke in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

Aspect 22. The method of aspect 21, wherein administration of the regimen results in a relative risk reduction (RRR) in the occurrence of clinical ischemic stroke events in the patient is at least 25%, without increased bleeding, versus a placebo group.

Aspect 23. The method of aspect 21 or aspect 22, wherein the clinical benefit of reducing the incidence rates of the clinical ischemic stroke by the regimen is maintained throughout a treatment period of 90 days.

Aspect 24. The method of any one of the preceding aspects, wherein administration of the regimen reduces the patient's FXI clotting activity by about 7% to about 20% relative to baseline.

Aspect 25. The method of any one of the preceding aspects, wherein administration of the regimen results is a prolongation of activated partial thromboplastin time (aPTT) ranging from about 27% to about 64% relative to baseline.

Aspect 26. The method of any one of the preceding aspects, where the administration does not result in a statistically significant increase in major bleeding complications.

Aspect 27. The method of any one of the preceding aspects, wherein the pharmaceutical composition comprising milvexian (or pharmaceutically acceptable salt or solvate thereof) is a solid oral pharmaceutical composition.

Aspect 28. The method of aspect 27, wherein the solid oral pharmaceutical composition is a tablet.

Aspect 29. The method of aspect 28, wherein the tablet is an immediate release tablet.

Aspect 30. The method of aspect 28 or aspect 29, wherein the tablet has a disintegration time in water of less than 20 seconds.

Aspect 31. The method of any one of the preceding aspects, wherein the administration results in a milvexian plasma half-life ranging from about 13 hours to about 16 hours.

Aspect 32. The method of any one of the preceding aspects, wherein the administration results in milvexian plasma concentration reaching steady-state at about 3 days to 6 days.

Aspect 33. The method of any one of the preceding aspects, wherein the pharmaceutical composition comprising milvexian (or pharmaceutically acceptable salt or solvate thereof) is administered without regard to the timing of food intake.

Aspect 34. The method of any one of the preceding aspects, wherein the human patient is unable to swallow a tablet dosage form.

Aspect 35. The method of any one of aspects 28-34, wherein the tablet is dispersed in an aqueous medium to form an aqueous dispersion.

Aspect 36. The method of aspect 35, wherein the aqueous medium is water, saline, phosphate buffer, vegetable juice, or fruit juice, including, for example, apple sauce.

Aspect 37. The method of aspect 35 or aspect 36, wherein the aqueous dispersion is administered to the human patient via a nasogastric tube or spoon.

Aspect 38. The method of aspect any one of aspects 28-37, wherein the pharmaceutical composition is administered orally as an aqueous dispersion by dispersing the oral tablet in an aqueous medium in less than 1 minute.

### EXAMPLES

### Example 1A. a Phase 3, Randomized, Double-Blind, Placebo-Controlled, Event-Driven Study to Demonstrate the Efficacy and Safety of Milvexian, an Oral Factor XIa Inhibitor, after a Recent Acute Coronary Syndrome

This is a Phase 3 multicenter, randomized, double-blind, placebo-controlled, parallel-group, event driven, superiority, group-sequential study to evaluate the efficacy and safety of milvexian in participants enrolled within 7 days of an ACS (defined as STEMI or NSTEMI or UA with biomarker positivity), who have undergone cardiac catheterization with PCI or who are being managed conservatively with or without catheterization, and who are receiving antiplatelet therapy standard-of-care as determined by the investigator.

### Study Population

Approximately 16,000 participants are randomized within 7 days of an ACS event to reach the prespecified number of 875 participants with one or more primary efficacy outcome events.

Inclusion Criteria: Each potential participant must satisfy all of the following criteria to be enrolled in the study:

### Age

1. ≥18 years of age.

### Type of Participant and Disease Characteristics

2. Participants must have an index event that meets all 3 of the following criteria within 7 days prior to randomization:
a) Clinical syndrome consistent with spontaneous cardiac ischemia
b) Diagnosis of ACS (i.e., STEMI, non-STEMI, or UA)
c) Cardiac biomarker elevation (e.g., troponin I, troponin T, CK-MB) above the upper limit of normal as determined by the local laboratory.

3. Participants must have at least 2 of the following risk factors: |
a) Age 65 or older
b) Diabetes mellitus
c) History of a prior MI (other than index ACS event)
d) Multivessel CAD (Note: this may be a history of multivessel CAD or a history of single vessel CAD with the index ACS event being in a different coronary artery, establishing multivessel CAD.)
e) History of CABG surgery prior to index ACS event
f) History of PAD or cerebrovascular disease (eg., carotid atherosclerosis, intracranial artery stenosis. Please note that participants with a history of TIA or stroke are excluded from enrollment.)
g) Conservative management (i.e., no PCI or CABG after index ACS event)
h) Any one or more of the following high-risk angiographic features
   a. Total stent length of >30 mm
   b. Thrombotic target lesion
   c. Bifurcation lesion treated with more than one stent
   d. Calcified target lesion treated with atherectomy
   e. Treatment of obstructive left main or proximal left anterior descending artery for index ACS (or clinical diagnosis of an anterior STEMI)

### Weight

Not applicable.

### Sex and Contraceptive/Barrier Requirements

4. All female participants of childbearing potential must have a negative highly sensitive serum β-hCG or urine test at screening.

5. A female participant must not be pregnant, breastfeeding, or planning to become pregnant until 4 days (5 half-lives) after the last dose of study intervention.

6. A female participant must be
a. Not of childbearing potential
b. Of childbearing potential and practicing a highly effective method of contraception (failure rate of <1% per year when used consistently and correctly) and agrees to remain on a highly effective method until 4 days after last dose (5 half-lives) after the last dose of study intervention—the end of relevant exposure.

7. A female participant using hormonal contraceptives should use an additional nonhormonal contraceptive method (above that required in Inclusion Criterion 6b) until 4 days after the last dose (5 half-lives) of study intervention—the end of relevant exposure.

Exclusion Criteria: Any potential participant who meets any of the following criteria will be excluded from participating in the study:

### Medical Conditions

1. MI secondary to ischemia due to either increased oxygen demand or decreased supply (Type 2 MI) or periprocedural MI as the index ACS event.

2. Minimal or no obstructive CAD on angiography performed for the index ACS event prior to any PCI (i.e., <50% stenosis visually as determined by the investigator).

3. Planned CABG or staged PCI after randomization (Note: participants may be evaluated for enrollment after completion of a planned staged PCI).

4. Any condition that requires chronic anticoagulation at the discretion of the investigator and/or local guidelines. Note: Participants with an indication for chronic antiplatelet therapy after placement of a bio-prosthetic non-mechanical valve (eg, transcatheter aortic valve replacement) do not satisfy this exclusion criterion and are eligible for study participation.

5. Conditions with a significant increased risk of bleeding (e.g., clinically significant bleeding within previous 3 months, known bleeding diathesis, etc.)

6. Requires dialysis on a permanent basis or has an eGFR <15 mL/min/1.73 m² at screening.

7. Current active liver disease (eg, acute hepatitis and known cirrhosis), including participants receiving antiviral treatment for hepatitis

8. Active cancer undergoing chemotherapy or radiation or treatment with immunotherapy

9. CABG performed during index event

10. History of ischemic stroke or TIA

11. Killip Class 3 or 4 at the time of randomization

12. History of any significant drug allergy (such as anaphylaxis, Stevens-Johnson Syndrome, toxic epidermal necrolysis, DRESS)

13. Known allergies, hypersensitivity, or intolerance to milvexian or its excipients (refer to the milvexian IB)

14. Known aPTT prolongation >1.5× the ULN or known congenital FXI deficiency Prior/Concomitant Therapy

15. Planned use of any disallowed therapies, ConcomitantTherapy, including isoniazid (INH)

### Prior/Concurrent Clinical Study Experience

16. Received an investigational study intervention or used an invasive investigational medical device within 4 weeks before the planned first dose of study intervention or is currently enrolled in an investigational study

### Diagnostic Assessments

17. Any of the following laboratory results, based on local laboratory, meeting the criteria specified below prior to randomization, confirmed by repeat:
Platelet count <75,000/μL
ALT >3×ULN
Total bilirubin ≥1.5×ULN unless an alternative causative factor such as Gilbert's Syndrome is identified
Hemoglobin <8.0 g/dL

### Other Exclusions

18. Employee of the investigator or study site, with direct involvement in the proposed study or other studies under the direction of that investigator or study site, as well as family members of the employees or the investigator

19. Any condition for which, in the opinion of the investigator, participation would not be in the best interest of the participant (eg, compromise the well-being) or that could prevent, limit, or confound the protocol-specified assessments or has a life expectancy of <12 months

20. At the time of screening, any participant who will not consider following the study contact schedule, or not allow a contact to the participant, or to the designated family members or health care practitioner, to determine any endpoint events and/or vital status, up until the end of the study should they prematurely discontinue study medication or withdraw from study participation.

21. Mental condition or cognitive impairment/dementia that makes the participant unable to understand the nature, potential risks and benefits of the study.

22. Participants who are incarcerated, including prisoners or participants compulsorily detained for treatment of psychiatric disease.

23. Known current substance abuse.

### Study Treatment and Visits

Participants receive either milvexian (25 mg twice daily orally) or a matching placebo on a background of standard-of-care antiplatelet therapy. Randomization is stratified by the intended, investigator-determined antiplatelet strategy into either 1) DAPT for >90 days (with or without de-escalation to SAPT), 2) DAPT for ≤90 days with de-escalation to SAPT, or 3) SAPT. The total study duration is approximately 3.5 years under current protocol assumptions.

### Background Antiplatelet Therapy:

Planned use SAPT or DAPT and duration of DAPT will be at the discretion of the Investigator, based on standard-of-care guidelines and will be recorded at randomization.

SAPT can be low-dose aspirin (≤100 mg per day) or a P2Y12 inhibitor.

P2Y12 inhibitor (alone or in combination with aspirin) can be clopidogrel, ticagrelor or prasugrel.

P2Y12 inhibitor may be changed or stopped. Such changes must be recorded in the eCRF.

De-escalation from DAPT to SAPT can drop either aspirin or P2Y12 inhibitor and must be recorded in the eCRF.

For example, background antiplatelet therapy regimen and duration may consist of: SAPT comprising ≤ASA 100 mg daily until end of study; SAPT comprising clopidogrel 75 mg daily until end of study; DAPT comprising ≤ASA 100 mg daily plus clopidogrel 75 mg daily for 90 days followed by clopidogrel 75 mg daily until the end of study; or all of the foregoing.

This study will include 3 phases: a screening phase up to 7 days after the index ACS event and prior to randomization, a double-blind treatment phase, and a 30-day follow-up phase. Treatment will begin on the day of randomization and continue until the target number of participants with one or more primary efficacy outcome events has been achieved (followed by an EOT visit [within 30 days after GTED]). Thus, treatment duration with study intervention will be variable depending on when the participant is randomized. It is anticipated that the last participant randomized will have a minimum of approximately 3 months of treatment with study intervention by the time of the GTED.

Study intervention may be taken without regard to the timing of food intake. For participants who are unable to swallow medication, study intervention can be dissolved in water and given via a NG tube or in applesauce.

The milvexian dosage form is a film-coated, direct-compression tablet having the formulation described in Table 1 below:

TABLE 1
Film-coated Tablet of Milvexian at 100 mg Strength

| | Amount | | |
|---|---|---|---|
| | | Amount per dose | Amount per dose |
| Component | wt. % | (25 mg, Ex. 17) | (100 mg, Ex. 18) |
| SDP^{b} | 22.22 | 33.33 | 133.3 |
| Silicified Microcrystalline cellulose | 43.07 | 64.6 | 258.4 |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Core Tablet Weight | 100 | 150 | 600 |
| OpadryQX 321A220063 Yellow^{c} | 3.0 | 4.5 | 18.0 |
| | | 154.5 | 618 |
| Tablet hardness | | Average 60N to | Average 140N to |
| | | 120N (Ph. | 220N (Ph. |
| | | Eur. 2.9.8), | Eur. 2.9.8), |
| | | preferably 90N | preferably 180N |
| Tablet friability | | <0.5% | <0.5% |
| Disintegration time in water | | 13 | 18 |
| (seconds) | | | |

a. The film-coated, direct-compression immediate release tablet of milvexian was prepared according to provisional application US 63/483,486, which is incorporated herein by its entirety.
^{b}SDP means spray dried power comprising spray-dried amorphous solid dispersion of milvexian in HPMC-AS-MG grade polymer in a weight ratio of 3:1 (milvexian:HPMC-AS-MG).
^{c}OpadryQX 321A220063 Yellow is composed of Macrogol (PEG) polyvinyl alcohol grafted copolymer, talc, titanium dioxide, glycerol monocaprylocaprate Type I, polyvinyl alcohol and iron oxide yellow.

As used herein, the term “disintegration time” refers to the time required for the tablet to break into particles under a given set of conditions. In some embodiments, the disintegration time is determined using the apparatus described in Eur. Ph. (PTZ-E Pharma Test, Hainburg, Germany), in distilled water at 37° C. using disks.

In some embodiments in which the solid oral pharmaceutical composition is a tablet, the tablet has a disintegration time in water of less than 60 seconds at 37° C.

In some embodiments in which the solid oral pharmaceutical composition is a tablet, the tablet has a disintegration time in water of less than 20 seconds at 37° C.

In other embodiments in which the solid oral pharmaceutical composition is a tablet, the tablet has a disintegration time in water of less than 15 seconds at 37° C.

In other embodiments in which the solid oral pharmaceutical composition is a tablet, the tablet has a disintegration time in water of less than 10 seconds at 37° C.

The SDP (spray-dried powder) in the tablets (Ex. 17, Ex. 18) is prepared as follows. A solution containing about 12.3 wt. % of milvexian P1.Acetone (equivalent to about 11.25 wt. % of milvexian free form) and about 3.75 wt. % of HPMC-AS MG (AQOAT® AS-MG, Shin-Etsu Chemical Co., Ltd. (Niigata, Japan)) in a solvent mixture containing 80/20 w/w % DCM/MeOH is prepared. The clear solution at 21° C. is spray dried using a Buchi B-290 spray dryer with a 35 Kg/hr drying gas flow-rate capacity, set of the following parameters: Atomization gas flow rate at 25 mm (301 L/hr); feed rate at 7.7 g/min; inlet/outlet temperatures at 67/44° C., condenser temperature of −19° C., spray nozzle orifice diameter of 0.7 mm, and spray nozzle cap diameter of 1.4 mm. The spray drying process proceeds for 11 min to give 11.5 g (89% yield) of wet SDP. The wet SDP is subject to drying for 24 hours in a vacuum oven (Heraeus, Model VT6130 M) at 40° C., with nitrogen flow, and a vacuum of approximately 200 mbar to give 10.7 g (83% yield) of desired dry SDP.

The SDP product is a white powder having an assay of 98.8% and a purity of 99.9% by HPLC. The PXRD diffraction pattern shows a halo pattern with no crystalline peaks indicating the product is amorphous.

### Efficacy Evaluations

The primary, secondary and exploratory efficacy endpoint events encompass the following individual events, often grouped in composites in various combinations, including ACM; CV death; MI; UA; “all stroke” or “any stroke” (ischemic, hemorrhagic, or unknown cause); ischemic stroke; ALI; major vascular (non-traumatic) limb amputation; symptomatic VTE (PE, DVT); ischemia-driven coronary revascularization; stent thrombosis; hospitalization for any reason, categorized as (1) planned or unplanned, (2) for arterial or venous thrombotic event or neither; TIA. An independent CEC will adjudicate efficacy outcome events as defined in the CEC charter (Table 2):

TABLE 2
Primary and Secondary Endpoints

| Objective | Endpoint |
|---|---|
| Primary | |
| To demonstrate that milvexian is superior to | Time to the first |
| placebo, in addition to standard-of-care, in | occurrence of MACE |
| reducing the risk of MACE (the composite of | |
| CV death, MI, and ischemic stroke) | |
| Secondary | |
| To demonstrate that milvexian is superior to | Time to the first |
| placebo, in addition to standard-of-care, in | occurrence of MAVE |
| reducing the risk of MAVE (the composite of | |
| CV death, MI, ischemic stroke, MALE, and | |
| symptomatic VTE) | |
| To determine whether milvexian reduces the | Time to the first |
| risk of the composite of ACM, MI, and | occurrence of ACM, MI, |
| ischemic stroke compared to placebo | and ischemic stroke |
| To determine whether milvexian reduces the | Time to CV death |
| risk of CV death compared to placebo | |
| To determine whether milvexian reduces the | Time to ACM |
| risk of ACM compared to placebo | |

### Pharmacokinetic Evaluations

Plasma samples are collected from approximately 5,000 participants. The concentration of milvexian is measured in samples collected from participants who were treated with milvexian using a validated, specific, and sensitive (e.g., LC-MS/MS) method by or under the supervision of the sponsor.

Residual plasma PK samples may be stored for future analysis and for metabolite profiling. Based on the individual plasma concentration-time data and using the actual dose and sampling times, PK parameters (e.g., apparent clearance, etc.) for exposure-response analysis of milvexian and associated variables are derived using population PK modeling.

### Pharmacodynamic and Biomarker Evaluations

Plasma samples for PD and biomarker assays are collected in approximately 1,000 participants in the study. The PD assay is aPTT. Exploratory biomarkers of disease pathophysiology are proteomics and D-dimer.

### Safety Evaluations

The bleeding endpoints encompass the following individual events, often grouped in composites in various combinations, including BARC categories 2, 3a, 3b, 3c, 4 and 5; ISTH categories major and CRNM; GUSTO categories (1) severe or life threatening and (2) moderate; and TIMI categories non-CABG-related major, CABG-related major, minor, and requiring medical attention. These bleeding outcome events are adjudicated by an independent CEC as defined in the CEC charter. Overall safety and tolerability are assessed via evaluation of AEs, clinical laboratory tests, and vital signs.

### Statistical Methods

Summaries by treatment group using appropriate descriptive statistics are provided for all study variables including demographic and baseline characteristics. No imputation is applied, unless specified otherwise in the SAP. Descriptive statistics such as mean, median, standard deviation, minimum, and maximum are used to summarize continuous variables. Counts and percentages are used to summarize categorical variables. The Kaplan-Meier method is used to summarize time-to-event variables. Graphical data displays may also be used to summarize the data.

Unless stated otherwise, all statistical tests are interpreted at a nominal (that is, without adjustment for multiplicity) 2-sided significance level of 0.05 and all CIs at a nominal 2-sided level of 95%.

The stratified log-rank test is used for testing the primary hypothesis. To control the family-wise type I error rate at alpha of 0.05 (2-sided) in testing for primary and secondary efficacy outcomes, if superiority of milvexian over placebo on the primary efficacy outcome is established, superiority of milvexian over placebo on secondary efficacy outcomes is tested sequentially using a closed testing procedure in the predefined hierarchical order.

The stratified Cox regression is used to estimate the HR and 95% CIs for the treatment effect on the primary efficacy endpoint.

An interim analysis for futility is planned when approximately 60% of target number of participants with the primary efficacy events are observed. As the study will not stop for superiority, the type I error rate does not need to be adjusted.

### Example 1B. a Phase 3, Randomized, Double-Blind, Double-Dummy, Parallel Group, Active-Controlled Study to Evaluate the Efficacy and Safety of Milvexian, an Oral Factor XIa Inhibitor, Versus Apixaban in Participants with Atrial Fibrillation

This is a Phase 3, prospective, randomized, double-blind, parallel-group, active-controlled, multicenter, event driven study comparing the efficacy and safety of milvexian to apixaban for the prevention of stroke and non-CNS systemic embolism in participants with atrial fibrillation.

### Study Population

Approximately 15,500 participants with AF are randomized to reach the pre-specified number of required endpoint events.

### Inclusion Criteria

Each potential participant must satisfy all of the following criteria to be enrolled in the study:

### Age

1. Minimum age of 18 years (or the legal age of consent in the jurisdiction in which the study is taking place)

### Type of Participant and Disease Characteristics

2. Medically stable and appropriate for chronic antithrombotic treatment on the basis of physical examination, medical history, vital signs, and clinical laboratory tests performed as part of standard of care or at screening. Any abnormalities in physical examination, medical history or vital signs must be consistent with the underlying illness in the study population. If the results of the serum chemistry panel, hematology, or urinalysis are outside the normal reference ranges, the participant may be included only if the investigator judges the abnormalities or deviations from normal to be not clinically significant or to be appropriate and reasonable for the population under study. This determination must be recorded in the participant's source documents and initialed by the principal investigator or appropriately qualified designee.

3. Atrial fibrillation or flutter, paroxysmal or sustained not due to a reversible cause, and eligible to receive anticoagulation therapy:

i. Atrial fibrillation or flutter must be documented by ECG evidence (eg, 12-lead ECG, rhythm strip, Holter, pacemaker interrogation) within 30 days before randomization. In addition, participants must have medical evidence of atrial fibrillation within 1 year before and at least 1 day before the qualifying ECG evidence. This could be obtained from a notation in the participant's record (eg, medical chart, hospital discharge summary).

ii. If electrical cardioversion or ablation is planned, the investigator plans to treat the patient with anticoagulation for the duration of the trial.

4. Participant must satisfy one or both of the following categories of risk factors (a or b):
a. One or more of the following risk factors:
   i. Age >75 years at the time of screening
   ii. History of a clinical symptomatic stroke of any type (ischemic, hemorrhagic, lacunar, or undetermined), including silent brain infarcts and cerebral microbleeds (CMB):
   Ischemic stroke must be >7 days prior to first dose of study intervention. Hemorrhagic strokes, and hemorrhagic transformations must have occurred >3 months prior and are allowed per investigator discretion. A prior neurology consultation is recommended to ascertain if the patient is appropriate for anticoagulation. (See Exclusion Criterion 2 for history of subarachnoid hemorrhage, subdural hematoma, or spinal cord hemorrhage)
   Notes:
   CMBs are defined as rounded foci of <10 mm in size that appear hypointense and distinct from vascular flow voids, leptomeningeal hemosiderosis, or non-hemorrhagic subcortical mineralization on T2*-weighted MRI.
   Participants who received thrombolysis and/or mechanical thrombectomy for any indications other than stroke with or without stenting can be randomized, if they have post-thrombolytic therapy/post-thrombectomy INR ≤1.5 and aPTT ≤1.4 times the ULN prior to study randomization.
b. Two or more of the following risk factors:
   i. Age between 65 and 74 years, inclusive, at the time of screening
   ii. Hypertension (defined as use of antihypertensive medications within 6 months before the screening visit or persistent systolic blood pressure above 140 mmHg or diastolic blood pressure above 90 mmHg)
   iii. Diabetes mellitus (defined as a history of diabetes mellitus and current use of antidiabetic medications)
   iv. Atherosclerotic vascular disease meeting one or more of the following criteria:
      Lower extremity PAD defined as one or both of the following:
      a) a documented history of a resting ankle-brachial index (ABI) of <0.85 b) prior major vascular (non-traumatic) amputation (ankle or above), peripheral bypass, or peripheral percutaneous or surgical intervention for limb ischemia
   AND/OR
   CAD defined as one of the following:
      a) a recent or past MI, excluding periprocedural or definite Type 2 MI
      b) history of coronary revascularization, either percutaneous (PCI) or surgical (CABG)
   v. Symptomatic heart failure with a history of heart failure as the primary cause of a hospitalization (regardless of ejection fraction) or a documented left ventricular ejection fraction <40% (regardless of history of heart failure hospitalization)

5. For participants receiving VKA, the INR must be ≤2.0 at the time of randomization with the first dose of study intervention taken the same day

### Weight

Not applicable.

### Sex and Contraceptive/Barrier Requirements

All female participants of childbearing potential must have a negative highly sensitive serum (β-human chorionic gonadotropin [β-hCG]) or urine test up to 2 days before the first dose of study intervention.

6. A female participant must agree not to be pregnant, breastfeeding, or planning to become pregnant while enrolled in this study until 4 days (five half-lives) after the last dose of study intervention.

7. A female participant must be (as defined in Appendix 5, Contraceptive and Barrier Guidance)
a. Not of childbearing potential; or
b. Of childbearing potential and practicing a highly effective method of contraception (failure rate of <1% per year when used consistently and correctly) and agrees to remain on a highly effective method until 4 days (5 half lives) after the last dose of study intervention—the end of relevant exposure.

8. A female participant using hormonal contraceptives should use an additional nonhormonal contraceptive method (above that required in the inclusion criterion 8b) until 4 days after the last dose (five half-lives) of study intervention—the end of relevant exposure.

### Exclusion Criteria

Any potential participant who meets any of the following criteria will be excluded from participating in the study:

### Medical Conditions

1. History of ischemic stroke, but only if within 7 days of stroke onset. An ischemic stroke with onset 8 or more days prior to randomization is permitted.

2. History of any of the following only if within 3 months of randomization: primary hemorrhagic stroke, hemorrhagic transformation of an ischemic stroke, subarachnoid hemorrhage, subdural hematoma, spinal cord hemorrhage.

3. A prior disabling stroke defined by a current mRS of ≥3. Refer to mRS scoring in Study Specific Materials.

4. Hemodynamically significant valve disease that will potentially require surgical valve replacement during the study as determined by the investigator

5. Any condition other than AF that requires chronic anticoagulation at the discretion of the investigator and/or local guidelines, such as mechanical heart valves. However, participants with an indication for chronic antiplatelet therapy after placement of a bio-prosthetic nonmechanical valve (eg, transcatheter aortic valve replacement [TAVR]) do not satisfy this exclusion criterion and are eligible for study participation

6. Any condition that, in the opinion of the investigator, contraindicates anticoagulant therapy, or would, with anticoagulation, have an unacceptable risk of bleeding, or would interfere with the study endpoint assessments, eg, large cerebral infarct volume, active/recent major bleeding, known hereditary bleeding diathesis.

7. Known presence of atrial myxoma or left ventricular thrombus

8. Active endocarditis

9. Current active liver disease (eg, acute hepatitis, known cirrhosis), including participants receiving antiviral treatment for hepatitis

10. Require dialysis at time of randomization

11. Patients with eGFR <25 mL/min/1.73 m² at screening

12. History of any significant drug allergy (such as anaphylaxis, Stevens-Johnson Syndrome, toxic epidermal necrolysis, drug reaction with eosinophilia and systemic symptoms [DRESS]).

13. Hospitalized for acute heart failure at the time of randomization.

14. Known allergies, hypersensitivity, or intolerance to milvexian or its excipients (refer to the milvexian IB).

15. Unable to swallow medications, as determined by the investigator at screening

### Prior/Concomitant Therapy

16. Planned use of any disallowed therapies including isoniazid (INH).

Disallowed therapies:
Isoniazid;
Aspirin >100 mg/day >7 days of consecutive use;
Concomitant use of omeprazole or esomeprazole with clopidogrel; Additional chronic anticoagulants;
The concomitant use of a combined P-gp and strong CYP3A4 inhibitor (e.g., atazanavir, clarithromycin, itraconazole, ketoconazole, ritonavir, saquinavir) within 7 days of receiving study intervention and during the study;

The concomitant use of a combined P-gp and strong CYP3A4/5 inducer (e.g., carbamazepine, phenytoin, rifampin) within 7 days of receiving study intervention and during the study.

Prior/Concurrent Clinical Study Experience 17. Received an investigational intervention or used an invasive investigational medical device within 4 weeks before the planned first dose of study intervention or is currently enrolled in an investigational interventional study.

### Diagnostic Assessments

18. Any of the following laboratory results, based on local laboratory, outside of the ranges specified below prior to randomization, confirmed by repeat:
a. Platelet count <50,000 mm³
b. ALT >3×ULN
c. Total bilirubin ≥1.5×ULN unless an alternative causative factor such as Gilbert's syndrome is identified
d. Hemoglobin <8.0 g/dL

### Other Exclusions

19. Any condition for which, in the opinion of the investigator, participation would not be in the best interest of the participant (eg, compromise the well-being) or that could prevent, limit, or confound the protocol-specified assessments or has a life expectancy of <12 months.

20. At the time of screening, any participant who will not consider following the study contact schedule, or not allow a contact to the participant, or to the designated family members or health care practitioner, to determine any endpoint events and/or vital status, up until the end of the study should they prematurely discontinue study intervention or withdraw from study participation.

21. Participants who are incarcerated, including prisoners or subjects compulsorily detained for treatment of psychiatric disease.

22. Known current substance abuse that could impact study compliance 23. Employee of the investigator or study site, with direct involvement in the proposed study or other studies under the direction of that investigator or study site, as well as family members of the employees or the investigator

### Dose Selection

The 100 mg twice daily dose regimen of milvexian chosen for this study was selected based on the totality of data, including data from the Phase 1 and Phase 2 studies and considerations of using an active comparator (apixaban) in the study design, and thrombotic event pathophysiology in patients with atrial fibrillation (AF; ie, primarily embolism from the left atrial appendage). Both the milvexian Phase 2 studies (AXIOMATICTKR and AXIOMATIC-SSP) primarily used twice daily dosing regimens. With a half-life of about 13 to 16 hours, repeating twice daily dosing provides a lower peak to trough ratio relative to the same total dose administered once a day according to the meta-analysis using applicant's proprietary algorithms (MBMA) and patient data. Madabushi et al., Review: Role of Model-Informed Drug Development Approaches in the Lifecycle of Drug Development and Regulatory Decision-Making, Pharmaceutical Research, 2022, vol. 39, pp. 1669-1680.

In the AXIOMATIC-TKR study (see Example 2), milvexian doses of 50-200 mg twice daily were superior to enoxaparin 40 mg once daily for the prevention of total venous thromboembolism (VTE) with only modest gains in efficacy for the 100 and 200 mg twice daily doses (enoxaparin 40 mg-21.4%, milvexian 50 mg-11.3%, 100 mg-9.0%, 200 mg-7.6%). Historical Phase 3 studies of VTE prevention in orthopedic surgery demonstrated that apixaban 2.5 mg twice daily and rivaroxaban 10 mg once daily were superior to enoxaparin 40 mg once daily. No fatal bleeding and no increases in symptomatic intracranial hemorrhage were observed with milvexian. No dose-response relationship was observed for bleeding events in either the AXIOMATIC-TKR or AXIOMATIC-SSP studies (see Examples 2 and 3, respectively), suggesting a lower bleeding liability for any dose of milvexian compared with Factor Xa inhibitors (i.e., both rivaroxaban and apixaban showed dose-related bleeding in Phase 2 orthopedic surgery studies and in combination with antiplatelets after an ACS).

In the development of currently approved direct oral anticoagulants (DOACs), studies of VTE prevention were used as a benchmark for predicting the prevention of cardioembolic events related to AF.

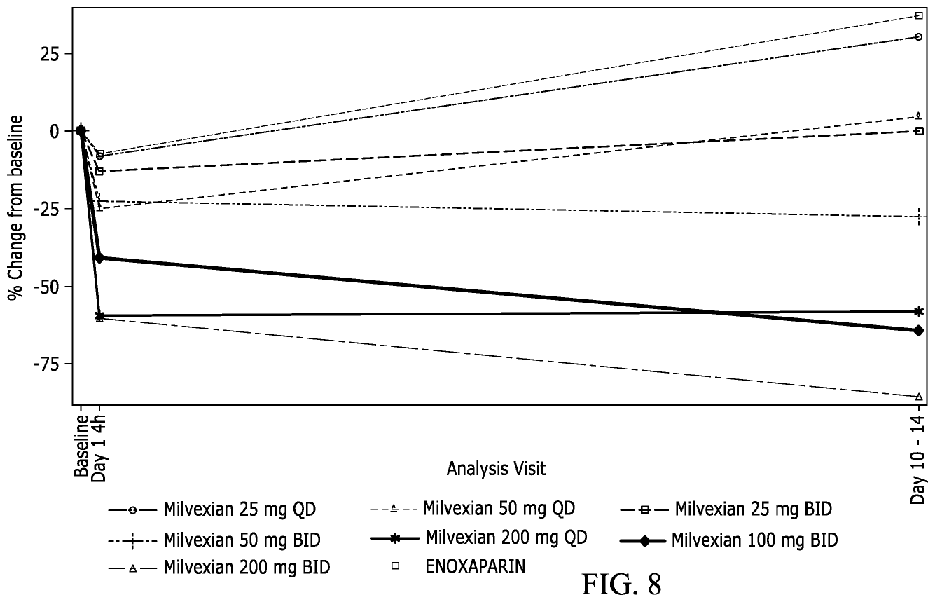

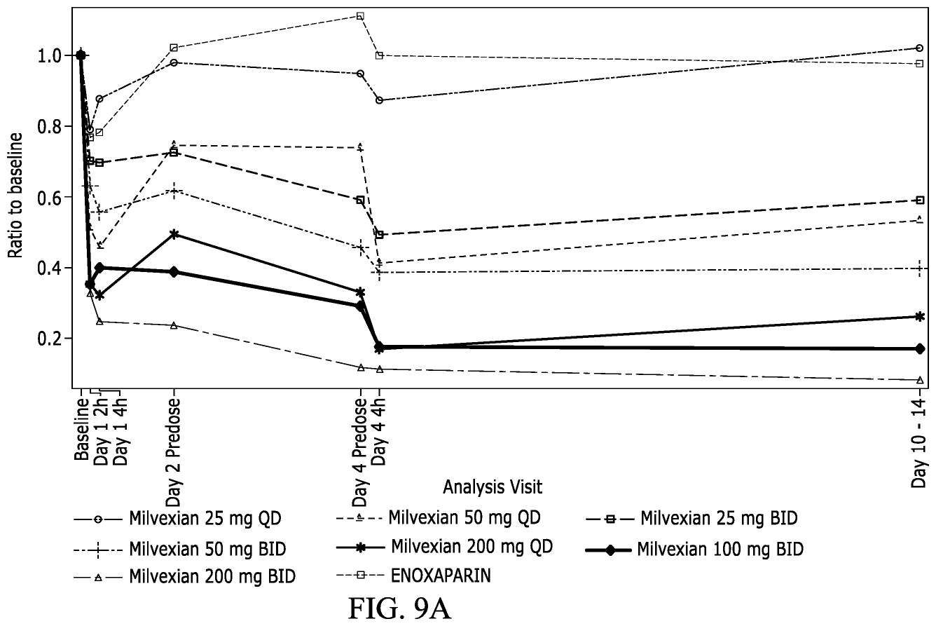

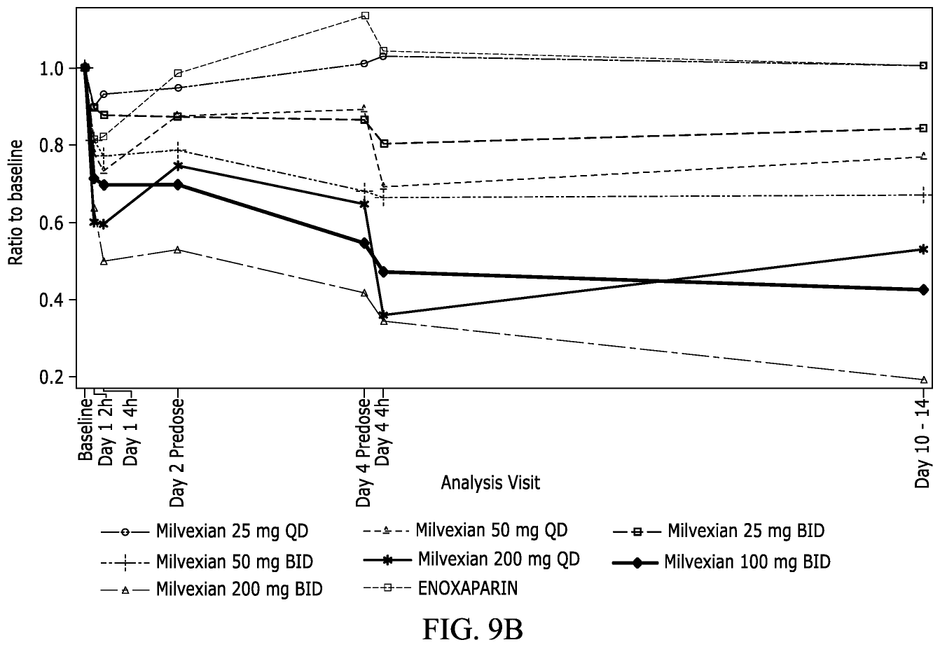

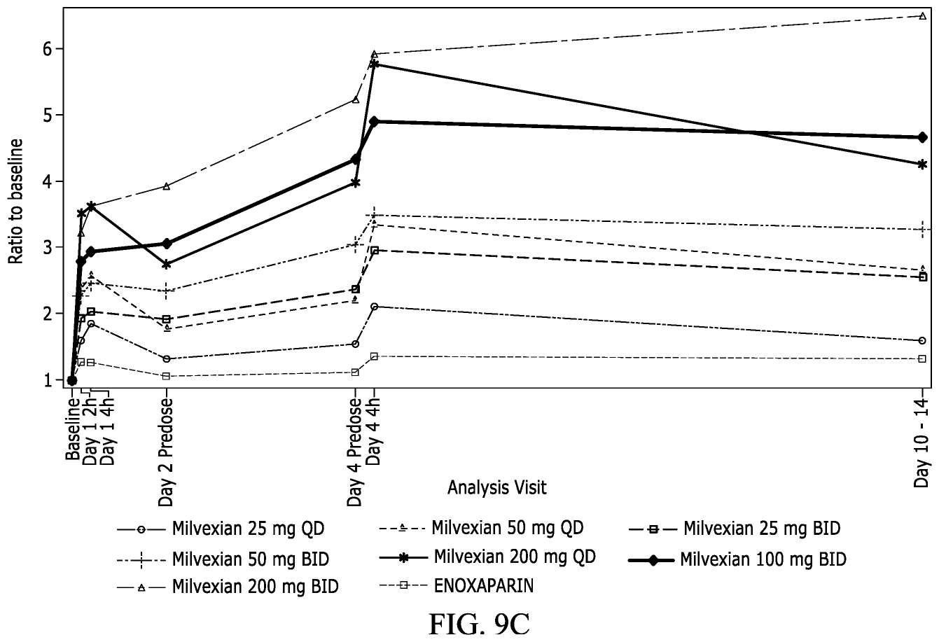

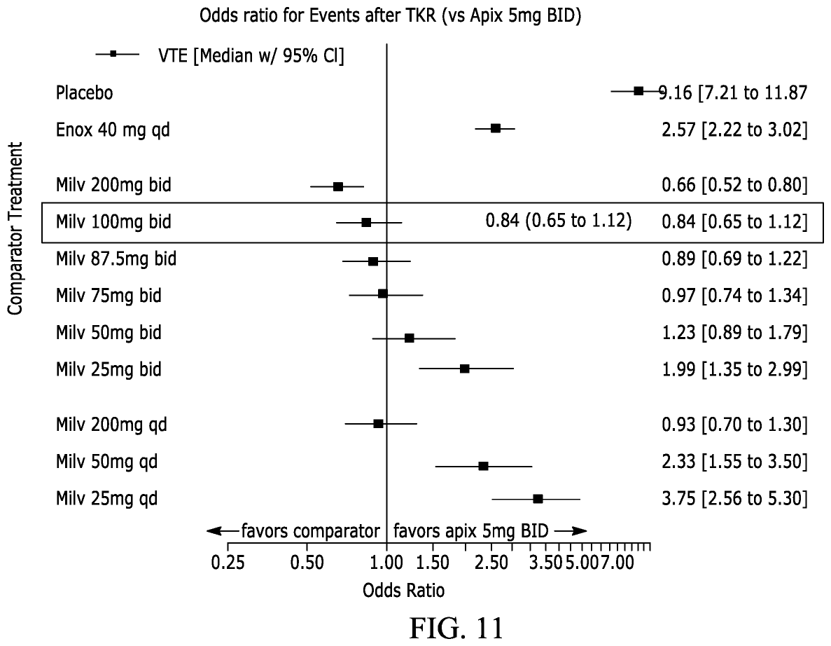

To guide selection of an efficacious dose of milvexian, a model based meta-analysis (MBMA) was performed. MBMA is a method used to integrate data from multiple studies using mathematical models to quantitatively describe the effect of treatments on trial outcomes. MBMA facilitates a quantitative comparison of the therapeutic landscape. A total 45 studies of VTE prevention, involving studies for currently approved DOACS, and milvexian AXIOMATIC-TKR and AXIOMATIC-SSP, were used to perform a MBMA. These included 54 treatment arms for 10 different treatments including placebo in a total of 5,434 trial participants. These randomized clinical trials were combined with the AXIOMATIC-TKR Phase 2 study results (applicant's proprietary information) to develop a non-linear mixed effect, Eₘₐₓ dose-response MBMA model (algorithm codes written by the applicant). Covariates included in the model were indication, geographic location, year of each trial, drug dosing frequency, and dosing information. Eₘₐₓ values were assumed to be shared across treatments for a single endpoint, while Ed₅₀ was estimated for each treatment separately. Random effects account for unexplained variability across trials by endpoint. This MBMA model provided a quantitative understanding of VTE rates for multiple milvexian doses compared against approved anti-thrombotic treatments (apixaban and Enoxaparin). Modeling results comparing milvexian against apixaban 5 mg BID as odds ratio of VTE are summarized in the forest plot in FIG. 8 where an odds ratio <1 favors milvexian. These results show that milvexian 100 mg twice daily (BID) exhibited an odds ratio of 0.84 [0.65,1.12] (median [95% confidence interval, CI]) compared to apixaban 5 mg BID. The upper 95% CI bound of 1.12 falls within the boundary of non-inferiority for milvexian 100 mg BID against apixaban 5 mg BID. Lower doses were projected to have inferior efficacy as compared to apixaban 5 mg BID (See FIG. 11, odds ratio plot for milvexian at various BID doses vs apixaban at 5 mg BID in reducing VTE rates).

Considering both bleeding observations, efficacy projections, and other considerations, milvexian 100 mg twice daily was selected for confirming the primary hypothesis of the Phase 3 study.

### Study Drug

At randomization on Day 1, participants are randomly assigned in double blind manner in a 1:1 ratio to receive either milvexian 100 mg (twice daily) or apixaban 5.0 mg twice daily. The apixaban dose is 2.5 mg twice daily for participants with at least 2 of the following characteristics: age ≥80 years, body weight ≤60 kg, or serum creatinine ≥1.5 mg/dL. Study intervention may be taken without regard to food intake.

The milvexian dosage form is a film-coated, direct-compression immediate release tablet^{a} having the formulation described in the Table 3 below:

TABLE 3
Film-coated Tablet of Milvexian at 100 mg Strength

| | Amount | | |
|---|---|---|---|
| | | Amount per dose | Amount per dose |
| Component | wt. % | (25 mg, Ex. 17) | (100 mg, Ex. 18) |
| SDP^{b} | 22.22 | 33.33 | 133.3 |
| Silicified Microcrystalline cellulose | 43.07 | 64.6 | 258.4 |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Core Tablet Weight | 100 | 150 | 600 |
| OpadryQX 321A220063 Yellow^{c} | 3.0 | 4.5 | 18.0 |
| | | 154.5 | 618 |
| Tablet hardness | | Average 60N to | Average 140N to |
| | | 120N (Ph. | 220N (Ph. |
| | | Eur. 2.9.8), | Eur. 2.9.8), |
| | | preferably 90N | preferably 180N |
| Tablet friability | | <0.5% | <0.5% |
| Disintegration time in water | | 13 | 18 |
| (seconds) | | | |

a. The film-coated, direct-compression immediate release tablet of milvexian was prepared according to provisional application US 63/483,486, which is incorporated herein by its entirety.
^{b}SDP means spray dried power comprising spray-dried amorphous solid dispersion of milvexian in HPMC-AS-MG grade polymer in a weight ratio of 3:1 (milvexian:HPMC-AS-MG).
^{c}OpadryQX 321A220063 Yellow is composed of Macrogol (PEG) polyvinyl alcohol grafted copolymer, talc, titanium dioxide, glycerol monocaprylocaprate Type I, polyvinyl alcohol and iron oxide yellow.

The SDP (spray-dried powder) in the tablets (Ex. 17, Ex. 18) is prepared as follows. A solution containing about 12.3 wt. % of milvexian P1.Acetone (equivalent to about 11.25 wt. % of milvexian free form) and about 3.75 wt. % of HPMC-AS MG (AQOAT® AS-MG, Shin-Etsu Chemical Co., Ltd. (Niigata, Japan)) in a solvent mixture containing 80/20 w/w % DCM/MeOH is prepared. The clear solution at 21° C. is spray dried using a Buchi B-290 spray dryer with a 35 Kg/hr drying gas flow-rate capacity, set of the following parameters: Atomization gas flow rate at 25 mm (301 L/hr); feed rate at 7.7 g/min; inlet/outlet temperatures at 67/44° C., condenser temperature of −19° C., spray nozzle orifice diameter of 0.7 mm, and spray nozzle cap diameter of 1.4 mm. The spray drying process proceeds for 11 min to give 11.5 g (89% yield) of wet SDP. The wet SDP is subject to drying for 24 hours in a vacuum oven (Heraeus, Model VT6130 M) at 40° C., with nitrogen flow, and a vacuum of approximately 200 mbar to give 10.7 g (83% yield) of desired dry SDP.

The SDP product is a white powder having an assay of 98.8% and a purity of 99.9% by HPLC. The PXRD diffraction pattern shows a halo pattern with no crystalline peaks indicating the product is amorphous.

### Efficacy Evaluations

The primary efficacy endpoint event is the composite of stroke and non-CNS systemic embolism. Other secondary efficacy endpoint events include: (1) the composite of CV death, MI, stroke, and non-CNS systemic embolism, (2) CV death, (3) the composite of all-cause death, MI, stroke and non-CNS systemic embolism, and (4) the composite of CV death, MI, stroke, any unanticipated revascularization (including amputation for ischemic limb), and urgent hospitalization for vascular cause of ischemic nature (including thrombotic events: deep vein thrombosis (DVT) and pulmonary embolism [PE]).

All efficacy events occurring from the time of randomization through the End-of-Study contact are collected.

All primary and secondary efficacy events are assessed as time to the first occurrence of the event from randomization through the global targeted endpoint date (GTED), which is defined as the date when the projected target number of primary efficacy endpoint events has been achieved.

Exploratory efficacy events include the components collected for the primary and secondary endpoints either alone and/or in combination. Thus, exploratory objectives include:

To explore if milvexian when compared to apixaban reduces the risk of:
a) The composite of All Cause Death, MI, stroke, and ALI
b) Stroke subtypes
c) MI
d) All Cause Death
e) Fatal or disabling stroke
f) Non-CNS SE
g) Other individual components and the subtypes of a component not included above

To characterize the PK of milvexian;

To explore the exposure-response relationships for key efficacy and safety endpoints and PD endpoints; and

To collect medical resource utilization data to be incorporated in economic modeling for participants treated with milvexian compared to apixaban.

### Pharmacokinetic Evaluations

Plasma samples are collected from approximately 5,000 participants. Samples are analyzed from participants in the PK subset to determine concentration of milvexian using a validated, specific, and sensitive (eg, liquid chromatography-mass spectrometry/mass spectrometry) method.

Based on the individual plasma concentration-time data and using the actual dose and sampling times, PK parameters for exposure-response analysis of milvexian and associated variables may be derived using population PK modeling.

### Pharmacodynamic and Biomarker Evaluations

Plasma samples for PD and biomarkers assays are collected in approximately 1,000 participants in the study.

The PD assay is aPTT. Exploratory biomarkers of disease pathophysiology are proteomics and D-dimer.

### Safety Evaluations

The principal safety endpoint family includes ISTH major bleeding events and the composite of ISTH major and CRNM bleeding events. Bleeding events are assessed by other criteria (GUSTO, BARC and TIMI). All safety events occurring from the time of randomization through the EOS contact are collected. Bleeding events are assessed as time to the first occurrence of the event from randomization through 2 days after the last dose of study intervention. Overall safety and tolerability assessment will include evaluation of adverse events, clinical laboratory tests, vital signs, and physical examinations.

In particular, the safety endpoints are (1) Time to the first occurrence of ISTH major bleeding; and (2) Time to the first occurrence of the composite of ISTH major and CRNM bleeding.

### Statistical Considerations:

Event-driven; variable treatment duration, at least minimum of exposure for 13 weeks (3 months)
Non-inferiority margin: 1.37 (67% retention)
HR: 1.00 for milvexian compared to apixaban
Alpha: 2.5% (one-sided for NI); 5% (two-sided) for superiority
Relative risk: 0.75 for ISTH major bleeding
Power: 90% for ST/SE, 90% for ISTH major bleeding
Enrollment period/total study duration: 3.5/4 years
Sample Size: approximately 15,500
Number of events: 430 for ST/SE; 530 for ISTH major bleeding.

The components of the composite primary efficacy endpoint will be summarized (with estimates and 95% confidence intervals) to better understand the contribution of component endpoints on the primary efficacy endpoint. Furthermore, cumulative event rates of the primary efficacy endpoint over time will be estimated using the Kaplan-Meier method.

### Example 2. Prevention of New Ischemic Stroke or New Covert Brain Infarction Following Acute Ischemic Stroke or Transient Ischemic Attack

The AXIOMATIC-SSP Antithrombotic treatment with Factor Xia inhibition to Optimize Management of Acute Thromboembolic events in Secondary Stroke Prevention) study was a multi-center, Phase 2, randomized, double-blind, placebo-controlled, dose-ranging study of milvexian for the prevention of new ischemic stroke or new covert brain infarction in participants receiving ASA and clopidogrel following an acute ischemic stroke or TIA. The primary objective was to estimate the dose-response relationship of milvexian in participants with ischemic stroke or TIA treated with ASA and clopidogrel by assessing the composite of new ischemic stroke during the treatment period and new covert brain infarction detected by magnetic resonance imaging (MRI) at Day 90 (assessed by central review). The target randomization ratio by the end of the study was 2:1:1:1:1:1 (placebo, milvexian 25 mg QD, 25 mg BID, 50 mg BID, 100 mg BID, and 200 mg BID). Two additional dosing arms, milvexian 50 mg QD and 100 mg QD were included in the original design, but the protocol was amended (revised protocol 05) to close randomization to these arms to simplify the study design while maintaining scientific rigor to attain trial objectives, minimize the number of study participants exposed to the new investigational drug, reduce the burden on participants and study centers, and streamline study procedures. Due to the early termination of enrollment in the 50 mg QD and 100 mg QD treatment arms and the limited number of participants in both groups, these arms are not included in the presented analyses for efficacy, but are included in baseline characteristics (combined milvexian), subject disposition, and safety.

The study enrolled participants ≥40 years of age who had ischemic stroke or TIA. Ischemic stroke was defined as a neurological deficit attributable to a non-lacunar, acute brain infarction detected by neuroimaging and relevant to the clinical symptoms and National Institutes of Health Stroke Score (NIHSS) ≤7 at the time of randomization. TIA was defined as acute onset neurological deficit attributable to focal ischemia of the brain by history or examination, with complete resolution of the deficit and no brain infarction on neuroimaging and ABCD score ≥6 or presence of motor symptoms. Evidence of relevant intracranial or cervical arterial atherosclerotic plaque, ulceration or thrombus in a feeding artery documented by imaging was required regardless of whether the index event was ischemic stroke or TIA, and all participants were required to have a Modified Rankin Score ≤3 before the index event. Eligible participants were screened for potential inclusion into the study as soon as possible after presentation and randomized within 48 hours of the index event. Following informed consent, participants received ASA 100 mg QD and a single loading dose of clopidogrel 300 mg if not already given as standard of care. Participants were then randomized to receive milvexian or placebo, plus open-label uncoated ASA 100 mg in combination with clopidogrel 75 mg QD for the next 21 days. Participants continued treatment from Day 22 through Day 90 with milvexian or placebo, plus open-label 100 mg uncoated ASA only. A baseline MRI was performed within 48 hours of the onset of the index event and prior to randomization. A second MRI was performed on Day 90±6 days. All participants were required to remain on study treatment until the Day 90 MRI was performed up to Day 96.

From 27 Jan. 2019 to 24 Dec. 2021, 2,799 participants were enrolled and 2,366 participants were randomized into 8 treatment arms. The 200 mg BID arm was opened on 5 Jun. 2021 after approximately 1,387 participants were enrolled and after the DMC reviewed unblinded data to assess the safety of opening the dose arm. The randomization of participants to the 50 mg QD (N=22) and 100 mg QD (N=18) treatment arms was terminated with the implementation of revised protocol amendment 05 as previously described. Overall, the mean (standard deviation [SD]) age of participants was 69.6 (±10.81) years. The majority of the participants was male and white. Overall, most index events (75.7%) were ischemic stroke, with an NIHSS ≤5 in approximately 96% of randomized participants. TIA represented 24.1% of index events; 53.4% of the TIA participants had an ABCD2 score of ≥6. The baseline disease characteristics were balanced among the treatment groups, with the exception of NIHSS score 6 to 7 in the 200 mg BID arm (4.7%) which was higher than in the other groups (range: 2.1% to 4.0%). More participants with NIHSS score 6 or 7 were assigned to the highest dose group due to the staggered opening of that treatment arm and broadening the NIHSS inclusion from ≤5 to ≤7 which occurred with the same protocol amendment. The most common comorbidities/risk factors were hypertension, diabetes, history of cigarette smoking, and hypercholesterolemia. All baseline conditions were well balanced across treatment arms.

The dose-response relationship between milvexian treatment (including placebo) for the primary composite endpoint of symptomatic ischemic stroke during the study period and new covert brain infarction on MRI at Day 90 was analyzed based on a generalized Multiple Comparison Procedure-Modelling (MCP-Mod) analysis, and results were presented by treatment arm.

In the AXIOMATIC-TKR study, the milvexian dose of 25 mg twice daily showed comparable efficacy to enoxaparin with a favorable safety profile. In the AXIOMATIC-SSP study the 25 mg twice daily milvexian dose was numerically better than placebo for preventing clinical ischemic strokes (HR 0.69, 95% CI 0.36-1.30) with no further improvements in efficacy observed at the higher doses.

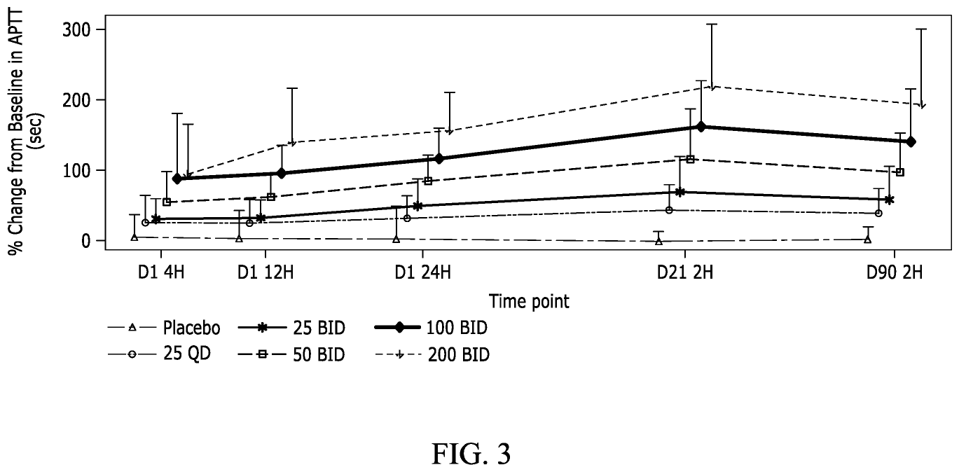

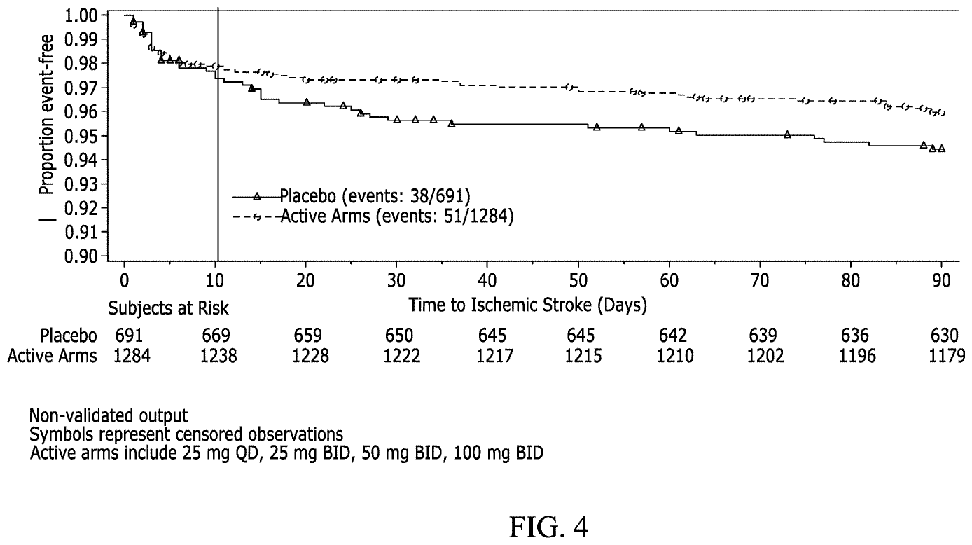

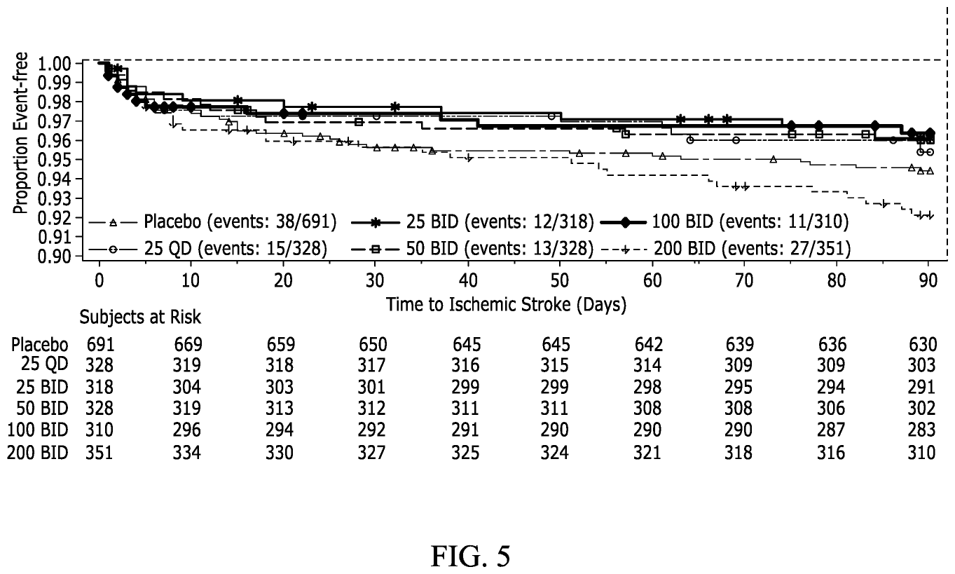

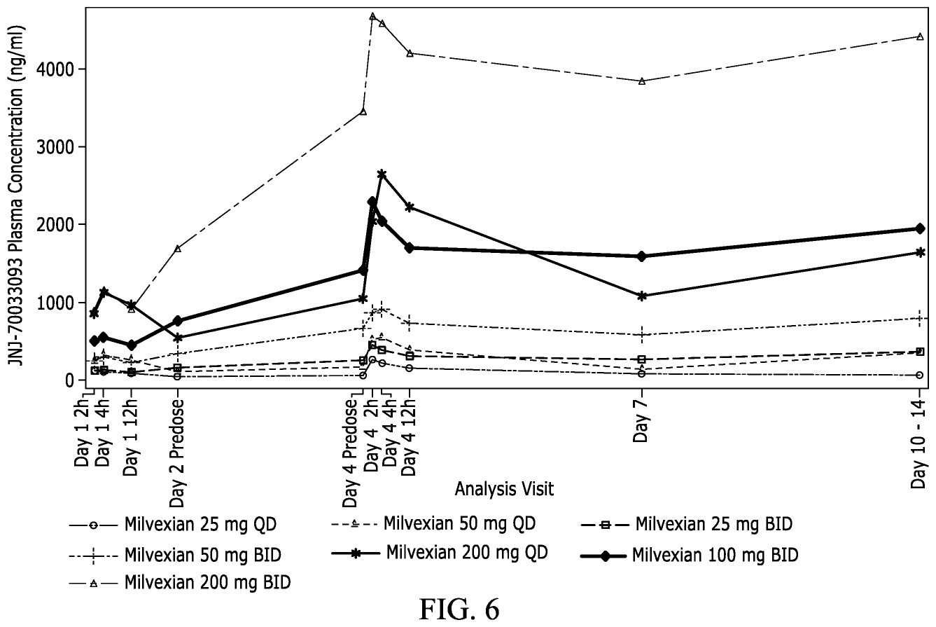

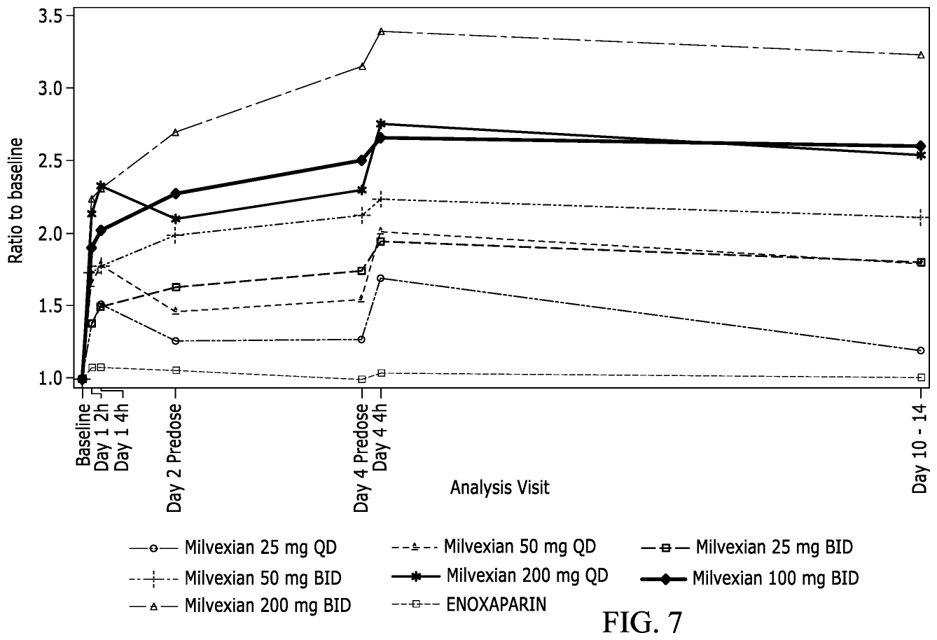

The primary endpoint was numerically lower at the 50 mg and 100 mg BD doses but there was no trend in dose-response. Milvexian numerically reduced the incident rates of clinical ischemic stroke events (acute IS and excluding covert brain infarction) in the ITT population at all milvexian doses except 200 mg twice daily (see Table 4 below). Milvexian is effective at reducing the clinical ischemic strokes in patients after an ischemic stroke or TIA at doses from 25 mg BID to 100 mg BID and these doses demonstrated about 30% relative risk reduction (RRR) in the occurrence of ischemic stroke in the treatment groups versus the placebo groups (See FIG. 3).

TABLE 4
Summary of Clinical Ischemic Stroke and Undetermined Stroke-All Randomized Participants

| Symptomatic | Placebo | 25 QD | 25 BID | 50 BID | 100 BID | 200 BID |
|---|---|---|---|---|---|---|
| Stroke Events | N = 691 | N = 328 | N = 318 | N = 328 | N = 310 | N = 351 |
| Subjects With Event n | 38 (5.5) | 15 (4.6) | 12 (3.8) | 13 (4.0) | 11 (3.5) | 27 (7.7) |
| Incidence Rate (n/N %) | | | | | | |
| Subjects With | 38 (5.5) | 15 (4.6) | 12 (3.8) | 13 (4.0) | 11 (3.5) | 27 (7.7) |
| Ischemic Stroke n | | | | | | |
| Incidence Rate (n/N %) | | | | | | |
| Subjects With | 0 (0) | 0 (0) | 0 (0) | 0 (0) | 0 (0) | 0 (0) |
| Undetermined Stroke n | | | | | | |
| Incidence Rate (n/N %) | | | | | | |
| Subjects With | 0 (0) | 0 (0) | 0 (0) | 0 (0) | 0 (0) | 0 (0) |
| Hemorragic Stroke n | | | | | | |
| Incidence Rate (n/N %) | | | | | | |
| (95% CI) (1) | (3.8, 7.2) | (2.3, 6.8) | (1.7, 5.9) | (1.9, 6.1) | (1.5, 5.6) | (4.9, 10.5) |
| Relative Risk (RR) | | 0.83 | 0.69 | 0.72 | 0.65 | 1.40 |
| 95% CI for RR(2) | | (0.46, 1.49) | (0.36, 1.30) | (0.39, 1.33) | (0.33, 1.25) | (0.87, 2.25) |

Clinical events are included up to Day 21
(1) Wald 95% CI within group
(2)95% CIs for RR are constructed using Wald Confidence Limits
Program Source: BMS_GBS\CV010\OZA75318\Biostatistics\Production\Tables\CSR\rt-ef-clinstk.sas

A sub-group analysis of symptomatic ischemic stroke by stroke-subtype, confirmed that the majority of new ischemic stroke events were due to large vessel atherosclerosis across all milvexian treatment arms and placebo.

The rate of the secondary efficacy endpoint composite event of new ischemic stroke, MI, and all-cause death was lower in milvexian than placebo at dose levels of 25 mg QD to 100 mg BID (Table 5). The relative risk of this composite event in milvexian compared with placebo ranged from 0.78 to 0.85 over the 25 mg QD to 100 mg BID range, but there was no apparent dose-response trend.

TABLE 5
Summary of Event Rate of Composite of New Ischemic Stroke,
MI and All Cause Death - All Randomized Subjects

| New Ischemic Stroke, MI and | Placebo | 25 QD | 25 BID | 50 BID | 100 BID | 200 BID |
|---|---|---|---|---|---|---|
| All Cause Death n (n/N %) | N = 691 | N = 328 | N = 318 | N = 328 | N = 310 | N = 351 |
| Subjects With Event n | 42 (6.1) | 17 (5.2) | 15 (4.7) | 16 (4.9) | 16 (5.2) | 33 (9.4) |
| (n/N %) | | | | | | |
| Ischemic Stroke n (n/N %) | 38 (5.5) | 15 (4.6) | 12 (3.8) | 13 (4.0) | 11 (3.5) | 27 (7.7) |
| Undetermined Stroke n | 0 (0) | 0 (0) | 0 (0) | 0 (0) | 0 (0) | 0 (0) |
| (n/N %) | | | | | | |
| Myocardial Infarction n | 2 (0.3) | 1 (0.3) | 2 (0.6) | 1 (0.3) | 2 (0.6) | 3 (0.9) |
| (n/N %) | | | | | | |
| All Cause Death n (n/N %) | 5 (0.7) | 3 (0.9) | 3 (0.9) | 2 (0.6) | 5 (1.6) | 4 (1.1) |
| (95% CI) (1) | (4.3, 7.9) | (2.8, 7.6) | (2.4, 7.0) | (2.5, 7.2) | (2.7, 7.6) | (6.3, 12.5) |
| Relative Risk (RR) | | 0.85 | 0.78 | 0.80 | 0.85 | 1.55 |
| vs placebo (2) | | | | | | |
| 95% CI for RR vs | | (0.49, 1.47) | (0.44, 1.38) | (0.46, 1.41) | (0.49, 1.49) | (1.0, 2.4) |
| placebo (2) | | | | | | |

Clinical events and deaths are included up to Day 90. The randomization of subjects to the 50 mg QD (N = 22) and 100 mg QD (N = 18) treatment arms was terminated with the implementation of a revised protocol.
(1) Wald 95% CI within group
(2) 95% CIs for RR are constructed using Wald Confidence Limits

Rates of participants completing the treatment period were similar between the placebo and milvexian treatment arms over the dose range of 25 mg QD to 100 mg BID. 20.7% to 25.8% of participants discontinued treatment early over the milvexian dose range of 25 mg QD to 100 mg BID, compared with 23.4% of participants in the placebo arm. The 200 mg BID arm had a higher rate of early treatment discontinuation (31.6%) primarily due to AEs. Similarly, the overall most common reason for participants not completing the study treatment period in the other dose arms was AEs.

SAS® version 9 or higher was used for statistical analyses, tabulations, and graphical presentations. The DoseFinding package for R (version 3.1.3 or higher) was also used in the conduct of the multiple comparisons modelling analyses.

A subset of the ITT Population that includes subjects with at least one PD endpoint (total 1995 subjects) assessed following the first dose of milvexian. Percent (%) change from baseline for aPTT and Factor XI clotting activity are summarized by treatment group and nominal time points. In addition, Exposure-Response relationship (E-R) analyses was conducted to explore the relationship of milvexian exposure to the percent (%) change from baseline in aPTT and Factor XI clotting activity using data collected from all treated subjects (total 2334 subjects). Dose-dependent increases in aPTT and dose-dependent decreases in the Factor XI clotting activity were observed for the milvexian treated groups.

The summary statistics for Activated Partial Thromboplastin Time (aPTT) and percent change from baseline for the pharmacodynamic population is as in the Table 6 below:

TABLE 6
summary statistics for Activated Partial Thromboplastin Time (aPTT)
and percent change from baseline for the pharmacodynamic population

| Visit | | | | | | | | | | |
|---|---|---|---|---|---|---|---|---|---|---|
| Lab Test: aPTT (seconds) | | | | | | | | | | |
| Time Point | | | | | | | | | | |
| Value | | | | | | Percent Change from Baseline | | | | |
| Treatment Group | | | | | | | | | | |
| N | Mean | SE | Min | Median | Max | Mean | SE | Min | Median | Max |

BASELINE Pre-dose

| | | | | | | |
|---|---|---|---|---|---|---|
| placebo | 645 | 26.46 | 0.14 | 14.7 | 25.90 | 55.9 |
| 25 mg BID | 284 | 27.19 | 0.36 | 19.1 | 26.25 | 105.7 |

DAY 1 4 h

| | | | | | | | | | | | |
|---|---|---|---|---|---|---|---|---|---|---|---|
| placebo | 575 | 27.56 | 0.37 | 18.7 | 25.90 | 121.4 | 5.18 | 1.35 | −55.3 | 0.4 | 383.7 |
| 25 mg BID | 260 | 35.06 | 0.5 | 19.9 | 34.65 | 92.1 | 31.05 | 1.79 | −34.4 | 27.06 | 121.4 |

DAY 1 12 h

| | | | | | | | | | | | |
|---|---|---|---|---|---|---|---|---|---|---|---|
| placebo | 570 | 27.20 | 0.56 | 14.6 | 25.90 | 319.5 | 3.21 | 1.67 | −55.8 | −0.17 | 839.7 |
| 25 mg BID | 246 | 35.27 | 0.40 | 23.8 | 34.30 | 64.7 | 32.67 | 1.57 | −37.2 | 30.41 | 141.2 |

DAY 1 24 h

| | | | | | | | | | | | |
|---|---|---|---|---|---|---|---|---|---|---|---|
| placebo | 582 | 26.95 | 0.48 | 14.7 | 25.75 | 251.9 | 3.35 | 1.91 | −55.5 | −1.22 | 847.0 |
| 25 mg BID | 259 | 39.68 | 0.59 | 25.7 | 38.60 | 121.3 | 49.41 | 2.40 | −67.5 | 44.40 | 354.3 |

DAY 21 anytime

| | | | | | | | | | | | |
|---|---|---|---|---|---|---|---|---|---|---|---|
| placebo | 559 | 25.98 | 0.14 | 14.7 | 25.50 | 46.9 | −0.78 | 0.59 | −56.1 | −1.27 | 97.4 |
| 25 mg BID | 237 | 45.20 | 0.79 | 25.9 | 43.80 | 138.1 | 69.60 | 3.28 | −66.7 | 64.26 | 425.1 |

DAY 90 anytime

| | | | | | | | | | | | |
|---|---|---|---|---|---|---|---|---|---|---|---|
| placebo | 503 | 26.77 | 0.19 | 15.2 | 26.00 | 74.0 | 2.47 | 0.77 | −54.0 | 0.83 | 184.6 |
| 25 mg BID | 226 | 42.15 | 0.73 | 23.2 | 41.85 | 125.3 | 58.30 | 3.20 | −55.5 | 54.37 | 452.0 |

The FXI clotting activity observed in this study was as follows:

TABLE 7
Summary Statistics for Factor XI Clotting Activity Measurements
and Percent Change from Baseline - Pharmacodynamic Population

| Visit | | | | | | | | | | |
|---|---|---|---|---|---|---|---|---|---|---|
| Lab Test: FXI Clotting Activity (%) | | | | | | | | | | |
| Time Point | | | | | | | | | | |
| Value | | | | | | Percent Change | | | | |
| Treatment Group | | | | | | | | | | |
| N | Mean | SE | Min | Median | Max | Mean | SE | Min | Median | Max |

BASELINE Pre-dose

| | | | | | | |
|---|---|---|---|---|---|---|
| placebo | 645 | 113.0 | 0.93 | 10.0 | 113.0 | 150.0 |
| 25 mg BID | 281 | 112.9 | 1.27 | 37.0 | 112.0 | 150.0 |

DAY 1 4 h

| | | | | | | | | | | | |
|---|---|---|---|---|---|---|---|---|---|---|---|
| placebo | 569 | 111.6 | 1.00 | 10.0 | 113.0 | 150.0 | 2.19 | 2.78 | −89.5 | −0.93 | 1150 |
| 25 mg BID | 254 | 103.3 | 1.43 | 10.0 | 100.0 | 150.0 | −7.21 | 1.24 | −89.5 | −8.48 | 186.5 |

DAY 1 12 h

| | | | | | | | | | | | |
|---|---|---|---|---|---|---|---|---|---|---|---|
| placebo | 570 | 111.2 | 0.95 | 30.0 | 111.0 | 150.0 | 4.62 | 3.39 | −80.0 | −0.86 | 1170 |
| 25 mg BID | 241 | 103.1 | 1.41 | 36.0 | 101.0 | 150.0 | −6.89 | 1.34 | −63.3 | −7.63 | 202.7 |

DAY 1 24 h

| | | | | | | | | | | | |
|---|---|---|---|---|---|---|---|---|---|---|---|
| placebo | 581 | 114.2 | 0.92 | 33.0 | 115.0 | 150.0 | 5.01 | 2.55 | −71.1 | 0.88 | 1190 |
| 25 mg BID | 253 | 100.9 | 1.27 | 58.0 | 98.0 | 150.0 | −9.69 | 1.13 | −38.3 | −11.30 | 159.5 |

DAY 21 anytime

| | | | | | | | | | | | |
|---|---|---|---|---|---|---|---|---|---|---|---|
| placebo | 554 | 117.5 | 1.43 | 10.0 | 117.0 | 711 | 8.73 | 3.21 | −92.3 | 2.27 | 1340 |
| 25 mg BID | 233 | 90.52 | 1.38 | 44.0 | 88.0 | 148.0 | −18.1 | 1.24 | −67.2 | −19.10 | 121.6 |

DAY 90 anytime

| | | | | | | | | | | | |
|---|---|---|---|---|---|---|---|---|---|---|---|
| placebo | 503 | 112.0 | 0.98 | 10.0 | 111.0 | 150.0 | 4.48 | 3.46 | −91.6 | −1.04 | 1320 |
| 25 mg BID | 223 | 91.05 | 1.54 | 29.0 | 89.0 | 150.0 | −17.7 | 1.90 | −72.1 | −20.16 | 305.4 |

Nominal time points were used for PD biomarker data analysis. For post-baseline time points, N represents subjects with a baseline and a post-baseline value for that time point. Values greater than the upper limit of quantification were set to the upper limit values for summary statistics. Baseline=Non-missing result with a collection date-time less than or equal to the date-time of the first active dose of study medication in each treatment group at time point “BASELINE-PRE DOSE”. The randomization of subjects to the 50 mg QD (N=22) and 100 mg QD (N=18) treatment arms was terminated with the implementation of revised protocol 05.

### Example 3. Prevention of Venous Thromboembolism after Total Knee Arthroplasty

The AXIOMATIC-TKR (Antithrombotic treatment with FXIa inhibition to Optimize Management of Acute Thromboembolic events in TKR) study was a prospective, randomized, open-label, study drug-dose blind, active-controlled, multicenter, dose-ranging, blinded endpoint study of milvexian in participants undergoing primary unilateral elective TKR surgery. The primary objective was to determine the efficacy of milvexian in preventing total venous thromboembolism (VTE) events, defined as proximal and/or distal deep vein thrombosis (DVT) (asymptomatic confirmed by venography assessment or objectively confirmed symptomatic), nonfatal pulmonary embolism, or any death, during the treatment period. The primary hypothesis testing evaluated whether the total VTE rate in the combined BID doses of milvexian was <30%. Assessment of the dose-response for the occurrence of any bleeding was a secondary objective. Participants were randomized 1:1:1:1:1:1:2 to milvexian 25 mg QD, 200 mg QD, 25 mg BID, 50 mg BID, 100 mg BID, 200 mg BID or to enoxaparin 40 mg QD subcutaneous for 10 to 14 days. After the first interim analysis, randomization into the 25 mg QD dosing group was discontinued and milvexian 50 mg QD dosing was initiated. The milvexian used in AXIOMATIC-TKR phase II trial was formulated as SDD capsule formulations in 25 mg and 100 mg strength as described in WO2020210629.

A total of 1,242 participants were randomized and included in the intention-to-treat analysis set. The median age of the study population was 68 years. The mean duration of treatment exposure was 11.7 days in the combined milvexian groups and in the enoxaparin group. Total VTE was reported in 108 (13.6%) of the 796 combined milvexian participants and in 63 (12.2%) of 518 participants assigned to a milvexian BID dose regimen, compared to 54 (21.4%) of 252 enoxaparin participants (relative risk ratio [RR] 0.64, 95% CI 0.48-0.85 for combined milvexian vs. enoxaparin, and RR 0.57, 95% CI 0.41-0.79 for milvexian BID vs. enoxaparin). There was a statistically significant reduction (P<0.0001) of total VTE for the pooled milvexian BID dose regimens compared to the prespecified target of 30%. Milvexian demonstrated statistically significant reductions in total VTE compared to enoxaparin at each of the total daily dose regimens ≥100 mg. A statistically significant dose response trend was observed for the BID regimens (P=0.0004); post-hoc analyses also demonstrated a significant dose response trend (P=0.0003) for the once-daily dose groups.

The pharmacodynamics analysis set was defined as subjects who have received at least 1 dose of study drug and at least one valid blood sample drawn for PD analysis. PD analyses were performed on the PD analysis set, defined as subjects who had at least one valid blood sample drawn for PD. Descriptive statistics (N, mean, SD, median, range, CV (%) and IQ range) were used to summarize PD response at each nominal sampling time point by treatment group. Changes from baseline were summarized when applicable. PD response outside of limit of quantitation were not to be imputed and were to be noted in the summary statistics.

The pharmacodynamic results from this study are shown in the Tables 8-11 below.

TABLE 8
Summary of Activated Partial Thromboplastin Time (aPTT) and Ratio to Baseline aPTT, at Nominal
Sampling Time Day 1 Through Day 14, by Dose and Treatment Group; PD Analysis Set

| Measured Value | | | | | | Ratio to Baseline | | | | | |
|---|---|---|---|---|---|---|---|---|---|---|---|
| N | Mean | SD | Med | Min | Max | N | Mean | SD | Med | Min | Max |

| | |
|---|---|
| Analysis set: PD | |
| analysis set | |
| Milvexian 25 mg QD | 33 |
| Milvexian 50 mg QD | 150 |
| Milvexian 25 mg BID | 148 |
| Milvexian 50 mg BID | 148 |
| Milvexian 200 mg QD | 147 |
| Milvexian 100 mg BID | 149 |
| Milvexian 200 mg BID | 148 |
| ENOXAPARIN | 296 |

Activated Partial Thromboplastin Time (sec)

| | | | | | | | | | | | | |
|---|---|---|---|---|---|---|---|---|---|---|---|---|
| Milvexian 25 mg QD | | | | | | | | | | | | |
| Baseline | 33 | 28.08 | 7.398 | 26.3 | 21.7 | 66.8 | | | | | | |
| Day 1 2 h | 32 | 37.54 | 9.082 | 37.5 | 21.8 | 57.7 | 32 | 1.37 | 0.324 | 1.37 | 0.6 | 1.9 |
| Day 1 4 h | 31 | 40.47 | 12.697 | 40.3 | 21.5 | 93.4 | 31 | 1.48 | 0.475 | 1.51 | 0.5 | 3.4 |
| Day 2 Predose | 33 | 34.71 | 7.051 | 33.6 | 25.1 | 60.3 | 33 | 1.27 | 0.288 | 1.26 | 0.5 | 2.2 |
| Day 4 Predose | 23 | 35.67 | 6.695 | 34.1 | 25.8 | 53.1 | 23 | 1.35 | 0.26 | 1.26 | 0.9 | 2 |
| Day 4 4 h | 22 | 44.2 | 8.045 | 44.15 | 25.7 | 56.6 | 22 | 1.68 | 0.316 | 1.69 | 1.1 | 2.2 |
| Day 10-14 | 33 | 35.37 | 8.222 | 35 | 24.4 | 53 | 33 | 1.29 | 0.338 | 1.19 | 0.7 | 2.2 |
| Milvexian 50 mg QD | | | | | | | | | | | | |
| Baseline | 146 | 27.63 | 5.459 | 26.45 | 21.6 | 63 | | | | | | |
| Day 1 2 h | 142 | 44.59 | 24.738 | 43.05 | 22.9 | 284.7 | 139 | 1.64 | 0.832 | 1.65 | 0.5 | 9.5 |
| Day 1 4 h | 141 | 47.85 | 10.625 | 46.2 | 25.2 | 117.7 | 137 | 1.77 | 0.374 | 1.78 | 0.8 | 3.5 |
| Day 2 Predose | 137 | 39.77 | 9.427 | 39 | 27.2 | 112 | 134 | 1.46 | 0.312 | 1.46 | 0.8 | 3.8 |
| Day 4 Predose | 88 | 43.64 | 9.575 | 41.85 | 28.1 | 78.7 | 86 | 1.58 | 0.339 | 1.54 | 0.7 | 2.6 |
| Day 4 4 h | 86 | 58.08 | 26.937 | 52 | 28.8 | 237.5 | 84 | 2.13 | 1.014 | 2.01 | 0.7 | 8.8 |
| Day 10-14 | 142 | 49.29 | 14.338 | 48.35 | 23.3 | 107.3 | 138 | 1.82 | 0.551 | 1.79 | 0.6 | 4.5 |
| Milvexian 25 mg BID | | | | | | | | | | | | |
| Baseline | 146 | 28.58 | 17.823 | 26.4 | 19.3 | 228.9 | | | | | | |
| Day 1 2 h | 143 | 38.84 | 13.15 | 36.4 | 20.6 | 130.8 | 141 | 1.42 | 0.358 | 1.38 | 0.6 | 2.7 |
| Day 1 4 h | 141 | 40.07 | 10.923 | 39.4 | 22.8 | 121.4 | 139 | 1.49 | 0.376 | 1.49 | 0.2 | 3.4 |
| Day 2 Predose | 143 | 44.94 | 12.083 | 43.2 | 26.9 | 101.4 | 142 | 1.66 | 0.45 | 1.62 | 0.4 | 3.9 |
| Day 4 Predose | 91 | 46.75 | 8.125 | 45.7 | 27.6 | 65.6 | 89 | 1.77 | 0.322 | 1.74 | 0.7 | 2.6 |
| Day 4 4 h | 90 | 52.02 | 11.905 | 51.05 | 32.2 | 140.2 | 88 | 1.97 | 0.439 | 1.94 | 0.7 | 5 |
| Day 10-14 | 142 | 49.76 | 14.681 | 47.8 | 25.1 | 149.6 | 140 | 1.85 | 0.559 | 1.8 | 0.5 | 6 |
| Milvexian 50 mg BID | | | | | | | | | | | | |
| Baseline | 140 | 27.82 | 6.679 | 26.45 | 20 | 68.7 | | | | | | |
| Day 1 2 h | 139 | 44.12 | 10.867 | 44.5 | 21.6 | 73.7 | 135 | 1.64 | 0.44 | 1.72 | 0.5 | 2.5 |
| Day 1 4 h | 134 | 47.83 | 10.936 | 47.1 | 24.6 | 118.6 | 129 | 1.76 | 0.361 | 1.77 | 0.5 | 3.1 |
| Day 2 Predose | 138 | 54.52 | 28.131 | 50.55 | 34.8 | 358.9 | 132 | 2.02 | 0.921 | 1.99 | 0.7 | 11.3 |
| Day 4 Predose | 86 | 57.84 | 10.983 | 55.95 | 38.7 | 113.6 | 84 | 2.13 | 0.469 | 2.12 | 0.9 | 4.7 |
| Day 4 4 h | 88 | 61.72 | 13.663 | 59 | 40.6 | 124.8 | 85 | 2.28 | 0.536 | 2.23 | 0.9 | 4.7 |
| Day 10-14 | 139 | 59.26 | 26.042 | 56.4 | 24.3 | 277.4 | 133 | 2.17 | 0.89 | 2.11 | 0.5 | 10.5 |
| Milvexian 200 mg QD | | | | | | | | | | | | |
| Baseline | 143 | 28.24 | 8.593 | 26.2 | 20.2 | 95.7 | | | | | | |
| Day 1 2 h | 138 | 56.93 | 16.773 | 56.1 | 21 | 130.7 | 136 | 2.11 | 0.658 | 2.13 | 0.4 | 5.2 |
| Day 1 4 h | 137 | 67.39 | 43.329 | 61.7 | 20 | 400 | 133 | 2.49 | 1.691 | 2.33 | 0.4 | 15.8 |
| Day 2 Predose | 142 | 56.77 | 13.35 | 54.85 | 26.8 | 159.1 | 139 | 2.09 | 0.555 | 2.1 | 0.6 | 5.7 |
| Day 4 Predose | 92 | 62.16 | 12.416 | 60.65 | 39.5 | 122.8 | 91 | 2.29 | 0.601 | 2.3 | 0.7 | 5.1 |
| Day 4 4 h | 87 | 73.4 | 14.797 | 70.5 | 45 | 114.8 | 86 | 2.69 | 0.701 | 2.75 | 0.6 | 4.2 |
| Day 10-14 | 142 | 71.87 | 37.06 | 67.55 | 24.2 | 400 | 139 | 2.63 | 1.189 | 2.54 | 0.6 | 10 |
| Milvexian 100 mg BID | | | | | | | | | | | | |
| Baseline | 142 | 27.86 | 8.85 | 26.3 | 10.7 | 93.1 | | | | | | |
| Day 1 2 h | 142 | 50.92 | 15.283 | 51.6 | 22.6 | 126.1 | 137 | 1.89 | 0.624 | 1.9 | 0.4 | 5.1 |
| Day 1 4 h | 141 | 56.19 | 22.927 | 54.1 | 22.4 | 282.4 | 136 | 2.08 | 0.959 | 2.02 | 0.5 | 11.8 |
| Day 2 Predose | 140 | 61.63 | 13.832 | 59.9 | 38.9 | 156.5 | 136 | 2.32 | 0.603 | 2.27 | 0.6 | 5.8 |
| Day 4 Predose | 87 | 67.53 | 12.679 | 66 | 39.9 | 113.4 | 83 | 2.53 | 0.605 | 2.5 | 0.9 | 5.8 |
| Day 4 4 h | 88 | 70.64 | 12.13 | 69.05 | 38.6 | 106.5 | 84 | 2.67 | 0.681 | 2.66 | 1 | 6.9 |
| Day 10-14 | 143 | 73.19 | 33.524 | 69.7 | 29.3 | 367.5 | 137 | 2.77 | 1.448 | 2.6 | 0.7 | 16.6 |
| Milvexian 200 mg BID | | | | | | | | | | | | |
| Baseline | 144 | 26.86 | 5.046 | 25.8 | 19.1 | 67.1 | | | | | | |
| Day 1 2 h | 142 | 57.62 | 21.958 | 55.6 | 22.5 | 200 | 138 | 2.19 | 0.912 | 2.24 | 0.8 | 9 |
| Day 1 4 h | 141 | 67.04 | 42.407 | 59.9 | 32.2 | 400 | 137 | 2.53 | 1.493 | 2.31 | 0.9 | 14.6 |
| Day 2 Predose | 139 | 71.58 | 15.528 | 68.4 | 40 | 122.5 | 136 | 2.7 | 0.481 | 2.7 | 1.2 | 4.1 |
| Day 4 Predose | 94 | 84.3 | 20.554 | 81.75 | 46.6 | 223 | 93 | 3.21 | 0.646 | 3.15 | 1.8 | 7 |
| Day 4 4 h | 91 | 89.45 | 21.847 | 87 | 46.1 | 224.8 | 89 | 3.43 | 0.822 | 3.39 | 1.7 | 8.2 |
| Day 10-14 | 141 | 86.07 | 19.029 | 85.1 | 27.3 | 160.2 | 137 | 3.26 | 0.734 | 3.23 | 1 | 5.4 |
| ENOXAPARIN | | | | | | | | | | | | |
| Baseline | 283 | 27.5 | 10.508 | 26 | 19.5 | 179 | | | | | | |
| Day 1 2 h | 146 | 37.03 | 53.325 | 27.5 | 20.9 | 400 | 138 | 1.36 | 1.882 | 1.07 | 0.3 | 16.7 |
| Day 1 4 h | 148 | 29.14 | 10.133 | 27.75 | 20.8 | 120.2 | 140 | 1.08 | 0.318 | 1.07 | 0.1 | 4.2 |
| Day 2 Predose | 177 | 28.11 | 6.552 | 27.4 | 20.3 | 97.7 | 169 | 1.05 | 0.188 | 1.05 | 0.3 | 2.6 |
| Day 4 Predose | 107 | 26.03 | 2.434 | 25.8 | 20.7 | 34.8 | 100 | 0.99 | 0.159 | 0.99 | 0.1 | 1.3 |
| Day 4 4 h | 105 | 27.37 | 8.122 | 26.1 | 20 | 105.9 | 98 | 1.04 | 0.293 | 1.03 | 0.1 | 3.4 |
| Day 10-14 | 277 | 27.8 | 10.632 | 25.9 | 19.8 | 154.2 | 265 | 1.04 | 0.365 | 1 | 0.1 | 4.5 |

Key: SD = standard deviation.
Note:
N for measured value is the number of subjects with a non-missing value for the parameter at the specified time point. N for ratio to baseline is the number of subjects with non-missing values at both baseline and the postbaseline time point.

TABLE 9
Summary of FXI Clotting Activity (FXI:c) and % Change from Baseline, at Nominal
Sampling Time Day 1 Through Day 14, by Dose and Treatment Group; PD Analysis Set

| Measured Value | | | | | | % Change Ratio From Baseline | | | | | |
|---|---|---|---|---|---|---|---|---|---|---|---|
| N | Mean | SD | Med | Min | Max | N | Mean | SD | Med | Min | Max |

| | |
|---|---|
| Analysis set: PD | |
| analysis set | |
| Milvexian 25 mg QD | 33 |
| Milvexian 50 mg QD | 150 |
| Milvexian 25 mg BID | 148 |
| Milvexian 50 mg BID | 148 |
| Milvexian 200 mg QD | 147 |
| Milvexian 100 mg BID | 149 |
| Milvexian 200 mg BID | 148 |
| ENOXAPARIN | 296 |

Factor XI Activity (fraction of 1) Milvexian 25 mg QD

| | | | | | | | | | | | | |
|---|---|---|---|---|---|---|---|---|---|---|---|---|
| Baseline | 33 | 1 | 0.242 | 1.02 | 0.1 | 1.3 | | | | | | |
| Day 1 4 h | 33 | 0.94 | 0.186 | 0.93 | 0.6 | 1.3 | 33 | 20.23 | 160.332 | −7.95 | −36.6 | 910 |
| Day 10-14 | 33 | 1.24 | 0.332 | 1.37 | 0.1 | 1.5 | 33 | 63 | 235.501 | 30.36 | −90 | 1360 |
| Milvexian 50 mg QD | | | | | | | | | | | | |
| Baseline | 145 | 1.02 | 0.216 | 1 | 0.1 | 1.5 | | | | | | |
| Day 1 4 h | 143 | 0.77 | 0.199 | 0.77 | 0.1 | 1.4 | 139 | −21.81 | 35.249 | −24.83 | −89.5 | 350 |
| Day 10-14 | 142 | 1.04 | 0.267 | 1.03 | 0.4 | 1.5 | 137 | 4.89 | 33.959 | 4.67 | −61 | 270 |
| Milvexian 25 mg BID | | | | | | | | | | | | |
| Baseline | 143 | 1.08 | 0.21 | 1.07 | 0.5 | 1.5 | | | | | | |
| Day 1 4 h | 144 | 0.94 | 0.208 | 0.92 | 0.4 | 1.5 | 139 | −12.6 | 12.451 | −12.87 | −62.7 | 31.7 |
| Day 10-14 | 140 | 1.07 | 0.243 | 1.06 | 0.5 | 1.5 | 135 | 0.92 | 24.303 | 0 | −52.3 | 69 |
| Milvexian 50 mg BID | | | | | | | | | | | | |
| Baseline | 143 | 1.05 | 0.226 | 1.07 | 0.1 | 1.5 | | | | | | |
| Day 1 4 h | 143 | 0.81 | 0.2 | 0.82 | 0.1 | 1.4 | 139 | −15.8 | 84.259 | −22.64 | −91.2 | 950 |
| Day 10-14 | 138 | 0.81 | 0.266 | 0.82 | 0.2 | 1.5 | 133 | −18.47 | 55.202 | −27.66 | −76.2 | 530 |
| Milvexian 200 mg QD | | | | | | | | | | | | |
| Baseline | 146 | 1.02 | 0.222 | 1.02 | 0.1 | 1.5 | | | | | | |
| Day 1 4 h | 140 | 0.46 | 0.243 | 0.42 | 0.1 | 1.5 | 139 | −51.66 | 37.899 | −59.38 | −88.1 | 310 |
| Day 10-14 | 141 | 0.52 | 0.337 | 0.44 | 0.1 | 1.5 | 141 | −42.13 | 79.293 | −58.12 | −89.5 | 800 |
| Milvexian 100 mg BID | | | | | | | | | | | | |
| Baseline | 145 | 1.05 | 0.224 | 1.04 | 0.1 | 1.5 | | | | | | |
| Day 1 4 h | 145 | 0.65 | 0.249 | 0.64 | 0.1 | 1.5 | 142 | −37.59 | 21.387 | −40.88 | −74.2 | 50 |
| Day 10-14 | 140 | 0.44 | 0.224 | 0.37 | 0.1 | 1.5 | 137 | −56.89 | 24.129 | −64.34 | −88.8 | 47.1 |
| Milvexian 200 mg BID | | | | | | | | | | | | |
| Baseline | 143 | 1.08 | 0.226 | 1.09 | 0.2 | 1.5 | | | | | | |
| Day 1 4 h | 143 | 0.48 | 0.235 | 0.43 | 0.1 | 1.2 | 138 | −51.85 | 57.702 | −60.28 | −85.9 | 582.4 |
| Day 10-14 | 139 | 0.21 | 0.21 | 0.15 | 0.1 | 1.5 | 134 | −79.12 | 20.343 | −85.64 | −93.1 | 16 |
| ENOXAPARIN | | | | | | | | | | | | |
| Baseline | 97 | 1.16 | 0.203 | 1.16 | 0.6 | 1.5 | | | | | | |
| Day 1 4 h | 14 | 1.06 | 0.195 | 1.07 | 0.7 | 1.4 | 14 | −7.24 | 8.112 | −7.24 | −18.6 | 4.6 |
| Day 10-14 | 10 | 1.36 | 0.11 | 1.39 | 1.2 | 1.5 | 9 | 36.64 | 27.762 | 37.21 | −2.9 | 91.8 |

Key: SD = standard deviation.
Note:
N for measured value is the number of subjects with a non-missing value for the parameter at the specified time point. N for percent change from baseline is the number of subjects with non-missing values at both baseline and the postbaseline time point.

TABLE 10
Summary of Thrombin Generation Assay (TGA) Parameter: Peak Height Ratio to Baseline at Nominal
Sampling Time Day 1 Through Day 14, by Dose and Treatment Group; PD Analysis Set

| Measured Value | | | | | | Ratio to Baseline | | | | | |
|---|---|---|---|---|---|---|---|---|---|---|---|
| N | Mean | SD | Med | Min | Max | N | Mean | SD | Med | Min | Max |

| | |
|---|---|
| Analysis set: PD | |
| analysis set | |
| Milvexian 25 mg QD | 33 |
| Milvexian 50 mg QD | 150 |
| Milvexian 25 mg BID | 148 |
| Milvexian 50 mg BID | 148 |
| Milvexian 200 mg QD | 147 |
| Milvexian 100 mg BID | 149 |
| Milvexian 200 mg BID | 148 |
| ENOXAPARIN | 296 |

ETP Peak Height (nmol/L) Milvexian 25 mg QD

| | | | | | | | | | | | | |
|---|---|---|---|---|---|---|---|---|---|---|---|---|
| Baseline | 33 | 196.02 | 82.421 | 184.77 | 23.3 | 472.5 | | | | | | |
| Day 1 2 h | 33 | 169.82 | 86.004 | 163.21 | 9 | 436.2 | 33 | 0.96 | 0.676 | 0.79 | 0.1 | 4 |
| Day 1 4 h | 33 | 160.09 | 70.07 | 156.09 | 0 | 318.1 | 33 | 1.02 | 0.837 | 0.88 | 0 | 4.6 |
| Day 2 Predose | 33 | 194.29 | 71.612 | 178.68 | 106.1 | 464.8 | 33 | 1.32 | 1.502 | 0.98 | 0.6 | 8 |
| Day 4 Predose | 23 | 207.77 | 78.581 | 180.35 | 102.3 | 387 | 23 | 1.24 | 1.125 | 0.95 | 0.6 | 6.2 |
| Day 4 4 h | 23 | 186.96 | 89.057 | 175.48 | 56.5 | 431.6 | 23 | 1.09 | 0.967 | 0.87 | 0.3 | 5.3 |
| Day 10-14 | 33 | 211.62 | 133.964 | 182.84 | 0 | 571.9 | 33 | 1.42 | 1.542 | 1.02 | 0 | 7.8 |
| Milvexian 50 mg QD | | | | | | | | | | | | |
| Baseline | 132 | 216.32 | 70.87 | 203.22 | 0.7 | 483.6 | | | | | | |
| Day 1 2 h | 136 | 123.13 | 77.074 | 114.25 | 0 | 361.3 | 126 | 2.95 | 26.654 | 0.51 | 0 | 299.7 |
| Day 1 4 h | 135 | 104.09 | 65.45 | 93.81 | 0 | 315.4 | 125 | 1.71 | 13.797 | 0.46 | 0 | 154.7 |
| Day 2 Predose | 134 | 168.16 | 77.43 | 160.31 | 0 | 444 | 122 | 3.08 | 25.135 | 0.75 | 0 | 278.4 |
| Day 4 Predose | 84 | 147.71 | 80.199 | 147.29 | 15.2 | 379.2 | 79 | 3.61 | 25.185 | 0.74 | 0.1 | 224.6 |
| Day 4 4 h | 81 | 90.66 | 71.173 | 85.17 | 0 | 382 | 76 | 0.47 | 0.389 | 0.41 | 0 | 2 |
| Day 10 - 14 | 137 | 120.68 | 87.476 | 102.19 | 0 | 408.2 | 127 | 2.52 | 21.687 | 0.53 | 0 | 244.9 |
| Milvexian 25 mg BID | | | | | | | | | | | | |
| Baseline | 137 | 234.13 | 77.471 | 229.53 | 4.8 | 428.1 | | | | | | |
| Day 1 2 h | 144 | 160.09 | 79.939 | 158.14 | 0 | 400.1 | 136 | 0.71 | 0.384 | 0.7 | 0 | 3.3 |
| Day 1 4 h | 139 | 158.43 | 74.639 | 156.24 | 0 | 402.9 | 131 | 0.71 | 0.511 | 0.7 | 0 | 5.4 |
| Day 2 Predose | 137 | 161.28 | 68.385 | 158.31 | 0 | 365.7 | 129 | 1.06 | 3.783 | 0.73 | 0 | 43.5 |
| Day 4 Predose | 92 | 151.22 | 94.22 | 129.13 | 0 | 455.6 | 88 | 1.08 | 4.281 | 0.59 | 0 | 40.7 |
| Day 4 4 h | 90 | 123.59 | 84.546 | 120.37 | 0 | 433.2 | 86 | 0.96 | 4.101 | 0.49 | 0 | 38.4 |
| Day 10-14 | 136 | 143.34 | 84.215 | 128.09 | 0 | 401.9 | 129 | 0.98 | 3.824 | 0.59 | 0 | 43.8 |
| Milvexian 50 mg BID | | | | | | | | | | | | |
| Baseline | 136 | 226.99 | 79.172 | 208.71 | 0 | 539.8 | | | | | | |
| Day 1 2 h | 139 | 136.17 | 85.036 | 129.86 | 0 | 542.2 | 131 | 0.84 | 2.837 | 0.63 | 0 | 32.9 |
| Day 1 4 h | 136 | 123.8 | 73.611 | 116.88 | 0 | 371.1 | 128 | 1.14 | 6.199 | 0.56 | 0 | 70.4 |
| Day 2 Predose | 136 | 133.91 | 68.406 | 131.12 | 0 | 326.9 | 128 | 1.64 | 11.838 | 0.62 | 0 | 134.5 |
| Day 4 Predose | 85 | 106.19 | 67.273 | 92.64 | 0 | 337.2 | 80 | 1.78 | 8.355 | 0.46 | 0 | 63.5 |
| Day 4 4 h | 86 | 92.36 | 70.283 | 80.31 | 0 | 354.1 | 82 | 1.75 | 9.154 | 0.39 | 0 | 75.7 |
| Day 10-14 | 136 | 97.53 | 76.57 | 81.88 | 0 | 376.6 | 128 | 0.96 | 4.235 | 0.4 | 0 | 41.1 |
| Milvexian 200 mg QD | | | | | | | | | | | | |
| Baseline | 138 | 206.51 | 81.998 | 195.52 | 0 | 439.3 | | | | | | |
| Day 1 2 h | 135 | 87.44 | 76.029 | 70 | 0 | 340 | 128 | 0.65 | 1.627 | 0.35 | 0 | 14 |
| Day 1 4 h | 138 | 78.48 | 68.935 | 63.95 | 0 | 370.5 | 130 | 0.47 | 1.07 | 0.32 | 0 | 11.1 |
| Day 2 Predose | 141 | 105.18 | 64.059 | 101.59 | 0 | 312.9 | 133 | 0.72 | 1.562 | 0.49 | 0 | 16.9 |
| Day 4 Predose | 89 | 79.52 | 67.782 | 69.47 | 0 | 368 | 86 | 0.46 | 0.553 | 0.33 | 0 | 3.4 |
| Day 4 4 h | 87 | 43.49 | 43.655 | 36.9 | 0 | 227.4 | 85 | 0.24 | 0.302 | 0.17 | 0 | 1.7 |
| Day 10-14 | 139 | 72.82 | 70.636 | 49.34 | 0 | 395.4 | 132 | 0.47 | 0.809 | 0.26 | 0 | 5.5 |
| Milvexian 100 mg BID | | | | | | | | | | | | |
| Baseline | 139 | 226.32 | 85.671 | 208.99 | 0 | 627.6 | | | | | | |
| Day 1 2 h | 140 | 109.97 | 80.255 | 83.74 | 0 | 383.9 | 133 | 3.38 | 33.099 | 0.35 | 0 | 382.2 |
| Day 1 4 h | 136 | 100.31 | 68.142 | 90.09 | 0 | 333.7 | 129 | 2.52 | 23.357 | 0.4 | 0 | 265.7 |
| Day 2 Predose | 139 | 99.64 | 67.58 | 89.19 | 0 | 362.9 | 132 | 1.77 | 15.265 | 0.39 | 0 | 175.8 |
| Day 4 Predose | 87 | 70.96 | 58.873 | 63.85 | 0 | 285 | 81 | 0.32 | 0.289 | 0.29 | 0 | 1.4 |
| Day 4 4 h | 86 | 55.94 | 54.304 | 47.34 | 0 | 258.1 | 81 | 0.24 | 0.235 | 0.18 | 0 | 1.1 |
| Day 10-14 | 140 | 57.6 | 57.165 | 37.89 | 0 | 281.2 | 132 | 0.27 | 0.274 | 0.17 | 0 | 1.8 |
| Milvexian 200 mg BID | | | | | | | | | | | | |
| Baseline | 141 | 225.67 | 88.918 | 208.91 | 4.6 | 512.3 | | | | | | |
| Day 1 2 h | 143 | 86.72 | 74.645 | 67.9 | 0 | 368.8 | 136 | 0.41 | 0.358 | 0.33 | 0 | 2.1 |
| Day 1 4 h | 145 | 68.67 | 57.469 | 58.6 | 0 | 273.7 | 138 | 0.45 | 1.526 | 0.25 | 0 | 17.8 |
| Day 2 Predose | 141 | 64.57 | 49.137 | 57.71 | 0 | 216.3 | 134 | 0.42 | 1.743 | 0.24 | 0 | 20.3 |
| Day 4 Predose | 93 | 39.31 | 41.083 | 29.78 | 0 | 191.1 | 91 | 0.33 | 1.58 | 0.12 | 0 | 15.1 |
| Day 4 4 h | 91 | 30.92 | 29.586 | 26.59 | 0 | 128.2 | 87 | 0.26 | 1.158 | 0.11 | 0 | 10.9 |
| Day 10-14 | 140 | 29.94 | 36.819 | 19.39 | 0 | 279.3 | 136 | 0.18 | 0.528 | 0.08 | 0 | 5.8 |
| ENOXAPARIN | | | | | | | | | | | | |
| Baseline | 271 | 214.6 | 83.91 | 202.17 | 0 | 511.9 | | | | | | |
| Day 1 2 h | 143 | 171.15 | 94.909 | 158.05 | 0 | 437.8 | 129 | 1.58 | 9.418 | 0.77 | 0 | 107.7 |
| Day 1 4 h | 153 | 183.54 | 101.228 | 160.49 | 0 | 657.4 | 139 | 1.71 | 10.848 | 0.78 | 0 | 128.6 |
| Day 2 Predose | 172 | 232.14 | 90.677 | 214.37 | 0 | 582 | 155 | 2.83 | 22.001 | 1.02 | 0 | 274.9 |
| Day 4 Predose | 106 | 250.47 | 107.259 | 227.87 | 0 | 602.9 | 95 | 3.73 | 24.922 | 1.11 | 0 | 244 |
| Day 4 4 h | 105 | 210.21 | 96.606 | 196.02 | 20.5 | 530.3 | 95 | 3.2 | 21.371 | 1 | 0.1 | 209.3 |
| Day 10-14 | 270 | 213.56 | 129.563 | 201.82 | 0 | 616.6 | 253 | 1.98 | 15.467 | 0.98 | 0 | 246.8 |

Key: SD = standard deviation.
Note:
N for measured value is the number of subjects with a non-missing value for the parameter at the specified time point. N for ratio to baseline is the number of subjects with non-missing values at both baseline and the postbaseline time point.

TABLE 11
Summary of Thrombin Generation Assay (TGA) Parameter: Lagtime, and Ratio to Baseline at
Nominal Sampling Time Day 1 Through Day 14, by Dose and Treatment Group; PD Analysis Set

| Measured Value | | | | | | Ratio to Baseline | | | | | |
|---|---|---|---|---|---|---|---|---|---|---|---|
| N | Mean | SD | Med | Min | Max | N | Mean | SD | Med | Min | Max |

| | |
|---|---|
| Analysis set: PD | |
| analysis set | |
| Milvexian 25 mg QD | 33 |
| Milvexian 50 mg QD | 150 |
| Milvexian 25 mg BID | 148 |
| Milvexian 50 mg BID | 148 |
| Milvexian 200 mg QD | 147 |
| Milvexian 100 mg BID | 149 |
| Milvexian 200 mg BID | 148 |
| ENOXAPARIN | 296 |

ETP Lag Time (min) Milvexian 25 mg QD

| | | | | | | | | | | | | |
|---|---|---|---|---|---|---|---|---|---|---|---|---|
| Baseline | 33 | 6.14 | 1.511 | 5.71 | 4.3 | 11.7 | | | | | | |
| Day 1 2 h | 33 | 10.82 | 5.061 | 9.58 | 4.2 | 28.2 | 33 | 1.77 | 0.699 | 1.59 | 0.9 | 4 |
| Day 1 4 h | 33 | 15.78 | 20.492 | 10.53 | 4.1 | 120 | 33 | 2.61 | 3.501 | 1.85 | 0.9 | 20.4 |
| Day 2 Predose | 33 | 8.43 | 2.437 | 7.78 | 4.9 | 16.1 | 33 | 1.41 | 0.434 | 1.32 | 0.8 | 3 |
| Day 4 Predose | 23 | 9.64 | 3.442 | 8.89 | 6.1 | 23.6 | 23 | 1.6 | 0.41 | 1.54 | 0.9 | 2.6 |
| Day 4 4 h | 23 | 12.85 | 4.327 | 12.09 | 6.2 | 25.8 | 23 | 2.13 | 0.559 | 2.1 | 1.2 | 3.2 |
| Day 10-14 | 33 | 14.17 | 19.833 | 10.33 | 5.1 | 120 | 33 | 2.34 | 3.161 | 1.59 | 0.9 | 19 |
| Milvexian 50 mg QD | | | | | | | | | | | | |
| Baseline | 132 | 6.12 | 2.674 | 5.76 | 1.7 | 29.8 | | | | | | |
| Day 1 2 h | 136 | 17.13 | 19.55 | 12.67 | 2.7 | 120 | 126 | 2.74 | 2.166 | 2.38 | 0.6 | 15.6 |
| Day 1 4 h | 135 | 21.22 | 21.734 | 14.22 | 3.3 | 120 | 125 | 3.55 | 3.278 | 2.56 | 0.6 | 20.1 |
| Day 2 Predose | 134 | 12.91 | 16.759 | 9.86 | 1 | 120 | 122 | 2.11 | 1.829 | 1.77 | 0.2 | 16.3 |
| Day 4 Predose | 84 | 14.23 | 8.639 | 11.78 | 5 | 57.2 | 79 | 2.55 | 1.604 | 2.2 | 0.4 | 12.3 |
| Day 4 4 h | 81 | 30.43 | 31.179 | 17.9 | 5.8 | 120 | 76 | 5.59 | 7.516 | 3.34 | 0.8 | 58.6 |
| Day 10-14 | 137 | 20.89 | 21.62 | 14.44 | 3.6 | 120 | 127 | 3.48 | 3.162 | 2.65 | 0.4 | 20.9 |
| Milvexian 25 mg BID | | | | | | | | | | | | |
| Baseline | 137 | 5.85 | 1.565 | 5.57 | 2.7 | 11 | | | | | | |
| Day 1 2 h | 144 | 14.36 | 17.155 | 10.22 | 4 | 120 | 136 | 2.47 | 2.677 | 1.93 | 0.7 | 25.1 |
| Day 1 4 h | 139 | 15.83 | 20.752 | 11.11 | 2.6 | 120 | 131 | 2.8 | 3.852 | 2.03 | 0.6 | 28.4 |
| Day 2 Predose | 137 | 12.92 | 11.098 | 10.46 | 5 | 120 | 129 | 2.16 | 1.208 | 1.91 | 0.7 | 12.4 |
| Day 4 Predose | 92 | 19.58 | 24.82 | 13.06 | 4.6 | 120 | 88 | 3.33 | 3.31 | 2.37 | 0.9 | 18.3 |
| Day 4 4 h | 90 | 19.99 | 18.412 | 15.79 | 2.1 | 120 | 86 | 3.44 | 2.284 | 2.95 | 0.5 | 16.4 |
| Day 10-14 | 136 | 17.35 | 14.248 | 14.47 | 5.6 | 120 | 129 | 2.97 | 2.013 | 2.55 | 1.1 | 17.5 |
| Milvexian 50 mg BID | | | | | | | | | | | | |
| Baseline | 136 | 7.2 | 12.435 | 5.44 | 1.3 | 120 | | | | | | |
| Day 1 2 h | 139 | 15.96 | 18.595 | 11.67 | 1.3 | 120 | 132 | 2.63 | 2.533 | 2.26 | 0.1 | 22.2 |
| Day 1 4 h | 136 | 16.82 | 16.362 | 13.22 | 3.4 | 120 | 129 | 2.92 | 2.656 | 2.46 | 0.1 | 25.1 |
| Day 2 Predose | 136 | 17.64 | 19.756 | 12.78 | 2.3 | 120 | 129 | 3.11 | 3.151 | 2.33 | 0.1 | 22.2 |
| Day 4 Predose | 85 | 23.32 | 23.724 | 16.22 | 3.4 | 120 | 81 | 3.93 | 3.866 | 3.04 | 0.1 | 27.7 |
| Day 4 4 h | 86 | 27.12 | 24.18 | 18.62 | 5.6 | 120 | 83 | 4.58 | 3.581 | 3.49 | 0.1 | 20.4 |
| Day 10-14 | 136 | 27.19 | 27.038 | 18.28 | 3 | 120 | 129 | 4.17 | 3.366 | 3.27 | 0.2 | 23.2 |
| Milvexian 200 mg QD | | | | | | | | | | | | |
| Baseline | 138 | 7.4 | 11.006 | 5.67 | 2.3 | 120 | | | | | | |
| Day 1 2 h | 135 | 32.2 | 34.996 | 18.89 | 3.8 | 120 | 129 | 5.35 | 5.569 | 3.51 | 0.6 | 23.5 |
| Day 1 4 h | 138 | 33.59 | 32.879 | 20.06 | 2.7 | 120 | 131 | 5.46 | 4.979 | 3.62 | 0.3 | 23 |
| Day 2 Predose | 141 | 21.62 | 22.082 | 15.02 | 1.7 | 120 | 134 | 3.57 | 3.119 | 2.75 | 0.1 | 20.7 |
| Day 4 Predose | 89 | 33.22 | 32.198 | 20.89 | 6.8 | 120 | 87 | 5.67 | 5.272 | 3.97 | 0.1 | 32.7 |
| Day 4 4 h | 87 | 49.34 | 38.655 | 31.22 | 9.2 | 120 | 86 | 8.49 | 6.215 | 5.76 | 0.1 | 28.4 |
| Day 10-14 | 139 | 38.94 | 34.87 | 24 | 4.9 | 120 | 133 | 6.54 | 5.634 | 4.25 | 0.3 | 27.7 |
| Milvexian 100 mg BID | | | | | | | | | | | | |
| Baseline | 139 | 7.8 | 14.189 | 5.44 | 2.7 | 120 | | | | | | |
| Day 1 2 h | 140 | 20.81 | 21.312 | 14.45 | 2.8 | 120 | 134 | 3.48 | 3.167 | 2.78 | 0.2 | 20.8 |
| Day 1 4 h | 136 | 23.44 | 24.082 | 15.52 | 4.4 | 120 | 130 | 3.79 | 3.234 | 2.93 | 0.4 | 20 |
| Day 2 Predose | 139 | 23.71 | 23.561 | 16.78 | 2.6 | 120 | 133 | 3.95 | 3.448 | 3.06 | 0.6 | 21.2 |
| Day 4 Predose | 87 | 35.22 | 32.86 | 22.89 | 5.2 | 120 | 82 | 6.04 | 5.312 | 4.33 | 1 | 27.3 |
| Day 4 4 h | 86 | 39.24 | 34.166 | 27.11 | 8.2 | 120 | 81 | 6.8 | 5.12 | 4.9 | 1.9 | 24.5 |
| Day 10-14 | 140 | 37.89 | 32.398 | 25.59 | 5.4 | 120 | 134 | 6.33 | 5.209 | 4.66 | 0.2 | 28.4 |
| Milvexian 200 mg BID | | | | | | | | | | | | |
| Baseline | 141 | 6.32 | 2.909 | 5.67 | 3 | 24.4 | | | | | | |
| Day 1 2 h | 143 | 26.62 | 27.822 | 17.67 | 1.7 | 120 | 136 | 4.27 | 4.091 | 3.22 | 0.3 | 23.5 |
| Day 1 4 h | 145 | 33.79 | 33.567 | 20.54 | 4.1 | 120 | 138 | 5.35 | 4.697 | 3.61 | 0.8 | 22.5 |
| Day 2 Predose | 141 | 34.22 | 31.4 | 22.78 | 7.7 | 120 | 134 | 5.5 | 4.445 | 3.93 | 1.4 | 28 |
| Day 4 Predose | 93 | 49.14 | 40.314 | 27.56 | 8.5 | 120 | 91 | 8.16 | 6.324 | 5.23 | 1.8 | 30 |
| Day 4 4 h | 91 | 48.99 | 37.974 | 31.11 | 8.1 | 120 | 87 | 8.45 | 6.116 | 5.91 | 1.2 | 31.3 |
| Day 10-14 | 140 | 51.76 | 38.828 | 34.17 | 1.7 | 120 | 136 | 8.48 | 6.137 | 6.49 | 0.3 | 30 |
| ENOXAPARIN | | | | | | | | | | | | |
| Baseline | 271 | 7.03 | 7.237 | 6.11 | 1.7 | 120 | | | | | | |
| Day 1 2 h | 143 | 10.81 | 18.976 | 7.02 | 1.6 | 120 | 130 | 1.94 | 3.592 | 1.27 | 0.1 | 29.2 |
| Day 1 4 h | 153 | 8.39 | 9.72 | 6.83 | 2 | 120 | 140 | 1.37 | 0.735 | 1.26 | 0.2 | 8.4 |
| Day 2 Predose | 172 | 6.98 | 9.15 | 5.7 | 2 | 120 | 156 | 1.18 | 1.131 | 1.05 | 0 | 14.4 |
| Day 4 Predose | 106 | 7.44 | 11.195 | 5.96 | 2.4 | 120 | 95 | 1.48 | 2.972 | 1.11 | 0.4 | 29.9 |
| Day 4 4 h | 105 | 8.16 | 4.554 | 7.44 | 2.4 | 40.2 | 95 | 1.46 | 0.555 | 1.36 | 0.7 | 4.2 |
| Day 10-14 | 270 | 11.18 | 13.136 | 8.11 | 3.3 | 120 | 253 | 1.7 | 1.943 | 1.31 | 0.4 | 25.7 |

Key: SD = standard deviation.
Note:
N for measured value is the number of subjects with a non-missing value for the parameter at the specified time point. N for ratio to from baseline is the number of subjects with non-missing values at both baseline and the postbaseline time point.

### Example 4. Bioavailability Study of 25 mg and 100 mg Film Coated DC Tablet Against 25 Mg and 100 mg Comparative SDP Oral Capsule in Healthy Participants and Results

The first Phase 1 trial is an open-label, randomized, crossover study to evaluate the relative oral bioavailability, pharmacokinetics, and food effect after single dose (for Part 1, Part 3, and Part 4) or multiple-dose (for Part 2). Part 1 of this first Phase 1 study is to evaluate the relative bioavailability and food effect of a single dose of 200 mg milvexian administered as film coated DC tablet compared with 100 mg comparative SDP oral capsule under fasting and fed conditions. Part 2 of this first Phase 1 study is to characterize the pharmacokinetic (PK) of multiple twice daily administered doses for 5 days of milvexian administered as 200 mg milvexian administered as film coated DC tablet and comparative SDP oral capsules at 25 mg or 200 mg. The 100 mg and 25 mg capsule formulations (see Table 12 below) are described in WO 2020210629 of which the capsule comprises MCC and lactose anhydrous DC in a weight ratio of 1:1 binder (MCC) to filler (lactose anhydrous). The composition and physical properties for the 25 mg and 100 mg film coated DC tablets are provided in the Table 1 above.

Blood samples were drawn at predetermined time points following drug administration as specified in the clinical study protocols. Concentration of the samples are measured using a validated analytical method (Liquid Chromatography with Tandem Mass Spectroscopy). Individual subject pharmacokinetic parameters (e.g. Cₘₐₓ, AUCₗₐₛₜ, and AUC_{∞}) are derived by non-compartmental methods using Phoenix™ WinNonlin® (version 8.1, Pharsight, A Certara™ Company, L.P., Princeton, NJ, USA) software from the time-concentration profiles.

TABLE 12
SDP Capsule Formulation Composition of 100 mg and 25 mg

| | 100 mg SDP | 25 mg SDP |
|---|---|---|
| Component | capsule | capsule |
| Intragranular | | |
| SDP of 75% Compound | 133.3 | 33.33 |
| (I): HPMCAS* | | |
| Microcrystalline cellulose | 167.7 | 41.925 |
| (MCC) | | |
| Lactose anhydrous DC | 167.7 | 41.925 |
| Weight ratio of MCC to lactose | 1:1 | 1:1 |
| anhydrous | | |
| Croscarmellose sodium | 12.5 | 3.125 |
| Colloidal silicon dioxide | 2.50 | 0.625 |
| Magnesium stearate | 1.25 | 0.3125 |
| Total Intragranular | 485 | 121 |
| Extragranular | | |
| Croscarmellose sodium | 12.5 | 3.125 |
| Magnesium stearate | 2.5 | 0.625 |
| Total | 500 | 125 |

*SDP prepared according to the composition and method described in WO 2020210629

The treatment regimens for Part 1 and Part 2 are summarized in Table 13 below.

TABLE 13
Treatment Regimen used in Part 1 and Part 2

| Treatment | Formulation | Dose | Food |
|---|---|---|---|
| A (Part 1A) | 100 mg film-coated DC* | 200 mg (2 × 100 mg) | Fasted |
| | oral tablet | | |
| C (Part 1A) | 100 mg SDP oral capsule | 200 mg (2 × 100 mg) | Fasted |
| D (Part 1B) | 100 mg film-coated DC* | 200 mg (2 × 100 mg) | Fed |
| | oral tablet | | |
| F (Part 1B) | 100 mg SDP oral capsule | 200 mg (2 × 100 mg) | Fed |
| G (Part 2A) | 100 mg film-coated DC* | 200 mg (2 × 100 mg) | BID for |
| | oral tablet | | 5 days |
| H (Part 2A) | 100 mg SDP oral capsule | 200 mg (2 × 100 mg) | BID for |
| | | | 5 days |
| I (Part 2B) | 25 mg film-coated DC* | 25 mg (1 × 25 mg) | BID for |
| | oral tablet | | 5 days |
| J (Part 2B) | 25 mg SDP oral capsule | 25 mg (1 × 25 mg) | BID for |
| | | | 5 days |

*DC: direct compression
**RC: roller compaction

The absolute Bioavailability for the 100 mg SDP capsule comparative formulation is 52% for fasted and 72% for fed conditions for a 200 mg.

In Part 1 of single dose administration regimen, 100 mg film-coated DC tablet shows about 9.0% to about 11% as compared to the oral SDP capsule at 200 mg dose.

100 mg film-coated DC* oral tablet has lower food effects. To achieve better patient compliance, it is preferred to have milveixan being administered with or without food. A drug formulation with small food effects provide better patient compliance.

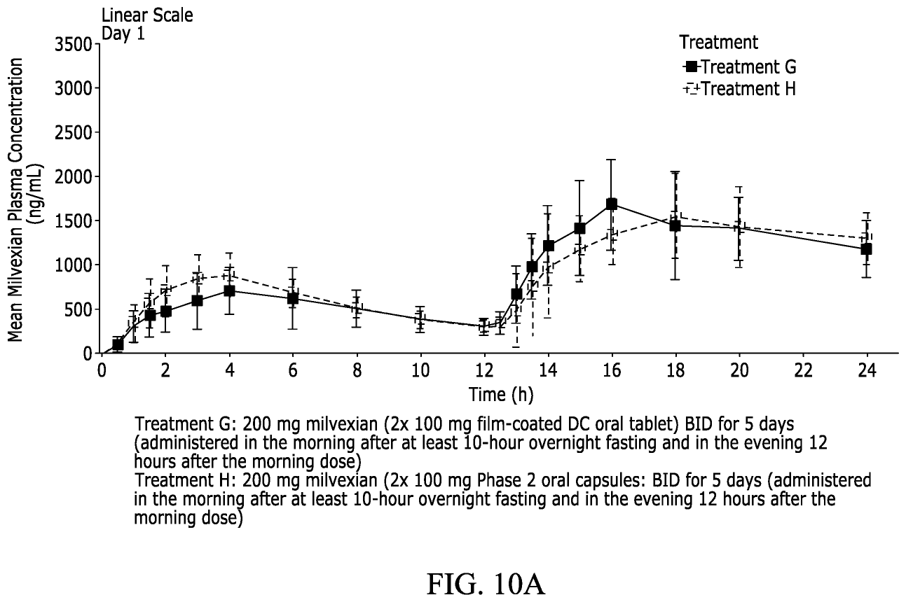

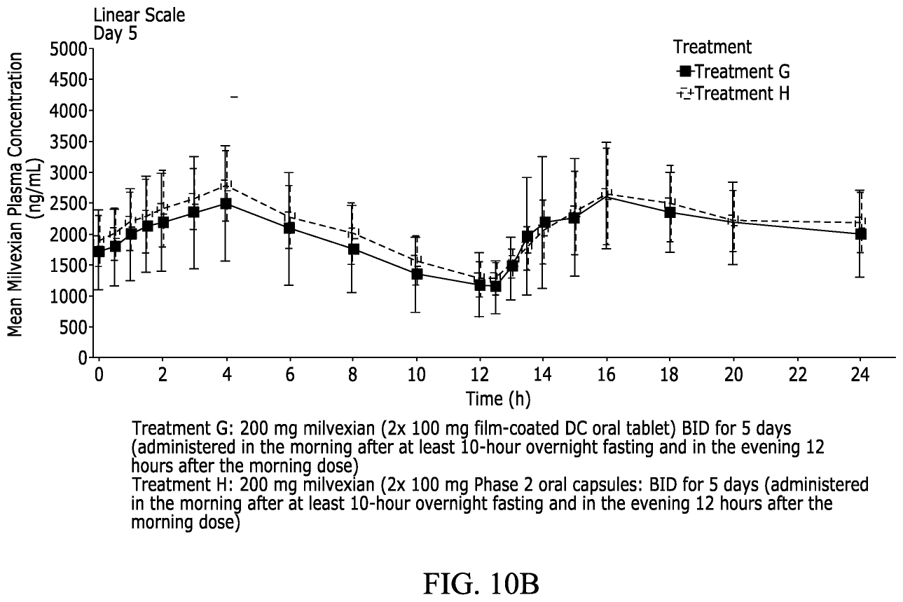

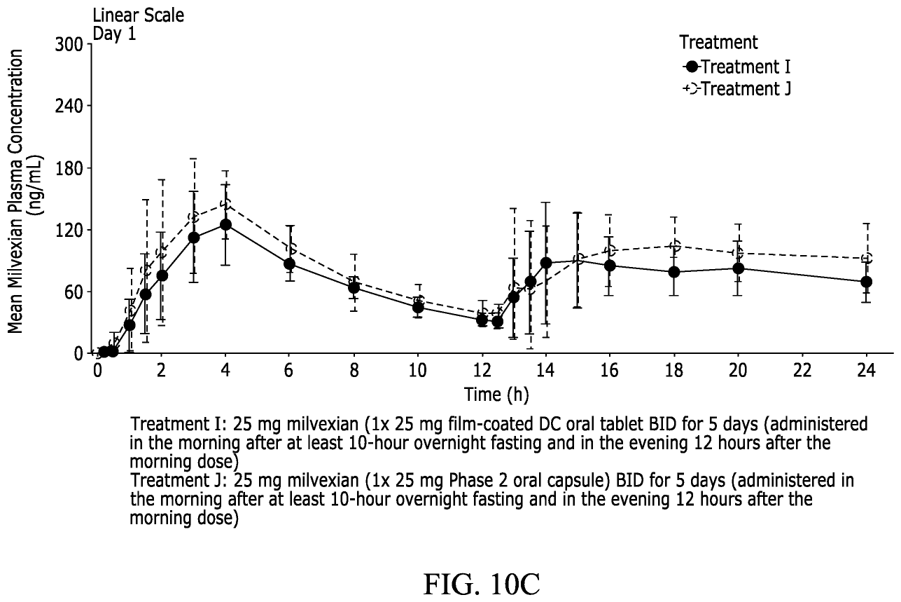

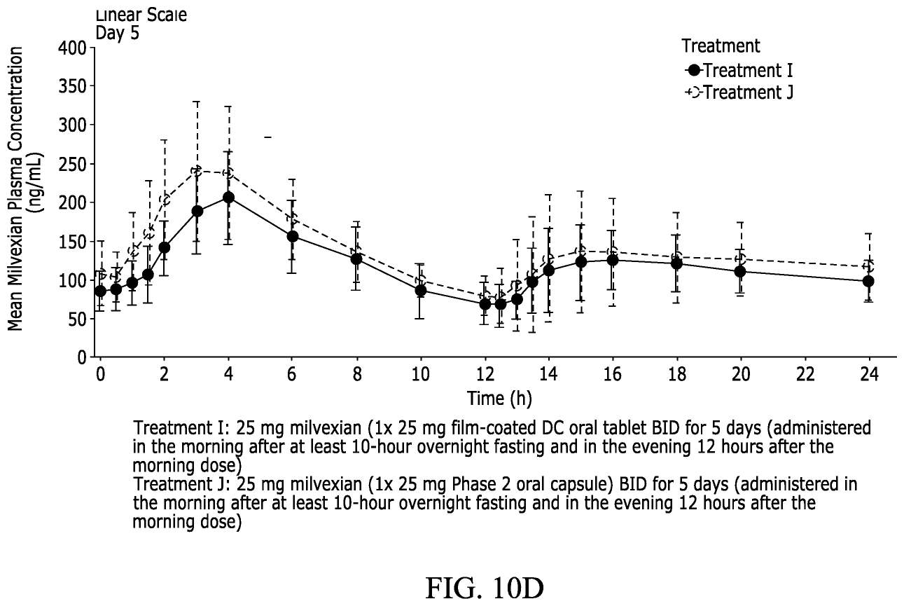

In Part 2 of multiple dose BID administration regimen, at 2×100 mg film-coated DC tablet shows about 5-7% lower bioavailability, as compared to the 2×100 mg SDP oral capsule. The 25 mg of the film-coated DC tablet (DC tablet) shows about 11-13% lower bioavailability, as compared to the 25 mg of SDP oral capsule) (See FIGS. 10A-10D for milvexian dosing curves as a function of time after BID administration).

In a second Phase 1 trial, Part 1 was an open-label, randomized, 3-way crossover study in healthy participants to evaluate the relative oral bioavailability, pharmacokinetics, and food effect of a single oral dose of 200 mg milvexian as 2×100 mg SDP DC tablet compared to 200 mg of 2×100 mg SDP granule capsule under fasting conditions and to assess the effect of food on the bioavailability of milvexian after a single dose of 200 mg milvexian as 2×100 mg SDP DC tablet. Part 2 was an open-label, randomized, 2-way crossover Study in healthy participants to evaluate the PK and relative bioavailability of a single oral dose of 50 mg milvexian as 2×25 mg SDP tablet compared to 50 mg milvexian as 2×25 mg SDP granule capsule in healthy participants under fasting conditions.

In the pooled analysis of PK (AUCinf and Cₘₐₓ) data generated under the two phase 1 trials under fed and fasting conditions for 2×100 mg and 1×25 mg SDP DC tablets and 25 mg and 100 mg SDP granule capsule formulations, the results demonstrated that the tablet exhibited smaller inter-participant PK variability in healthy participants than those of capsule

The results of clinical studies demonstrated that, for tablets with similar dissolution rates, AUC_{∞} (also known as AUCinf) of the 2×100 mg film-coated DC* oral tablet coated tablet relative to the 2×100 mg granule capsule formulations, met bioequivalence criteria.

### Example 5

Delayed cardiac repolarization is an undesirable side-effect of some non-antiarrhythmic drugs. Due to the potential clinical consequences of delayed cardiac repolarization, a rigorous characterization of a new pharmaceutical agent's ability to prolong QT/QTc interval was recommended.

Results from in vitro studies indicated that milvexian inhibited cardiac potassium (hERG/IKr) channel currents with weak to moderate potency at concentrations that greatly exceed unbound concentrations in the plasma of subjects treated with clinically relevant dose regimens of milvexian. The present study, which was conducted after the in vitro study results became available, evaluated the effect of milvexian on cardiac repolarization in healthy subjects, an important aspect of cardiovascular safety.

This placebo- and positive-controlled, TQT study was conducted to evaluate the effect of multiple-dose administration of milvexian on the QT/QTc interval duration and ECG morphology at therapeutic and supratherapeutic doses at steady state, in healthy adults.

| | |
|---|---|
| Objectives | Endpoints |

Primary

| | |
|---|---|
| To assess the effects of milvexian on the | QTcF, change from baseline in QTcF |
| QT/QTcF intervals in healthy participants | (ΔQTcF), placebo-corrected change from |
| when administered at therapeutic (100 mg | baseline in QTcF (ΔΔQTcF). |
| twice daily as capsule) and supratherapeutic | |
| (200 mg twice daily as solution) doses for 4 | |
| days. | |

Secondary

| | |
|---|---|
| To assess the effects of milvexian on other | HR, ΔHR, ΔΔHR, PR, ΔPR, ΔΔPR, QRS |
| ECG parameters and ECG intervals in | duration, ΔQRS duration, and ΔΔQRS |
| healthy participants when administered at | duration. |
| therapeutic (100 mg twice daily as capsule) | |
| and supratherapeutic (200 mg twice daily as | |
| solution) doses for 4 days. | |
| To assess the assay sensitivity by evaluating | QTc, baseline QTc, change from baseline in |
| the mean ΔQTc between moxifloxacin and | QTc (ΔQTc), placebo-corrected change |
| placebo (ΔΔQTc) on Day 4. | from baseline in QTc (ΔΔQTc) for QTcB |
| | and QTcP. |
| To assess the relationship between the | Characterization of the milvexian |
| systemic plasma concentrations of | concentration - ΔΔQTcF relationship. |
| milvexian and QT/QTc changes. | |

The upper bound of two-sided 90% CI for the largest difference in mean change from baseline in QTc intervals (ΔQTc; based on the primary correction method-QTcF) between milvexian and placebo (ΔΔQTc) at the therapeutic (100 mg twice daily as capsule) and supratherapeutic (200 mg twice daily as solution) doses was less than 10 milliseconds.

A total of 66 participants were enrolled in the study and randomly assigned to 1 of 4 possible treatment sequences. All 66 (100.0%) participants enrolled in the study were included in the safety and PD analysis set: 55 participants each in the milvexian 100 mg and 200 mg groups, 58 participants in the moxifloxacin group, and 57 participants in the placebo group.

Pharmacokinetic analysis was performed on the plasma concentrations of each participant. Sixty-one participants were included in the PK analysis set, i.e., all randomized participants who received at least one dose of active study intervention whose PK profiles allowed for accurate calculation of at least one PK parameter. The PK/PD analysis was based on the participants included in the PD analysis set who had at least one measurement of milvexian concentration.

All 66 (100.0%) participants enrolled in the study received at least one entire dose of study intervention.

Forty-five (68.2%) participants completed all 4 periods with study intervention as planned. Per study intervention, 52 participants received milvexian 100 mg capsule twice daily for 4 days, 54 participants received milvexian 200 mg solution twice daily for 4 days, 58 participants received a single dose of 400 mg moxifloxacin, and 56 participants received placebo for 4 days.

There were no consistent or clinically relevant changes over time in mean vital signs.

No treatment-emergent actual values of QTcF or QTcB >480 ms or changes from baseline >60 ms were observed during the study. No ECG abnormalities were reported as TEAE in the study.

Milvexian exposure (i.e., Cₘₐₓ and AUC over a dosing interval) was approximately 2— to 3-times higher on Day 4, compared with Day 1, when 100 mg was administered twice daily as a capsule (Treatment A) and 200 mg was administered twice daily as a solution (Treatment B).

### Pharmacodynamic Results:

On administration of 100 and 200 mg milvexian, the upper limits of the 2-sided 90% CIs for ΔΔQTcF over the Day 1 and Day 4 postdose measurement intervals were below the protocol specified 10-ms limit at all timepoints (i.e., highest upper limits of 5.16 ms and 4.57 ms for 100 mg and 200 mg milvexian, respectively), showing there was no effect of clinical or regulatory concern on the QT interval in accordance with the ICH E14 guideline. These results were confirmed based on the ΔΔQTcF at milvexian Tₘₐₓ, i.e., the upper limits of the 90% CI were below 10 ms at the Tₘₐₓ of milvexian after administration of 100 mg or 200 mg twice daily, from analyses that used milvexian Tₘₐₓ on Day 1 and Day 4 (combined) or milvexian Tₘₐₓ on Day 1 and Day 4 separately.

Study-specific and Bazett's correction methods also showed there was no effect of clinical or regulatory concern on the QT interval in accordance with the ICH E14 guideline. Assay sensitivity for moxifloxacin was also demonstrated when using Bazett's or the study-specific power correction methods for HR.

No treatment-emergent actual values of QTc >480 ms were observed during the study. No treatment-emergent changes from baseline of QTcF or QTcP >60 ms were observed during the study. One (1.7%) participant had a treatment-emergent change from baseline of QTcB >60 ms during the study (i.e., following moxifloxacin).

No consistent or clinically relevant changes over time were observed in HR, RR interval, PR interval, or QRS width. No clinically meaningful differences were observed between treatment groups

### Pharmacokinetics/Pharmacodynamics Results:

Based on the results of linear mixed effects modeling, no statistical significant relationship was observed between milvexian concentration and ΔΔQTcF (p=0.8454), ΔΔQTcP (p=0.7102), or ΔΔQTcB (p=0.8670). At Cₘₐₓ, no statistically significant effect of treatment (100 or 200 mg milvexian) on ΔΔQTc was observed (p>0.5).

### Conclusions:

No notable study limitations were identified by the sponsor.

Administration of multiple doses of 100 and 200 mg milvexian was generally safe and well tolerated in healthy adult participants.

The upper limits of all 2-sided 90% CIs of the time-matched differences between 100 mg milvexian and placebo and between 200 mg milvexian and placebo in changes from baseline in QTcF were below 10 ms. Hence, milvexian at therapeutic (100 mg) and supratherapeutic (200 mg) doses does not show evidence of QT/QTc interval prolongation of clinical or regulatory concern, according to the ICH E14 guideline.

Assay sensitivity was demonstrated by moxifloxacin as the positive control.

Based on the results of linear mixed effects modeling, no statistical significant relationship was observed between milvexian concentration and ΔΔQTcF (p=0.8454).

1. A method of treating or preventing a thrombotic condition in a human patient with a cardiovascular or cerebrovascular disease, wherein the method comprises administering to the human patient an immediate release tablet comprising 25 mg, 50 mg, or 100 mg of milvexian (or a pharmaceutically acceptable salt or solvate thereof), and a pharmaceutically acceptable excipient, optionally the immediate release tablet is administered together with an antiplatelet therapy, wherein the immediate release tablet is administered twice daily.

2. The method of claim 1, wherein administration of the immediate release tablet or a regimen comprising the immediate release tablet reduces factor XI (FXI) clotting activity by about 7% to about 20% relative to baseline, or by about 27% to 64% relative to baseline FXI clotting activity of the patient.

3. The method of claim 1, wherein administration of the immediate release tablet or a regimen comprising the immediate release tablet prolongates activated partial thromboplastin time (aPTT) ranging from about 27% to about 64% relative to baseline, or results in a prolongation of activated partial thromboplastin time (aPTT) in the patient with a ratio to baseline of 2.1 to 2.6.

4. The method of claim 1, wherein the administration of the immediate release tablet does not result in a statistically significant increase in major bleeding complications.

5. The method of claim 1, wherein the immediate release tablet has a disintegration time in water of less than 20 seconds.

6. The method of claim 1, wherein the administration results in a milvexian plasma half-life ranging from about 13 hours to about 16 hours.

7. The method of claim 1, wherein the administration results in a milvexian plasma concentration reaching steady-state at about 3 days to 6 days.

8. The method of claim 1, wherein the immediate release tablet is administered without regard to the timing of food intake.

9. The method of claim 1, wherein the thrombotic condition is a thromboembolic disorder selected from an arterial thromboembolic disorder; a venous thromboembolic disorder; or a thromboembolic disorder in the chambers of the heart or in the peripheral circulation.

10. The method of claim 1, wherein the human patient has a cerebrovascular disease.

11. The method of claim 10, wherein the cerebrovascular disease is selected from non-cardioembolic ischemic stroke or transient ischemic attack (TIA).

12. The method of claim 1, wherein the human patient has a cardiovascular disease.

13. The method of claim 12, wherein the cardiovascular disease is atrial fibrillation or flutter.

14. The method of claim 12, wherein the cardiovascular disease is acute coronary syndrome.

15. The method of claim 1, wherein the immediate release tablet comprises 50 mg or 100 mg of milvexian (or a pharmaceutically acceptable salt or solvate thereof).

16. The method of claim 9, wherein the thrombotic condition comprises arterial thromboembolic disorder associated with an acute coronary syndrome.

17. The method of claim 1, wherein the method comprises administering to the human patient a regimen comprising: (i) an immediate release tablet comprising 25 mg of milvexian (or pharmaceutically acceptable salt or solvate thereof); and (ii) an antiplatelet therapy selected from the group consisting of aspirin, a P2Y12 inhibitor, and combination thereof; wherein the immediate release tablet is administered twice daily.

18. The method of claim 17, wherein the antiplatelet therapy is a P2Y12 inhibitor.

19. The method of claim 17, wherein the antiplatelet therapy is aspirin.

20. The method of claim 1, wherein the milvexian administration results in no clinically significant corrected QT (QTc) interval prolongation.
